(19) United States
(12) Patent Application Publication
(10) Pub. No.: US 2022/0323497 A1
(43) Pub. Date: Oct. 13, 2022

(54) CELLS EXPRESSING CHIMERIC ANTIGEN RECEPTORS AND CHIMERIC STIMULATING RECEPTORS AND USES THEREOF

(71) Applicant: Eureka Therapeutics, Inc., Emeryville, CA (US)

(72) Inventors: Hongruo YUN, Emeryville, CA (US); Jun Cui, Emeryville, CA (US); Pengbo Zhang, Emeryville, CA (US); Yiyang Xu, Emeryville, CA (US); Lucas Horan, Emeryville, CA (US); Shaohua Xu, Emeryville, CA (US); Guangyan Xiong, Emeryville, CA (US); Shan Li, Emeryville, CA (US); Yixiang Xu, Emeryville, CA (US); Bradley Austin Heller, Emeryville, CA (US); Hongbing Zhang, Emeryville, CA (US); Hong Liu, Emeryville, CA (US)

(21) Appl. No.: 17/628,120

(22) PCT Filed: Jul. 24, 2020
(86) PCT No.: PCT/US2020/043629

§ 371 Date: Jan. 18, 2022

Related U.S. Application Data
(60) Provisional application No. 62/878,271, filed on Jul. 24, 2019.
(60) Provisional application No. 62/879,629, filed on Jul. 29, 2019.
(60) Provisional application No. 62/953,758, filed on Dec. 26, 2019.

(51) Int. Cl. A61K 35/17; C12N 5/0783; C12N 15/62; C12N 15/86; C07K 14/725; C07K 14/705; A61P 35/00

(52) U.S. Cl. CPC A61K 35/17; C12N 5/0636; C12N 15/625; C12N 15/86; C07K 14/7051; C07K 14/70578; A61P 35/00; C12N 2740/15042; C12N 2501/2302; C07K 2319/03; C07K 2319/02

(57) ABSTRACT

Described herein are immune cells comprising: a chimeric antigen receptor (CAR) that comprises (i) an extracellular target-binding domain comprising an antibody moiety; (ii) a transmembrane domain; and (iii) a primary signaling domain, and a chimeric stimulating receptor (CSR) that comprises (i) a ligand-binding module that is capable of binding or interacting with a target ligand; (ii) a transmembrane domain; and (iii) a CD30 costimulatory domain, in which the CSR in the immune cells lacks a functional primary signaling domain. Also provided herein are methods of using the same or compositions thereof for therapeutic treatment of cancers (e.g., hematological cancers or solid tumor cancers).

### CROSS-REFERENCES TO RELATED APPLICATIONS

This application claims priority to U.S. Provisional Application No. 62/878,271, filed Jul. 24, 2019, U.S. Provisional Application No. 62/879,629, filed Jul. 29, 2019, and U.S. Provisional Application No. 62/953,758, filed Dec. 26, 2019, the disclosures of which are hereby incorporated by reference in their entirety for all purposes.

### BACKGROUND

Adoptive T cell immunotherapy, in which a patient's own T lymphocytes are engineered to express chimeric antigen receptors (CARs), has shown great promise in treating hematological malignancies, but not so much in solid tumors. In addition, CAR by itself is generally not efficacious enough, especially for solid tumors, even with the commonly used costimulatory fragments such as CD28 or 4-1BB, no matter if expressed in cis or in trans. Therefore, more efficacious and longer-lasting T cell immunotherapies are needed.

CD30 is a member of the TNF receptor superfamily of receptor proteins. Most of the homology between TNF receptor family members occurs in the extracellular domain, with little homology in the cytoplasmic domain. This suggested that different members of the TNF receptor family might utilize distinct signaling pathways. Consistent with this hypothesis, the TNF receptor type 1 and Fas have been shown to interact with a set of intracellular signaling molecules through a 65-amino acid domain termed a death domain, whereas the TNF receptor type 2 and CD40 have been found to associate with members of the tumor necrosis factor receptor-associated factor (TRAF) family of signal transducing molecules.

The membrane bound form of CD30 is a 120-kDa, 595-amino acid glycoprotein with a 188-amino acid cytoplasmic domain. Cross-linking of CD30 with either antibodies or with CD30 ligand produces a variety of effects in cells, including augmenting the proliferation of primary T cells following T cell receptor engagement and induction of the NF-kB transcription factor. CD30 was originally identified as an antigen expressed on the surface of Hodgkin's lymphoma cells. Subsequently, CD30 was shown to be expressed by lymphocytes with an activated phenotype, cells on the periphery of germinal centers, and CD45RO1 (memory) T cells. CD30 may also play a role in the development of T helper 2 type cells. The T cell activation properties of the TNF receptor family member 4-1BB have been shown to involve the specific ability of its cytoplasmic domain to associate with the tyrosine kinase p56lck. The sequence of the cytoplasmic domain of CD30 shows little sequence similarity to any of these receptors; CD30 lacks an obvious death domain or a p56lck-binding site.

### SUMMARY

The present invention provides, among other things, chimeric stimulating receptors (CSRs) that use a costimulatory domain from CD30 (also referred to herein as a CD30 costimulatory domain). As described in detail herein, T cells with CSRs containing a costimulatory domain from CD30 express far less PD-1, an inhibitor of T cell activation, than T cells with CSRs containing a costimulatory domain from, e.g., CD28 or 4-1BB, and at the same time demonstrate equal cytotoxic potential. In some embodiments, T cells with CSRs containing a costimulatory domain from CD30 express far less PD-1 than T cells with CSRs containing a costimulatory domain from Dap10. The examples suggest that the costimulatory domain from CD30 ameliorates the functional unresponsiveness that leads to T cell exhaustion, also called anergy, and subsequently, provides superior persistence of tumor cell killing and increased tumor infiltration as compared to the commonly used costimulatory domains such as CD28. It is unexpected since CD30 lacks a p56lck-binding site that is thought to be crucial for CSR costimulation.

In one aspect, the disclosure features an immune cell comprising: (a) a chimeric antigen receptor (CAR) comprising: (i) an extracellular target-binding domain comprising an antibody moiety (a CAR antibody moiety); (ii) a transmembrane domain (a CAR transmembrane domain); and (iii) a primary signaling domain, and (b) a chimeric stimulating receptor (CSR) comprising: (i) a ligand-binding module that is capable of binding or interacting with a target ligand; (ii) a transmembrane domain (a CSR transmembrane domain); and (iii) a CD30 costimulatory domain, wherein the CSR lacks a functional primary signaling domain.

In some embodiments, the CD30 costimulatory domain comprises a sequence that can bind to an intracellular TRAF signaling protein. In some embodiments, the sequence that can bind to an intracellular TRAF signaling protein corresponds to residues 561-573 or 578-586 of a full-length CD30 having the sequence of SEQ ID NO:65. In some embodiments, the CD30 costimulatory domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to residues 561-573 or 578-586 of SEQ ID NO:65. In some embodiments, the CD30 costimulatory domain comprises a sequence that is at least 70%, 75%, 80%, 85%, 90%, 95%, or 100% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:75.

In some embodiments of this aspect, the CSR comprises more than one CD30 costimulatory domain. In some embodiments, the CSR further comprises at least one costimulatory domain which comprises the intracellular sequence of a costimulatory molecule that is different from CD30. The costimulatory molecule that is different from CD30 can be selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, a ligand that specifically binds with CD83, and Dap10.

In some embodiments, the CAR further comprises a costimulatory domain (a CAR costimulatory domain). The CAR costimulatory domain can be derived from the intracellular domain of a costimulatory receptor. The costimulatory receptor can be selected from the group consisting of CD30, CD27, CD28, 4-1BB (CD137), OX40, CD40, PD-1, ICOS, lymphocyte function-associated antigen-1 (LFA-1), CD2, CD7, LIGHT, NKG2C, B7-H3, a ligand that specifically binds with CD83, and Dap10.

In some embodiments, the ligand-binding module of the CSR is derived from the extracellular domain of a receptor. In some embodiments, the ligand-binding module of the CSR comprises an antibody moiety (a CSR antibody moiety). The CSR antibody moiety can be a single chain antibody fragment. The CAR antibody moiety can be a single chain antibody fragment. In some embodiments, the CAR antibody moiety and/or the CSR antibody moiety is a single chain Fv (scFv), a single chain Fab, a single chain Fab′, a single domain antibody fragment, a single domain multispecific antibody, an intrabody, a nanobody, or a single chain immunokine. In some embodiments, the CAR antibody moiety and/or the CSR antibody moiety is a single domain multispecific antibody. In some embodiments, the single domain multispecific antibody is a single domain bispecific antibody. In some embodiments, the CAR antibody moiety and/or the CSR antibody moiety is a single chain Fv (scFv). In some embodiments, the scFv is a tandem scFv.

In some embodiments, the CAR antibody moiety and/or the CSR antibody moiety specifically binds to a disease-related antigen. The disease-related antigen is a cancer-related antigen. The disease-related antigen is a virus-related antigen. In some embodiments, the CAR antibody moiety and/or the CSR antibody moiety specifically binds to a cell surface antigen. The cell surface antigen can be selected from the group consisting of protein, carbohydrate, and lipid. The cell surface antigen can be CD19, CD20, CD22, CD47, CD158e, GPC3, ROR1, ROR2, BCMA, GPRC5D, FcRL5, MUC16, MCT4, PSMA, or a variant or mutant thereof.

In some embodiments, the CAR antibody moiety and the CSR antibody moiety specifically bind to the same antigen. In particular embodiments, the CAR antibody moiety and the CSR antibody moiety specifically bind to different epitopes on the same antigen.

In some embodiments, the CAR antibody moiety and/or the CSR antibody moiety specifically binds to a MHC-restricted antigen. In some embodiments, the MHC-restricted antigen is a complex comprising a peptide and an MHC protein, and the peptide is derived from a protein selected from the group consisting of WT-1, AFP, HPV16-E7, NY-ESO-1, PRAME, EBV-LMP2A, HIV-1, KRAS, FoxP3, Histone H3.3, PSA, and a variant or mutant thereof.

In some embodiments of this aspect, the CAR antibody moiety binds to CD19, and the ligand-binding module of the CSR binds to CD19. In some embodiments, the CAR antibody moiety binds to CD22, and the ligand-binding module of the CSR binds to CD22. In some embodiments, the CAR antibody moiety binds to CD20, and the ligand-binding module of the CSR binds to CD20. In some embodiments, the CAR antibody moiety binds to CD19, and the ligand-binding module of the CSR binds to CD22. In some embodiments, the CAR antibody moiety binds to CD19, and the ligand-binding module of the CSR binds to CD20. In some embodiments, the CAR antibody moiety binds to CD22, and the ligand-binding module of the CSR binds to CD20. In some embodiments, the CAR antibody moiety binds to CD22, and the ligand-binding module of the CSR binds to CD19. In some embodiments, the CAR antibody moiety binds to CD20, and the ligand-binding module of the CSR binds to CD19. In some embodiments, the CAR antibody moiety binds to CD20, and the ligand-binding module of the CSR binds to CD22. In some embodiments, the CAR antibody moiety and/or the ligand-binding module of the CSR binds to both CD19 and CD22. In some embodiments, the CAR antibody moiety and/or the ligand-binding module of the CSR binds to both CD19 and CD20. In some embodiments, the CAR antibody moiety and/or the ligand-binding module of the CSR binds to both CD20 and CD22. In some embodiments, the CAR antibody moiety and/or the ligand-binding module of the CSR binds to CD19, CD20, and CD22.

In some embodiments of this aspect, the CAR antibody moiety specifically binds to a complex comprising an alpha-fetoprotein (AFP) peptide and an MHC class I protein. In some embodiments, the ligand-binding module of the CSR specifically binds to glypican 3 (GPC3). In some embodiments, the CAR antibody moiety binds to a complex comprising an AFP peptide and an MHC class I protein, and the ligand-binding module of the CSR binds to GPC3.

In some embodiments, both the CAR antibody moiety and the ligand-binding module of the CSR bind to GPC3. In particular embodiments, the CAR antibody moiety and the ligand-binding module of the CSR specifically bind to different epitopes on GPC3.

In some embodiments, the CAR transmembrane domain is the transmembrane domain of CD30. In some embodiments, the CAR transmembrane domain is the transmembrane domain of CD8. In some embodiments, the CAR transmembrane domain and/or the CSR transmembrane domain is derived from the transmembrane domain of a TCR co-receptor or a T cell co-stimulatory molecule. The TCR co-receptor or T cell co-stimulatory molecule can be selected from the group consisting of CD8, 4-1BB, CD27, CD28, CD30, OX40, CD3E, CD3ζ, CD45, CD4, CD5, CD9, CD16, CD22, CD33, CD37, CD64, CD80, CD86, CD134, CD137, CD154, and Dap10. In certain embodiments, the TCR co-receptor or T cell co-stimulatory molecule is CD30 or CD8. In some embodiments, the T cell co-stimulatory molecule can be CD30. In some embodiments, the TCR co-receptor is CD8.

In some embodiments, the CAR transmembrane domain and/or the CSR transmembrane domain is the transmembrane domain of CD8, 4-1BB, CD27, CD28, CD30, OX40, CD3E, CD3ζ, CD45, CD4, CD5, CD9, CD16, CD22, CD33, CD37, CD64, CD80, CD86, CD134, CD137, CD154, or Dap10. In certain embodiments, the CAR transmembrane domain and/or the CSR transmembrane domain is the transmembrane domain of CD30 or CD8. In certain embodiments, the CAR transmembrane domain and/or the CSR transmembrane domain is the transmembrane domain of CD30. In certain embodiments, the CSR transmembrane domain is the transmembrane domain of CD30. In certain embodiments, the CAR transmembrane domain and/or the CSR transmembrane domain is the transmembrane domain of CD8. In certain embodiments, the CAR transmembrane domain and/or the CSR transmembrane domain comprises an amino acid sequence selected from the group consisting of SEQ ID NOS:66-71.

In some embodiments of this aspect, the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d. In some embodiments, the primary signaling domain comprises a sequence derived from the intracellular signaling sequence of CD3ζ. In some embodiments, the primary signaling domain comprises the intracellular signaling sequence of CD3ζ. In certain embodiments, the primary signaling domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:77.

In some embodiments of this aspect, the CAR in the immune cell further comprises a peptide linker between the extracellular target-binding domain and the transmembrane domain of the CAR. In some embodiments, the CAR in the immune cell further comprises a peptide linker between the transmembrane domain and the costimulatory domain of the CAR. In some embodiments, the CAR in the immune cell further comprises a peptide linker between the costimulatory domain and the primary signaling domain of the CAR. In some embodiments, the CSR in the immune cell further comprises a peptide linker between the ligand-binding module and the transmembrane domain of the CSR. In some embodiments, the CSR in the immune cell further comprises a peptide linker between the transmembrane domain and the CD30 costimulatory domain of the CSR.

In some embodiments of this aspect, the expression of the CSR is inducible. In some embodiments, the expression of the CSR is inducible upon activation of the immune cell. In some embodiments, the immune cell is selected from the group consisting of a cytotoxic T cell, a helper T cell, a natural killer T cell, and a suppressor T cell.

In another aspect, the disclosure features one or more nucleic acids encoding the CAR and CSR comprised by the immune cell described herein, wherein the CAR and CSR each consist of one or more polypeptide chains encoded by the one or more nucleic acids.

In another aspect, the disclosure features one or more vectors comprising the one or more nucleic acids described above.

In another aspect, the disclosure features a pharmaceutical composition comprising: (a) the immune cell described herein, the nucleic acid(s) described herein, or the vector(s) described herein, and (b) a pharmaceutically acceptable carrier or diluent.

In another aspect, the disclosure features a method of killing target cells, comprising: contacting one or more target cells with the immune cell described herein under conditions and for a time sufficient so that the immune cells mediate killing of the target cells, wherein the target cells express an antigen specific to the immune cell, and wherein the immune cell expresses a low cell exhaustion level upon contacting the target cells. In some embodiments, the immune cell expresses a low cell exhaustion level of an exhaustion marker selected from the group consisting of PD-1, TIM-3, TIGIT, and LAG-3. In certain embodiments, the immune cell is a T cell. In certain embodiments, the immune cell expresses a low cell exhaustion level of PD-1. In some embodiments, the ratio of PD-1 from immune cells (e.g., CD8⁺ T cells, CD4⁺ T cells) expressing a 1^{st} generation CAR (e.g., αAFP-CD8T-z-CAR) and CD30-CSR to PD-1 from immune cells expressing the 1^{st} generation CAR alone is between 0.05 and 0.5 (e.g., between 0.05 and 0.45, between 0.05 and 0.4, between 0.05 and 0.35, between 0.05 and 0.3, between 0.05 and 0.25, between 0.05 and 0.2, between 0.05 and 0.15, between 0.05 and 0.1, between 0.1 and 0.45, between 0.15 and 0.45, between 0.2 and 0.45, between 0.25 and 0.45, between 0.3 and 0.45, between 0.35 and 0.45, or between 0.4 and 0.45). In some embodiments, the ratio of PD-1 from immune cells (e.g., CD8⁺ T cells, CD4⁺ T cells) expressing a 2nd generation CAR (e.g., aAFP-CD28z-CAR) and CD30-CSR to PD-1 from immune cells expressing the 2nd generation CAR alone is between 0.05 and 0.5 (e.g., between 0.05 and 0.45, between 0.05 and 0.4, between 0.05 and 0.35, between 0.05 and 0.3, between 0.05 and 0.25, between 0.05 and 0.2, between 0.05 and 0.15, between 0.05 and 0.1, between 0.1 and 0.45, between 0.15 and 0.45, between 0.2 and 0.45, between 0.25 and 0.45, between 0.3 and 0.45, between 0.35 and 0.45, or between 0.4 and 0.45). In certain embodiments, the immune cell expresses a low cell exhaustion level of TIM-3. In certain embodiments, the immune cell expresses a low cell exhaustion level of TIGIT. In certain embodiments, the immune cell expresses a low cell exhaustion level of LAG-3. In some embodiments, the ratio of LAG-3 from immune cells (e.g., CD8⁺ T cells, CD4⁺ T cells) expressing a 2nd generation CAR (e.g., αAFP-CD28z-CAR) and CD30-CSR to LAG-3 from immune cells expressing the 2nd generation CAR alone is between 0.1 and 0.9 (e.g., between 0.1 and 0.8, between 0.1 and 0.7, between 0.1 and 0.6, between 0.1 and 0.5, between 0.1 and 0.4, between 0.1 and 0.3, between 0.1 and 0.2, between 0.2 and 0.9, between 0.3 and 0.9, between 0.4 and 0.9, between 0.5 and 0.9, between 0.6 and 0.9, between 0.7 and 0.9, or between 0.8 and 0.9).

In some embodiments, the immune cell expresses a lower level of PD-1, TIM-3, TIGIT, or LAG-3 than corresponding immune cell expressing a CSR comprising a CD28 costimulatory domain. In some embodiments, the immune cell expresses a lower level of PD-1 than the corresponding CD28 CSR immune cell, and wherein the ratio of PD-1 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of TIM-3 than the corresponding CD28 CSR immune cell, and wherein the ratio of TIM-3 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of LAG-3 than the corresponding CD28 CSR immune cell, and wherein the ratio of LAG-3 expression level of the immune cell to the corresponding CD28 CSR immune cell is 0.9, 0.8, 0.7, 0.6, 0.5, 0.4, 0.3, 0.2, 0.1 or lower. In some embodiments, the immune cell expresses a lower level of TIGIT than the corresponding CD28 CSR immune cell, and wherein the ratio of TIGIT expression level of the immune cell to the corresponding CD28 CSR immune cell is 0.9, 0.8, 0.7, 0.6, 0.5, 0.4, 0.3, 0.2, 0.1 or lower.

In some embodiments, the immune cell expresses a lower level of PD-1, TIM-3, TIGIT, or LAG-3 than corresponding immune cell expressing a CSR comprising a 4-1BB costimulatory domain. In some embodiments, the immune cell expresses a lower level of PD-1 than the corresponding 4-1BB CSR immune cell, and wherein the ratio of PD-1 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of TIM-3 than the corresponding 4-1BB CSR immune cell, and wherein the ratio of TIM-3 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of LAG-3 than the corresponding 4-1BB CSR immune cell, and wherein the ratio of LAG-3 expression level of the immune cell to the corresponding 4-1BB CSR immune cell is 0.9, 0.8, 0.7, 0.6, 0.5, 0.4, 0.3, 0.2, 0.1 or lower. In some embodiments, the immune cell expresses a lower level of TIGIT than the corresponding 4-1BB CSR immune cell, and wherein the ratio of TIGIT expression level of the immune cell to the corresponding 4-1BB CSR immune cell is 0.9, 0.8, 0.7, 0.6, 0.5, 0.4, 0.3, 0.2, 0.1 or lower.

In some embodiments, the immune cell expresses a lower level of PD-1, TIM-3, TIGIT, or LAG-3 than corresponding immune cell expressing a CSR comprising a Dap10 costimulatory domain. In some embodiments, the immune cell expresses a lower level of PD-1 than the corresponding Dap10 CSR immune cell, and wherein the ratio of PD-1 expression level of the immune cell to the corresponding Dap10 CSR immune cell is 0.9, 0.8, 0.7, 0.6, 0.5, 0.4, 0.3, 0.2, 0.1 or lower. In some embodiments, the immune cell expresses a lower level of TIM-3 than the corresponding Dap10 CSR immune cell, and wherein the ratio of TIM-3 expression level of the immune cell to the corresponding Dap10 CSR immune cell is 0.9, 0.8, 0.7, 0.6, 0.5, 0.4, 0.3, 0.2, 0.1 or lower. In some embodiments, the immune cell expresses a lower level of LAG-3 than the corresponding Dap10 CSR immune cell, and wherein the ratio of LAG-3 expression level of the immune cell to the corresponding Dap10 CSR immune cell is 0.9, 0.8, 0.7, 0.6, 0.5, 0.4, 0.3, 0.2, 0.1 or lower. In some embodiments, the immune cell expresses a lower level of TIGIT than the corresponding Dap10 CSR immune cell, and wherein the ratio of TIGIT expression level of the immune cell to the corresponding Dap10 CSR immune cell is 0.9, 0.8, 0.7, 0.6, 0.5, 0.4, 0.3, 0.2, 0.1 or lower.

In some embodiments of this aspect, the target cells are cancer cells. The cancer cells can be from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer. The cancer cells can be hematological cancer cells. The cancer cells can be solid tumor cells.

In some embodiments, the target cells are virus-infected cells. The virus-infected cells can be from a viral infection caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).

In another aspect, the disclosure features a method of treating a disease, the method comprising a step of administering to a subject the immune cell described herein, the nucleic acid(s) described herein, or the vector(s) described herein, or the pharmaceutical composition described herein to the subject. In some embodiments, the disease is a viral infection. In some embodiments, the disease is cancer. The cancer can be a hematological cancer. The cancer can be a solid tumor cancer.

In some embodiments, the subject has a higher density of the immune cell described herein in the solid tumor cancer than in the rest of the subject's body.

In some embodiments, the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.

In another aspect, the disclosure features a method for preventing and/or reversing T cell exhaustion in a subject, comprising administering to the subject the nucleic acid(s) described herein, the vector(s) described herein, or the pharmaceutical composition described herein comprising the nucleic acid(s) or the vector(s) to the subject. In some embodiments, the method decreases the expression of an exhaustion marker in a T cell. The exhaustion marker can be selected from the group consisting of PD-1, TIM-3, TIGIT, and LAG-3.

In another aspect, the disclosure features a method of treating a solid tumor cancer in a subject with increased tumor infiltration or immune cell expansion as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell described herein. In another aspect, the disclosure features a method of treating a solid tumor cancer in a subject with increased tumor infiltration or immune cell expansion as compared to treating the same type of solid tumor cancer with immune cells expressing a CAR and a CSR comprising a Dap10 costimulatory domain, wherein the method comprises administering to the subject corresponding immune cells expressing the same CAR and a corresponding CSR comprising a CD30 costimulatory domain, and wherein the corresponding immune cells comprise the immune cell described herein. In some embodiments, experiments can be conducted in animals, e.g., mice, to compare the effects of the immune cells in treating a solid tumor cancer by using one group of immune cells comprising a CAR and a CSR with a CD30 costimulatory domain and another group of immune cells comprising the same CAR and a corresponding CSR with a non-CD30 costimulatory domain, e.g., a 4-1BB costimulatory domain, a CD28 costimulatory domain, or a Dap10 costimulatory domain.

In some embodiments of the methods described herein, the ratio of the number of tumor cells infiltrated by immune cells expressing a 2nd generation CAR (e.g., αAFP-CD28z-CAR, αGPC3-CD28z-CAR) and CD30-CSR to the number of tumor cells infiltrated by immune cells expressing the 2nd generation CAR alone is between 1 and 20 (e.g., between 1 and 18, between 1 and 16, between 1 and 14, between 1 and 12, between 1 and 10, between 1 and 8, between 1 and 6, between 1 and 4, between 1 and 2, between 2 and 20, between 4 and 20, between 6 and 20, between 8 and 20, between 10 and 20, between 12 and 20, between 14 and 20, between 16 and 20, or between 18 and 20).

In some embodiments of the methods described herein, the ratio of the blood concentration of immune cells expressing a 2nd generation CAR (e.g., αAFP-CD28z-CAR, αGPC3-CD28z-CAR) and CD30-CSR to the blood concentration of immune cells expressing the 2nd generation CAR alone is between 1 and 5 (e.g., between 1 and 4, between 1 and 3, between 1 and 2, between 2 and 5, between 3 and 5, or between 4 and 5).

In another aspect, the disclosure features a method of treating a solid tumor cancer in a subject with increased tumor regression as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell described herein. In another aspect, the disclosure features a method of treating a solid tumor cancer in a subject with increased tumor regression as compared to treating the same type of solid tumor cancer with immune cells expressing a CAR and a CSR comprising a Dap10 costimulatory domain, wherein the method comprises administering to the subject corresponding immune cells expressing the same CAR and a corresponding CSR comprising a CD30 costimulatory domain, and wherein the corresponding immune cells comprise the immune cell described herein. In some embodiments, experiments can be conducted in animals, e.g., mice, to compare the effects of the immune cells on tumor regression by using one group of immune cells comprising a CAR and a CSR with a CD30 costimulatory domain and another group of immune cells comprising the same CAR and a corresponding CSR with a non-CD30 costimulatory domain, e.g., a 4-1BB costimulatory domain, a CD28 costimulatory domain, or a Dap10 costimulatory domain.

In another aspect, the disclosure features a method for generating central memory T cells in a subject, comprising administering to the subject the nucleic acid(s) described herein, the vector(s) described herein, or the pharmaceutical composition described herein comprising the nucleic acid(s) or the vector(s) to the subject.

In some embodiments, the method increases the number of central memory T cells and/or the percentage of central memory T cells among all T cells in the subject.

In another aspect, the disclosure provides a method for generating central memory T cells in vitro comprising: contacting one or more target cells with the immune cell described herein under conditions and for a time sufficient so that the immune cell develops into central memory T cells, wherein the target cells express an antigen specific to the immune cell.

In some embodiments, the method increases the number of central memory T cells and/or the percentage of central memory T cells among all T cells descended from the immune cell.

In some embodiments, the method generates higher number of central memory T cells and/or higher percentage of central memory T cells than corresponding immune cell expressing a CSR comprising a CD28 costimulatory domain.

In some embodiments, the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 costimulatory domain.

In some embodiments of the methods described herein, immune cells (e.g., CD8⁺ T cells) expressing a 1st generation CAR (e.g., αAFP-CD8T-z-CAR) and CD30-CSR generates more central memory T cells than immune cells (e.g., CD8⁺ T cells) expressing the 1^{st} generation CAR alone. For example, in some embodiments, the ratio of the number of central memory T cells generated by immune cells (e.g., CD8⁺ T cells) expressing a 1^{st} generation CAR (e.g., αAFP-CD8T-z-CAR) and CD30-CSR to the number of central memory T cells generated by immune cells (e.g., CD8⁺ T cells) expressing the 1^{st} generation CAR alone is between 5 and 1000 (e.g., between 5 and 900, between 5 and 800, between 5 and 700, between 5 and 600, between 5 and 500, between 5 and 400, between 5 and 300, between 5 and 200, between 5 and 100, between 5 and 50, between 5 and 10, between 10 and 1000, between 50 and 1000, between 100 and 1000, between 200 and 1000, between 300 and 1000, between 400 and 1000, between 500 and 1000, between 600 and 1000, between 700 and 1000, between 800 and 1000, or between 900 and 1000). For example, in some embodiments, the ratio of the number of central memory T cells generated by immune cells (e.g., CD8⁺ T cells) expressing a 1st generation CAR (e.g., αAFP-CD8T-z-CAR) and CD30-CSR to the number of central memory T cells generated by immune cells (e.g., CD8⁺ T cells) expressing the 1^{st} generation CAR alone is between 1.5 and 8000 (e.g., between 1.5 and 7000, between 1.5 and 6000, between 1.5 and 5000, between 1.5 and 4000, between 1.5 and 3000, between 1.5 and 2000, between 1.5 and 1000, between 1.5 and 500, between 1.5 and 100, between 10 and 8000, between 500 and 8000, between 1000 and 8000, between 2000 and 8000, between 3000 and 8000, between 4000 and 8000, between 5000 and 8000, between 6000 and 8000, or between 7000 and 8000).

In some embodiments of the methods described herein, immune cells (e.g., CD8⁺ T cells) expressing a 2nd generation CAR (e.g., αAFP-CD28z-CAR) and CD30-CSR generates more central memory T cells than immune cells (e.g., CD8⁺ T cells) expressing the 2nd generation CAR alone. For example, in some embodiments, the ratio of the number of central memory T cells generated by immune cells (e.g., CD8⁺ T cells) expressing a 2nd generation CAR (e.g., αAFP-CD28z-CAR) and CD30-CSR to the number of central memory T cells generated by immune cells (e.g., CD8⁺ T cells) expressing the 2nd generation CAR alone is between 0.5 and 3500 (e.g., between 0.5 and 3000, between 0.5 and 2500, between 0.5 and 2000, between 0.5 and 1500, between 0.5 and 1000, between 0.5 and 500, between 0.5 and 100, between 0.5 and 50, between 50 and 3500, between 100 and 3500, between 500 and 3500, between 1000 and 3500, between 1500 and 3500, between 2000 and 3500, between 2500 and 3500, or between 3000 and 3500). For example, in some embodiments, the ratio of the number of central memory T cells generated by immune cells (e.g., CD8⁺ T cells) expressing a 2nd generation CAR (e.g., αAFP-CD28z-CAR) and CD30-CSR to the number of central memory T cells generated by immune cells (e.g., CD8⁺ T cells) expressing the 2nd generation CAR alone is between 1.5 and 20,000 (e.g., between 1.5 and 18,000, between 1.5 and 16,000, between 1.5 and 14,000, between 1.5 and 12,000, between 1.5 and 10,000, between 1.5 and 8,000, between 1.5 and 6,000, between 1.5 and 4,000, between 1.5 and 2,000, between 1.5 and 1,800, between 1.5 and 1,600, between 1.5 and 1,400, between 1.5 and 1,200, between 1.5 and 1,000, between 1.5 and 800, between 1.5 and 600, between 1.5 and 400, between 1.5 and 200, between 1.5 and 100, between 100 and 20,000, between 200 and 20,000, between 400 and 20,000, between 600 and 20,000, between 800 and 20,000, between 1000 and 20,000, between 1,200 and 20,000, between 1,400 and 20,000, between 1,600 and 20,000, between 1,800 and 20,000, between 2,000 and 20,000, between 4,000 and 20,000, between 6,000 and 20,000, between 8,000 and 20,000, between 10,000 and 20,000, between 12,000 and 20,000, between 14,000 and 20,000, between 16,000 and 20,000, or between 18,000 and 20,000).

In some embodiments, the central memory T cells express high levels of CCR7 and low levels of CD45RA.

In some embodiments, the central memory T cells are CD8⁺ T cells.

### Definitions

The scope of present invention is defined by the claims appended hereto and is not limited by particular embodiments described herein; those skilled in the art, reading the present disclosure, will be aware of various modifications that may be equivalent to such described embodiments, or otherwise within the scope of the claims.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context.

In order that the present invention may be more readily understood, certain terms are first defined below. Additional definitions for the following terms and other terms are set forth throughout the specification.

Administration: As used herein, the term “administration” refers to the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human) may be bronchial (including by bronchial instillation), buccal, enteral, interdermal, intra-arterial, intradermal, intragastric, intrahepatic, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Affinity matured (or affinity matured antibody): As used herein, refers to an antibody with one or more alterations in one or more CDRs (or, in some embodiments, framework regions) thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., 1992, BioTechnology 10:779-783 describes affinity maturation by V_{H} and V_{L} domain shuffling. Random mutagenesis of CDR and/or framework residues is described by: Barbas et al., 1994, Proc. Nat. Acad. Sci., U.S.A. 91:3809-3813; Schier et al., 1995, Gene 169: 147-155; Yelton et al., 1995. J. Immunol. 155:1994-2004; Jackson et al., 1995, J. Immunol. 154(7):3310-9; and Hawkins et al., 1992, J. Mol. Biol. 226:889-896. Selection of binders with improved binding properties is described by Thie et al., 2009, Methods Mol. Bio. 525:309-22.

Agent: As used herein may refer to a compound or entity of any chemical class including, for example, polypeptides, nucleic acids, saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a mouse, a rat, a rabbit, a pig, a cow, a deer, a sheep, a goat, a cat, a dog, or a monkey). In some embodiments, animals include, but are not limited to, mammals, birds, reptiles, amphibians, fish, insects, and/or worms. In some embodiments, an animal may be a transgenic animal, genetically engineered animal, and/or a clone.

Antibody moiety: As used herein, this term encompasses full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

Antigen-binding fragment or Antigen-binding portion: The term “antigen-binding fragment” or “antigen-binding portion,” as used herein, refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

Biological activity: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Bispecific antibody: As used herein, refers to a bispecific binding agent in which at least one, and typically both, of the binding moieties is or comprises an antibody moiety. A variety of different bispecific antibody structures are known in the art. In some embodiments, each binding moiety in a bispecific antibody that is or comprises an antibody moiety includes V_{H} and/or V_{L} regions; in some such embodiments, the V_{H} and/or V_{L} regions are those found in a particular monoclonal antibody. In some embodiments, where the bispecific antibody contains two antibody moieties, each includes V_{H} and/or V_{L} regions from different monoclonal antibodies.

The term “bispecific antibody” as used herein also refers to a polypeptide with two discrete binding moieties, each of which binds a distinct target. In some embodiments, a bispecific binding antibody is a single polypeptide; in some embodiments, a bispecific binding antibody is or comprises a plurality of peptides which, in some such embodiments may be covalently associated with one another, for example by cross-linking. In some embodiments, the two binding moieties of a bispecific binding antibody recognize different sites (e.g., epitopes) of the same target (e.g., antigen); in some embodiments, they recognize different targets. In some embodiments, a bispecific binding antibody is capable of binding simultaneously to two targets, which are of different structure.

Carrier: As used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: As used herein, the term “CDR” or “complementarity determining region” is intended to mean the non-contiguous antigen combining sites found within the variable region of both heavy and light chain polypeptides. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); A1-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plackthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic AcidsRes., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
‘Residue numbering follows the nomenclature of MacCallum et al., supra
⁴Residue numbering follows the nomenclature of Lefranc et al., supra
⁵Residue numbering follows the nomenclature of Honegger and Plückthun, supra

Chimeric antigen receptors (CARs): As used herein, refers to an artificially constructed hybrid single-chain protein or single-chain polypeptide containing an extracellular target-binding (e.g., antigen-binding) domain, linked directly or indirectly to a transmembrane domain (“TM domain”, e.g., the transmembrane domain of a costimulatory molecule), which is in turn linked directly or indirectly to an intracellular signaling domain (ISD) comprising a primary immune cell signaling domain (e.g., one involved in T cell or NK cell activation). The extracellular target-binding domain can be a single-chain variable fragment derived from an antibody (scFv). In addition to scFvs, other single chain antigen binding domains can be used in CAR, e.g., tandem scFvs, single-domain antibody fragments (VHHs or sdAbs), single domain bispecific antibodies (BsAbs), intrabodies, nanobodies, immunokines in a single chain format, and Fab, Fab′, or (Fab′)2 in single chain formats. The extracellular target-binding domain can be joined to the TM domain via a flexible hinge/spacer region. The intracellular signaling domain (ISD) comprises a primary signaling sequence, or primary immune cell signaling sequence, which can be from an antigen-dependent, TCR-associated T cell activation molecule, e.g., a portion of the intracellular domain of TCRζ, FcRγ, FcRβ, CD3γ, CD3δ, CD3ε, CD3ζ, CD5, CD22, CD79a, CD79b, or CD66d. The ISD can further comprise a costimulatory signaling sequence; e.g., a portion of the intracellular domain of an antigen-independent, costimulatory molecule such as CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds CD83, Dap10, or the like. Characteristics of CARs include their ability to redirect immune cell (e.g., T cell or NK cell) specificity and reactivity toward a selected target in either MHC-restricted (in cases of TCR-mimic antibodies) or non-MHC-restricted (in cases of antibodies against cell surface proteins) manners, exploiting the antigen-binding properties of monoclonal antibodies. The non-MHC-restricted antigen recognition gives immune cells (e.g., T cells or NK cells) expressing CARs the ability to recognize antigen independent of antigen processing, thus bypassing a major mechanism of tumor escape.

There are currently three generations of CARs. The “first generation” CARs are typically single-chain polypeptides composed of a scFv as the antigen-binding domain fused to a transmembrane domain fused to the cytoplasmic/intracellular domain, which comprises a primary immune cell signaling sequence such as the intracellular domain from the CD3ζ chain, which is the primary transmitter of signals from endogenous TCRs. The “first generation” CARs can provide de novo antigen recognition and cause activation of both CD4⁺ and CD8⁺ T cells through their CD3ζ chain signaling domain in a single fusion molecule, independent of HLA-mediated antigen presentation. The “second generation” CARs add intracellular domains from various costimulatory molecules (e.g., CD28, 4-1BB, ICOS, OX40) to the primary immune cell signaling sequence of the CAR to provide additional signals to the T cell. Thus, the “second generation” CARs comprise fragments that provide costimulation (e.g., CD28 or 4-IBB) and activation (e.g., CD3ζ). Preclinical studies have indicated that the “second generation” CARs can improve the antitumor activity of T cells. For example, robust efficacy of the “second generation” CAR modified T cells was demonstrated in clinical trials targeting the CD19 molecule in patients with chronic lymphoblastic leukemia (CLL) and acute lymphoblastic leukemia (ALL). The “third generation” CARs comprise those that provide multiple costimulation (e.g., CD28 and 4-1BB) and activation (e.g., CD3ζ). Examples of CAR T therapies are described, see, e.g., U.S. Pat. No. 10,221,245 describing CAR CTL019 which has an anti-CD19 extracellular target-binding domain, a transmembrane domain from CD8, a costimulatory domain from 4-1BB, and a primary signaling domain from CD3ζ, as well as U.S. Pat. No. 9,855,298 which describes a CAR having an anti-CD19 extracellular target-binding domain, a costimulatory domain from CD28, and a primary signaling domain from CD3ζ.

Adoptive cell therapy: Adoptive cell therapy is a therapeutic approach that typically includes isolation and ex vivo expansion and/or manipulation of immune cells (e.g., NK cells or T cells) and subsequent administration of these cells to a patient, for example for the treatment of cancer. Administered cells may be autologous or allogeneic. Cells may be manipulated to express engineered receptors (including CAR and CSR) in any one of the known ways, including, for example, by using RNA and DNA transfection, viral transduction, electroporation, all of which are technologies known in the art.

The term “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL) and CAR and/or CSR modified lymphocytes. In another embodiment, the adoptive cell therapeutic composition comprises a cell type selected from a group consisting of T cells, CD8⁺ cells, CD4⁺ cells, NK-cells, delta-gamma T cells, regulatory T cells, and peripheral blood mononuclear cells. In another embodiment, TILs, T cells, CD8⁺ cells, CD4⁺ cells, NK-cells, delta-gamma T cells, regulatory T cells, or peripheral blood mononuclear cells form the adoptive cell therapeutic composition. In one embodiment, the adoptive cell therapeutic composition comprises T cells.

In some embodiments, the CAR expressed in the cell is a first generation, second generation, or third generation CAR, as described above. In accordance with the presently disclosed subject matter, the CARs of the engineered immune cells provided herein comprise an extracellular antigen-binding domain, a transmembrane domain, and an intracellular domain. WO 2019/032699 describes T cells co-expressing a CAR and an inducible bispecific antibody.

Comparable: As used herein, refers to two or more agents, entities, situations, sets of conditions, etc. that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Control: As used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. As used herein, a “control” may refer to a “control antibody”. A “control antibody” may be a human, chimeric, humanized, CDR-grafted, multispecific, or bispecific antibody as described herein, an antibody that is different as described herein, or a parental antibody. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Corresponding to: As used herein designates the position/identity of an amino acid residue in a polypeptide of interest. Those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids.

Detection entity/agent: As used herein, refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., 3H, 14C, 18F, 19F, 32P, 35S, 135I, 125I, 123I, 64Cu, 187Re, 111In, 90Y, 99mTc, 177Lu, 89Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Effector function: As used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: As used herein refers to a cell of the immune system that mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Engineered: As used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some particular such embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by those in the art, progeny of an engineered polynucleotide or cell are typically still referred to as “engineered” even though the actual manipulation was performed on a prior entity. Furthermore, as will be appreciated by those skilled in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.) are well known in the art and described in various general and more specific references that are cited and/or discussed throughout the present specification. See e.g., Sambrook et al., Molecular Cloning: A Laboratory Manual(2d ed., Cold Spring Harbor Laboratory Press, Cold Spring Harbor, N.Y., 1989).

Epitope: As used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized). An antibody moiety described herein may bind to an epitope comprising between 7 and 50 amino acids (e.g., between 7 and 50 contiguous amino acids), e.g., between 7 and 45, between 7 and between 7 and 40, between 7 and 35, between 7 and 30, between 7 and 25, between 7 and 20, between 7 and 15, between 7 and 10, between 10 and 50, between 15 and 50, between 20 and 50, between 25 and 50, between 30 and 50, between 35 and 50, between 40 and 50, between 45 and 50, between 10 and 45, between 15 and 40, between 20 and 35, or between 25 and 30 amino acids.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression cassette: As used herein, refers to a nucleic acid construct that, when introduced into a host cell, results in transcription and/or translation of an RNA or polypeptide, respectively.

Heterologous: As used herein, refers to a polynucleotide or polypeptide that does not naturally occur in a host cell or a host organism. A heterologous polynucleotide or polypeptide may be introduced into the host cell or host organism using well-known recombinant methods, e.g., using an expression cassette comprising the heterologous polynucleotide optionally linked to a promoter.

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Host cell: As used herein, refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic and is selected from the following cells: CHO (e.g., CHO Kl, DXB-1 1 CHO, Veggie-CHO), COS (e.g., COS-7), retinal cell, Vero, CV1, kidney (e.g., HEK293, 293 EBNA, MSR293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, a host cell comprises one or more viral genes, e.g., a retinal cell that expresses a viral gene (e.g., a PER.C6™ cell).

Human antibody: As used herein, is intended to include antibodies having variable and constant regions generated (or assembled) from human immunoglobulin sequences. In some embodiments, antibodies (or antibody moieties) may be considered to be “human” even though their amino acid sequences include residues or elements not encoded by human germline immunoglobulin sequences (e.g., include sequence variations, for example, that may (originally) have been introduced by random or site-specific mutagenesis in vitro or by somatic mutation in vivo), for example in one or more CDRs and in particular CDR3. Human antibodies, human antibody moieties, and their fragments can be isolated from human immune cells or generated recombinantly or synthetically, including semi-synthetically.

Humanized: As is known in the art, the term “humanized” is commonly used to refer to antibodies (or moieties) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody moiety) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a CHI, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Improve, increase, or reduce: As used herein, or grammatical equivalents thereof, indicate values that are relative to a baseline measurement, such as a measurement in the same individual prior to initiation of a treatment described herein, or a measurement in a control individual (or multiple control individuals) in the absence of the treatment described herein. A “control individual” is an individual afflicted with the same form of disease or injury as the individual being treated. In some embodiments, the methods for treating a cancer (e.g., a hematological cancer or a solid tumor cancer) using an immune cell described herein may increase cell apoptosis (e.g., increase tumor cell apoptosis) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual. In some embodiments, the methods for treating a cancer (e.g., a hematological cancer or a solid tumor cancer) using an immune cell described herein may reduce tumor size (e.g., reduce tumor size) in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to the individual prior to receiving treatment or to a control individual.

In vitro: As used herein refers to events that occur in an artificial environment, e.g., in a test tube or reaction vessel, in cell culture, etc., rather than within a multi-cellular organism.

In vivo: As used herein refers to events that occur within a multi-cellular organism, such as a human and a non-human animal. In the context of cell-based systems, the term may be used to refer to events that occur within a living cell (as opposed to, for example, in vitro systems).

Isolated: As used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: As used herein, refers to the dissociation constant of a binding agent (e.g., an antibody agent or binding component thereof) from a complex with its partner (e.g., the epitope to which the antibody agent or binding component thereof binds).

k_{off}: As used herein, refers to the off rate constant for dissociation of a binding agent (e.g., an antibody agent or binding component thereof) from a complex with its partner (e.g., the epitope to which the antibody agent or binding component thereof binds).

kₒₙ: As used herein, refers to the on rate constant for association of a binding agent (e.g., an antibody agent or binding component thereof) with its partner (e.g., the epitope to which the antibody agent or binding component thereof binds).

Linker: As used herein, is used to refer to that portion of a multi-element polypeptide that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker has between 3 and 7 amino acids, between 7 and 15 amino acids, or between 20 and 30 (e.g., between 20 and 25 or between 25 and 30) amino acids. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al., 1993, Proc. Natl. Acad. Sci. U.S.A. 90:6444-6448; Poljak, R. J. et al., 1994, Structure 2:1121-1123).

Multivalent binding antibody (or multispecific antibody): As used herein, refers an antibody capable of binding to two or more antigens, which can be on the same molecule or on different molecules. Multivalent binding antibodies as described herein are, in some embodiments, engineered to have the two or more antigen binding sites, and are typically not naturally occurring proteins. Multivalent binding antibodies as described herein refer to antibodies capable of binding two or more related or unrelated targets. Multivalent binding antibodies may be composed of multiple copies of a single antibody moiety or multiple copies of different antibody moieties. Such antibodies are capable of binding to two or more antigens and may be tetravalent or multivalent. Multivalent binding antibodies may additionally comprise a therapeutic agent, such as, for example, an immunomodulator, toxin or an RNase. Multivalent binding antibodies as described herein are, in some embodiments, capable of binding simultaneously to at least two targets that are of different structure, e.g., two different antigens, two different epitopes on the same antigen, or a hapten and/or an antigen or epitope. Multivalent binding antibodies of the present invention may be monospecific (capable of binding one antigen) or multispecific (capable of binding two or more antigens), and may be composed of two heavy chain polypeptides and two light chain polypeptides. Each binding site, in some embodiments, is composed of a heavy chain variable domain and a light chain variable domain with a total of six CDRs involved in antigen binding per antigen binding site.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds.

In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control said gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Physiological conditions: As used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Polypeptide: As used herein, refers to any polymeric chain of amino acids. In some embodiments, the amino acids are joined to each other by peptide bonds or modified peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is synthetically designed and/or produced. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids.

In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class.

In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30 to 40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or comprise a characteristic sequence element) that shows very high sequence identity, often greater than 90% or even 95%, 96%, 97%, 98%, or 99%. Such a conserved region usually encompasses at least three to four and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice-versa, and/or fragments may be present in a different order in the polypeptide of interest than in the parent), so that the polypeptide of interest is a derivative of its parent polypeptide.

Prevent or prevention: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, is intended to refer to polypeptides (e.g., antibodies or antibody moieties) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., 1997, TIB Tech. 15:62-70; Azzazy H., and Highsmith W. E., 2002, Clin. Biochem. 35:425-45; Gavilondo J. V., and Larrick J. W., 2002, BioTechniques 29:128-45; Hoogenboom H., and Chames P., 2000, Immunol. Today 21:371-8), antibodies isolated from an animal (e.g., a mouse) that is transgenic for human immunoglobulin genes (see e.g., Taylor, L. D., et al., 1992, Nucl. Acids Res. 20:6287-95; Kellermann S-A., and Green L. L., 2002, Curr. Opin. Biotech. 13:593-7; Little, M. et al., 2000, Immunol. Today 21:364-70; Murphy, A. J. et al., 2014, Proc. Natd. Acad. Sci. U.S.A. 111(14):5153-8) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the V_{H} and V_{L} regions of the recombinant antibodies are sequences that, while originating from and related to germline V_{H} and V_{L} sequences, may not naturally exist within the germline antibody repertoire in vivo.

Reference: As used herein describes a standard, control, or other appropriate reference against which a comparison is made as described herein. For example, in some embodiments, a reference is a standard or control agent, animal, individual, population, sample, sequence, series of steps, set of conditions, or value against which an agent, animal, individual, population, sample, sequence, series of steps, set of conditions, or value of interest is compared. In some embodiments, a reference is tested and/or determined substantially simultaneously with the testing or determination of interest. In some embodiments, a reference is a historical reference, optionally embodied in a tangible medium. Typically, as would be understood by those skilled in the art, a reference is determined or characterized under conditions comparable to those utilized in the assessment of interest.

Specific binding: As used herein, refers to a binding agent's ability to discriminate between possible partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations. In some embodiments, specific binding is assessed by determining the difference in binding affinity between cognate and non-cognate targets. For example, a binding agent may have a binding affinity for a cognate target that is about 3-fold, 4-fold, 5-fold, 6-fold, 7-fold, 8-fold, 9-fold, 10-fold or more than binding affinity for a non-cognate target.

Specificity: As is known in the art, “specificity” is a measure of the ability of a particular ligand to distinguish its binding partner from other potential binding partners.

Subject: As used herein, means any mammal, including humans. In certain embodiments of the present invention the subject is an adult, an adolescent or an infant. In some embodiments, terms “individual” or “patient” are used and are intended to be interchangeable with “subject.” Also contemplated by the present invention are the administration of the pharmaceutical compositions and/or performance of the methods of treatment in utero.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial sequence homology: As used herein, the phrase “substantial homology” to refers to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized as follows:

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | He | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xie | J |
| Unspecified or unknown | Xaa | X |
| amino acid | | |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., 1990, J. Mol. Biol., 215(3):403-410; Altschul et al., 1996, Meth. Enzymology 266:460-480; Altschul et al., 1997, Nucleic Acids Res. 25:3389-3402; Baxevanis et al, Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Surface plasmon resonance: As used herein, refers to an optical phenomenon that allows for the analysis of specific binding interactions in real-time, for example through detection of alterations in protein concentrations within a biosensor matrix, such as by using a BIAcore system (Pharmacia Biosensor AB, Uppsala, Sweden and Piscataway, N.J.). For further descriptions, see Jonsson, U. et al., 1993, Ann. Biol. Clin. 51:19-26; Jonsson, U. et al., 1991, Biotechniques 11:620-627; Johnsson, B. et al., 1995, J. Mol. Recognit. 8:125-131; and Johnsson, B. et al., 1991, Anal. Biochem. 198:268-277.

Therapeutic agent: As used herein, generally refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, is meant an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”), in its broadest sense, refers to any administration of a substance (e.g., provided compositions) that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide.

In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 insertions or deletions, and often has no insertions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

Vector: As used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Wild type: As used herein, the term “wild type” has its art-understood meaning that refers to an entity having a structure and/or activity as found in nature in a “normal” (as contrasted with mutant, variant, diseased, altered, etc.) state or context. Those of ordinary skill in the art will appreciate that wild type genes and polypeptides often exist in multiple different forms (e.g., alleles).

### BRIEF DESCRIPTION OF THE DRAWINGS

FIG. 1: T cell-mediated short-term target cell killing by T cells expressing (1) anti-AFP-CD28z-CAR; (2) anti-AFP-CD28z-CAR+anti-GPC3-CD30-CSR; (3) anti-AFP-CD8T-z-CAR; or (4) anti-AFP-CD8T-z-CAR+anti-GPC3-CD30-CSR.

FIGS. 2A and 2B: T cells expressing (1) anti-AFP-CD28z-CAR; (2) anti-AFP-CD28z-CAR+anti-GPC3-CD30-CSR; (3) anti-AFP-CD8T-z-CAR; or (4) anti-AFP-CD8T-z-CAR+anti-GPC3-CD30-CSR had much higher IFNγ (FIG. 2A) and Granzyme B (FIG. 2B) (both indicators for T cell activities/killing capability) secretion levels than corresponding CAR T cells without CSR.

FIGS. 3A and 3B: The results of T cell survival and killing of target HepG2 (A2⁺/AFP⁺/GPC3⁺) cells mediated by T cells expressing 1^{st} generation CAR constructs: (1) anti-AFP-CD8T-z-CAR; (2) anti-AFP-CD8T-z-CAR+anti-GPC3-CD28-CSR; or (3) anti-AFP-CD8T-z-CAR+anti-GPC3-CD30-CSR. T cells expressing anti-AFP-CD8T-z-CAR+anti-GPC3-CD28-CSR or anti-AFP-CD8-z-CAR+anti-GPC3-CD30-CSR survived much better than mock-transduced T cells and T cells expressing only the corresponding CARs (FIG. 3A). Further, T cells expressing anti-AFP-CD8T-z-CAR+anti-GPC3-CD28-CSR or anti-AFP-CD8-z-CAR+anti-GPC3-CD30-CSR killed many more target cells than T cell expressing only the corresponding CARs (FIG. 3B).

FIGS. 3C and 3D: The results of T cell survival and killing of target HepG2 (A2⁺/AFP⁺/GPC3⁺) cells mediated by T cells expressing 2^{nd} generation CAR constructs: (1) anti-AFP-CD28z-CAR; (2) anti-AFP-CD28z-CAR+anti-GPC3-CD28-CSR; or (3) anti-AFP-CD28z-CAR+anti-GPC3-CD30-CSR. T cells expressing anti-AFP-CD28z-CAR+anti-GPC3-CD28-CSR or anti-AFP-CD28z-CAR+anti-GPC3-CD30-CSR survived much better than mock-transduced T cells and T cells expressing only the corresponding CARs (FIG. 3C). Further, T cells expressing anti-AFP-CD28z-CAR+anti-GPC3-CD28-CSR or anti-AFP-CD28z-CAR+anti-GPC3-CD30-CSR killed many more target cells than T cell expressing only the corresponding CARs (FIG. 3D).

FIG. 4: Images of tumor sections stained with anti-CD3 antibody to visualize T cells in tumors dissected from mice adminstered with: (1) mock-transduced T cells; (2) T cells expressing αAFP-CD28z-CAR; (3) T cells expressing αAFP-CD28z-CAR+αGPC3-CD28-CSR; or (4) T cells expressing αAFP-CD28z-CAR+αGPC3-CD30-CSR. The blue cells are tumor cells which represent all the cells in the “Mock” image, while the brown cells are T cells which represent less than 5% of all the cells in the “αAFP-CD28z-CAR” image, about a third of all the cells in the “αAFP-CD28z-CAR+αGPC3-CD28-CSR” image, and about half of all the cells in the “αAFP-CD28z-CAR+αGPC3-CD30-CSR” image.

FIG. 5: Quantification of the percentages of CD3⁺ cells (T cells) among all cells (including tumor cells and CD3⁺ cells) in multiple tumor sections from HepG-implanted mice that were later treated with T cells expressing (1) αAFP-CD28z-CAR; (2) αAFP-CD28z-CAR+αGPC3-CD28-CSR; or (3) αAFP-CD28z-CAR+αGPC3-CD30-CSR.

FIG. 6: Images of tumor sections stained with anti-CD3 antibody to visualize T cells in tumors dissected from mice adminstered with: (1) mock-transduced T cells; (2) T cells expressing αGPC3-CD28z-CAR; or (3) T cells expressing αGPC3-CD28z-CAR+αGPC3-CD30-CSR. The blue cells are tumor cells which represent all the cells in the “Mock” image, while the brown cells are T cells which represent about a quarter of all the cells in the “αGPC3-CD28z-CAR” image and about half of all the cells in the “αGPC3-CD28z-CAR+αGPC3-CD30-CSR” image.

FIG. 7: Quantification of the percentages of CD3⁺ cells (T cells) among all cells (including tumor cells and CD3⁺ cells) in multiple tumor sections from HepG-implanted mice that were later treated with T cells expressing (Group 1) αGPC3-CD28z-CAR; (Group 2) αGPC3-CD30T-CD28-CSR; or (Group 3) αGPC3-CD28z-CAR+αGPC3-CD30-CSR.

FIGS. 8A and 8B: T cells expressing anti-CD19-CD8T-41BBz-CAR+anti-CD19-CD28T-CD30-CSR or anti-CD19-CD8T-z-CAR+anti-CD19-CD30-CSR had higher IFNγ (an indicator for T cell activities/killing capability) secretion level than corresponding T cells expressing anti-CD19-CD8T-41BBz-CAR+anti-CD19-CD28T-41BB-CSR or anti-CD19-CD8T-z-CAR+anti-CD19-CD28-CSR.

FIG. 9: T cells expressing anti-ROR1-CD8T-41BBz-CAR+anti-ROR1-CD28T-CD30-CSR (“tCD30”) had significant ROR1-specific cell killing capability against all six tested cancer cell lines (measured by IFNγ release level) as compared to the mock-transduced T cells, and their cell killing capability is comparable to or better than that of corresponding CAR T cells co-expressing a CSR comprising a 4-1BB costimulatory domain (“t41BB”).

FIGS. 10A-10D: Survival of αROR1-CD8T-41BBz-CAR+αROR1-CD28T-CD30-CSR T cells (“tCD30”) and αROR1-CD8T-41BBz-CAR+αROR1-CD28T-41BB-CSR T cells (“t41BB”) over multiple challenges by cancer cell lines MDA-MB-231, A549, H1975, and H1703, respectively. The Total Cell #s shown are T cell numbers.

### DETAILED DESCRIPTION OF THE EMBODIMENTS

Adoptive T cell immunotherapy, in which a patient's own T lymphocytes are engineered to express chimeric antigen receptors (CARs), has shown great promise in treating hematological malignancies, but not so much in solid tumors. In addition, CAR by itself is generally not efficacious enough, especially for solid tumors, even with the commonly used costimulatory fragments, no matter if expressed in cis or in trans. Therefore, more efficacious and longer-lasting T cell immunotherapies are needed.

We disclose herein that co-expression of CAR and CSR, in particular a CSR comprising a CD30 costimulatory fragment, will benefit any CAR T cell that targets a low-density antigen. Most MHC-restricted peptide antigens and solid tumor antigens are of low-density. However, even some blood cancer related cell-surface antigens, e.g., CD22, are of low-density. When used to treat solid tumors, T cells expressing CAR and CD30-CSR have increased tumor infiltration.

The present invention relates to the discovery of CSRs that use a costimulatory domain from CD30 (also referred to herein as a CD30 costimulatory domain) and T cells expressing these CSRs and CARs have far less expression of PD-1, an inhibitor of T cell activation, than T cells with the same CARs and CSRs containing a costimulatory domain from, e.g., CD28 or 4-1BB. In some embodiments, T cells with CSRs containing a costimulatory domain from CD30 express far less PD-1 than T cells with CSRs containing a costimulatory domain from Dap10. The T cells with CARs and CSRs comprising a CD30 costimulatory domain provide superior persistence of tumor cell killing. The invention also provides the use of such T cells to treat cancer (e.g., a hematological cancer or a solid tumor cancer).

I. Chimeric Antigen Receptors (CARs)

The disclosure provides immune cells comprising: a chimeric antigen receptor (CAR) and a chimeric stimulating receptor (CSR). The CAR comprises (i) an extracellular target-binding domain comprising an antibody moiety (a CAR antibody moiety); (ii) a transmembrane domain (a CAR transmembrane domain); and (iii) a primary signaling domain. In some embodiments, the CAR further comprises a costimulatory domain (a CAR costimulatory domain). In some embodiments, the CAR costimulatory domain is derived from the intracellular domain of a costimulatory receptor, for example, a costimulatory receptor selected from the group consisting of CD30, CD27, CD28, 4-1BB (CD137), OX40, CD40, PD-1, ICOS, lymphocyte function-associated antigen-1 (LFA-1), CD2, CD7, LIGHT, NKG2C, B7-H₃, a ligand that specifically binds with CD83, and Dap10. Exemplary sequences of CARs described herein can be found in the Informal Sequence Listing table, e.g., SEQ ID NOS:1-12. In some embodiments, the CARs with myc-tags are used in in vitro and pre-clinical assays. For in vivo use, i.e., in vivo use in humans, the corresponding CAR constructs without myc-tags are used.

In some embodiments, a spacer domain may be present between the extracellular target-binding domain and the transmembrane domain of the CAR. In some embodiments, a spacer domain may be present between the transmembrane domain and the costimulatory domain of the CAR, if present. In some embodiments, a spacer domain may be present between the costimulatory domain (if present) and the primary signaling domain of the CAR. In some embodiments, a spacer domain may be present between the transmembrane domain and the primary signaling domain of the CAR. The spacer domain can be any oligo- or polypeptide that functions to link two parts of the CAR. A spacer domain may comprise up to about 300 amino acids, including for example about 10 to about 100, or about 25 to about 50 amino acids.

II. Chimeric Stimulating Receptors (CSRs)

The disclosure provides a chimeric stimulating receptor (CSR), also called chimeric signaling receptor by us, comprising: (i) a ligand-binding module that is capable of binding or interacting with a target ligand; (ii) a transmembrane domain (a CSR transmembrane domain); and (iii) a CD30 costimulatory domain, wherein the CSR lacks a functional primary signaling domain. The CSRs described herein specifically binds to a target ligand (such as a cell surface antigen or a peptide/MHC complex) and is capable of stimulating an immune cell on the surface of which it is functionally expressed upon target ligand binding. The CSR comprises a ligand-binding module that provides the ligand-binding specificity, a transmembrane module, and a CD30 costimulatory immune cell signaling module that allows for stimulating the immune cell. The CSR lacks a functional primary immune cell signaling sequence. In some embodiments, the CSR lacks any primary immune cell signaling sequence. In some embodiments, the CSR comprises a single polypeptide chain comprising the ligand-binding module, transmembrane module, and CD30 costimulatory signaling module. In some embodiments, the CSR comprises a first polypeptide chain and a second polypeptide chain, wherein the first and second polypeptide chains together form the ligand-binding module, transmembrane module, and CD30 costimulatory signaling module. In some embodiments, the first and second polypeptide chains are separate polypeptide chains, and the CSR is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond. In some embodiments, the expression of the CSR in the CAR plus CSR immune cell is inducible. In some embodiments, the expression of the CSR in the CAR plus CSR immune cell is inducible upon signaling through the CAR. Exemplary sequences of CSRs described herein can be found in the Informal Sequence Listing table, e.g., SEQ ID NOS:13-42. In some embodiments, the CSRs with myc-tags are used in in vitro and pre-clinical assays. For in vivo use, i.e., in vivo use in humans, the corresponding CSR constructs without myc-tags are used.

The CD30 costimulatory domain of the CSR can comprise a sequence that can bind to an intracellular TRAF signaling protein. In some embodiments, the sequence that can bind to an intracellular TRAF signaling protein corresponds to residues 561-573 or 578-586 of a full-length CD30 having the sequence of SEQ ID NO:65. In certain embodiments, the CD30 costimulatory domain comprises a sequence that is at least 80%, 85%, 90%, 95%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to residues 561-573 or 578-586 of SEQ ID NO:65. In certain embodiments, the CD30 costimulatory domain comprises a sequence that is at least 70%, 75%, 80%, 85%, 90%, 95%, or 100% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identical to the sequence of SEQ ID NO:75. As described herein, immune T cells with a CAR and a CSR that comprises a costimulatory domain from CD30 express far less PD-1, an inhibitor of T cell activation, than T cells with the same CAR and a corresponding CSR that does not have a CD30 costimulatory domain, e.g., a costimulatory domain from, e.g., CD28, 4-1BB, or Dap10. T cells with a CSR containing a costimulatory domain from CD30 also demonstrate persistence in cytotoxic potential. The costimulatory domain from CD30 may ameliorate the functional unresponsiveness that leads to T cell exhaustion, i.e., anergy. The ability of a CD30 costimulatory domain to provide T cells with superior persistence of tumor cell killing is unexpected since CD30 lacks a p56lck-binding site that is thought to be crucial for costimulation.

The CSR can comprise more than one CD30 costimulatory domain. In addition to the CD30 costimulatory domain, in some embodiments, the CSR further comprises at least one costimulatory domain which comprises the intracellular sequence of a costimulatory molecule that is different from CD30. In particular embodiments, the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, a ligand that specifically binds with CD83, and Dap10.

In some embodiments, a spacer domain may be present between the ligand-binding module and the transmembrane domain of the CSR. In some embodiments, a spacer domain may be present between the transmembrane domain and the CD30 costimulatory domain of the CSR. The spacer domain can be any oligo- or polypeptide that functions to link two parts of the CAR. A spacer domain may comprise up to about 300 amino acids, including for example about 10 to about 100, or about 25 to about 50 amino acids.

Target Antigen

In some embodiments, the extracellular target-binding domain of the CAR and the ligand-binding module of the CSR can target the same target antigen. In other embodiments, the extracellular target-binding domain of the CAR and the ligand-binding module of the CSR can target different target antigens. In some embodiments, the ligand-binding module of the CSR is derived from the extracellular domain of a receptor. The ligand-binding module of the CSR can comprise an antibody moiety (a CSR antibody moiety). The CSR antibody moiety and/or the CAR antibody moiety can be a single chain antibody fragment. In some embodiments, the CAR antibody moiety and/or the CSR antibody moiety is a single chain Fv (scFv), a single chain Fab, a single chain Fab′, a single domain antibody fragment, a single domain multispecific antibody, an intrabody, a nanobody, or a single chain immunokine. In certain embodiments, the CAR antibody moiety and/or the CSR antibody moiety is a single domain multispecific antibody, e.g., a single domain bispecific antibody. In certain embodiments, the CAR antibody moiety and/or the CSR antibody moiety is a single chain Fv (scFv), e.g., a tandem scFv. In some embodiments, the CAR antibody moiety and/or the CSR antibody moiety specifically binds to a disease-related antigen. The disease-related antigen can be a cancer-related antigen or a virus-related antigen.

The CAR antibody moiety and/or the CSR antibody moiety can specifically bind to a cell surface antigen. A cell surface antigen can be selected from the group consisting of protein, carbohydrate, and lipid. In certain embodiments, the cell surface antigen is CD19, CD20, CD22, CD47, CD158e, GPC3, ROR1, ROR2, BCMA, GPRC5D, FcRL5, MUC16, MCT4, PSMA, or a variant or mutant thereof. The CAR antibody moiety and/or the CSR antibody moiety can specifically bind to an MHC-restricted antigen. The MHC-restricted antigen can be a complex comprising a peptide and an MHC protein, and the peptide can be derived from a protein selected from the group consisting of WT-1, AFP, HPV16-E7, NY-ESO-1, PRAME, EBV-LMP2A, HIV-1, KRAS, FoxP3, Histone H3.3, PSA, and a variant or mutant thereof.

In some embodiments, the CAR antibody moiety binds to CD19, and wherein the ligand-binding module of the CSR binds to CD19. In some embodiments, the CAR antibody moiety binds to CD22, and the ligand-binding module of the CSR binds to CD22. In some embodiments, the CAR antibody moiety binds to CD20, and the ligand-binding module of the CSR binds to CD20. In some embodiments, the CAR antibody moiety binds to CD19, and the ligand-binding module of the CSR binds to CD22. In some embodiments, the CAR antibody moiety binds to CD19, and the ligand-binding module of the CSR binds to CD20. In some embodiments, the CAR antibody moiety binds to CD22, and the ligand-binding module of the CSR binds to CD20. In some embodiments, the CAR antibody moiety binds to CD22, and the ligand-binding module of the CSR binds to CD19. In some embodiments, the CAR antibody moiety binds to CD20, and the ligand-binding module of the CSR binds to CD19. In some embodiments, the CAR antibody moiety binds to CD20, and the ligand-binding module of the CSR binds to CD22. In some embodiments, the CAR antibody moiety and/or the ligand-binding module of the CSR binds to both CD19 and CD22. In some embodiments, the CAR antibody moiety and/or the ligand-binding module of the CSR binds to both CD19 and CD20. In some embodiments, the CAR antibody moiety and/or the ligand-binding module of the CSR binds to both CD20 and CD22. In some embodiments, the CAR antibody moiety and/or the ligand-binding module of the CSR binds to CD19, CD20, and CD22.

In some embodiments, the CAR antibody moiety specifically binds to a complex comprising an alpha-fetoprotein (AFP) peptide and an MHC class I protein. In some embodiments, the ligand-binding module of the CSR specifically binds to glypican 3 (GPC3). In some embodiments, the CAR antibody moiety binds to a complex comprising an AFP peptide and an MHC class I protein, and the ligand-binding module of the CSR binds to GPC3.

In some embodiments, according to any of the CARs or CSRs described herein comprising an antibody moiety that specifically binds to a target antigen, the antibody moiety comprises the CDRs or variable domains (V_{H} and/or V_{L} domains) of an antibody moiety specific for the target antigen. In some embodiments, the antibody moiety comprises the CDRs or variable domains (V_{H} and/or V_{L} domains) of an antibody moiety specific for CD19 (see, e.g., WO2017/066136A2). In some embodiments, the antibody moiety comprises the CDRs or variable domains (V_{H} and/or V_{L} domains) of an antibody moiety specific for CD19 (e.g., V_{H} domain comprising, consisting essentially of, or consisting of the amino acid sequence of SEQ ID NO:102 and/or V_{L} domain comprising, consisting essentially of, or consisting of the amino acid sequence of SEQ ID NO:103, or CDRs contained therein). In some embodiments, the antibody moiety comprises the CDRs or variable domains (V_{H} and/or V_{L} domains) of an antibody moiety specific for CD20 (e.g., V_{H} domain comprising, consisting essentially of, or consisting of the amino acid sequence of SEQ ID NO:104 and/or V_{L} domain comprising, consisting essentially of, or consisting of the amino acid sequence of SEQ ID NO:105, or CDRs contained therein). In some embodiments, the antibody moiety comprises the CDRs or variable domains (V_{H} and/or V_{L} domains) of an antibody moiety specific for CD22 (see, e.g., U.S. Ser. No. 62/650,955 filed Mar. 30, 2018 and PCT/US2019/025032, filed Mar. 29, 2019), the contents of which are incorporated herein by reference in their entirety). In some embodiments, the antibody moiety comprises the CDRs or variable domains (V_{H} and/or V_{L} domains) of an antibody moiety specific for CD22 (e.g., V_{H} domain comprising, consisting essentially of, or consisting of the amino acid sequence of SEQ ID NO:98 and/or V_{L} domain comprising, consisting essentially of, or consisting of the amino acid sequence of SEQ ID NO:99, or CDRs contained therein). In some embodiments, the antibody moiety comprises the CDRs or variable domains (V_{H} and/or V_{L} domains) of an antibody moiety specific for CD22 (e.g., V_{H} domain comprising, consisting essentially of, or consisting of the amino acid sequence of SEQ ID NO:100 and/or V_{L} domain comprising, consisting essentially of, or consisting of the amino acid sequence of SEQ ID NO:101, or CDRs contained therein). In some embodiments, the antibody moiety comprises the CDRs or variable domains (V_{H} and/or V_{L} domains) of an antibody moiety specific for CD47 (see, e.g., WO2018/200585A1). In some embodiments, the antibody moiety comprises the CDRs or variable domains (V_{H} and/or V_{L} domains) of an antibody moiety specific for CD47 (e.g., V_{H} domain comprising, consisting essentially of, or consisting of the amino acid sequence of SEQ ID NO:106 and/or V_{L} domain comprising, consisting essentially of, or consisting of the amino acid sequence of SEQ ID NO:107, or CDRs contained therein).

In some embodiments, the antibody moiety comprises the CDRs or variable domains (V_{H} and/or V_{L} domains) of an antibody moiety specific for GPC3 (see, e.g., WO2018/200586A1, the contents of which are incorporated herein by reference in their entirety). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:108 and/or V_{L} domain comprising, consisting essentially of, or consisting of the amino acid sequence of SEQ ID NO:109, or CDRs contained therein). In some embodiments, the antibody moiety comprises the CDRs or variable domains (V_{H} and/or V_{L} domains) of an antibody moiety specific for GPC3 (e.g., V_{H} domain comprising, consisting essentially of, or consisting of the amino acid sequence of SEQ ID NO:110 and/or V_{L} domain comprising, consisting essentially of, or consisting of the amino acid sequence of SEQ ID NO:111, or CDRs contained therein). In some embodiments, the antibody moiety comprises the CDRs or variable domains (V_{H} and/or V_{L} domains) of an antibody moiety specific for ROR1 (see, e.g., WO2016/187220 and WO2016/187216). In some embodiments, the antibody moiety comprises the CDRs or variable domains (V_{H} and/or V_{L} domains) of an antibody moiety specific for ROR2 (see, e.g., WO2016/142768). In some embodiments, the antibody moiety comprises the CDRs or variable domains (V_{H} and/or V_{L} domains) of an antibody moiety specific for BCMA (see, e.g., WO2016/090327 and WO2016/090320). In some embodiments, the antibody moiety comprises the CDRs or variable domains (V_{H} and/or V_{L} domains) of an antibody moiety specific for GPRC5D (see, e.g., WO2016/090329 and WO2016/090312). In some embodiments, the antibody moiety comprises the CDRs or variable domains (V_{H} and/or V_{L} domains) of an antibody moiety specific for FCRL5 (see, e.g., WO2016/090337). In some embodiments, the antibody moiety comprises the CDRs or variable domains (V_{H} and/or V_{L} domains) of an antibody moiety specific for MUC16 (see, e.g., U.S. Ser. No. 62/845,065, filed May 8, 2019 and U.S. Ser. No. 62/768,730, filed Nov. 16, 2018 the contents of which are incorporated herein by reference in their entirety). In some embodiments, the antibody moiety comprises the CDRs or variable domains (V_{H} and/or V_{L} domains) of an antibody moiety specific for MCT4 (see, e.g., PCT/US2019/023402, filed Mar. 21, 2019, the contents of which are incorporated herein by reference in their entirety). In some embodiments, the antibody moiety comprises the CDRs or variable domains (V_{H} and/or V_{L} domains) of an antibody moiety specific for PSMA (see, e.g., PCT/US2019/037534, filed Jun. 17, 2019, the contents of which are incorporated herein by reference in their entirety). In some embodiments, the antibody moiety comprises the CDRs or variable domains (V_{H} and/or V_{L} domains) of an antibody moiety specific for a WT-1 peptide/MHC complex (see, e.g., WO2012/135854, WO2015/070078, and WO2015/070061). In some embodiments, the antibody moiety comprises the CDRs or variable domains (V_{H} and/or V_{L} domains) of an antibody moiety specific for an AFP peptide/MHC complex (see, e.g., WO2016/161390). In some embodiments, the antibody moiety comprises the CDRs or variable domains (V_{H} and/or V_{L} domains) of an antibody moiety specific for a HPV16-E7 peptide/MHC complex (see, e.g., WO2016/182957). In some embodiments, the antibody moiety comprises the CDRs or variable domains (V_{H} and/or V_{L} domains) of an antibody moiety specific for a NY-ESO-1 peptide/MHC complex (see, e.g., WO2016/210365). In some embodiments, the antibody moiety comprises the CDRs or variable domains (V_{H} and/or V_{L} domains) of an antibody moiety specific for a PRAME peptide/MHC complex (see, e.g., WO2016/191246). In some embodiments, the antibody moiety comprises the CDRs or variable domains (V_{H} and/or V_{L} domains) of an antibody moiety specific for a EBV-LMP2A peptide/MHC complex (see, e.g., WO2016/201124). In some embodiments, the antibody moiety comprises the CDRs or variable domains (V_{H} and/or V_{L} domains) of an antibody moiety specific for a KRAS peptide/MHC complex (see, e.g., WO2016/154047). In some embodiments, the antibody moiety comprises the CDRs or variable domains (V_{H} and/or V_{L} domains) of an antibody moiety specific for a PSA peptide/MHC complex (see, e.g., WO2017/015634). In some embodiments, the antibody moiety comprises the CDRs or variable domains (V_{H} and/or V_{L} domains) of an antibody moiety specific for a FoxP3 peptide/MHC complex (see, e.g., PCT/US2019/018112 filed Feb. 14, 2018, the contents of which are incorporated herein by reference in their entirety). In some embodiments, the antibody moiety comprises the CDRs or variable domains (V_{H} and/or V_{L} domains) of an antibody moiety specific for a Histone H3.3 peptide/MHC complex (see, e.g., WO2018/132597). In some embodiments, the antibody moiety comprises the CDRs or variable domains (V_{H} and/or V_{L} domains) of an antibody moiety specific for a HIV-1 peptide/MHC complex (see, e.g., WO2018057967). In some embodiments, the antibody moiety is a scFv (single chain variable fragment) comprising a V_{H} domain and a V_{L} domain. In some embodiments, the scFv comprises an antigen-binding module that specifically binds to a complex comprising a peptide and an MHC protein, known as a peptide/MHC complex.

Table A lists exemplary proteins whose fragments or peptides can be targeted by the CAR and CSR. Also listed are possible diseases, specifically possible cancers (e.g., solid tumor cancers) that such T cells can treat.

TABLE A

| CAR Target (Peptide-MHC | | |
|---|---|---|
| Complex, including mutant | CSR Target | Exemplary |
| peptide, other than GPC3, | (All Cell Surface | Cancers to be |
| PSMA, and ROR1) | Protein) | Treated |
| AFP, GPC3 | GPC3 | Liver Cancer |
| KRAS | MSLN, ROR1 | Pancreatic Cancer |
| PSA, PSMA, ROR1 | PSMA, ROR1 | Prostate Cancer |
| NY-ESO-1, PRAME | ROR2 | Melanoma |
| KRAS, PRAME | ROR2 | Gastrointestinal |
| | | Cancers |
| PRAME, PSA, ROR1 | HER2, EpCAM, | Breast Cancers |
| | ROR1 | (including |
| | | Metastatic Breast |
| | | Cancer) |
| WT1, NY-ESO-1, ROR1 | MUC1, MUC16, | Ovarian Cancer |
| | FRα, ROR1. | |
| KRAS | EGFR | Colorectal Cancers |
| | | (including |
| | | Metastatic |
| | | Colorectal Cancer) |
| Histone H3.3 | EGFR, EGFRvIII | Glioblastoma |
| KRAS, NY-ESO-1, ROR1 | HER3, DLL3, | Lung Cancer |
| | c-Met, ROR1 | |
| PRAME | ROR2, CD70 | Renal Cell |
| | | Carcinoma |

Extracellular Target-Binding Domain/Ligand-Binding Module

An extracellular target-binding domain of a CAR and/or a ligand-binding module of a CSR described herein may comprise an antibody moiety or an antigen-binding fragment thereof. In certain embodiments, the extracellular target-binding domain can be a single-chain variable fragment derived from an antibody (scFv), a tandem scFv, a single-domain antibody fragment (VHHs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. The extracellular target-binding domain can be joined to the TM domain via a flexible hinge/spacer region.

scFv and Tandem scFv

An extracellular target-binding domain of a CAR and/or a ligand-binding module of a CSR described herein may comprise an antibody moiety that is a single chain Fv (scFv) antibody. An scFv antibody may comprise a light chain variable region and a heavy chain variable region, in which the light chain variable region and the heavy chain variable region may be joined using recombinant methods by a synthetic linker to make a single polypeptide chain. In some embodiments, the scFv may have the structure “(N-terminus) light chain variable region-linker-heavy chain variable region (C-terminus),” in which the heavy chain variable region is joined to the C-terminus of the light chain variable region by way of a linker. In other embodiments, the scFv may have the structure “(N-terminus) heavy chain variable region-linker-light chain variable region (C-terminus),” in which the light chain variable region is joined to the C-terminus of the heavy chain variable region by way of a linker. A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine.

An extracellular target-binding domain of a CAR and/or a ligand-binding module of a CSR may comprise an antibody moiety that is a tandem scFv comprising a first scFv and a second scFv (also referred to herein as a “tandem scFv multispecific antibody”). In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target ligand, and b) a second scFv. In some embodiments, the target ligand is CD22 and the first scFv specifically binds to an extracellular region of CD22. In some embodiments, the target ligand is CD19 and the first scFv specifically binds to an extracellular region of CD19. In some embodiments, the target ligand is an alpha-fetoprotein (AFP) peptide and the first scFv specifically binds to an extracellular region of the AFP peptide.

In some embodiments, the second scFv specifically binds to another antigen. In some embodiments, the second scFv specifically binds to an antigen on the surface of a cancer cell. In some embodiments, the second scFv specifically binds to an antigen on the surface of a cell that does not express CD22. In some embodiments, the second scFv specifically binds to an antigen on the surface of a cell that does not express CD19. In some embodiments, the second scFv specifically binds to an antigen on the surface of a cell that does not express AFP peptide. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3δ, CD3ζ, CD28, CD16a, CD56, CD68, GDS2D, OX40, GITR, CD137, CD27, CD40L and HVEM.

In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic. In some embodiments, the tandem scFv multispecific antibody further comprises at least one (such as at least about any of 2, 3, 4, 5, or more) additional scFv.

In some embodiments, there is provided a tandem scFv multispecific (e.g., bispecific) antibody comprising a) a first scFv that specifically binds to an extracellular region of a target antigen, and b) a second scFv, wherein the tandem scFv multispecific antibody is a tandem di-scFv or a tandem tri-scFv. In some embodiments, the tandem scFv multispecific antibody is a tandem di-scFv. In some embodiments, the tandem scFv multispecific antibody is a bispecific T-cell engager.

In some embodiments, the tandem di-scFv bispecific antibody binds to an extracellular region of a target antigen or a portion thereof with a Kd between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the tandem di-scFv bispecific antibody binds to an extracellular region of a target antigen or a portion thereof with a Kd between about 1 nM to about 500 nM (such as about any of 1, 10, 25, 50, 75, 100, 150, 200, 250, 300, 350, 400, 450, or 500 nM, including any ranges between these values).

A variety of technologies are known in the art for designing, constructing, and/or producing multispecific antibodies. Multispecific antibodies may be constructed that either utilize the full immunoglobulin framework (e.g., IgG), single chain variable fragment (scFv), or combinations thereof. Bispecific antibodies may be composed of two scFv units in tandem as described above. In the case of anti-tumor immunotherapy, bispecific antibodies that comprise two single chain variable fragments (scFvs) in tandem may be designed such that an scFv that binds a tumor antigen is linked with an scFv that engages T cells, i.e., by binding CD3 on the T cells. Thus, T cells are recruited to a tumor site to mediate killing of the tumor cells. Bispecific antibodies can be made, for example, by combining heavy chains and/or light chains that recognize different epitopes of the same or different antigen. In some embodiments, by molecular function, a bispecific binding agent binds one antigen (or epitope) on one of its two binding arms (one V_{H}/V_{L} pair), and binds a different antigen (or epitope) on its second arm (a different V_{H}/V_{L} pair). By this definition, a bispecific binding agent has two distinct antigen binding arms (in both specificity and CDR sequences), and is monovalent for each antigen to which it binds. In certain embodiments, a bispecific binding agent according to the present invention comprises a first and a second scFv. In some certain embodiments, a first scFv is linked to the C-terminal end of a second scFv. In some certain embodiments, a second scFv is linked to the C-terminal end of a first scFv. In some certain embodiments, scFvs are linked to each other via a linker (e.g., SRGGGGSGGGGSGGGGSLEMA (SEQ ID NO:78)). In some certain embodiments, scFvs are linked to each other without a linker.

A linker may be a polypeptide including 2 to 200 (e.g., 2, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200) amino acids. Suitable linkers may contain flexible amino acid residues such as glycine and serine. In certain embodiments, a linker may contain motifs, e.g., multiple or repeating motifs, of GS, GGS, GGGGS (SEQ ID NO:79), GGSG (SEQ ID NO:80), or SGGG (SEQ ID NO:81). In some embodiments, a linker may have the sequence GSGS (SEQ ID NO:82), GSGSGS (SEQ ID NO:83), GSGSGSGS (SEQ ID NO:84), GSGSGSGSGS (SEQ ID NO:85), GGSGGS (SEQ ID NO:86), GGSGGSGGS (SEQ ID NO:87), GGSGGSGGSGGS (SEQ ID NO:88). GGSG (SEQ ID NO:89), GGSGGGSG (SEQ ID NO:90), or GGSGGGSGGGSG (SEQ ID NO:91). In other embodiments, a linker may also contain amino acids other than glycine and serine, e.g., SRGGGGSGGGGSGGGGSLEMA (SEQ ID NO:78).

Transmembrane Domain (TM)

The transmembrane domain of the CAR and/or the CSR may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. For example, the transmembrane domain derived from CD30 lacks a binding site for the p56lck kinase, a common motif in the TNF receptor family. In some embodiments, a transmembrane region of particular use in this invention may be derived from (i.e., comprise at least the transmembrane region(s) of) CD8, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:66. In some embodiments, a transmembrane region of particular use in this invention may be derived from (i.e., comprise at least the transmembrane region(s) of) CD30, e.g., a transmembrane region comprising a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the sequence of SEQ ID NO:70.

In certain embodiments, the transmembrane domain may be chosen based on the target antigen. For example, a CAR containing an antibody moiety specific for an AFP peptide/MHC complex and a transmembrane domain derived from CD8 appeared to have better in vitro killing properties than a corresponding CAR containing a transmembrane domain derived from CD30. This result was not observed in a CAR containing an antibody moiety specific for CD19.

In some embodiments, the transmembrane domain may be synthetic, in which case it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CAR or CSR described herein. In some embodiments, the linker is a glycine-serine doublet. In some embodiments, the linker between the CAR's extracellular target binding domain and/or the CSR's ligand-binding module and the transmembrane domain comprises a partial extracellular domain (ECD) of a molecule such as the same as or a different molecule from the transmembrane domain's original molecule. For example, the linker connecting a transmembrane domain derived from or comprising CD8 or CD30 can comprise an ECD of CD8 or CD30, respectively or alternatively.

In some embodiments, the transmembrane domain that naturally is associated with one of the sequences in the intracellular signaling domain of the CAR or CSR is used. In some embodiments, the transmembrane domain can be selected or modified by amino acid substitution to avoid binding of such domains to the transmembrane domains of the same or different surface membrane proteins to minimize interactions with other members of the receptor complex.

Intracellular Signaling Domain

The intracellular signaling domain of the CAR and/or CSR is responsible for activation of at least one of the normal effector functions of the immune cell in which the CAR and CSR have been placed in. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such a truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FcRβ, CD3γ, CD3δ, CD3ε, CD3C, CD5, CD22, CD79a, CD79b, and CD66d. In some embodiments, an ITAM containing primary signaling sequence is derived from CD3C.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR of the invention. For example, the intracellular signaling domain of the CAR can comprise a CD3ζ primary intracellular signaling sequence and a costimulatory signaling sequence.

In some embodiments, the intracellular signaling domain is capable of activating an immune cell. In some embodiments, the intracellular signaling domain comprises a primary signaling sequence and a costimulatory signaling sequence. In some embodiments, the primary signaling sequence comprises a CD3ζ intracellular signaling sequence. In some embodiments, the costimulatory signaling sequence comprises a CD30 intracellular signaling sequence.

III. Multispecific Antibodies

An extracellular target-binding domain of a CAR and/or a ligand-binding module of a CSR may comprise an antibody moiety that is a multispecific antibody. A multispecific antibody may comprise a first binding moiety and a second binding moiety (such as a second antigen-binding moiety). Multispecific antibodies are antibodies that have binding specificities for at least two different antigens or epitopes (e.g., bispecific antibodies have binding specificities for two antigens or epitopes). Multispecific antibodies with more than two specificities are also contemplated. For example, trispecific antibodies can be prepared (see, e.g., Tutt et al., J. Immunol. 147: 60 (1991)). It is to be appreciated that one of skill in the art could select appropriate features of individual multispecific antibodies described herein to combine with one another to form a multispecific antibodies of the invention.

Thus, for example, in some embodiments, there is provided a multispecific (e.g., bispecific) antibody comprising a) a first binding moiety that specifically binds to an extracellular region of a first target antigen, and b) a second binding moiety (such as an antigen-binding moiety). In some embodiments, the second binding moiety specifically binds to a different target antigen. In some embodiments, the second binding moiety specifically binds to an antigen on the surface of a cell, such as a cytotoxic cell. In some embodiments, the second binding moiety specifically binds to an antigen on the surface of a lymphocyte, such as a T cell, an NK cell, a neutrophil, a monocyte, a macrophage, or a dendritic cell. In some embodiments, the second binding moiety specifically binds to an effector T cell, such as a cytotoxic T cell (also known as cytotoxic T lymphocyte (CTL) or T killer cell).

In some embodiments, the second binding moiety specifically binds to a tumor antigen. Examples of tumor antigens include, but are not limited to, alpha fetoprotein (AFP), CA15-3, CA27-29, CA19-9, CA-125, calretinin, carcinoembryonic antigen, CD34, CD99, CD117, chromogranin, cytokeratin, desmin, epithelial membrane protein (EMA), Factor VIII, CD31 FL1, glial fibrillary acidic protein (GFAP), gross cystic disease fluid protein (GCDFP-15), HMB-45, human chorionic gonadotropin (hCG), inhibin, keratin, CD45, a lymphocyte marker, MART-1 (Melan-A), Myo D1, muscle-specific actin (MSA), neurofilament, neuron-specific enolase (NSE), placental alkaline phosphatase (PLAP), prostate-specific antigen, S100 protein, smooth muscle actin (SMA), synaptophysin, thyroglobulin, thyroid transcription factor-1, tumor M2-PK, and vimentin.

In some embodiments, the second antigen-binding moiety in a bispecific antibody binds to CD3. In some embodiments, the second antigen-binding moiety specifically binds to CD3g. In some embodiments, the second antigen-binding moiety specifically binds to an agonistic epitope of CD3g. The term “agonistic epitope”, as used herein, means (a) an epitope that, upon binding of the multispecific antibody, optionally upon binding of several multispecific antibodies on the same cell, allows said multispecific antibodies to activate T cell receptor (TCR) signaling and induce T cell activation, and/or (b) an epitope that is solely composed of amino acid residues of the epsilon chain of CD3 and is accessible for binding by the multispecific antibody, when presented in its natural context on T cells (i.e., surrounded by the TCR, the CD37 chain, etc.), and/or (c) an epitope that, upon binding of the multispecific antibody, does not lead to stabilization of the spatial position of CD3F relative to CD3γ.

In some embodiments, the second antigen-binding moiety binds specifically to an antigen on the surface of an effector cell, including for example CD3γ, CD3δ, CD3ε, CD3ζ, CD28, CD16a, CD56, CD68, GDS2D, OX40, GITR, CD137, CD27, CD40L and HVEM. In other embodiments, the second antigen-binding moiety binds to a component of the complement system, such as C1q. C1q is a subunit of the C1 enzyme complex that activates the serum complement system. In other embodiments, the second antigen-binding moiety specifically binds to an Fc receptor. In some embodiments, the second antigen-binding moiety specifically binds to an Fcγ receptor (FcγR). The FcγR may be an FcγRIII present on the surface of natural killer (NK) cells or one of FcγRI, FcγRIIA, FcγRIIBI, FcγRIIB2, and FcγRIIIB present on the surface of macrophages, monocytes, neutrophils and/or dendritic cells. In some embodiments, the second antigen-binding moiety is an Fc region or functional fragment thereof. A “functional fragment” as used in this context refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. A functional Fc fragment is capable of competitively inhibiting the binding of the original, full-length Fc portion to an FcR such as the activating FcγRI. In some embodiments, a functional Fc fragment retains at least 30%, 40%, 50%, 60%, 70%, 80%, 90% or 95% of its affinity to an activating FcγR. In some embodiments, the Fc region or functional fragment thereof is an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g., FcγRIIB1/B2 (CD32B)).

In some embodiments, the multispecific antibodies allow killing of antigen-presenting target cells and/or can effectively redirect CTLs to lyse target-presenting target cells. In some embodiments, the multispecific (e.g., bispecific) antibodies of the present invention show an in vitro EC50 ranging from 10 to 500 ng/ml, and is able to induce redirected lysis of about 50% of the target cells through CTLs at a ratio of CTLs to target cells of from about 1:1 to about 50:1 (such as from about 1:1 to about 15:1, or from about 2:1 to about 10:1).

In some embodiments, the multispecific (e.g., bispecific) antibody is capable of cross-linking a stimulated or unstimulated CTL and the target cell in such a way that the target cell is lysed. This offers the advantage that no generation of target-specific T cell clones or common antigen presentation by dendritic cells is required for the multispecific antibody to exert its desired activity. In some embodiments, the multispecific antibody of the present invention is capable of redirecting CTLs to lyse the target cells in the absence of other activating signals. In some embodiments, the second antigen-binding moiety specifically binds to CD3 (e.g., specifically binds to CD3ε), and signaling through CD28 and/or IL-2 is not required for redirecting CTLs to lyse the target cells.

Methods for measuring the preference of the multispecific antibody to simultaneously bind to two antigens (e.g., antigens on two different cells) are within the normal capabilities of a person skilled in the art. For example, when the second binding moiety specifically binds to the second antigen, the multispecific antibody may be contacted with a mixture of first antigen⁺/second antigen⁻ cells and first antigen⁻/second antigen⁺ cells. The number of multispecific antibody-positive single cells and the number of cells cross-linked by multispecific antibodies may then be assessed by microscopy or fluorescence-activated cell sorting (FACS) as known in the art.

In some embodiments, the multispecific antibody is, for example, a diabody (db), a single-chain diabody (scDb), a tandem scDb (Tandab), a linear dimeric scDb (LD-scDb), a circular dimeric scDb (CD-scDb), a di-diabody, a tandem scFv, a tandem di-scFv (e.g., a bispecific T cell engager), a tandem tri-scFv, a tri(a)body, a bispecific Fab2, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fc, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a single-domain antibody fragment (VHHs or sdAbs), a single domain bispecific antibody (BsAbs), an intrabody, a nanobody, an immunokine in a single chain format, a heteromultimeric antibody, or a heteroconjugate antibody. In some embodiments, the multispecific antibody is a single chain antibody fragment. In some embodiments, the multispecific antibody is a tandem scFv (e.g., a tandem di-scFv, such as a bispecific T cell engager).

IV. Antibody-Drug Conjugates

In some embodiments, there is provided an immunoconjugate comprising an antibody moiety and a therapeutic agent (also referred to herein as an “antibody-drug conjugate”, or “ADC”). In some embodiments, therapeutic agent is a toxin that is either cytotoxic, cytostatic, or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos, Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986):603-605 (1986); Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought thereby.

Therapeutic agents used in immunoconjugates (e.g., an ADC) include, for example, daunomycin, doxorubicin, methotrexate, and vindesine (Rowland et al., Cancer Immunol. Immunother. 21:183-187 (1986)). Toxins used in immunoconjugates include bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Immunoconjugates (e.g., an ADC) of an antibody moiety and one or more small molecule toxins, such as a calicheamicin, maytansinoids, dolastatins, aurostatins, a trichothecene, and CC1065, and the derivatives of these toxins that have toxin activity, are also contemplated herein.

In some embodiments, there is provided an immunoconjugate (e.g., an ADC) comprising a therapeutic agent that has an intracellular activity. In some embodiments, the immunoconjugate is internalized and therapeutic agent is a cytotoxin that blocks the protein synthesis of the cell, therein leading to cell death. In some embodiments, therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where therapeutic agent is a cytotoxin comprising a polypeptide having a ribosome-inactivating activity, the immunoconjugate must be internalized upon binding to the target cell in order for the protein to be cytotoxic to the cells.

In some embodiments, there is provided an immunoconjugate (e.g., an ADC) comprising a therapeutic agent that acts to disrupt DNA. In some embodiments, therapeutic agent that acts to disrupt DNA is, for example, selected from the group consisting of enediyne (e.g., calicheamicin and esperamicin) and non-enediyne small molecule agents (e.g., bleomycin, methidiumpropyl-EDTA-Fe(II)).

The present invention further contemplates an immunoconjugate (e.g., an ADC) formed between the antibody moiety and a compound with nucleolytic activity (e.g., a ribonuclease or a DNA endonuclease such as a deoxyribonuclease; DNase).

In some embodiments, the immunoconjugate comprises an agent that acts to disrupt tubulin. Such agents may include, for example, rhizoxin/maytansine, paclitaxel, vincristine and vinblastine, colchicine, auristatin dolastatin 10 MMAE, and peloruside A.

In some embodiments, the immunoconjugate (e.g., an ADC) comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the immunoconjugate (e.g., an ADC) comprises a highly radioactive atom. A variety of radioactive isotopes are available for the production of radioconjugated antibodies. Examples include 211At, 131I, 125I, 90Y, 186Re, 188Re, 153Sm, 212Bi, 32P, 212Pb and radioactive isotopes of Lu.

In some embodiments, the antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, an immunoconjugate (e.g., an ADC) may comprise an antibody moiety conjugated to a prodrug-activating enzyme. In some such embodiments, a prodrug-activating enzyme converts a prodrug to an active drug, such as an anti-viral drug. Such immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; proteases, such as Serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, therapeutic portion of the immunoconjugates (e.g., an ADC) may be a nucleic acid. Nucleic acids that may be used include, but are not limited to, antisense RNA, genes or other polynucleotides, including nucleic acid analogs such as thioguanine and thiopurine.

The present application further provides immunoconjugates (e.g., an ADC) comprising an antibody moiety attached to an effector molecule, wherein the effector molecule is a label, which can generate a detectable signal, indirectly or directly. These immunoconjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example, 99Tc or 123I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

In some embodiments, the immunoconjugate is detectable indirectly. For example, a secondary antibody that is specific for the immunoconjugate and contains a detectable label can be used to detect the immunoconjugate.

V. Immune Cells

The present invention provides immune cells comprising: a chimeric antigen receptor (CAR) that comprises (i) an extracellular target-binding domain comprising an antibody moiety; (ii) a transmembrane domain; and (iii) a primary signaling domain, and a chimeric stimulating receptor (CSR) that comprises (i) a ligand-binding module that is capable of binding or interacting with a target ligand; (ii) a transmembrane domain; and (iii) a CD30 costimulatory domain, in which the CSR in the immune cells lacks a functional primary signaling domain. In some embodiments, the immune cell comprises one or more nucleic acids encoding the CAR and CSR, wherein the CAR and CSR are expressed from the nucleic acid and localized to the immune cell surface. In some embodiments, the immune cell is a T cell. In some embodiments, the immune cell is selected from the group consisting of a cytotoxic T cell, a helper T cell, a natural killer T cell, and a suppressor T cell. In some embodiments, the immune cell is modified to block or decrease the expression of one or more of the endogenous TCR subunits of the immune cell. For example, in some embodiments, the immune cell is an αβ T cell modified to block or decrease the expression of the TCR α and/or β chains or the immune cell is a γδ T cell modified to block or decrease the expression of the TCR γ and/or δ chains. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In exemplary embodiments, the cell of the present disclosure is an immune cell or a cell of the immune system. Accordingly, the cell may be a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In exemplary aspects, the cell is a T lymphocyte. In exemplary aspects, the T lymphocyte is CD8⁺, CD4⁺, CD8⁺/CD4⁺, or a T-regulatory (T-reg) cell. In exemplary embodiments, the T lymphocyte is genetically engineered to silence the expression of an endogenous TCR. In exemplary aspects, the cell is a natural killer (NK) cell.

For example, in some embodiments, there is provided an immune cell (such as a T cell) comprising one or more nucleic acids encoding a CAR and a CSR according to any of the CARs and CSRs described herein, wherein the CAR and CSR are expressed from the nucleic acid and localized to the immune cell surface. In some embodiments, the nucleic acid sequence is contained in a vector. Vectors may be selected, for example, from the group consisting of mammalian expression vectors and viral vectors (such as those derived from retroviruses, adenoviruses, adeno-associated viruses, herpes viruses, and lentiviruses). In some embodiments, one or more of the vectors is integrated into the host genome of the immune cell. In some embodiments, the nucleic acid sequence is under the control of a promoter. In some embodiments, the promoter is inducible. In some embodiments, the promoter is operably linked to the 5′ end of the nucleic acid sequence. In some embodiments, the immune cell is selected from the group consisting of a cytotoxic T cell, a helper T cell, a natural killer T cell, and a suppressor T cell.

Thus, in some embodiments, there is provided a immune cell (such as a T cell) expressing on its surface a CAR and CSR described herein, wherein the immune cell comprises: a nucleic acid sequence encoding a CAR polypeptide chain of the CAR and a CSR polypeptide chain of the CSR, wherein the CAR polypeptide chain and the CSR polypeptide chain are expressed from the nucleic acid sequence as a single polypeptide chain. The single polypeptide chain is then cleaved to form a CAR polypeptide chain and a CSR polypeptide chain, and the CAR polypeptide chain and the CSR polypeptide chain localize to the surface of the immune cell.

In other embodiments, there is provided a immune cell (such as a T cell) expressing on its surface a CAR and CSR described herein, wherein the immune cell comprises: a CAR nucleic acid sequence encoding a CAR polypeptide chain of the CAR, and a CSR nucleic acid sequence encoding a CSR polypeptide chain of the CSR, wherein the CAR polypeptide chain is expressed from the CAR nucleic acid sequence to form the CAR, wherein the CSR polypeptide chain is expressed from the CSR nucleic acid sequence to form the CSR, and wherein the CAR and CSR localize to the surface of the immune cell.

VI. Fc Variants

In some embodiments, CARs and/or CSRs described herein may comprise a variant Fc region, wherein the variant Fc region may comprise at least one amino acid modification relative to a reference Fc region (or parental Fc region or a wild-type Fc region). Amino acid modifications may be made in an Fc region to alter effector function and/or to increase serum stability of the CAR and/or CSR. CARs and/or CSRs comprising variant Fc regions may demonstrate an altered affinity for an Fc receptor (e.g., an FcγR), provided that the variant Fc regions do not have a substitution at positions that make a direct contact with Fc receptor based on crystallographic and structural analysis of Fc-Fc receptor interactions such as those disclosed by Sondermann et al., 2000, Nature, 406:267-273. Examples of positions within the Fc region that make a direct contact with an Fc receptor such as an FcγR are amino acids 234-239 (hinge region), amino acids 265-269 (B/C loop), amino acids 297-299 (C′/E loop), and amino acids 327-332 (F/G) loop. In some embodiments, CARs and/or CSRs comprising variant Fc regions may comprise a modification of at least one residue that makes a direct contact with an FcγR based on structural and crystallographic analysis.

Amino acid modifications in Fc regions to create variant Fc regions that, e.g., alter affinity for activating and/or inhibitory receptors, lead to improved effector function such as, e.g., Antibody-Dependent Cell-Mediated Cytotoxicity (ADCC) and Complement Dependent Cytotoxicity (CDC), increase binding affinity for C1q, reduce or eliminate FcR binding, increase half-life are known in the art (see, e.g., U.S. Pat. Nos. 9,051,373, 9,040,041, 8,937,158, 8,883,973, 8,883,147, 8,858,937, 8,852,586, 8,809,503, 8,802,823, 8,802,820, 8,795,661, 8,753,629, 8,753,628, 8,735,547, 8,735,545, 8,734,791, 8,697,396, 8,546,543, 8,475,792, 8,399,618, 8,394,925, 8,388,955, 8,383,109, 8,367,805, 8,362,210, 8,338,574, 8,324,351, 8,318,907, 8,188,231, 8,124,731, 8,101,720, 8,093,359, 8,093,357, 8,088,376, 8,084,582, 8,039,592, 8,012,476, 7,799,900, 7,790,858, 7,785,791, 7,741,072, 7,704,497, 7,662,925, 7,416,727, 7,371,826, 7,364,731, 7,335,742, 7,332,581, 7,317,091, 7,297,775, 7,122,637, 7,083,784, 6,737,056, 6,538,124, 6,528,624 and 6,194,551).

In some embodiments, a variant Fc region may have different glycosylation patterns as compared to a parent Fc region (e.g., aglycosylated). In some embodiments, different glycosylation patterns may arise from expression in different cell lines, e.g., an engineered cell line.

CARs and/or CSRs described herein may comprise variant Fc regions that bind with a greater affinity to one or more FcγRs. Such CARs and/or CSRs preferably mediate effector function more effectively as discussed infra. In some embodiments, CARs and/or CSRs described herein may comprise variant Fc regions that bind with a weaker affinity to one or more FcγRs. Reduction or elimination of effector function may be desirable in certain cases, for example, in the case of CARs and/or CSRs whose mechanism of action involves blocking or antagonism but not killing of the cells bearing a target antigen. In some embodiments, increased effector function may be directed to tumor cells and cells expressing foreign antigens.

VII. Nucleic Acids

Nucleic acid molecules encoding the CARs and CSRs described herein are also contemplated. In some embodiments, according to any of the CARs and CSRs described herein, there is provided a nucleic acid (or a set of nucleic acids) encoding the CARs and CSRs.

The present invention also provides vectors in which a nucleic acid of the present invention is inserted.

In brief summary, the expression of a CAR and/or CSR described herein by a nucleic acid encoding the CAR and/or CSR can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405.

An exemplary inducible promoter system for use in the present invention is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli. (See, Brown et al., Cell 49:603-612 (1987). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

Another exemplary inducible promoter system for use in the present invention is the nuclear-factor of the activated T-cell (NFAT) system. The NFAT family of transcription factors are important regulators of T cell activation. NFAT response elements are found, for example, in the IL-2 promoter (see for example Durand, D. et. al., Molec. Cell. Biol. 8, 1715-1724 (1988); Clipstone, N A, Crabtree, G R. Nature. 1992 357(6380): 695-7; Chmielewski, M., et al. Cancer research 71.17 (2011): 5697-5706; and Zhang, L., et al. Molecular therapy 19.4 (2011): 751-759). In some embodiments, an inducible promoter described herein comprises one or more (such as 2, 3, 4, 5, 6, or more) NFAT response elements. In some embodiments, the inducible promoter comprises 6 NFAT response elements, for example, comprising the nucleotide sequence of SEQ ID NO:112. In some embodiments, an inducible promoter described herein comprises one or more (such as 2, 3, 4, 5, 6, or more) NFAT response elements linked to a minimal promoter, such as a minimal TA promoter. In some embodiments, the minimal TA promoter comprises the nucleotide sequence of SEQ ID NO:113. In some embodiments, the inducible promoter comprises the nucleotide sequence of SEQ ID NO: 114.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a CAR and/or CSR according to any of the CARs and CSRs described herein. In some embodiments, the nucleic acid comprises one or more nucleic acid sequences encoding all of the polypeptide chains of the CAR. In some embodiments, the nucleic acid comprises one or more nucleic acid sequences encoding all of the polypeptide chains of the CSR. In some embodiments, the nucleic acid comprises one or more nucleic acid sequences encoding all of the polypeptide chains of the CAR and the CSR. In some embodiments, each of the one or more nucleic acid sequences is contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

For example, in some embodiments, the CAR is a monomer comprising a single CAR polypeptide chain and the CSR is a monomer comprising a single CSR polypeptide chain, and the nucleic acid comprises a first nucleic acid sequence encoding the CAR polypeptide chain, and a second nucleic acid sequence encoding the CSR polypeptide chain. In some embodiments, the first nucleic acid sequence is contained in a first vector and the second nucleic acid sequence is contained in a second vector. In some embodiments, the first and second nucleic acid sequences are contained in one vector. In some embodiments, the first nucleic acid sequence is under the control of a first promoter, and the second nucleic acid sequence is under the control of a second promoter. In some embodiments, the first and second promoters have the same sequence. In some embodiments, the first and second nucleic acid sequences are expressed as a single transcript under the control of a single promoter in a multicistronic vector. See for example Kim, J H, et al., PLoS One 6(4):e18556, 2011. In some embodiments, one or more of the promoters are inducible. In some embodiments, the nucleic acid sequence encoding the CSR polypeptide chain is operably linked to an inducible promoter. In some embodiments, the inducible promoter comprises one or more elements responsive to immune cell activation, such as NFAT response elements.

In some embodiments, the first and second nucleic acid sequences have similar (such as substantially or about the same) expression levels in a host cell (such as a T cell). In some embodiments, the first and second nucleic acid sequences have expression levels in a host cell (such as a T cell) that differ by at least about two (such as at least about any of 2, 3, 4, 5, or more) times. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

Thus, in some embodiments, there is provided a nucleic acid encoding a) a CAR polypeptide chain according to any of the CARs described herein; and b) a CSR polypeptide chain according to any of the CSRs described herein. In some embodiments, the nucleic acid sequence is contained in a vector (such as a lentiviral vector). In some embodiments, the portion of the nucleic acid encoding the CAR polypeptide chain is under the control of a first promoter, and the portion of the nucleic acid encoding the CSR polypeptide chain is under the control of a second promoter. In some embodiments, the first promoter is operably linked to the 5′ end of the CAR nucleic acid sequence. In some embodiments, the second promoter is operably linked to the 5′ end of the CSR nucleic acid sequence. In some embodiments, only one promoter is used. In some embodiments, there is nucleic acid linker selected from the group consisting of an internal ribosomal entry site (IRES) and a nucleic acid encoding a self-cleaving 2A peptide (such as P2A, T2A, E2A, or F2A) linking the 3′ end of the CAR nucleic acid sequence to the 5′ end of the CSR nucleic acid sequence, or the 5′ end of the promoter that is linked to the CSR, if the promoter specific to the CAR is present. In some embodiments, there is nucleic acid linker selected from the group consisting of an internal ribosomal entry site (IRES) and a nucleic acid encoding a self-cleaving 2A peptide (such as P2A, T2A, E2A, or F2A) linking the 3′ end of the CSR nucleic acid sequence to the 5′ end of the CAR nucleic acid sequence, or the 5′ end of the promoter that is linked to the CAR, if the promoter specific to the CAR is present. In some embodiments, the CAR nucleic acid sequence and the CSR nucleic acid sequence are transcribed as a single RNA under the control of one promoter.

Thus, in some embodiments, there is provided two nucleic acids, wherein a first nucleic acid encodes a CAR polypeptide chain according to any of the CARs described herein; and a second nucleic acid encodes a CSR polypeptide chain according to any of the CSRs described herein. In some embodiments, the two nucleic acids are contained in two vectors (such as lentiviral vectors).

In some embodiments, the first and/or second promoters are inducible. In some embodiments, the first and/or second vectors are viral vectors (such as lentiviral vectors). It is to be appreciated that embodiments where any of the nucleic acid sequences are swapped are also contemplated, such as where the CAR nucleic acid sequence is swapped with the CSR nucleic acid sequence.

VII. CAR and CSR Production

Provided CARs and/or CSRs or portions thereof, or nucleic acids encoding them, may be produced by any available means. Methods for production are well-known in the art. Technologies for generating antibodies (e.g., scFv antibodies, monoclonal antibodies, and/or polyclonal antibodies) are available in the art. It will be appreciated that a wide range of animal species can be used for the production of antisera, e.g., mouse, rat, rabbit, pig, cow, deer, sheep, goat, cat, dog, monkey, and chicken. The choice of animal may be decided upon the ease of manipulation, costs or the desired amount of sera, as would be known to one of skill in the art. It will be appreciated that antibodies can also be produced transgenically through the generation of a mammal or plant that is transgenic for the immunoglobulin heavy and light chain sequences of interest (e.g., a transgenic rodent transgenic for human immunoglobulin heavy and light chain genes). In connection with the transgenic production in mammals, antibodies can be produced in, and recovered from, the milk of goats, cows, or other mammals (see, e.g., U.S. Pat. Nos. 5,827,690, 5,756,687, 5,750,172, and 5,741,957; herein incorporated by reference in their entireties). Alternatively, antibodies may be made in chickens, producing IgY molecules (Schade et al., 1996, ALTEX 13(5):80-85).

Although embodiments employing CARs and/or CSRs that contain human antibodies having, i.e., human heavy and light chain variable region sequences including human CDR sequences, are extensively discussed herein, the present invention also provides CARs and/or CSRs that contain non-human antibodies. In some embodiments, non-human antibodies comprise human CDR sequences from an antibody as described herein and non-human framework sequences. Non-human framework sequences include, in some embodiments, any sequence that can be used for generating synthetic heavy and/or light chain variable regions using one or more human CDR sequences as described herein, including, e.g., sequences generated from mouse, rat, rabbit, pig, cow, deer, sheep, goat, cat, dog, monkey, chicken, etc. In some embodiments, a provided CAR or CSR includes an antibody generated by grafting one or more human CDR sequences as described herein onto a non-human framework sequence (e.g., a mouse or chicken framework sequence). In many embodiments, provided CARs or CSRs comprise or are human antibodies (e.g., a human monoclonal antibody or fragment thereof, human antigen-binding protein or polypeptide, human multispecific antibody (e.g., a human bispecific antibody), a human polypeptide having one or more structural components of a human immunoglobulin polypeptide).

In some embodiments, antibodies suitable for the present invention are subhuman primate antibodies. For example, general techniques for raising therapeutically useful antibodies in baboons may be found, for example, in International Patent Application Publication No. 1991/11465 and in Losman et al., 1990, Int. J. Cancer 46:310. In some embodiments, antibodies (e.g., monoclonal antibodies) may be prepared using hybridoma methods (Milstein and Cuello, 1983, Nature 305(5934):537-40). In some embodiments, antibodies (e.g., monoclonal antibodies) may also be made by recombinant methods (see, e.g., U.S. Pat. No. 4,166,452).

Many of the difficulties associated with generating antibodies by B-cell immortalization can be overcome by engineering and expressing CAR or CSR components in E. coli or yeast using phage display. To ensure the recovery of high affinity antibodies a combinatorial immunoglobulin library must typically contain a large repertoire size. A typical strategy utilizes mRNA obtained from lymphocytes or spleen cells of immunized mice to synthesize cDNA using reverse transcriptase. The heavy and light chain genes are amplified separately by PCR and ligated into phage cloning vectors. Two different libraries may be produced, one containing the heavy chain genes and one containing the light chain genes. The libraries can be naïve or they can be semi-synthetic, i.e., with all amino acids (with the exception of cysteine) equally likely to be present at any given position in a CDR. Phage DNA is isolated from each library, and the heavy and light chain sequences are ligated together and packaged to form a combinatorial library. Each phage contains a random pair of heavy and light chain cDNAs and upon infection of E. coli directs the expression of the polypeptides in a CAR or CSR in infected cells. To identify a CAR or CSR that recognizes the antigen of interest, the phage library is plated, and the CAR or CSR molecules present in the plaques are transferred to filters. The filters are incubated with radioactively labeled antigen and then washed to remove excess unbound ligand. A radioactive spot on the autoradiogram identifies a plaque that contains a CAR or CSR that binds the antigen. Alternatively, identification of a CAR or CSR that recognizes the antigen of interest may be achieved by iterative binding of phage to the antigen, which is bound to a solid support, for example, beads or mammalian cells followed by removal of non-bound phage and by elution of specifically bound phage. In such embodiments, antigens are first biotinylated for immobilization to, for example, streptavidin-conjugated Dynabeads M-280. The phage library is incubated with the cells, beads or other solid support and non-binding phage is removed by washing. CAR or CSR phage clones that bind the antigen of interest are selected and tested for further characterization.

Once selected, positive clones may be tested for their binding to the antigen of interest expressed on the surface of live cells by flow cytometry. Briefly, phage clones may be incubated with cells (e.g., engineered to express the antigen of interest, or those that naturally express the antigen) that either do or do not express the antigen. The cells may be washed and then labeled with a mouse anti-M13 coat protein monoclonal antibody. Cells may be washed again and labeled with a fluorescent-conjugated secondary antibody (e.g., FITC-goat (Fab)₂ anti-mouse IgG) prior to flow cytometry. Cloning and expression vectors that are useful for producing a human immunoglobulin phage library can be obtained, for example, from Stratagene Cloning Systems (La Jolla, Calif.).

A similar strategy may be employed to obtain high-affinity scFv clones. A library with a large repertoire may be constructed by isolating V-genes from non-immunized human donors using PCR primers corresponding to all known V_{H}, Vκ and Vλ gene families. Following amplification, the Vκ and Vλ pools may be combined to form one pool. These fragments may be ligated into a phagemid vector. An scFv linker (e.g., (G₄S)n) may be ligated into the phagemid upstream of the V_{L} fragment (or upstream of the V_{H} fragment as so desired). The V_{H} and linker-V_{L} fragments (or V_{L} and linker-V_{H} fragments) may be amplified and assembled on the JH region. The resulting V_{H}-linker-V_{L} (or V_{L}-linker-V_{H}) fragments may be ligated into a phagemid vector. The phagemid library may be panned using filters, as described above, or using immunotubes (Nunc; Maxisorp). Similar results may be achieved by constructing a combinatorial immunoglobulin library from lymphocytes or spleen cells of immunized rabbits and by expressing the scFv in P. pastoris (see, e.g., Ridder et al., 1995, Biotechnology, 13:255-260). Additionally, following isolation of appropriate scFv antibodies, higher binding affinities and slower dissociation rates may be obtained through affinity maturation processes such as mutagenesis and chain-shuffling (see, e.g., Jackson et al., 1998, Br. J. Cancer, 78:181-188); Osbourn et al., 1996, Immunotechnology, 2:181-196).

Human antibodies may be produced using various techniques, i.e., introducing human Ig genes into transgenic animals in which the endogenous Ig genes have been partially or completely inactivated can be exploited to synthesize human antibodies. In some embodiments, human antibodies may be made by immunization of non-human animals engineered to make human antibodies in response to antigen challenge with human antigen.

Provided CARs and CSRs may be also produced, for example, by utilizing a host cell system engineered to express a CAR- or CSR-encoding nucleic acid. Alternatively or additionally, provided CARs may be partially or fully prepared by chemical synthesis (e.g., using an automated peptide synthesizer or gene synthesis of CAR- or CSR-encoding nucleic acids). CARs and/or CSRs described herein may be expressed using any appropriate vector or expression cassette. A variety of vectors (e.g., viral vectors) and expression cassettes are known in the art and cells into which such vectors or expression cassettes may be introduced may be cultured as known in the art (e.g., using continuous or fed-batch culture systems). In some embodiments, cells may be genetically engineered; technologies for genetically engineering cells to express engineered polypeptides are well known in the art (see, e.g., Ausabel et al., eds., 1990, Current Protocols in Molecular Biology (Wiley, New York)).

CARs and/or CSRs described herein may be purified, i.e., using filtration, centrifugation, and/or a variety of chromatographic technologies such as HPLC or affinity chromatography. In some embodiments, fragments of provided CARs and/or CSRs are obtained by methods that include digestion with enzymes, such as pepsin or papain, and/or by cleavage of disulfide bonds by chemical reduction.

It will be appreciated that provided CARs and/or CSRs may be engineered, produced, and/or purified in such a way as to improve characteristics and/or activity of the CARs and/or CSRs. For example, improved characteristics include, but are not limited to, increased stability, improved binding affinity and/or avidity, increased binding specificity, increased production, decreased aggregation, decreased nonspecific binding, among others. In some embodiments, provided CARs and/or CSRs may comprise one or more amino acid substitutions (e.g., in a framework region in the context of an immunoglobulin or fragment thereof (e.g., an scFv antibody)) that improve protein stability, antigen binding, expression level, or provides a site or location for conjugation of a therapeutic, diagnostic or detection agent.

Purification Tag

In some embodiments, a purification tag may be joined to a CAR and/or CSR described herein. A purification tag refers to a peptide of any length that can be used for purification, isolation, or identification of a polypeptide. A purification tag may be joined to a polypeptide (e.g., joined to the N- or C-terminus of the polypeptide) to aid in purifying the polypeptide and/or isolating the polypeptide from, e.g., a cell lysate mixture. In some embodiments, the purification tag binds to another moiety that has a specific affinity for the purification tag. In some embodiments, such moieties which specifically bind to the purification tag are attached to a solid support, such as a matrix, a resin, or agarose beads. Examples of a purification tag that may be joined to a CAR or CSR include, but are not limited to, a hexa-histidine peptide, a hemagglutinin (HA) peptide, a FLAG peptide, and a myc peptide. In some embodiments, two or more purification tags may be joined to a CAR or CSR, e.g., a hexa-histidine peptide and a HA peptide. A hexa-histidine peptide (HHHHHH (SEQ ID NO:93)) binds to nickel-functionalized agarose affinity column with micromolar affinity. In some embodiments, an HA peptide includes the sequence YPYDVPDYA (SEQ ID NO:94) or YPYDVPDYAS (SEQ ID NO:95). In some embodiments, an HA peptide includes integer multiples of the sequence YPYDVPDYA (SEQ ID NO:94) or YPYDVPDYAS (SEQ ID NO:95) in tandem series, e.g., 3xYPYDVPDYA or 3xYPYDVPDYAS. In some embodiments, a FLAG peptide includes the sequence DYKDDDDK (SEQ ID NO:96). In some embodiments, a FLAG peptide includes integer multiples of the sequence DYKDDDDK (SEQ ID NO:96) in tandem series, e.g., 3xDYKDDDDK. In some embodiments, a myc peptide includes the sequence EQKLISEEDL (SEQ ID NO:97). In some embodiments, a myc peptide includes integer multiples of the sequence EQKLISEEDL in tandem series, e.g., 3xEQKLISEEDL.

IX. Therapeutic and Detection Agents

A therapeutic agent or a detection agent may be attached to a CAR or CSR described herein. Therapeutic agents may be any class of chemical entity including, for example, but not limited to, proteins, carbohydrates, lipids, nucleic acids, small organic molecules, non-biological polymers, metals, ions, radioisotopes, etc. In some embodiments, therapeutic agents for use in accordance with the present invention may have a biological activity relevant to the treatment of one or more symptoms or causes of cancer. In some embodiments, therapeutic agents for use in accordance with the present invention may have a biological activity relevant to modulation of the immune system and/or enhancement of T-cell mediated cytotoxicity. In some embodiments, therapeutic agents for use in accordance with the present invention have one or more other activities.

A detection agent may comprise any moiety that may be detected using an assay, for example due to its specific functional properties and/or chemical characteristics. Non-limiting examples of such agents include enzymes, radiolabels, haptens, fluorescent labels, phosphorescent molecules, chemiluminescent molecules, chromophores, luminescent molecules, photoaffinity molecules, colored particles or ligands, such as biotin.

Many detection agents are known in the art, as are systems for their attachment to proteins and peptides (see, for e.g., U.S. Pat. Nos. 5,021,236; 4,938,948; and 4,472,509). Examples of such detection agents include paramagnetic ions, radioactive isotopes, fluorochromes, NMR-detectable substances, X-ray imaging agents, among others. For example, in some embodiments, a paramagnetic ion is one or more of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), and/or bismuth (III).

The radioactive isotope may be one or more of actinium-225, astatine-211, bismuth-212, carbon-14, chromium-51, chlorine-36, cobalt-57, cobalt-58, copper-67, Europium-152, gallium-67, hydrogen-3, iodine-123, iodine-124, iodine-125, iodine-131, indium-111, iron-59, lead-212, lutetium-177, phosphorus-32, radium-223, radium-224, rhenium-186, rhenium-188, selenium-75, sulphur-35, technicium-99m, thorium-227, yttrium-90, and zirconium-89. Radioactively labeled CARs or CSRs may be produced according to well-known technologies in the art.

A fluorescent label may be or may comprise one or more of Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

X. Methods of Treatment

The compositions of the invention can be administered to individuals (e.g., mammals such as humans) to treat diseases including viral infections and cancers (e.g., a hematological cancer or a solid tumor cancer).

Cancers that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated include, but are not limited to, carcinoma, blastoma, sarcoma, melanoma, neuroendocrine tumors, and glioma, and certain leukemia or lymphoid malignancies, benign and malignant tumors, and malignancies e.g., sarcomas, carcinomas, melanomas, and gliomas. Adult tumors/cancers and pediatric tumors/cancers are also included.

Solid tumors contemplated for treatment by any of the methods described herein include CNS tumors, such as glioma (e.g., brainstem glioma and mixed gliomas), glioblastoma (also known as glioblastoma multiforme), astrocytoma (such as high-grade astrocytoma), pediatric glioma or glioblastoma (such as pediatric high-grade glioma (HGG) and diffuse intrinsic pontine glioma (DIPG)), CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In some embodiments, the cancer is pediatric glioma. In some embodiments, the pediatric glioma is a low-grade glioma. In some embodiments, the pediatric glioma is a high-grade glioma (HGG). In some embodiments, the pediatric glioma is glioblastoma multiforme. In some embodiments, the pediatric glioma is diffuse intrinsic pontine glioma (DIPG). In some embodiments, the DIPG is grade II. In some embodiments, the DIPG is grade III. In some embodiments, the DIPG is grade IV.

Additional solid tumors contemplated for treatment include fibrosarcoma, myxosarcoma, liposarcoma, chondrosarcoma (such as clear-cell chondrosarcoma), chondroblastoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, melanoma, cancer of the uterus (e.g., endometrial carcinoma), and urothelial cancers (e.g., squamous cell carcinoma, transitional cell carcinoma, adenocarcinoma, ureter cancer, and urinary bladder cancer).

Hematologic cancers contemplated for treatment by any of the methods described herein include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of other cancers include, without limitation, acute lymphoblastic leukemia (ALL), Hodgkin's lymphoma, non-Hodgkin's lymphoma, B cell chronic lymphocytic leukemia (CLL), multiple myeloma, follicular lymphoma, mantle cell lymphoma, pro-lymphocytic leukemia, hairy cell leukemia, common acute lymphocytic leukemia, and null-acute lymphoblastic leukemia.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

In some embodiments of any of the methods for treating cancer (e.g., a hematological cancer or a solid tumor cancer), the CAR and CSR are conjugated to a cell (such as an immune cell, e.g., a T cell) prior to being administered to the individual. Thus, for example, there is provided a method of treating cancer (e.g., a hematological cancer or a solid tumor cancer) in an individual comprising a) conjugating a CAR and CSR described herein or an antibody moiety thereof to a cell (such as an immune cell, e.g., a T cell) to form a CAR+CSR/cell conjugate, and b) administering to the individual an effective amount of a composition comprising the CAR+CSR/cell conjugate. In some embodiments, the cell is derived from the individual. In some embodiments, the cell is not derived from the individual. In some embodiments, the CAR and CSR are conjugated to the cell by covalent linkage to a molecule on the surface of the cell. In some embodiments, the CAR and CSR are conjugated to the cell by non-covalent linkage to a molecule on the surface of the cell. In some embodiments, the CAR and CSR are conjugated to the cell by insertion of a portion of the CAR and a portion of the CSR into the outer membrane of the cell.

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

In some embodiments, the efficacy of treatment may be measured as the percentage tumor growth inhibition (% TGI), which may be calculated using the equation 100−(T/C×100), where T is the mean relative tumor volume of the treated tumor, and C is the mean relative tumor volume of a non-treated tumor. In some embodiments, the % TGI is about 2%, about 4%, about 6, about 8%, 10%, about 20%, about 30%, about 40%, about 50%, about 60%, about 70%, about 80%, about 90%, about 91%, about 92%, about 93%, about 94%, about 95%, or more than 95%.

XI. Preparation of CAR Plus CSR Immune Cells

The present invention in one aspect provides immune cells (such as lymphocytes, for example T cells) expressing a CAR and a CSR according to any of the embodiments described herein. Exemplary methods of preparing immune cells (such as T cells) expressing a CAR and a CSR (CAR plus CSR immune cells, such as CAR plus CSR T cells) are provided herein.

In some embodiments, a CAR plus CSR immune cell (such as a CAR plus CSR T cell) can be generated by introducing one or more nucleic acids (including for example a lentiviral vector) encoding a CAR (such as any of the CARs described herein) that specifically binds to a target antigen (such as a disease-associated antigen) and a CSR (such as any of the CSRs described herein) that specifically binds to a target ligand into the immune cell. The introduction of the one or more nucleic acids into the immune cell can be accomplished using techniques known in the art, such as those described herein for Nucleic Acids. In some embodiments, the CAR plus CSR immune cells (such as CAR plus CSR T cells) of the invention are able to replicate in vivo, resulting in long-term persistence that can lead to sustained control of a disease associated with expression of the target antigen (such as cancer or viral infection).

In some embodiments, the invention relates to administering a genetically modified T cell expressing a CAR that specifically binds to a target antigen according to any of the CARs described herein and a CSR that specifically binds to a target ligand according to any of the CSRs described herein for the treatment of a patient having or at risk of developing a disease and/or disorder associated with expression of the target antigen (also referred to herein as a “target antigen-positive” or “TA-positive” disease or disorder), including, for example, cancer or viral infection, using lymphocyte infusion. In some embodiments, autologous lymphocyte infusion is used in the treatment. Autologous PBMCs are collected from a patient in need of treatment and T cells are activated and expanded using the methods described herein and known in the art and then infused back into the patient.

In some embodiments, there is provided a T cell expressing a CAR that specifically binds to a target antigen according to any of the CARs described herein and a CSR that specifically binds to a target ligand according to any of the CSRs described herein (also referred to herein as an “CAR plus CSR T cell”). The CAR plus CSR T cells of the invention can undergo robust in vivo T cell expansion and can establish target antigen-specific memory cells that persist at high levels for an extended amount of time in blood and bone marrow. In some embodiments, the CAR plus CSR T cells of the invention infused into a patient can eliminate target antigen-presenting cells, such as target antigen-presenting cancer or virally-infected cells, in vivo in patients having a target antigen-associated disease. In some embodiments, the CAR plus CSR T cells of the invention infused into a patient can eliminate target antigen-presenting cells, such as target antigen-presenting cancer or virally-infected cells, in vivo in patients having a target antigen-associated disease that is refractory to at least one conventional treatment.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of T cell lines available in the art may be used. In some embodiments of the present invention, T cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed, and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO′ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable CAR, CSR and optionally SSE, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

XI. Genetic Modification

In some embodiments, the CAR plus CSR immune cells (such as CAR plus CSR T cells) of the invention are generated by transducing immune cells (such as T cells prepared by the methods described herein) with one or more viral vectors encoding a CAR as described herein and a CSR as described herein. Viral vector delivery systems include DNA and RNA viruses, which have either episomal or integrated genomes after delivery to the immune cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11: 167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); and Yu et al., Gene Therapy 1:13-26 (1994). In some embodiments, the CAR plus CSR immune cell comprises the one or more vectors integrated into the CAR plus CSR immune cell genome. In some embodiments, the one or more viral vectors are lentiviral vectors. In some embodiments, the CAR plus CSR immune cell is a CAR plus CSR T cell comprising the lentiviral vectors integrated into its genome.

In some embodiments, the CAR plus CSR immune cell is a T cell modified to block or decrease the expression of one or both of its endogenous TCR chains. For example, in some embodiments, the CAR plus CSR immune cell is an αβ T cell modified to block or decrease the expression of the TCR α and/or β chains, or the CAR plus CSR immune cell is a γδ T cell modified to block or decrease the expression of the TCR γ and/or δ chains. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, CAR plus CSR T cells with reduced expression of one or both of the endogenous TCR chains of the T cell are generated using the CRISPR/Cas system. For a review of the CRISPR/Cas system of gene editing, see for example Jian W & Marraffini L A, Annu. Rev. Microbiol. 69, 2015; Hsu P D et al., Cell, 157(6):1262-1278, 2014; and O'Connell M R et al., Nature 516: 263-266, 2014. In some embodiments, CAR plus CSR T cells with reduced expression of one or both of the endogenous TCR chains of the T cell are generated using TALEN-based genome editing.

XIII. Enrichment

In some embodiments, there is provided a method of enriching a heterogeneous cell population for a CAR plus CSR immune cell according to any of the CAR plus CSR immune cells described herein.

A specific subpopulation of CAR plus CSR immune cells (such as CAR plus CSR T cells) that specifically bind to a target antigen and target ligand can be enriched for by positive selection techniques. For example, in some embodiments, CAR plus CSR immune cells (such as CAR plus CSR T cells) are enriched for by incubation with target antigen-conjugated beads and/or target ligand-conjugated beads for a time period sufficient for positive selection of the desired CAR plus CSR immune cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of CAR plus CSR immune cells present at low levels in the heterogeneous cell population, use of longer incubation times, such as 24 hours, can increase cell yield. Longer incubation times may be used to isolate CAR plus CSR immune cells in any situation where there are few CAR plus CSR immune cells as compared to other cell types. The skilled artisan would recognize that multiple rounds of selection can also be used in the context of this invention.

For isolation of a desired population of CAR plus CSR immune cells by positive selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of CAR plus CSR immune cells that may weakly express the CAR and/or CSR.

In some of any such embodiments described herein, enrichment results in minimal or substantially no exhaustion of the CAR plus CSR immune cells. For example, in some embodiments, enrichment results in fewer than about 50% (such as fewer than about any of 45, 40, 35, 30, 25, 20, 15, 10, or 5%) of the CAR plus CSR immune cells becoming exhausted. Immune cell exhaustion can be determined by any means known in the art, including any means described herein.

In some of any such embodiments described herein, enrichment results in minimal or substantially no terminal differentiation of the CAR plus CSR immune cells. For example, in some embodiments, enrichment results in fewer than about 50% (such as fewer than about any of 45, 40, 35, 30, 25, 20, 15, 10, or 5%) of the CAR plus CSR immune cells becoming terminally differentiated. Immune cell differentiation can be determined by any means known in the art, including any means described herein.

In some of any such embodiments described herein, enrichment results in minimal or substantially no internalization of CARs and/or CSRs on the CAR plus CSR immune cells. For example, in some embodiments, enrichment results in less than about 50% (such as less than about any of 45, 40, 35, 30, 25, 20, 15, 10, or 5%) of CARs and/or CSRs on the CAR plus CSR immune cells becoming internalized. Internalization of CARs and/or CSRs on CAR plus CSR immune cells can be determined by any means known in the art, including any means described herein.

In some of any such embodiments described herein, enrichment results in increased proliferation of the CAR plus CSR immune cells. For example, in some embodiments, enrichment results in an increase of at least about 10% (such as at least about any of 20, 30, 40, 50, 60, 70, 80, 90, 100, 200, 300, 400, 500, 1000% or more) in the number of CAR plus CSR immune cells following enrichment.

Thus, in some embodiments, there is provided a method of enriching a heterogeneous cell population for CAR plus CSR immune cells expressing a CAR that specifically binds to a target antigen and a CSR that specifically binds to a target ligand comprising: a) contacting the heterogeneous cell population with a first molecule comprising the target antigen or one or more epitopes contained therein and/or a second molecule comprising the target ligand or one or more epitopes contained therein to form complexes comprising the CAR plus CSR immune cell bound to the first molecule and/or complexes comprising the CAR plus CSR immune cell bound to the second molecule; and b) separating the complexes from the heterogeneous cell population, thereby generating a cell population enriched for the CAR plus CSR immune cells. In some embodiments, the first and/or second molecules are immobilized, individually, to a solid support. In some embodiments, the solid support is particulate (such as beads). In some embodiments, the solid support is a surface (such as the bottom of a well). In some embodiments, the first and/or second molecules are labelled, individually, with a tag. In some embodiments, the tag is a fluorescent molecule, an affinity tag, or a magnetic tag. In some embodiments, the method further comprises eluting the CAR plus CSR immune cells from the first and/or second molecules and recovering the eluate.

XIV. Effector Cell Therapy

The present application also provides methods of using immune cells as described herein to redirect the specificity of an effector cell (such as a primary T cell) to a cancer cell. Thus, the present invention also provides a method of stimulating an effector cell-mediated response (such as a T cell-mediated immune response) to a target cell population or tissue comprising cancer cells in a mammal, comprising the step of administering to the mammal an effector cell (such as a T cell) that expresses a CAR and a CSR as described herein. In some embodiments, “stimulating” an immune cell refers to eliciting an effector cell-mediated response (such as a T cell-mediated immune response), which is different from activating an immune cell.

Effector cells (such as T cells) expressing a CAR and a CSR as described herein can be infused to a recipient in need thereof. The infused cell is able to kill cancer cells in the recipient. In some embodiments, unlike antibody therapies, effector cells (such as T cells) are able to replicate in vivo resulting in long-term persistence that can lead to sustained tumor control.

In some embodiments, the effector cells are T cells that can undergo robust in vivo T cell expansion and can persist for an extended amount of time. In some embodiments, the T cells of the invention develop into specific memory T cells that can be reactivated to inhibit any additional tumor formation or growth.

The effector cells (such as T cells) of the invention may also serve as a type of vaccine for ex vivo immunization and/or in vivo therapy in a mammal. In some embodiments, the mammal is a human.

With respect to ex vivo immunization, at least one of the following occurs in vitro prior to administering the cell into a mammal: i) expansion of the cells, ii) introducing nucleic acid(s) encoding a CAR and a CSR to the cells, and/or iii) cryopreservation of the cells. Ex vivo procedures are well-known in the art. Briefly, cells are isolated from a mammal (preferably a human) and genetically modified (i.e., transduced or transfected in vitro) with vector(s) expressing a CAR and a CSR disclosed herein. The cell can be administered to a mammalian recipient to provide a therapeutic benefit. The mammalian recipient may be a human and the cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present invention. Other suitable methods are known in the art; therefore, the present invention is not limited to any particular method of ex vivo expansion of the cells. Briefly, ex vivo culture and expansion of T cells comprises: (1) collecting T cells from peripheral blood mononuclear cells (PBMC); and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient. The effector cells (such as T cells) of the present invention may be administered either alone, or as a pharmaceutical composition in combination with diluents and/or with other components such as IL-2 or other cytokines or cell populations. Briefly, pharmaceutical compositions of the present invention may comprise effector cells (such as T cells), in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, effector cell (such as T cell) compositions are formulated for administration by intravenous, intrathecal, intracranial, intracerebral, or intracerebroventricular route.

The precise amount of the effector cell (such as CAR T cell) compositions of the present invention to be administered can be determined by a physician with consideration of individual differences in age, weight, tumor size, extent of infection or metastasis, and condition of the patient (subject). In some embodiments, a pharmaceutical composition comprising the effector cells (such as T cells) is administered at a dosage of about 10⁴ to about 10⁹ cells/kg body weight, such any of about 10⁴ to about 10⁵, about 10⁵ to about 10⁶, about 10⁶ to about 10⁷, about 10⁷ to about 10⁸, or about 10⁸ to about 10⁹ cells/kg body weight, including all integer values within those ranges. Effect cell (such as T cell) compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

In some embodiments, it may be desired to administer activated effector cells (such as T cells) to a subject and then subsequently redraw blood (or have an apheresis performed), activate T cells therefrom according to the present invention, and reinfuse the patient with these activated and expanded T cells. This process can be carried out multiple times every few weeks. In some embodiments, T cells can be activated from blood draws of from 10 cc to 400 cc. In some embodiments, T cells are activated from blood draws of 20 cc, 30 cc, 40 cc, 50 cc, 60 cc, 70 cc, 80 cc, 90 cc, or 100 cc.

The administration of the effector cells (such as T cells) may be carried out in any convenient manner, including by injection, ingestion, transfusion, implantation or transplantation. The compositions described herein may be administered to a patient subcutaneously, intradermally, intratumorally, intranodally, intramedullary, intramuscularly, intrathecally, intracranially, intracerebrally, intracerebroventricularly, by intravenous (i.v.) injection, or intraperitoneally. In some embodiments, the effector cell (such as T cell) compositions of the present invention are administered to a patient by intradermal or subcutaneous injection. In some embodiments, the effector cell (such as T cell) compositions of the present invention are administered by i.v. injection. In some embodiments, the effector cell (such as T cell) compositions of the present invention are administered by intrathecal injection. In some embodiments, the effector cell (such as T cell) compositions of the present invention are administered by intracranial injection. In some embodiments, the effector cell (such as T cell) compositions of the present invention are administered by intracerebral injection. In some embodiments, the effector cell (such as T cell) compositions of the present invention are administered by intracerebroventricular injection. The compositions of effector cell (such as T cell) may be injected directly into a tumor, lymph node, or site of infection.

XV. Methods of Diagnosis and Imaging Using CARs and CSRs

Labeled CARs and CSRs can be used for diagnostic purposes to detect, diagnose, or monitor a cancer. For example, the CARs and CSRs described herein can be used in in situ, in vivo, ex vivo, and in vitro diagnostic assays or imaging assays.

Additional embodiments of the invention include methods of diagnosing a cancer (e.g., a hematological cancer or a solid tumor cancer) in an individual (e.g., a mammal such as a human). The methods comprise detecting antigen-presenting cells in the individual. In some embodiments, there is provided a method of diagnosing a cancer (e.g., a hematological cancer or a solid tumor cancer) in an individual (e.g., a mammal, such as a human) comprising (a) administering an effective amount of a labeled antibody moiety according to any of the embodiments described above to the individual; and (b) determining the level of the label in the individual, such that a level of the label above a threshold level indicates that the individual has the cancer. The threshold level can be determined by various methods, including, for example, by detecting the label according to the method of diagnosing described above in a first set of individuals that have the cancer and a second set of individuals that do not have the cancer, and setting the threshold to a level that allows for discrimination between the first and second sets. In some embodiments, the threshold level is zero, and the method comprises determining the presence or absence of the label in the individual. In some embodiments, the method further comprises waiting for a time interval following the administering of step (a) to permit the labeled antibody moiety to preferentially concentrate at sites in the individual where the antigen is expressed (and for unbound labeled antibody moiety to be cleared). In some embodiments, the method further comprises subtracting a background level of the label. Background level can be determined by various methods, including, for example, by detecting the label in the individual prior to administration of the labeled antibody moiety, or by detecting the label according to the method of diagnosing described above in an individual that does not have the cancer.

Antibody moieties of the invention can be used to assay levels of antigen-presenting cell in a biological sample using methods known to those of skill in the art. Suitable antibody labels are known in the art and include enzyme labels, such as, glucose oxidase; radioisotopes, such as iodine (131I, 125I, 123I, 121I), carbon (14C), sulfur (35S), tritium (3H), indium (115mIn, 113mIn, 112In, 111In), technetium (99Tc, 99mTc), thallium (201Ti), gallium (68Ga, 67Ga), palladium (103Pd), molybdenum (99Mo), xenon (133Xe), fluorine (18F), samarium (153Sm), lutetium (177Lu), gadolinium (159Gd), promethium (149Pm), lanthanum (140La), ytterbium (175Yb), holmium (166Ho), yttrium (90Y), scandium (47Sc), rhenium (186Re, 188Re), praseodymium (142Pr), rhodium (105Rh), and ruthenium (97Ru); luminol; fluorescent labels, such as fluorescein and rhodamine; and biotin.

Techniques known in the art may be applied to labeled antibody moieties of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Aside from the above assays, various in vivo and ex vivo assays are available to the skilled practitioner. For example, one can expose cells within the body of the subject to an antibody moiety which is optionally labeled with a detectable label, e.g., a radioactive isotope, and binding of the antibody moiety to the cells can be evaluated, e.g., by external scanning for radioactivity or by analyzing a sample (e.g., a biopsy or other biological sample) derived from a subject previously exposed to the antibody moiety.

XVI. Pharmaceutical Compositions

Also provided herein are CAR plus CSR immune cell compositions (such as pharmaceutical compositions, also referred to herein as formulations) comprising an immune cell (such as a T cell) presenting on its surface a CAR according to any of the CARs described herein and a CSR according to any of the CSRs described herein. In some embodiments, the CAR plus CSR immune cell composition is a pharmaceutical composition.

The composition may comprise a homogenous cell population comprising CAR plus CSR immune cells of the same cell type and expressing the same CAR and CSR, or a heterogeneous cell population comprising a plurality of CAR plus CSR immune cell populations comprising CAR plus CSR immune cells of different cell types, expressing different CARs, and/or expressing different CSRs. The composition may further comprise cells that are not CAR plus CSR immune cells.

Thus, in some embodiments, there is provided a CAR plus CSR immune cell composition comprising a homogeneous cell population of CAR plus CSR immune cells (such as CAR plus CSR T cells) of the same cell type and expressing the same CAR and CSR. In some embodiments, the CAR plus CSR immune cell is a T cell. In some embodiments, the CAR plus CSR immune cell is selected from the group consisting of a cytotoxic T cell, a helper T cell, a natural killer T cell, and a suppressor T cell. In some embodiments, the CAR plus CSR immune cell composition is a pharmaceutical composition.

In some embodiments, there is provided a CAR plus CSR immune cell composition comprising a heterogeneous cell population comprising a plurality of CAR plus CSR immune cell populations comprising CAR plus CSR immune cells of different cell types, expressing different CARs, and/or expressing different CSRs. In some embodiments, the CAR plus CSR immune cells are T cells. In some embodiments, each population of CAR plus CSR immune cells is, independently from one another, of a cell type selected from the group consisting of cytotoxic T cells, helper T cells, natural killer T cells, and suppressor T cells. In some embodiments, all of the CAR plus CSR immune cells in the composition are of the same cell type (e.g., all of the CAR plus CSR immune cells are cytotoxic T cells). In some embodiments, at least one population of CAR plus CSR immune cells is of a different cell type than the others (e.g., one population of CAR plus CSR immune cells consists of cytotoxic T cells and the other populations of CAR plus CSR immune cells consist of natural killer T cells). In some embodiments, each population of CAR plus CSR immune cells expresses the same CAR. In some embodiments, at least one population of CAR plus CSR immune cells expresses a different CAR than the others. In some embodiments, each population of CAR plus CSR immune cells expresses a different CAR than the others. In some embodiments, each population of CAR plus CSR immune cells expresses a CAR that specifically binds to the same target antigen. In some embodiments, at least one population of CAR plus CSR immune cells expresses a CAR that specifically binds to a different target antigen than the others (e.g., one population of CAR plus CSR immune cells specifically binds to a pMHC complex and the other populations of CAR plus CSR immune cells specifically bind to a cell surface receptor). In some embodiments, where at least one population of CAR plus CSR immune cells expresses a CAR that specifically binds to a different target antigen, each population of CAR plus CSR immune cells expresses a CAR that specifically binds to a target antigen associated with the same disease or disorder (e.g., each of the target antigens are associated with a cancer, such as breast cancer). In some embodiments, each population of CAR plus CSR immune cells expresses the same CSR. In some embodiments, at least one population of CAR plus CSR immune cells expresses a different CSR than the others. In some embodiments, each population of CAR plus CSR immune cells expresses a different CSR than the others. In some embodiments, each population of CAR plus CSR immune cells expresses a CSR that specifically binds to the same target ligand. In some embodiments, at least one population of CAR plus CSR immune cells expresses a CSR that specifically binds to a different target ligand than the others (e.g., one population of CAR plus CSR immune cells specifically binds to a pNMC complex and the other populations of CAR plus CSR immune cells specifically bind to a cell surface receptor). In some embodiments, where at least one population of CAR plus CSR immune cells expresses a CSR that specifically binds to a different target ligand, each population of CAR plus CSR immune cells expresses a CSR that specifically binds to a target ligand associated with the same disease or disorder (e.g., each of the target ligands are associated with a cancer, such as breast cancer). In some embodiments, the CAR plus CSR immune cell composition is a pharmaceutical composition.

Thus, in some embodiments, there is provided a CAR plus CSR immune cell composition comprising a plurality of CAR plus CSR immune cell populations according to any of the embodiments described herein, wherein all of the CAR plus CSR immune cells in the composition are of the same cell type (e.g., all of the CAR plus CSR immune cells are cytotoxic T cells), and wherein each population of CAR plus CSR immune cells expresses a different CAR than the others. In some embodiments, the CAR plus CSR immune cells are T cells. In some embodiments, the CAR plus CSR immune cells are selected from the group consisting of cytotoxic T cells, helper T cells, natural killer T cells, and suppressor T cells. In some embodiments, each population of CAR plus CSR immune cells expresses a CAR that specifically binds to the same target antigen. In some embodiments, at least one population of CAR plus CSR immune cells expresses a CAR that specifically binds to a different target antigen than the others (e.g., one population of CAR plus CSR immune cells specifically binds to a pMHC complex and the other populations of CAR plus CSR immune cells specifically bind to a cell surface receptor). In some embodiments, where at least one population of CAR plus CSR immune cells expresses a CAR that specifically binds to a different target antigen, each population of CAR plus CSR immune cells expresses a CAR that specifically binds to a target antigen associated with the same disease or disorder (e.g., each of the target antigens are associated with a cancer, such as breast cancer). In some embodiments, the CAR plus CSR immune cell composition is a pharmaceutical composition.

In some embodiments, there is provided a CAR plus CSR immune cell composition comprising a plurality of CAR plus CSR immune cell populations according to any of the embodiments described herein, wherein all of the CAR plus CSR immune cells in the composition are of the same cell type (e.g., all of the CAR plus CSR immune cells are cytotoxic T cells), and wherein each population of CAR plus CSR immune cells expresses a different CSR than the others. In some embodiments, the CAR plus CSR immune cells are T cells. In some embodiments, the CAR plus CSR immune cells are selected from the group consisting of cytotoxic T cells, helper T cells, natural killer T cells, and suppressor T cells. In some embodiments, each population of CAR plus CSR immune cells expresses a CSR that specifically binds to the same target ligand. In some embodiments, at least one population of CAR plus CSR immune cells expresses a CSR that specifically binds to a different target ligand than the others (e.g., one population of CAR plus CSR immune cells specifically binds to a pMHC complex and the other populations of CAR plus CSR immune cells specifically bind to a cell surface receptor). In some embodiments, where at least one population of CAR plus CSR immune cells expresses a CSR that specifically binds to a different target ligand, each population of CAR plus CSR immune cells expresses a CSR that specifically binds to a target ligand associated with the same disease or disorder (e.g., each of the target ligands are associated with a cancer, such as breast cancer). In some embodiments, the CAR plus CSR immune cell composition is a pharmaceutical composition.

In some embodiments, there is provided a composition comprising a plurality of CAR plus CSR immune cell populations according to any of the embodiments described herein, wherein at least one population of CAR plus CSR immune cells is of a different cell type than the others. In some embodiments, all of the populations of CAR plus CSR immune cells are of different cell types. In some embodiments, the CAR plus CSR immune cells are T cells. In some embodiments, each population of CAR plus CSR immune cells is, independently from one another, of a cell type selected from the group consisting of cytotoxic T cells, helper T cells, natural killer T cells, and suppressor T cells. In some embodiments, each population of CAR plus CSR immune cells expresses the same CAR. In some embodiments, at least one population of CAR plus CSR immune cells expresses a different CAR than the others. In some embodiments, each population of CAR plus CSR immune cells expresses a different CAR than the others. In some embodiments, each population of CAR plus CSR immune cells expresses a CAR that specifically binds to the same target antigen. In some embodiments, at least one population of CAR plus CSR immune cells expresses a CAR that specifically binds to a different target antigen than the others (e.g., one population of CAR plus CSR immune cells specifically binds to a pMHC complex and the other populations of CAR plus CSR immune cells specifically bind to a cell surface receptor). In some embodiments, where at least one population of CAR plus CSR immune cells expresses a CAR that specifically binds to a different target antigen, each population of CAR plus CSR immune cells expresses a CAR that specifically binds to a target antigen associated with the same disease or disorder (e.g., each of the target antigens are associated with a cancer, such as breast cancer). In some embodiments, each population of CAR plus CSR immune cells expresses the same CSR. In some embodiments, at least one population of CAR plus CSR immune cells expresses a different CSR than the others. In some embodiments, each population of CAR plus CSR immune cells expresses a different CSR than the others. In some embodiments, each population of CAR plus CSR immune cells expresses a CSR that specifically binds to the same target ligand. In some embodiments, at least one population of CAR plus CSR immune cells expresses a CSR that specifically binds to a different target ligand than the others (e.g., one population of CAR plus CSR immune cells specifically binds to a pMHC complex and the other populations of CAR plus CSR immune cells specifically bind to a cell surface receptor). In some embodiments, where at least one population of CAR plus CSR immune cells expresses a CSR that specifically binds to a different target ligand, each population of CAR plus CSR immune cells expresses a CSR that specifically binds to a target ligand associated with the same disease or disorder (e.g., each of the target ligands are associated with a cancer, such as breast cancer). In some embodiments, the CAR plus CSR immune cell composition is a pharmaceutical composition.

At various points during preparation of a composition, it can be necessary or beneficial to cryopreserve a cell. The terms “frozen/freezing” and “cryopreserved/cryopreserving” can be used interchangeably. Freezing includes freeze drying.

As is understood by one of ordinary skill in the art, the freezing of cells can be destructive (see Mazur, P., 1977, Cryobiology 14:251-272) but there are numerous procedures available to prevent such damage. For example, damage can be avoided by (a) use of a cryoprotective agent, (b) control of the freezing rate, and/or (c) storage at a temperature sufficiently low to minimize degradative reactions. Exemplary cryoprotective agents include dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59). In particular embodiments, DMSO can be used. Addition of plasma (e.g., to a concentration of 20-25%) can augment the protective effects of DMSO. After addition of DMSO, cells can be kept at 0° C. until freezing, because DMSO concentrations of 1% can be toxic at temperatures above 4° C.

In the cryopreservation of cells, slow controlled cooling rates can be critical and different cryoprotective agents (Rapatz et al., 1968, Cryobiology 5(1): 18-25) and different cell types have different optimal cooling rates (see e.g., Rowe and Rinfret, 1962, Blood 20:636; Rowe, 1966, Cryobiology 3(1):12-18; Lewis, et al., 1967, Transfusion 7(1):17-32; and Mazur, 1970, Science 168:939-949 for effects of cooling velocity on survival of stem cells and on their transplantation potential). The heat of fusion phase where water turns to ice should be minimal. The cooling procedure can be carried out by use of, e.g., a programmable freezing device or a methanol bath procedure. Programmable freezing apparatuses allow determination of optimal cooling rates and facilitate standard reproducible cooling.

In particular embodiments, DMSO-treated cells can be pre-cooled on ice and transferred to a tray containing chilled methanol which is placed, in turn, in a mechanical refrigerator (e.g., Harris or Revco) at −80° C. Thermocouple measurements of the methanol bath and the samples indicate a cooling rate of 1° to 3° C./minute can be preferred. After at least two hours, the specimens can have reached a temperature of −80° C. and can be placed directly into liquid nitrogen (−196° C.).

After thorough freezing, the cells can be rapidly transferred to a long-term cryogenic storage vessel. In a preferred embodiment, samples can be cryogenically stored in liquid nitrogen (−196° C.) or vapor (−1° C.). Such storage is facilitated by the availability of highly efficient liquid nitrogen refrigerators.

Further considerations and procedures for the manipulation, cryopreservation, and long-term storage of cells, can be found in the following exemplary references: U.S. Pat. Nos. 4,199,022; 3,753,357; and 4,559,298; Gorin, 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186; Livesey and Linner, 1987, Nature 327:255; Linner et al., 1986, J. Histochem. Cytochem. 34(9):1123-1135; Simione, 1992, J. Parenter. Sci. Technol. 46(6):226-32).

Following cryopreservation, frozen cells can be thawed for use in accordance with methods known to those of ordinary skill in the art. Frozen cells are preferably thawed quickly and chilled immediately upon thawing. In particular embodiments, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed on ice.

In particular embodiments, methods can be used to prevent cellular clumping during thawing. Exemplary methods include: the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc. [0162] As is understood by one of ordinary skill in the art, if a cryoprotective agent that is toxic to humans is used, it should be removed prior to therapeutic use. DMSO has no serious toxicity.

Exemplary carriers and modes of administration of cells are described at pages 14-15 of U.S. Patent Publication No. 2010/0183564. Additional pharmaceutical carriers are described in Remington: The Science and Practice of Pharmacy, 21 st Edition, David B. Troy, ed., Lippicott Williams & Wilkins (2005).

In particular embodiments, cells can be harvested from a culture medium, and washed and concentrated into a carrier in a therapeutically-effective amount. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, Ill.), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding a CAR and/or CSR and/or SSE described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

XVII. Dosage and Administration

The dose of the compositions administered to an individual (such as a human) may vary with the particular composition, the mode of administration, and the type of disease being treated. In some embodiments, the amount of the composition is sufficient to result in a complete response in the individual. In some embodiments, the amount of the composition is sufficient to result in a partial response in the individual. In some embodiments, the amount of the composition administered (for example when administered alone) is sufficient to produce an overall response rate of more than about any of 2%, 4%, 6%, 8%, 10%, 15%, 20%, 25%, 30%, 35%, 40%, 45%, 50%, 55%, 60%, 64%, 65%, 70%, 75%, 80%, 85%, or 90% among a population of individuals treated with the composition. Responses of an individual to the treatment of the methods described herein can be determined, for example, based on the percentage tumor growth inhibition (% TGI).

In some embodiments, the amount of the composition is sufficient to prolong overall survival of the individual. In some embodiments, the amount of the composition (for example when administered along) is sufficient to produce clinical benefit of more than about any of 2%, 4%, 6%, 8%, 10%, 15%, 20%, 25%, 30%, 35%, 40%, 45%, 50%, 60%, 70%, or 77% among a population of individuals treated with the composition.

In some embodiments, the amount of the composition is an amount sufficient to decrease the size of a tumor, decrease the number of cancer cells, or decrease the growth rate of a tumor by at least about any of 2%, 4%, 6%, 8%, 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the composition is below the level that induces a toxicological effect (i.e., an effect above a clinically acceptable level of toxicity) or is at a level where a potential side effect can be controlled or tolerated when the composition is administered to the individual. In some embodiments, the amount of the composition is close to a maximum tolerated dose (MTD) of the composition following the same dosing regimen. In some embodiments, the amount of the composition is more than about any of 80%, 90%, 95%, or 98% of the MTD. In some embodiments, the amount of the composition is included in a range of about 0.001 μg to about 1000 μg. In some embodiments of any of the above aspects, the effective amount of the composition is in the range of about 0.1 μg/kg to about 100 mg/kg of total body weight.

The compositions can be administered to an individual (such as human) via various routes, including, for example, intravenous, intra-arterial, intraperitoneal, intrapulmonary, oral, nasal, inhalation, intravesicular, intramuscular, intra-tracheal, subcutaneous, intraocular, intrathecal, intracranial, intracerebral, intracerebroventricular, transmucosal, and transdermal. In some embodiments, sustained continuous release formulation of the composition may be used. In some embodiments, the composition is administered intravenously. In some embodiments, the composition is administered intraarterially. In some embodiments, the composition is administered intraperitoneally. In some embodiments, the composition is administered intrathecally. In some embodiments, the composition is administered intracranially. In some embodiments, the composition is administered intracerebrally. In some embodiments, the composition is administered intracerebroventricularly. In some embodiments, the composition is administered nasally.

XVII. Manufacturing

In some embodiments of the invention, there is provided an article of manufacture containing materials useful for the treatment of a target antigen-positive disease such as cancer (for example adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer) or viral infection (for example infection by CMV, EBV, HBV, KSHV, HPV, MCV, HTLV-1, HIV-1, or HCV). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an immune cell presenting on its surface a CAR and a CSR of the invention. The label or package insert indicates that the composition is used for treating the particular condition. The label or package insert will further comprise instructions for administering the CAR plus CSR immune cell composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a target antigen-positive cancer (such as adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lung cancer, lymphoma, melanoma, mesothelioma, multiple myeloma, pancreatic cancer, pheochromocytoma, plasmacytoma, neuroblastoma, ovarian cancer, prostate cancer, sarcoma, stomach cancer, uterine cancer or thyroid cancer). In other embodiments, the package insert indicates that the composition is used for treating a target antigen-positive viral infection (for example infection by CMV, EBV, HBV, KSHV, HPV, MCV, HTLV-1, HIV-1, or HCV).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising a CAR plus CSR immune cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an immune cell presenting on its surface a CAR and a CSR. In some embodiments, the kit comprises a) a composition comprising an immune cell presenting on its surface a CAR and a CSR, and b) an effective amount of at least one other agent, wherein the other agent increases the expression of MHC proteins and/or enhances the surface presentation of peptides by MHC proteins (e.g., IFNγ, IFNβ, IFNα, or Hsp90 inhibitor). In some embodiments, the kit comprises a) a composition comprising an immune cell presenting on its surface a CAR and a CSR, and b) instructions for administering the CAR plus CSR immune cell composition to an individual for treatment of a target antigen-positive disease (such as cancer or viral infection). In some embodiments, the kit comprises a) a composition comprising an immune cell presenting on its surface a CAR and a CSR, b) an effective amount of at least one other agent, wherein the other agent increases the expression of MHC proteins and/or enhances the surface presentation of peptides by MHC proteins (e.g., IFNγ, IFNβ, IFNα, or Hsp90 inhibitor), and c) instructions for administering the CAR plus CSR immune cell composition and the other agent(s) to an individual for treatment of a target antigen-positive disease (such as cancer or viral infection). The CAR plus CSR immune cell composition and the other agent(s) can be present in separate containers or in a single container. For example, the kit may comprise one distinct composition or two or more compositions wherein one composition comprises the CAR plus CSR immune cell and another composition comprises the other agent.

In some embodiments, the kit comprises a) one or more compositions comprising a CAR and a CSR, and b) instructions for combining the CAR and CSR with immune cells (such as immune cells, e.g., T cells or natural killer cells, derived from an individual) to form a composition comprising the immune cells presenting on their surface the CAR and CSR, and administering the CAR plus CSR immune cell composition to the individual for treatment of a target antigen-positive disease (such as cancer or viral infection). In some embodiments, the kit comprises a) one or more compositions comprising a CAR and a CSR, and b) an immune cell (such as a cytotoxic cell). In some embodiments, the kit comprises a) one or more compositions comprising a CAR and a CSR, b) an immune cell (such as a cytotoxic cell), and c) instructions for combining the CAR and CSR with the immune cell to form a composition comprising the immune cell presenting on its surface the CAR and CSR, and administering the CAR plus CSR immune cell composition to an individual for the treatment of a target antigen-positive disease (such as cancer or viral infection).

In some embodiments, the kit comprises a nucleic acid (or set of nucleic acids) encoding a CAR and a CSR. In some embodiments, the kit comprises a) a nucleic acid (or set of nucleic acids) encoding a CAR and a CSR, and b) a host cell (such as an immune cell) for expressing the nucleic acid (or set of nucleic acids). In some embodiments, the kit comprises a) a nucleic acid (or set of nucleic acids) encoding a CAR and a CSR, and b) instructions for i) expressing the CAR and CSR in a host cell (such as an immune cell, e.g., a T cell), ii) preparing a composition comprising the host cell expressing the CAR and CSR, and iii) administering the composition comprising the host cell expressing the CAR and CSR to an individual for the treatment of a target antigen-positive disease (such as cancer or viral infection). In some embodiments, the host cell is derived from the individual. In some embodiments, the kit comprises a) a nucleic acid (or set of nucleic acids) encoding a CAR and a CSR, b) a host cell (such as an immune cell) for expressing the nucleic acid (or set of nucleic acids), and c) instructions for i) expressing the CAR and CSR in the host cell, ii) preparing a composition comprising the host cell expressing the CAR and CSR, and iii) administering the composition comprising the host cell expressing the CAR and CSR to an individual for the treatment of a target antigen-positive disease (such as cancer or viral infection).

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the CAR plus CSR immune cell compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of a CAR plus CSR immune cell composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the CAR and CSR, and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

Materials and Methods

### Cell Samples, Cell Lines, and Antibodies

The cell lines HepG2 (ATCC HB-8065; HLA-A2⁺, AFP⁺, GPC3⁺), SK-HEP-1 (ATCC HTB-52; HLA-A2⁺, AFP⁻), Raji (ATCC CCL-86; CD19⁺, CD22⁺), Nalm6 (ATCC CRL-1567; CD19⁺), Jurkat cells (ATCC TIB-152, CD20−, CD22−), RPMI-8226 (ATCC CRM-CCL-155, ROR1⁺), LNCaP (ATCC CRL-1740; PSMA⁺), and IM9 (ATCC CCL-159; HLA-A2⁺, NY-ESO-1⁺) were obtained from the American Type Culture Collection.

HepG2 is a hepatocellular carcinoma cell line that expresses AFP and GPC3; SK-HEP1 is a liver adenocarcinoma cell line that does not express AFP. SK-HEP1-AFP MG was generated by transducing the SK-HEP1 parental cell line with an AFP158 peptide expressing minigene cassette, which results in a high level of cell surface expression of AFP158/HLA-A*02:01 complex in SK-HEP1. SK-HEP1-AFP MG-GPC3 was generated by further transducing the SK-HEP1-AFP-MG cell line with an GPC3 expressing cassette, which results in a high level of cell surface expression of AFP158/HLA-A*02:01 complex and GPC3 in SK-HEP1. Raji is a Burkitt lymphoma cell line that expresses CD19 and CD22. Nalm6 is a leukemia cell line that also expresses CD19. Jurkat is an acute T cell lymphoma cell line that does not express CD22. RPMI-8226 cells are myeloma cells that express ROR1. The LNCaP prostate tumor cell line expresses PSMA. IM9 is a multiple myeloma cell line that expresses NY-ESO-1. All cell lines are cultured in RPMI 1640 or DMEM supplemented with 10% FBS and 2 mM glutamine at 37° C./5% CO₂.

Antibodies against human or mouse CD3, CD4, CD8, CD28, CCR7, CD45RA or myc tag are purchased from Invitrogen; anti-CD22 and CD20 antibodies are purchased from Biolegend.

The AFP158/HLA-A*02:01-specific antibody, the CD19-specific antibody, the CD20-specific and CD22-specific antibodies, the ROR1-specific antibody, the GPC3-specific antibody, the PSMA-specific antibody and the NY-ESO-1 antibody are developed and produced in house at Eureka Therapeutics. Flow cytometry data are collected using BD FACS Canto II and analyzed using FlowJo software package.

All peptides are purchased and synthesized by Elim Biopharma. Peptides are >90% pure. The peptides are dissolved in DMSO or diluted in saline at 10 mg/mL and frozen at −80° C. Biotinylated single chain AFP158/HLA-A*02:01 and control peptides/HLA-A*02:01 complex monomers are generated by refolding the peptides with recombinant HLA-A*02:01 and beta-2 microglobulin (02M). The monomers are biotinylated via the BSP peptide linked to the C-terminal end of HLA-A*02:01 extracellular domain (ECD) by the BirA enzyme. Fluorescence-labelled streptavidin is mixed with biotinylated peptide/HLA-A*02:01 complex monomer to form fluorescence-labelled peptide/HLA-A*02:01 tetramer.

Lentiviruses containing CARs are produced, for example, by transfection of 293T cells with vectors encoding the CARs. Primary human T cells are used for transduction after one-day stimulation with CD3/CD28 beads (Dynabeads®, Invitrogen) in the presence of interleukin-2 (IL-2) at 100 U/ml. Concentrated lentiviruses are applied to T cells in Retronectin- (Takara) coated 6-well plates for 96 hours. Transduction efficiencies of the anti-AFP/MHC CARs (or “anti-AFP CARs” or “anti-AFP-CARs”) and anti-AFP/MHC CAR plus anti-GPC3 CSR (or “anti-AFP-CAR+anti-GPC3-CSR”) constructs are assessed by flow cytometry. For anti-AFP CARs, a biotinylated AFP158/HLA-A*02:01 tetramer (“AFP158 tetramer”) with PE-conjugated streptavidin or anti-myc antibody respectively was used. For anti-GPC3 CSR, an anti-myc antibody was used. Repeat flow cytometry analyses are done on day 5 and every 3-4 days thereafter. For anti-CD19 CARs, the assay was performed using a PE-conjugated anti-CD19 anti-idiotype antibody.

Cell lines are transduced with a vector that encodes the CAR or both CAR and CSR, or with two vectors, one encoding CAR, one encoding CSR. Five days post-transduction, cell lysates are generated for western blot using an anti-myc antibody.

Tumor cytotoxicities are assayed by Cytox 96 Non-radioactive LDH Cytotoxicity Assay (Promega). CD3⁺ T cells are prepared from PBMC-enriched whole blood using EasySep Human T Cell Isolation Kit (StemCell Technologies) which negatively depletes CD14, CD16, CD19, CD20, CD36, CD56, CD66b, CD123, glycophorin A expressing cells. Human T cells are activated and expanded with, for example, CD3/CD28 Dynabeads (Invitrogen) according to manufacturer's protocol. Activated T cells (ATC) are cultured and maintained in RPMI 1640 medium with 10% FBS plus 100 U/ml IL-2 and used at day 7-14. Activated T cells (immune cells) and target cells are co-cultured at various effector-to-target ratios (e.g., 2.5:1 or 5:1) for 16 hours and assayed for cytotoxicities.

### Example 1A—Short-Term In Vitro Cancer Cell Killing Assay

Activated CAR+CD30-CSR T cells (T cells comprising a CAR and a CSR that comprises a CD30 costimulatory domain) and target cells are co-cultured at a 5:1 ratio with αCD19 or αAFP antibodies for 16 hours. Specific killing is determined by measuring LDH activity in culture supernatants. Tumor cytotoxicity is assayed by LDH Cytotoxicity Assay (Promega). Human T cells purchased from AllCells are activated and expanded with CD3/CD28 Dynabeads (Invitrogen) according to manufacturer's protocol. Activated T cells (ATC) are cultured and maintained in RPMI 1640 medium with 10% FBS plus 100 U/ml IL-2 and used at day 7-14. The T cells are >99% CD3⁺ by FACS analysis. Activated T cells (Effector cells) and the target cells, Nalm6 or HepG2 cells are co-cultured at a 5:1 ratio with different concentrations of αCD19 or αAFP antibodies, respectively for 16 hours. Cytotoxicities are then determined by measuring LDH activities in culture supernatants.

CAR+CD30-CSR T cells have higher killing efficacies than corresponding CAR T cells without CSR, and about the same killing efficacies as CAR+CD28 (or other costimulatory domain)-CSR T cells if not better.

### Example 1B—Short-Term In Vitro Cancer Cell Killing Assay

Assays comparing the short-term killing ability of the various CAR T cells (including 1^{st}-gen and 2nd-gen CAR T cells) are performed. Effector cells used in this example include the following:

1) CAR T cells without CSR;
2) CAR T cells with a CSR that comprises at least the intracellular CD30 costimulatory domain (CD30 IC domain), either with a CD30 transmembrane domain (referred to as “CAR+CD30-CSR T cells”) or a different costimulatory molecule's transmembrane (TM) domain, e.g., CD28 TM (referred to as “CAR+CD28T-CD30-CSR T cells”);
3) CAR T cells with a CSR that comprises at least intracellular CD28 costimulatory domain, either with a CD28 transmembrane domain (referred to as “CAR+CD28-CSR T cells”) or a different costimulatory molecule's transmembrane (TM) domain, e.g., CD30 TM (referred to as “CAR+CD30T-CD28-CSR T cells”);
4) CAR T cells with a CSR that comprises at least intracellular 4-1BB costimulatory domain, either with a 4-1BB TM domain (referred to as “CAR+41BB-CSR T cells”) or a different costimulatory molecule's TM domain, e.g., CD28 TM (referred to as “CAR+CD28T-41BB-CSR T cells”); and
5) CAR T cells with a CSR that comprises at least intracellular DAP10 costimulatory domain, either with a DAP10 TM domain (referred to as “CAR+DAP10-CSR T cells”) or a different costimulatory molecule's TM domain, e.g., CD28 TM (referred to as “CAR+CD28T-DAP10-CSR T cells”).

Other constructs or more detailed descriptions of constructs/T cells that can be used are disclosed herein, e.g., Example 9.

Activated effector cells and their corresponding target cells were co-cultured at an E:T ratio between 2:1 to 5:1 for 16-24 hours. Specific killing was determined by measuring LDH activity in culture supernatants. Tumor cytotoxicity was assayed by LDH Cytotoxicity Assay (Promega). Human T cells purchased from AllCells were activated and expanded with CD3/CD28 Dynabeads (Invitrogen) according to manufacturer's protocol. Activated T cells (ATC) were cultured and maintained in RPMI 1640 medium with 10% FBS plus 100 U/ml IL-2 and used at day 7-14. The T cells were >99% CD3⁺ by FACS analysis. Activated T cells (Effector cells) and the target cells e.g., HepG2 cells, were co-cultured at a 2:1 to 5:1 ratio 16-24 hours, typically 16 hours. Cytotoxicities were then determined by measuring LDH activities in culture supernatants.

The short-term killing ability of the various CAR T cells was also determined by measuring the amounts/levels of cytokines released from T cells upon engagement with target cells. The levels of cytokine release in the supernatant after 16 hour co-culture were quantified with Luminex Magpix technology using BioRad Bio-Plex kits or with ELISA. T cells with high cytotoxic potency secrete high levels of cytokines that were related to T cell activity, such as TNFα, GM-CSF, IFNγ, and IL-2.

CAR T cells with a CSR comprising at least the CD30 IC domain have higher killing efficacies than corresponding CAR T cells without CSR, and higher than or about the same killing efficacies as corresponding CAR T cells with CSRs that do not have a CD30 IC domain but have a different costimulatory molecule's IC domain, e.g., CD28, 4-1BB, or DAP10's IC domain.

### Example 2—Proliferation Potential and Persistence Assays

The proliferation and persistence of genetically modified T cells is crucial for the success of adoptive T-cell transfer therapies when treating cancers. To assay the effect of the CSR on T-cell proliferation and persistence we label T cells with the intracellular dye CFSE and observe the dilution of the dye as the T cells divide when stimulated with tumor cells. We are also able to measure persistence of the T cells by counting the number of CFSE-positive cells remaining at the indicated day.

Respective T cells are serum starved overnight and labeled with CFSE using CellTrace CFSE (Thermo Fisher C34554). 50,000 to 100,000 T cells are incubated at an effector cell to target cell ratio (E:T ratio) of 2:1 and flow cytometry is used to observe serial dilution of the CFSE dye as the T cells divide at the indicated day. The total number of T cells are counted with FACs.

CAR T cells with a CSR comprising at least the CD30 IC domain proliferate more than corresponding CAR T cells without CSR, and proliferate more than or about the same as corresponding CAR T cells with CSRs that do not have a CD30 IC domain but have a different costimulatory molecule's IC domain, e.g., CD28, 4-1BB, or DAP10's IC domain.

### Example 3A—In Vitro T Cell and Tumor Cell Counts after Multi-Week Engagements

A FACS based assay for counting target cells is used to compare the long-term killing potential of CAR+CSR T cells. Long-term killing by CAR+CD30-CSR T cells is also measured by co-culture with Raji cells. All CAR+CD30-CSR T cells show comparable survival post target cell engagement.

CAR+CD30-CSR T cells persist for longer period of time over multiple engagements of tumor cells and kill more tumor cells than corresponding CAR T cells without CSR, and about the same as CAR+CD28 (or other costimulatory domain)-CSR T cells if not better.

### Example 3B—Long-Term In Vitro T Cell and Target Cell Counts after Multi-Week Engagements

A FACS based assay for counting T cells and target cells is used to compare the long-term survival and target-cell killing potential of CAR+CD30-CSR T cells with CAR T cells without CSR or with CSRs comprising other costimulatory fragments. Typically, 50,000 to 100,000 T cells are incubated with target cells at an effector cell to target cell ratio (E:T ratio) of 2:1. The cells are rechallenged with target cells on various days, typically every 7 days after the first engagement. The numbers of remaining target cells and total T cells are quantified with FACS on various days after each target cell engagement.

CAR T cells with a CSR comprising at least the CD30 IC domain persist/survive for longer period of time over multiple engagements of tumor target cells and kill more tumor cells than corresponding CAR T cells without CSR do, and survive better and/or kill more tumor cells than or about the same as corresponding CAR T cells with CSRs that do not have a CD30 IC domain but have a different costimulatory molecule's IC domain, e.g., CD28, 4-1BB, or DAP10's IC domain.

### Example 4—In Vivo Cytokine Release

To determine the level of cytokine release in vivo, key cytokines, including those related to clinical cytokine release syndrome, are analyzed 16, 24, 48, and 72 hours after the NALM-6 tumor-bearing mice were administered CAR+CD30-CSR T cells. Cytokine levels were quantified with Luminex Magpix technology using BioRad Bio-Plex kits.

CAR+CD30-CSR T cells secrete higher levels of cytokines that are related to T cell activity, such as TNFα, GM-CSF, IFNγ, and IL-2, than corresponding CAR T cells without CSR. For example, CAR+CD30-CSR T cells secrete higher levels of cytokines that are related to T cell activity, such as TNFα, GM-CSF, IFNγ, and IL-2, than CAR+CD28 (or other costimulatory domain)-CSR T cells.

### Example 5A—Differentiation of T-Cell Subsets Over Time (CCR7/CD45RA)

Proliferation and survival of CAR+CD30-CSR T cells is measured before and after target cell engagement in two independent flow cytometric assays. FACS analysis of CAR+CD30-CSR T cells shows a greater level of expression of the T cell differentiation markers CCR7 and CD45RA compared to CAR+CD28 (or other costimulatory domain)-CSR T cells prior to target engagement.

CAR+CD30-CSR T cells have increased percentage of memory and naïve T cells as compared to CAR+CD28 (or other costimulatory domain)-CSR T cells.

### Example 5B—Differentiation of T Cell Subsets Over Time (CCR7/CD45RA) and Memory T Cell Quantification

CAR+CD30-CSR T cells develop into and maintain a high memory T cell population after target stimulation, including central memory and effector memory T cells. To determine the effect of expressing CAR+CD30-CSR on T cells' ability to develop into and maintain memory T cells as compared to expressing CAR only or CAR co-expressed with a CSR comprising a different costimulatory fragment, e.g., CD28, 4-1BB, or DAP10's IC domain, we measure the cell surface expression of memory T cell markers CCR7 and CD45RA. As known in the field, T cells with high CCR7 expression levels and low CD45RA expression levels are considered as central memory T cells, T cells with low CCR7 and low CD45RA expression levels are effector memory T cells, T cells with low CCR7 and high CD45RA expression levels are effector T cells, while T cells with high CCR7 and high CD45RA are naïve T cells which are the initial type of T cells before target/antigen challenge/recognition (Mahnke et al., Eur J Immunol. 43(11):2797-809, 2013). When in response to antigen encounter, naïve T cells proliferate and differentiate into effector cells, most of which carry out the job of destroying targets and then die, while a small pool of T cells ultimately develops into long-lived memory T cells which can store the T cell immunity against the specific target. Among the memory T cells, the central memory T cells are found to have longer lives than effector memory T cells and be capable of generating effector memory T cells, but not vice versa. Therefore, the ability to develop into and maintain memory T cells, especially central memory T cells, is an important and desired feature for potentially successful T cell therapies.

The effector cells expressing CAR constructs alone are incubated with target cells at an E:T ratio of 2:1 (e.g., 100,000 receptor⁺ T cells and 50,000 target cells in each well on a 96-well plate) for 7 days. The cells are then rechallenged with 50,000-100,000 target cells per well every 7 days.

The CAR+CD30-CSR and CAR+other CSR T cells are incubated with target cells at an E:T ratio of 1:2 (e.g., 25,000 receptor⁺ T cells and 50,000 target cells in each well) for 7 days. The cells are then rechallenged with 50,000-100,000 target cells per well every 7 days.

In some experiments, the CAR+CSR T cell and target cell mixtures are diluted 1:6 before the fourth and fifth target cell engagement (E4 and E5) to avoid the overcrowdedness of T cells due to the significant T cell expansion, so that only one sixth of the previously remaining cells are rechallenged with 50,000-100,000 target cells.

On selected days after each target cell engagement, the entire cell mixture in a well from each sample is stained with antibodies against CCR7 and CD45RA and analyzed by flow cytometry. Receptor⁺ T cell numbers are counted, and cells are grouped into various T cell types based on their CCR7 and CD45RA expression levels: central memory T cells (CD45RA-CCR7⁺), effector memory T cells (CD45RA⁻ CCR7⁻), effector T cells (CD45RA⁺ CCR7⁻), and naïve T cells (CD45RA⁺ CCR7⁺). Percentages of various types of T cells among the total number of receptor⁺ T cells are calculated. In some experiments, the cells are also stained with antibodies against CD8 or CD4 to determine the CD8-CD4 characteristics of the counted T cells.

Proliferation and survival of CAR or CAR+CSR T cells are measured before and after target cell engagement. CAR T cells with a CSR comprising at least the CD30 IC domain are able to develop into and maintain high numbers and high percentages of central memory T cells upon engagement with target calls, higher than T cells expressing CAR alone or co-expressing CAR and a CSR that does not have a CD30 IC domain but has a different costimulatory molecule's IC domain, e.g., CD28, 4-1BB, or DAP10's IC domain.

### Example 6—Expression of T Cell Exhaustion Markers in T Cells after Co-Culture with Target Cells

Molecules such as PD-1, LAG3, TIM3, and TIGIT are inhibitory receptors that accumulate on T cells as T cells lose function. Because of this phenomenon these molecules' expression is seen as a marker of exhausted T cells. To examine the level of exhaustion markers expressed on CAR+CSR-transduced cells upon antigen stimulation, CD3⁺ T cells are prepared from PBMC-enriched whole blood using EasySep Human T Cell Isolation Kit (StemCell Technologies) and activated with CD3/CD28 Dynabeads as above. The activated and expanded cell population is >99% CD3⁺ by flow cytometry. These cells are then transduced with lentiviral vectors encoding a CAR+CD30-CSR, with other CSR, or no CSR for 7-9 days. The transduced T cells (effector cells) are co-cultured with target cells for 16 hours at an effector-to-target ratio in the range of 1:1 to 2.5:1. Using antibodies to exhaustion marker PD-1, LAG3, TIGIT, or TIM3, the level of exhaustion markers, e.g., MFI levels, on the transduced T cells are analyzed by flow cytometry. In some experiments, the cells are incubated for longer times and rechallenged with target cells every 7 days, and exhaustion marker levels are measured on selected days after each target cell engagement.

Over a series of target cell engagements, CAR+CD30-CSR T cells have lower levels of T cell exhaustion markers than corresponding CAR T cells without CSR and the other tested costimulatory domain-CSR T cells, e.g., CAR+CD28 (or other costimulatory domain)-CSR T cells. CAR T cells with a CSR comprising at least the CD30 IC domain have lower levels of T cell exhaustion markers than corresponding CAR T cells without CSR do, and have lower levels of T cell exhaustion markers than corresponding CAR T cells with CSRs that do not have a CD30 IC domain but have a different costimulatory molecule's IC domain, e.g., CD28, 4-1BB, or DAP10's IC domain.

### Example 7—Tumor Cell Killing

Assays comparing the tumor cell killing ability of the various T cells are performed. Activated T cells and target cells are co-cultured at a 5:1 ratio for 16 hours. Specific killing is determined by measuring LDH activity in culture supernatants. Tumor cytotoxicity is assayed by a LDH Cytotoxicity Assay (Promega). Human T cells purchased from AllCells are activated and expanded with CD3/CD28 Dynabeads (Invitrogen) according to manufacturer's protocol. Activated T cells (ATC) are cultured and maintained in RPMI 1640 medium with 10% FBS plus 100 U/ml IL-2 and used at day 7-14. The T cells are >99% CD3⁺ by FACS analysis. Activated T cells (Effector cells) and the target cells, Nalm6 or HepG2 cells, are co-cultured at a 5:1 ratio for 16 hours. Cytotoxicities are then determined by measuring LDH activities in culture supernatants.

CAR+CD30-CSR T cells have higher in vivo tumor cell killing efficacies than corresponding CAR T cells without CSR and CAR+CD28 (or other costimulatory domain)-CSR T cells.

### Example 8A—In Vivo Tumor Infiltration/Penetration by T Cells

About 10⁷ HepG2 tumor cells are implanted subcutaneously in NSG mice and allowed to form a solid tumor mass 150 mm³. 5×10⁶ CAR+ T cells are injected i.v. into the tumor bearing mice. 3 weeks after T-cell dosing, the mice are sacrificed and tumors removed, fixed and sectioned onto slides. Tumor sections are stained with CD3 antibody to visualize the T cells that are present within the solid tumor. Quantification of the number of CD3⁺ cells can be used to score the tumor infiltration ability of the T cells (T-cell/mm²)

CAR+CD30-CSR T cells have higher in vivo tumor infiltration/penetration rates/levels (i.e., higher numbers of T cells/mm²) than corresponding CAR T cells without CSR or corresponding CAR+CD28 (or other costimulatory domain)-CSR T cells.

### Example 8B—In Vivo Tumor Infiltration by T Cells

About 10⁷ tumor cells used for an animal model, e.g., HepG2 cells for liver cancer animal model, Nalm6 or Raji cells for CD19⁺ lymphoma animal model, Jekol for ROR1⁺ lymphoma animal model, MDA-MB-231 cells for ROR1⁺ breast cancer animal model, RPMI8226 cells for ROR1V multiple myeloma animal model, A549 cells, H1975 cells, or H1703 cells for ROR1⁺ lung cancer animal model, are implanted subcutaneously in NSG mice and allowed to form a solid tumor, e.g., a solid tumor with the mass of about 150-250 mm³, over a period of time. About 5×10⁶ to 1×10⁷ various CAR T cells (e.g., CAR only, CAR+CD30 CSR, CAR+CD28-CSR, CAR+DAP10-CSR, CAR+4-1BB-CSR, or CAR+other costimulatory domain-CSR T cells), are injected i.v. into the tumor bearing mice. 10 days to 3 weeks after T-cell dosing, the mice are sacrificed and tumors removed, fixed and sectioned onto slides.

Immunohistochemistry is performed on tumor sections to stain for CD3, a T cell marker, to visualize the T cells that are present within the solid tumor, representing all the T cells that infiltrated the solid tumor (including those penetrated the tumor and those proliferated/expanded from the penetrated T cells). The CD3-positive and CD3-negative cells in these sections were quantified, e.g., with an automated immunohistochemistry imager and/or using the QuPath software, in order to determine the fraction of tumor mass infiltrated by T cells, expressed as % of all cells that are CD3⁺ cells (T cells) or number of T cells/mm² tumor section. Higher % of T cells among all cells or higher number of T cells/mm² indicates higher/increased tumor infiltration rates/levels by the T cells, which reflects a combination of tumor penetration and cell proliferative capacities of the T cells.

CAR T cells with a CSR comprising at least the CD30 IC domain have higher in vivo tumor infiltration rates/levels/capabilities than corresponding CAR T cells without CSR or corresponding CAR T cells with CSRs that do not have a CD30 IC domain but have a different costimulatory molecule's IC domain, e.g., CD28, 4-1BB, or DAP10's IC domain.

### Example 9—Constructs

For liver cancers including HCC:

Constructs: 1^{st}-gen and 2^{nd}-gen anti-AFP CARs co-expressed with anti-GPC3 CSRs comprising CD28 or CD30 co-stimulatory fragments.
Construct: 1^{st}-gen αAFP-CD8T-z-CAR: a 1^{st} generation CAR comprising anti-AFP/MHC EC, CD8 TM, and CD3zeta IC (NO co-stim).
Construct: 1^{st}-gen αAFP-CD8T-z-CAR+αGPC3-CD28-CSR: 1^{st}-gen anti-AFP-CD8T-z-CAR co-expressed with a CSR comprising anti-GPC3 EC, CD28 TM, and CD28 IC
Construct: 1^{st}-gen αAFP-CD8T-z-CAR+αGPC3-CD30-CSR: 1^{st}-gen anti-AFP-CD8T-z-CAR co-expressed with a CSR comprising anti-GPC3 EC, CD30 TM, and CD30 IC
Construct: 1^{st}-gen αAFP-CD8T-z-CAR+αGPC3-CD8T-CD30-CSR: 1^{st}-gen anti-AFP-CD8T-z-CAR co-expressed with a CSR comprising anti-GPC3 EC, CD8 TM, and CD30 IC
Construct: αAFP-CD8T-CD30z-CAR: a 2^{nd} generation CAR comprising anti-AFP/MHC EC, CD8 TM, CD30 IC, and CD3zeta IC
Construct: αAFP-CD8T-CD30z-CAR+αGPC3-CD28-CSR: anti-AFP-CD8T-CD30z-CAR co-expressed with a CSR comprising anti-GPC3 EC, CD28 TM, and CD28 IC
Construct: αAFP-CD8T-CD30z-CAR+αGPC3-CD30-CSR: anti-AFP-CD8T-CD30z-CAR co-expressed with a CSR comprising anti-GPC3 EC, CD30 TM, and CD30 IC
Construct: αAFP-CD8T-CD30z-CAR+αGPC3-CD8T-CD30-CSR: anti-AFP-CD8T-CD30z-CAR co-expressed with a CSR comprising anti-GPC3 EC, CD8 TM, and CD30 IC
Construct: αAFP-CD28z-CAR: a 2^{nd} generation CAR comprising anti-AFP/MHC EC, CD28 TM, CD28 IC, and CD3zeta IC
Construct: αAFP-CD28z-CAR+αGPC3-CD28-CSR: anti-AFP-CD28z-CAR co-expressed with a CSR comprising anti-GPC3 EC, CD28 TM, and CD28 IC
Construct: αAFP-CD28z-CAR+αGPC3-CD30-CSR: anti-AFP-CD28z-CAR co-expressed with a CSR comprising anti-GPC3 EC, CD30 TM, and CD30 IC
Construct: αAFP-CD28z-CAR+αGPC3-CD8T-CD30-CSR: anti-AFP-CD28z-CAR co-expressed with a CSR comprising anti-GPC3 EC, CD8 TM, and CD30 IC
Construct: αAFP-CD8T-41BBz-CAR: a 2^{nd} generation CAR comprising anti-AFP/MHC EC, CD8 TM, 4-1BB IC, and CD3zeta IC
Construct: αAFP-CD8T-41BBz-CAR+αGPC3-CD30-CSR: anti-AFP-CD8T-41BBz CAR co-expressed with a CSR comprising anti-GPC3 EC, CD30 TM and CD30 IC

### Construct: αAFP-CD8T-CD30z-CAR+αGPC3-CD30-CSR

Construct: αAFP-CD8T-CD30z-CAR+αGPC3-41BB-CSR: anti-AFP-CD8T-CD30z-CAR co-expressed with a CSR comprising anti-GPC3 EC, 4-1BB TM, and 4-1BB IC
Construct: αAFP-CD8T-CD30z-CAR+αGPC3-OX40-CSR: anti-AFP-CD8T-CD30z-CAR co-expressed with a CSR comprising anti-GPC3 EC, OX40 TM, and OX40 IC
Construct: αAFP-CD8T-CD30z-CAR+αGPC3-CD27-CSR: anti-AFP-CD8T-CD30z-CAR co-expressed with a CSR comprising anti-GPC3 EC, CD27 TM, and CD27 IC
Construct: αAFP-CD8T-CD30z-CAR+αGPC3-CD30-CSR: anti-AFP-CD8T-CD30z-CAR co-expressed with a CSR comprising anti-GPC3 EC, CD30 TM, and CD30 IC
Construct: αAFP-CD8T-CD30z-CAR+αGPC3-CD30T-CD28-CSR: anti-AFP-CD8T-CD30z-CAR co-expressed with a CSR comprising anti-GPC3 EC, CD30 TM, and CD28 IC
Construct: αAFP-CD8T-CD30z-CAR+αGPC3-CD30T-41BB-CSR: anti-AFP-CD8T-CD30z-CAR co-expressed with a CSR comprising anti-GPC3 EC, CD30 TM, and 4-1BB IC
Construct: αAFP-CD8T-CD30z-CAR+αGPC3-CD30T-OX40-CSR: anti-AFP-CD8T-CD30z-CAR co-expressed with a CSR comprising anti-GPC3 EC, CD30 TM, and OX40 IC
Construct: αAFP-CD8T-CD30z-CAR+αGPC3-CD30T-CD27-CSR: anti-AFP-CD8T-CD30z-CAR co-expressed with a CSR comprising anti-GPC3 EC, CD30 TM, and CD27 IC
Construct: αAFP-CD8T-CD30z-CAR+αGPC3-CD28T-CD30-CSR: anti-AFP-CD8T-CD30z-CAR co-expressed with a CSR comprising anti-GPC3 EC, CD28 TM, and CD30 IC
Construct: αAFP-CD8T-CD30z-CAR+αGPC3-CD28-CSR: anti-AFP-CD8T-CD30z-CAR co-expressed with a CSR comprising anti-GPC3 EC, CD28 TM, and CD28 IC
Construct: αAFP-CD8T-CD30z-CAR+αGPC3-CD28T-41BB-CSR: anti-AFP-CD8T-CD30z-CAR co-expressed with a CSR comprising anti-GPC3 EC, CD28 TM, and 4-1BB IC
Construct: αAFP-CD8T-CD30z-CAR+αGPC3-CD28T-OX40-CSR: anti-AFP-CD8T-CD30z-CAR co-expressed with a CSR comprising anti-GPC3 EC, CD28 TM, and OX40 IC
Construct: αAFP-CD8T-CD30z-CAR+αGPC3-CD28T-CD27-CSR: anti-AFP-CD8T-CD30z-CAR co-expressed with a CSR comprising anti-GPC3 EC, CD28 TM, and CD27 IC
Construct: αAFP-CD8T-CD30z-CAR+αGPC3-41BBT-CD30-CSR: anti-AFP-CD8T-CD30z-CAR co-expressed with a CSR comprising anti-GPC3 EC, 4-1BB TM, and CD30 IC
Construct: αAFP-CD8T-CD30z-CAR+αGPC3-41BB-CSR: anti-AFP-CD8T-CD30z-CAR co-expressed with a CSR comprising anti-GPC3 EC, 4-1BB TM, and 4-1BB IC
Construct: αAFP-CD8T-CD30z-CAR+αGPC3-41BBT-CD28-CSR: anti-AFP-CD8T-CD30z-CAR co-expressed with a CSR comprising anti-GPC3 EC, 4-1BB TM, and CD30 IC
Construct: αAFP-CD8T-CD30z-CAR+αGPC3-41BBT-OX40-CSR: anti-AFP-CD8T-CD30z-CAR co-expressed with a CSR comprising anti-GPC3 EC, 4-1BB TM, and 4-1BB IC
Construct: αAFP-CD8T-CD30z-CAR+αGPC3-41BBT-CD27-CSR: anti-AFP-CD8T-CD30z-CAR co-expressed with a CSR comprising anti-GPC3 EC, 4-1BB TM, and CD27 IC
Construct: αAFP-CD8T-CD30z-CAR+αGPC3-OX40T-CD30-CSR: anti-AFP-CD8T-CD30z-CAR co-expressed with a CSR comprising anti-GPC3 EC, OX40 TM, and CD30 IC
Construct: αAFP-CD8T-CD30z-CAR+αGPC3-OX40-CSR: anti-AFP-CD8T-CD30z-CAR co-expressed with a CSR comprising anti-GPC3 EC, OX40 TM, and OX40 IC
Construct: αAFP-CD8T-CD30z-CAR+αGPC3-OX40T-CD28-CSR: anti-AFP-CD8T-CD30z-CAR co-expressed with a CSR comprising anti-GPC3 EC, OX40 TM, and CD28 IC
Construct: αAFP-CD8T-CD30z-CAR+αGPC3-OX40T-41BB-CSR: anti-AFP-CD8T-CD30z-CAR co-expressed with a CSR comprising anti-GPC3 EC, OX40 TM, and 4-1BB IC
Construct: αAFP-CD8T-CD30z-CAR+αGPC3-OX40T-CD27-CSR: anti-AFP-CD8T-CD30z-CAR co-expressed with a CSR comprising anti-GPC3 EC, OX40 TM, and CD27 IC
Construct: αAFP-CD8T-CD30z-CAR+αGPC3-CD27T-CD30-CSR: anti-AFP-CD8T-CD30z-CAR co-expressed with a CSR comprising anti-GPC3 EC, CD27 TM, and CD30 IC
Construct: αAFP-CD8T-CD30z-CAR+αGPC3-CD27-CSR: anti-AFP-CD8T-CD30z-CAR co-expressed with a CSR comprising anti-GPC3 EC, CD27 TM, and CD27 IC
Construct: αAFP-CD8T-CD30z-CAR+αGPC3-CD27T-41BB-CSR: anti-AFP-CD8T-CD30z-CAR co-expressed with a CSR comprising anti-GPC3 EC, CD27 TM, and 4-1BB IC
Construct: αAFP-CD8T-CD30z-CAR+αGPC3-CD27T-OX40-CSR: anti-AFP-CD8T-CD30z-CAR co-expressed with a CSR comprising anti-GPC3 EC, CD27 TM, and OX40 IC
Construct: αAFP-CD8T-CD30z-CAR+αGPC3-CD27T-CD28-CSR: anti-AFP-CD8T-CD30z-CAR co-expressed with a CSR comprising anti-GPC3 EC, CD27 TM, and CD28 IC
Construct: αAFP-CD8T-CD30z-CAR+αGPC3-CD8T-CD30-CSR: anti-AFP-CD8T-CD30z-CAR co-expressed with a CSR comprising anti-GPC3 EC, CD8 TM, and CD30 IC
Construct: αAFP-CD8T-CD30z-CAR+αGPC3-CD8T-CD28-CSR: anti-AFP-CD8T-CD30z-CAR co-expressed with a CSR comprising anti-GPC3 EC, CD8 TM, and CD28 IC
Construct: αAFP-CD8T-CD30z-CAR+αGPC3-CD8T-41BB-CSR: anti-AFP-CD8T-CD30z-CAR co-expressed with a CSR comprising anti-GPC3 EC, CD8 TM, and 4-1BB IC
Construct: αAFP-CD8T-CD30z-CAR+αGPC3-CD8T-OX40-CSR: anti-AFP-CD8T-CD30z-CAR co-expressed with a CSR comprising anti-GPC3 EC, CD8 TM, and OX40 IC
Construct: αAFP-CD8T-CD30z-CAR+αGPC3-CD8T-CD27-CSR: anti-AFP-CD8T-CD30z-CAR co-expressed with a CSR comprising anti-GPC3 EC, CD8 TM, and CD27 IC
Constructs: 1^{st}-gen and 2^{nd}-gen anti-GPC3 CARs co-expressed with anti-GPC3 CSRs comprising CD28 or CD30 co-stimulatory fragments.
Construct: 1^{st}-gen αGPC3-CD8T-z-CAR: a 1st generation CAR comprising anti-GPC3 EC, CD8 TM, and CD3zeta IC (NO co-stim).
Construct: 1^{st}-gen αGPC3-CD8T-z-CAR+αGPC3-CD28-CSR: 1^{st}-gen anti-GPC3-CD8T-z-CAR co-expressed with a CSR comprising anti-GPC3 EC, CD28 TM, and CD28 IC
Construct: 1^{st}-gen αGPC3-CD8T-z-CAR+αGPC3-CD30-CSR: 1^{st}-gen anti-GPC3-CD8T-z-CAR co-expressed with a CSR comprising anti-GPC3 EC, CD30 TM, and CD30 IC
Construct: 1^{st}-gen αGPC3-CD8T-z-CAR+αGPC3-CD8T-CD30-CSR: 1^{st}-gen anti-GPC3-CD8T-z-CAR co-expressed with a CSR comprising anti-GPC3 EC, CD8 TM, and CD30 IC
Construct: αGPC3-CD8T-CD30z-CAR: a 2^{nd} generation CAR comprising anti-GPC3/MHC EC, CD8 TM, CD30 IC, and CD3zeta IC
Construct: αGPC3-CD8T-CD30z-CAR+αGPC3-CD28-CSR: anti-GPC3-CD8T-CD30z-CAR co-expressed with a CSR comprising anti-GPC3 EC, CD28 TM, and CD28 IC
Construct: αGPC3-CD8T-CD30z-CAR+αGPC3-CD30-CSR: anti-GPC3-CD8T-CD30z-CAR co-expressed with a CSR comprising anti-GPC3 EC, CD30 TM, and CD30 IC
Construct: αGPC3-CD8T-CD30z-CAR+αGPC3-CD8T-CD30-CSR: anti-GPC3-CD8T-CD30z-CAR co-expressed with a CSR comprising anti-GPC3 EC, CD8 TM, and CD30 IC
Construct: αGPC3-CD28z-CAR: a 2^{nd} generation CAR comprising anti-GPC3 EC, CD28 TM, CD28 IC, and CD3zeta IC
Construct: αGPC3-CD28z-CAR+αGPC3-CD28-CSR: anti-GPC3-CD28z-CAR co-expressed with a CSR comprising anti-GPC3 EC, CD28 TM, and CD28 IC
Construct: αGPC3-CD28z-CAR+αGPC3-CD30-CSR: anti-GPC3-CD28z-CAR co-expressed with a CSR comprising anti-GPC3 EC, CD30 TM, and CD30 IC
Construct: αGPC3-CD28z-CAR+αGPC3-CD30T-CD28-CSR: anti-GPC3-CD28z-CAR co-expressed with a CSR comprising anti-GPC3 EC, CD30 TM, and CD28 IC
Construct: αGPC3-CD28z-CAR+αGPC3-CD8T-CD30-CSR: anti-GPC3-CD28z-CAR co-expressed with a CSR comprising anti-GPC3 EC, CD8 TM, and CD30 IC

For blood cancers including leukemias and lymphomas:

Construct: 1^{st}-gen and 2^{nd}-gen anti-CD19 CARs co-expressed with anti-CD19 CSRs comprising CD28 or CD30 co-stimulatory fragments
Construct: 1^{st}-gen αCD19-CD8T-z-CAR: a 1st generation CAR comprising anti-CD19 EC, CD8 TM, and CD3zeta IC (NO co-stim)
Construct: 1^{st}-gen αCD19-CD8T-z-CAR+αCD19-CD28-CSR: 1^{st}-gen anti-CD19-CD8T-z-CAR co-expressed with a CSR comprising anti-CD19 EC, CD28 TM, and CD28 IC
Construct: 1^{st}-gen αCD19-CD8T-z-CAR+αCD19-CD30-CSR: 1^{st}-gen anti-CD19-CD8T-z-CAR co-expressed with a CSR comprising anti-CD19 EC, CD30 TM, and CD30 IC
Construct: 1^{st}-gen αCD19-CD8T-z-CAR+αCD19-CD28T-CD30-CSR: 1^{st}-gen anti-CD19-CD8T-z-CAR co-expressed with a CSR comprising anti-CD19 EC, CD28 TM, and CD30 IC
Construct: αCD19-CD30z-CAR: a 2^{nd} generation CAR comprising anti-CD19 EC, CD30 TM, CD30 IC, and CD3zeta IC
Construct: αCD19-CD30z-CAR+αCD19-CD28-CSR: anti-CD19-CD30z-CAR co-expressed with a CSR comprising anti-CD19 EC, CD28 TM, and CD28 IC
Construct: αCD19-CD30z-CAR+αCD19-CD30-CSR: anti-CD19-CD30z-CAR co-expressed with a CSR comprising anti-CD19 EC, CD30 TM, and CD30 IC
Construct: αCD19-CD28z-CAR: a 2^{nd} generation CAR comprising anti-CD19 EC, CD28 TM, CD28 IC, and CD3zeta IC
Construct: αCD19-CD28z-CAR+αCD19-CD28-CSR: anti-CD19-CD28z-CAR co-expressed with a CSR comprising anti-CD19 EC, CD28 TM, and CD28 IC
Construct: αCD19-CD28z-CAR+αCD19-CD30-CSR: anti-CD19-CD28z-CAR co-expressed with a CSR comprising anti-CD19 EC, CD30 TM, and CD30 IC
Construct: αCD19-CD8T-41BBz-CAR: a 2^{nd} generation CAR comprising anti-CD19 EC, CD8 TM, 41-BB IC, and CD3zeta IC
Construct: αCD19-CD8T-41BBz-CAR+αCD19-CD28-CSR: anti-CD19-CD8T-41BBz-CAR co-expressed with a CSR comprising anti-CD19 EC, CD28 TM, and CD28 IC
Construct: αCD19-CD8T-41BBz-CAR+αCD19-CD30-CSR: anti-CD19-CD8T-41BBz-CAR co-expressed with a CSR comprising anti-CD19 EC, CD30 TM, and CD30 IC
Construct: αCD19-CD8T-41BBz-CAR+αCD19-CD28T-CD30-CSR: anti-CD19-CD8T-41BBz-CAR co-expressed with a CSR comprising anti-CD19 EC, CD28 TM, and CD30 IC
Construct: αCD19-CD8T-41BBz-CAR+αCD19-CD28T-41BB-CSR: anti-CD19-CD8T-41BBz-CAR co-expressed with a CSR comprising anti-CD19 EC, CD28 TM, and 41BB IC
Construct: A 2^{nd}-gen anti-CD19 CD30 CAR co-expressed with anti-CD19 CD30 CSRs compared to the same CARs co-expressed with CSRs comprising other co-stimulatory fragments

### Construct: αCD19-CD30z-CAR+αCD19-CD30-CSR

Construct: αCD19-CD30z-CAR+αCD19-CD28-CSR: anti-CD19-CD30z-CAR co-expressed with a CSR comprising anti-CD19 EC, CD28 TM, and CD28 IC
Construct: αCD19-CD30z-CAR+αCD19-41BB-CSR: anti-CD19-CD30z-CAR co-expressed with a CSR comprising anti-CD19 EC, 4-1BB TM, and 4-1BB IC
Construct: αCD19-CD30z-CAR+αCD19-OX40-CSR: anti-CD19-CD30z-CAR co-expressed with a CSR comprising anti-CD19 EC, OX40 TM, and OX40 IC
Construct: αCD19-CD30z-CAR+αCD19-CD27-CSR: anti-CD19-CD30z-CAR co-expressed with a CSR comprising anti-CD19 EC, CD27 TM, and CD27 IC
Construct: αCD19-CD30z-CAR+αCD19-CD28T-CD30-CSR: anti-CD19-CD30z-CAR co-expressed with a CSR comprising anti-CD19 EC, CD28 TM, and CD30 IC
Construct: αCD19-CD30z-CAR+αCD19-41BBT-CD30-CSR: anti-CD19-CD30z-CAR co-expressed with a CSR comprising anti-CD19 EC, 4-1BB TM, and CD30 IC
Construct: αCD19-CD30z-CAR+αCD19-OX40T-CD30-CSR: anti-CD19-CD30z-CAR co-expressed with a CSR comprising anti-CD19 EC, OX40 TM, and CD30 IC
Construct: αCD19-CD30z-CAR+αCD19-CD27T-CD30-CSR: anti-CD19-CD30z-CAR co-expressed with a CSR comprising anti-CD19 EC, CD27 TM, and CD30 IC
Construct: αCD19-CD30z-CAR+αCD19-CD8T-CD30-CSR: anti-CD19-CD30z-CAR co-expressed with a CSR comprising anti-CD19 EC, CD8 TM, and CD30 IC
Construct: 2^{nd}-gen anti-CD22 CARs co-expressed with anti-CD22 CSRs comprising CD28 or CD30 co-stimulatory fragments
Construct: αCD22-CD30z-CAR: a 2^{nd} generation CAR comprising anti-CD22 EC, CD30 TM, CD30 IC, and CD3zeta IC
Construct: αCD22-CD30z-CAR+αCD22-CD28-CSR: anti-CD22-CD30z-CAR co-expressed with a CSR comprising anti-CD22 EC, CD28 TM, and CD28 IC
Construct: αCD22-CD30z-CAR+αCD22-CD30-CSR: anti-CD22-CD30z-CAR co-expressed with a CSR comprising anti-CD22 EC, CD30 TM, and CD30 IC
Construct: αCD22-CD28z-CAR: a 2^{nd} generation CAR comprising anti-CD22 EC, CD28 TM, CD28 IC, and CD3zeta IC
Construct: αCD22-CD28z-CAR+αCD22-CD28-CSR: anti-CD22-CD28z-CAR co-expressed with a CSR comprising anti-CD22 EC, CD28 TM, and CD28 IC
Construct: αCD22-CD28z-CAR+αCD22-CD30-CSR: anti-CD22-CD28z-CAR co-expressed with a CSR comprising anti-CD22 EC, CD30 TM, and CD30 IC
Construct: αCD22-CD8T-41BBz-CAR: a 2^{nd} generation CAR comprising anti-CD22 EC, CD8 TM, 41-BB IC, and CD3zeta IC
Construct: αCD22-CD8T-41BBz-CAR+αCD22-CD28-CSR: anti-CD22-CD8T-41BBz-CAR co-expressed with a CSR comprising anti-CD22 EC, CD28 TM, and CD28 IC
Construct: αCD22-CD8T-41BBz-CAR+αCD22-CD30-CSR: anti-CD22-CD8T-41BBz-CAR co-expressed with a CSR comprising anti-CD22 EC, CD30 TM, and CD30 IC
Construct: 2^{nd}-gen anti-CD22 CD30 CAR co-expressed with anti-CD22 CD30 CSRs compared to the same CARs co-expressed with CSRs comprising other co-stimulatory fragments.

### Construct: αCD22-CD30z-CAR+αCD22-CD30-CSR

Construct: αCD22-CD30z-CAR+αCD22-CD28-CSR: anti-CD22-CD30z-CAR co-expressed with a CSR comprising anti-CD22 EC, CD28 TM, and CD28 IC
Construct: αCD22-CD30z-CAR+αCD22-41BB-CSR: anti-CD22-CD30z-CAR co-expressed with a CSR comprising anti-CD22 EC, 4-1BB TM, and 4-1BB IC
Construct: αCD22-CD30z-CAR+αCD22-OX40-CSR: anti-CD22-CD30z-CAR co-expressed with a CSR comprising anti-CD22 EC, OX40 TM, and OX40 IC
Construct: αCD22-CD30z-CAR+αCD22-CD27-CSR: anti-CD22-CD30z-CAR co-expressed with a CSR comprising anti-CD22 EC, CD27 TM, and CD27 IC
Construct: αCD22-CD30z-CAR+αCD22-CD28T-CD30-CSR: anti-CD22-CD30z-CAR co-expressed with a CSR comprising anti-CD22 EC, CD28 TM, and CD30 IC
Construct: αCD22-CD30z-CAR+αCD22-41BBT-CD30-CSR: anti-CD22-CD30z-CAR co-expressed with a CSR comprising anti-CD22 EC, 4-1BB TM, and CD30 IC
Construct: αCD22-CD30z-CAR+αCD22-OX40T-CD30-CSR: anti-CD22-CD30z-CAR co-expressed with a CSR comprising anti-CD22 EC, OX40 TM, and CD30 IC
Construct: αCD22-CD30z-CAR+αCD22-CD27T-CD30-CSR: anti-CD22-CD30z-CAR co-expressed with a CSR comprising anti-CD22 EC, CD27 TM, and CD30 IC
Construct: αCD22-CD30z-CAR+αCD22-CD8T-CD30-CSR: anti-CD22-CD30z-CAR co-expressed with a CSR comprising anti-CD22 EC, CD8 TM, and CD30 IC
Construct: A bispecific 2^{nd}-gen anti-CD19, anti-CD22, CD30 CAR co-expressed with anti-CD19, anti-CD22, and/or anti-CD20, CD30 CSRs
Construct: αCD19-αCD22-CD30z-CAR: a bispecific 2^{nd} generation CAR comprising anti-CD19 EC, anti-CD22 EC, CD30 TM, CD30 IC, and CD3zeta IC
Construct: αCD22-αCD19-CD30z-CAR: a bispecific 2^{nd} generation CAR comprising anti-CD22 EC, anti-CD19 EC, CD30 TM, CD30 IC, and CD3zeta IC
Construct: αCD19-CD30z-CAR: a monospecific 2^{nd} generation CAR comprising anti-CD19 EC, CD30 TM, CD30 IC, and CD3zeta IC
Construct: αCD22-CD30z-CAR: a monospecific 2^{nd} generation CAR comprising anti-CD22 EC, CD30 TM, CD30 IC, and CD3zeta IC
Construct: αCD19-αCD22-CD30z-CAR+αCD19-CD30-CSR: bispecific anti-CD19-anti-CD22-CD30z-CAR co-expressed with a monospecific CSR comprising anti-CD19 EC, CD30 TM, and CD30 IC
Construct: αCD19-αCD22-CD30z-CAR+αCD22-CD30-CSR: bispecific anti-CD19-anti-CD22-CD30z-CAR co-expressed with a monospecific CSR comprising anti-CD22 EC, CD30 TM, and CD30 IC
Construct: αCD19-αCD22-CD30z-CAR+αCD20-CD30-CSR: bispecific anti-CD19-anti-CD22-CD30z-CAR co-expressed with a monospecific CSR comprising anti-CD20 EC, CD30 TM, and CD30 IC
Construct: αCD19-CD30z-CAR+αCD22-CD30-CSR: monospecific anti-CD19-CD30z-CAR co-expressed with a monospecific CSR comprising anti-CD22 EC, CD30 TM, and CD30 IC
Construct: αCD22-CD30z-CAR+αCD19-CD30-CSR: monospecific anti-CD22-CD30z-CAR co-expressed with a monospecific CSR comprising anti-CD19 EC, CD30 TM, and CD30 IC
Construct: αCD19-CD30z-CAR+αCD20-CD30-CSR: monospecific anti-CD19-CD30z-CAR co-expressed with a monospecific CSR comprising anti-CD20 EC, CD30 TM, and CD30 IC
Construct: αCD22-CD30z-CAR+αCD20-CD30-CSR: monospecific anti-CD22-CD30z-CAR co-expressed with a monospecific CSR comprising anti-CD20 EC, CD30 TM, and CD30 IC
Construct: αCD19-αCD22-CD30z-CAR+αCD22-αCD19-CD30-CSR: bispecific anti-CD19-anti-CD22-CD30z-CAR co-expressed with a bispecific CSR comprising anti-CD22 EC, anti-CD19 EC, CD30 TM, and CD30 IC
Construct: αCD19-αCD22-CD30z-CAR+αCD20-αCD19-CD30-CSR: bispecific anti-CD19-anti-CD22-CD30z-CAR co-expressed with a bispecific CSR comprising anti-CD20 EC, anti-CD19 EC, CD30 TM, and CD30 IC
Construct: αCD19-αCD22-CD30z-CAR+αCD22-αCD20-CD30-CSR: bispecific anti-CD19-anti-CD22-CD30z-CAR co-expressed with a bispecific CSR comprising anti-CD22 EC, anti-CD20 EC, CD30 TM, and CD30 IC
Construct: αCD22-CD30z-CAR+αCD22-αCD19-CD30-CSR: monospecific anti-CD22-CD30z-CAR co-expressed with a bispecific CSR comprising anti-CD22 EC, anti-CD19 EC, CD30 TM, and CD30 IC
Construct: a tri-specific 2^{nd}-gen anti-CD19, anti-CD22, anti-CD20, CD30 CAR co-expressed with anti-CD19, anti-CD22, and/or anti-CD20, CD30 CSRs
Construct: αCD19-αCD22-αCD20-CD30z-CAR: a tri-specific 2^{nd} generation CAR comprising anti-CD19 EC, anti-CD22 EC, anti-CD20 EC, CD30 TM, CD30 IC, and CD3zeta IC
Construct: αCD19-αCD22-CD30z-CAR: a bispecific 2^{nd} generation CAR comprising anti-CD19 EC, anti-CD22 EC, CD30 TM, CD30 IC, and CD3zeta IC
Construct: αCD19-CD30z-CAR: a monospecific 2^{nd} generation CAR comprising anti-CD19 EC, CD30 TM, CD30 IC, and CD3zeta IC
Construct: αCD22-CD30z-CAR: a monospecific 2^{nd} generation CAR comprising anti-CD22 EC, CD30 TM, CD30 IC, and CD3zeta IC
Construct: αCD19-αCD22-αCD20-CD30z-CAR+αCD19-CD30-CSR: tri-specific anti-CD19-anti-CD22-anti-CD20-CD30z-CAR co-expressed with a monospecific CSR comprising anti-CD19 EC, CD30 TM, and CD30 IC
Construct: αCD19-αCD22-αCD20-CD30z-CAR+αCD22-CD30-CSR: tri-specific anti-CD19-anti-CD22-anti-CD20-CD30z-CAR co-expressed with a monospecific CSR comprising anti-CD22 EC, CD30 TM, and CD30 IC
Construct: αCD19-αCD22-αCD20-CD30z-CAR+αCD20-CD30-CSR: tri-specific anti-CD19-anti-CD22-anti-CD20-CD30z-CAR co-expressed with a monospecific CSR comprising anti-CD20 EC, CD30 TM, and CD30 IC
Construct: αCD19-αCD22-CD30z-CAR+αCD20-CD30-CSR: bispecific anti-CD19-anti-CD22-CD30z-CAR co-expressed with a monospecific CSR comprising anti-CD20 EC, CD30 TM, and CD30 IC
Construct: αCD19-CD30z-CAR+αCD22-CD30-CSR: monospecific anti-CD19-CD30z-CAR co-expressed with a monospecific CSR comprising anti-CD22 EC, CD30 TM, and CD30 IC
Construct: αCD19-CD30z-CAR+αCD20-CD30-CSR: monospecific anti-CD19-CD30z-CAR co-expressed with a monospecific CSR comprising anti-CD20 EC, CD30 TM, and CD30 IC
Construct: αCD19-αCD22-αCD20-CD30z-CAR+αCD22-αCD19-CD30-CSR: tri-specific anti-CD19-anti-CD22-anti-CD20-CD30z-CAR co-expressed with a bispecific CSR comprising anti-CD22 EC, anti-CD19 EC, CD30 TM, and CD30 IC
Construct: αCD19-αCD22-αCD20-CD30z-CAR+αCD20-αCD19-CD30-CSR: tri-specific anti-CD19-anti-CD22-anti-CD20-CD30z-CAR co-expressed with a bispecific CSR comprising anti-CD20 EC, anti-CD19 EC, CD30 TM, and CD30 IC
Construct: αCD19-αCD22-αCD20-CD30z-CAR+αCD22-αCD20-CD30-CSR: tri-specific anti-CD19-anti-CD22-anti-CD22-CD30z-CAR co-expressed with a bispecific CSR comprising anti-CD22 EC, anti-CD20 EC, CD30 TM, and CD30 IC
Construct: αCD19-αCD22-CD30z-CAR+αCD20-αCD19-CD30-CSR: bispecific anti-CD19-anti-CD22-CD30z-CAR co-expressed with a bispecific CSR comprising anti-CD20 EC, anti-CD19 EC, CD30 TM, and CD30 IC
Construct: αCD19-αCD22-CD30z-CAR+αCD22-αCD20-CD30-CSR: bispecific anti-CD19-anti-CD22-CD30z-CAR co-expressed with a bispecific CSR comprising anti-CD22 EC, anti-CD20 EC, CD30 TM, and CD30 IC
Construct: αCD19-αCD22-αCD20-CD30z-CAR+αCD22-αCD20-αCD19-CD30-CSR: tri-specific anti-CD19-anti-CD22-anti-CD20-CD30z-CAR co-expressed with a tri-specific CSR comprising anti-CD22 EC, anti-CD20 EC, anti-CD19 EC, CD30 TM, and CD30 IC

### Construct: Anti-ROR1-CAR+Anti-ROR1-CSR

For solid tumors (neuroblastoma) and CLL (most common leukemia), mantle cell lymphoma (MCL, 5% of NHL):

Construct: αROR1-CD30z-CAR: anti-ROR1 EC, CD30 TM and CD30 IC, CD3zeta IC
Construct: αROR1-CD30z-CAR+αROR1-CD30-CSR: anti-ROR1 EC, CD30 TM and IC and CD3zeta IC+anti-ROR1 EC, CD30 TM and IC
Construct: αROR1-CD30z-CAR+αROR1-CD28-CSR: anti-ROR1 EC, CD30 TM and IC and CD3zeta IC+anti-ROR1 EC, CD28 TM and IC
Construct: αROR1-CD30z-CAR+αROR1-41BB-CSR: anti-ROR1 EC, CD30 TM and IC and CD3zeta IC+anti-ROR1 EC, 4-1BB TM and IC.
Construct: αROR1-CD28z-CAR: anti-ROR1 EC, CD28 TM and CD28 IC, CD3zeta IC
Construct: αROR1-CD28z-CAR+αROR1-CD30-CSR: anti-ROR1 EC, CD28 TM and CD28 IC, CD3zeta IC+anti-ROR1 EC, CD30 TM and IC.
Construct: αROR1-CD8T-CD30z-CAR: anti-ROR1 EC, CD8 TM, CD30 IC and CD3zeta IC
Construct: αROR1-CD8T-CD30z-CAR+αROR1-CD30-CSR: anti-ROR1 EC, CD8 TM, CD30 IC and CD3zeta IC+anti-ROR1 EC, CD30 TM and IC
Construct: αROR1-CD8T-41BBz-CAR: anti-ROR1 EC, CD8 TM, 4-1BB IC and CD3zeta IC
Construct: αROR1-CD8T-41BBz-CAR+αROR1-CD30-CSR: anti-ROR1 EC, CD8 TM, 4-1BB IC and CD3zeta IC+anti-ROR1 EC, CD30 TM and IC
Construct: αROR1-CD8T-41BBz-CAR+αROR1-CD28T-CD30-CSR: anti-ROR1 EC, CD8 TM, 4-1BB IC and CD3zeta IC+anti-ROR1 EC, CD28 TM and CD30 IC
Construct: αROR1-CD8T-41BBz-CAR+αROR1-CD28T-41BB-CSR: anti-ROR1 EC, CD8 TM, 4-1BB IC and CD3zeta IC+anti-ROR1 EC, CD28 TM and 41BB IC
Construct: αROR1-CD28T-41BBz-CAR: anti-ROR1 EC, CD28 TM, 4-1BB IC and CD3zeta IC
Construct: αROR1-CD28T-41BBz-CAR+αROR1-CD30-CSR: anti-ROR1 EC, CD28 TM, 4-1BB IC and CD3zeta IC+anti-ROR1 EC, CD30 TM and IC
Construct: anti-PSMA-CAR+anti-PSMA-CSR: 2^{nd} generation anti-PSMA1 CD30 or 4-1BB CAR co-expressed with anti-PMSA-CSRs comprising CD30 TM and IC.

Prostate cancer:

Construct: αPSMA-CD30z-CAR: anti-PSMA EC, CD30 TM and IC, CD3zeta IC
Construct: αPSMA-CD30z-CAR+αPSMA-CD30-CSR: anti-PSMA EC, CD30 TM and IC and CD3zeta IC+anti-PSMA EC, CD30 TM and IC
Construct: αPSMA-CD8T-CD30z-CAR: anti-PSMA EC, CD8 TM, CD30 IC, CD3zeta IC
Construct: αPSMA-CD8T-CD30z-CAR+αPSMA-CD30-CSR: anti-PSMA EC, CD8 TM, CD30 IC and CD3zeta IC+anti-PSMA EC, CD30 TM and IC
Construct: αPSMA-CD8T-41BBz-CAR: anti-PSMA EC, CD8 TM, 4-1BB IC, CD3zeta IC
Construct: αPSMA-CD8T-41BBz-CAR+αPSMA-CD30-CSR: anti-PSMA EC, CD8 TM, 4-1BB IC and CD3zeta IC+anti-PSMA EC, CD30 TM and IC
Construct: anti-NY-ESO-1/MHC CAR+anti-EGFR-CSR: 2^{nd} generation anti-NY-ESO-1/MHC CAR with CD30 or 4-1BB IC and CD3zeta IC+anti-EGFR-CD30-CSR
Construct: αNYESO1-CD30z-CAR: anti-NY-ESO-1/MHC EC, CD30 TM and IC, CD3zeta IC
Construct: αNYESO1-CD30z-CAR+αEGFR-CD30-CSR: anti-NY-ESO-1/MHC EC, CD30 TM and IC, CD3zeta IC+αEGFR EC, CD30 TM and IC-CSR.
Construct: αNYESO1-CD8T-CD30z-CAR: anti-NY-ESO-1/MHC EC, CD8 TM, CD30 IC, CD3zeta IC αNYESO1-CD8T-CD30z-CAR+αEGFR-CD30-CSR anti-NY-ESO-1/MHC EC, CD8 TM, CD30 IC, CD3zeta IC+αEGFR EC, CD30 TM and IC CSR.
Construct: αNYESO1-CD8T-41BBz-CAR: anti-NY-ESO-1/MHC EC, CD8 TM, 4-1BB IC, CD3zeta IC
Construct: αNYESO1-CD8T-41BBz-CAR+αEGFR-CD30-CSR: anti-NY-ESO-1/MHC EC, CD8 TM, 4-1BB IC, CD3zeta IC+αEGFR EC, CD30 TM and IC CSR.

### Example 10—Short-Term Killing of Target Cells by Anti-AFP/MHC CAR+Anti-GPC3-CSR T Cells

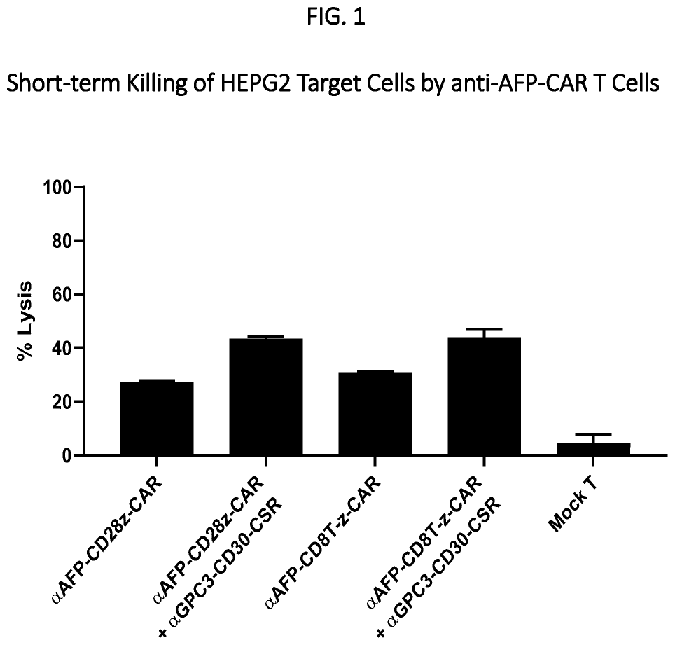

This example shows that CAR+CD30-CSR expressing T cells have higher specific tumor cell killing efficacies than CAR T cells without CSR. Primary T cells were mock-transduced (no DNA added) or transduced with lentiviral vectors encoding: (1) anti-AFP-CD28z-CAR (SEQ ID NO:7); (2) anti-AFP-CD28z-CAR+anti-GPC3-CD30-CSR (SEQ ID NO:7+SEQ ID NO:13, respectively); (3) anti-AFP-CD8T-z-CAR (SEQ ID NO: 1); or (4) anti-AFP-CD8T-z-CAR+anti-GPC3-CD30-CSR (SEQ ID NO:1+SEQ ID NO:13, respectively) for 7-9 days. The transduction efficiency was determined by staining with PE-labeled AFP158/HLA-A*02:01 tetramers (“AFP158 tetramers”). The CAR T cells were normalized to 35% CAR⁺ (or “receptor”) and tested for their abilities to kill cancer cells with a FACS-based assay. Activated T cells and target cells HepG2 (AFP⁺, HLA-A2⁺, GPC3⁺) were co-cultured at an effector-to-target ratio of 2:1. Specific lysis was determined by measuring LDH activity in culture supernatants after 16 hr incubation using the Cytox 96 Non-radioactive Cytotoxicity Assay (Promega). As shown in FIG. 1, T cells transduced with vectors encoding both CAR (the 1^{st}-generation: anti-AFP-CD8T-z-CAR or the 2^{nd}-generation: anti-AFP-CD28z-CAR) and CD30-CSR had higher in vitro tumor cell killing efficacies than corresponding CAR T cells without CSR.

### Example 11—Cytokine Production and Secretion by Anti-AFP/MHC CAR+Anti-GPC3-CSR T Cells

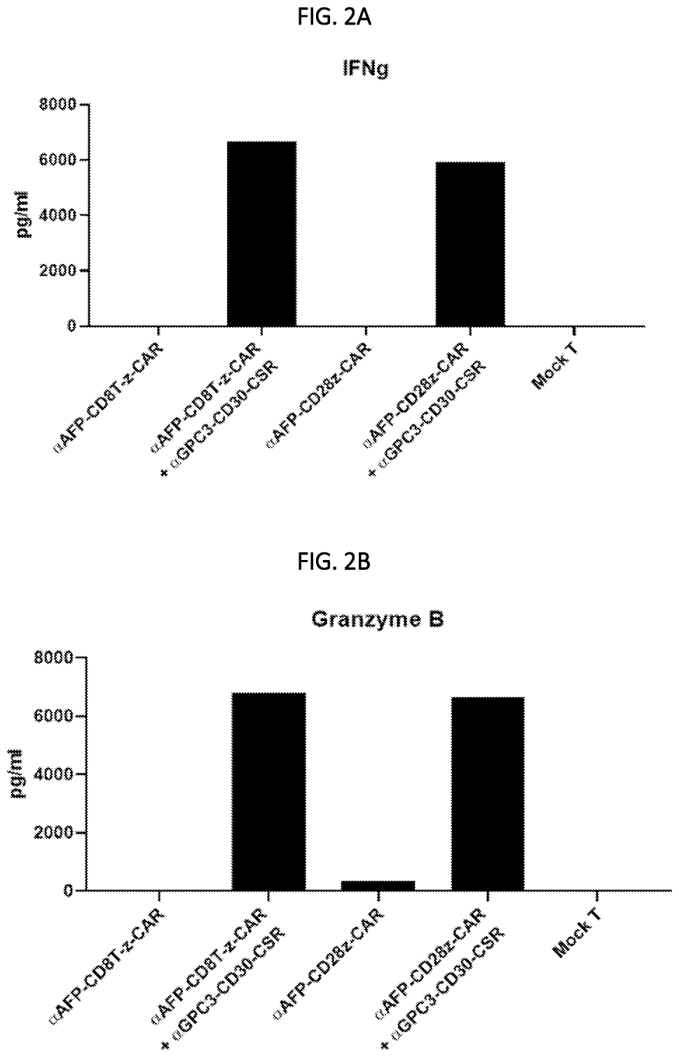

This example shows CAR+CD30-CSR expressing T cells have higher specific T cell activities than CAR T cells without CSR. IFNγ and Granzyme B are both indicators for T cell activities/killing capability. After transduction, 50,000 CAR⁺ anti-AFP-CAR T cells and anti-AFP-CAR+anti-GPC3-CD30-CSR T Cells were incubated with HepG2 target cells at an effector cell to target cell ratio (E:T ratio) of 1:1. The cells are rechallenged with 100,000 Hep2G target cells every 7 days after the first engagement. After three engagements, IFNγ and Granzyme B levels in the culture supernatants were quantified with ELISA MAX™ Deluxe Set Human IFNγ by BioLegend (San Diego, Calif.) and Human Granzyme B DuoSet ELISA by R&D Systems (Minneapolis, Minn.), respectively, and the results are shown in FIGS. 2A and 2B, respectively. Reactions that demonstrated an increase in cytotoxic potency in Example 10 also showed increases in the amounts of cytokines (IFNγ and Granzyme B) released. Specifically, T cells transduced with vectors encoding both CAR (the 1^{st}-generation: anti-AFP-CD8T-z-CAR or the 2^{nd}-generation: anti-AFP-CD28z-CAR) and CD30-CSR had much higher IFNγ and Granzyme B secretion levels than corresponding CAR T cells without CSR.

### Example 12—Long-Term Killing of Target Cells by Anti-AFP/MHC CAR+Anti-GPC3-CSR T Cells and T Cell Survival

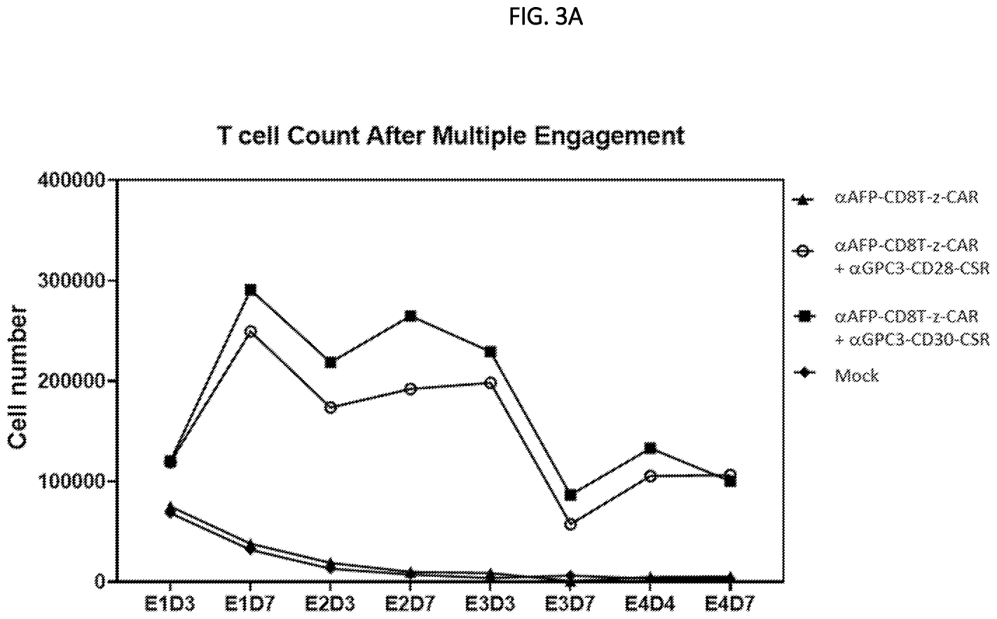

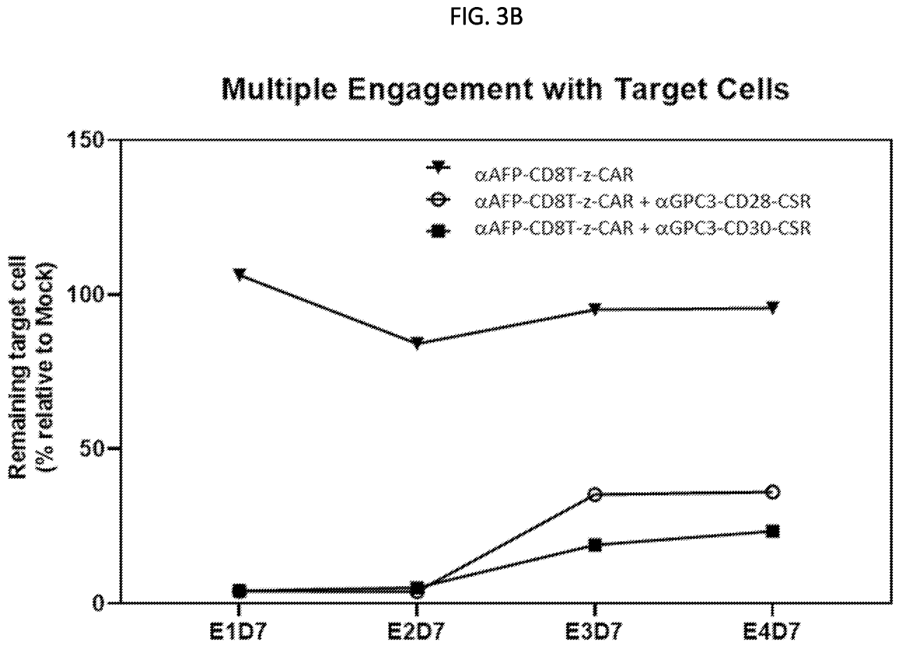

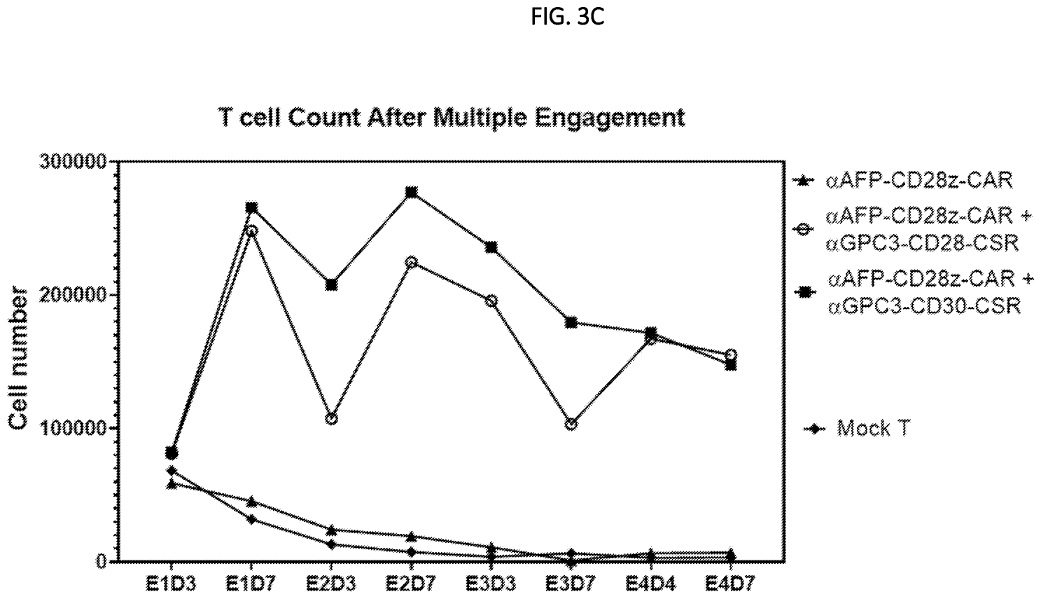

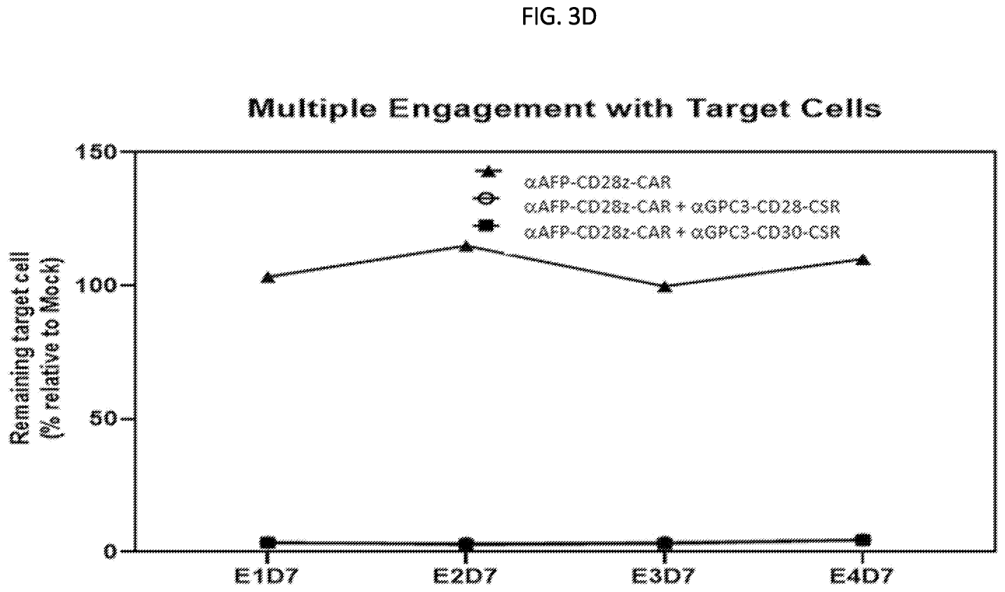

A FACS based assay for counting target cells was used to compare the long-term killing potential of CAR T cells. The effector cells used were primary T cells from donor subjects transduced with vectors encoding various CAR constructs. The effector cells were transduced with vectors encoding: 1^{st} generation CAR constructs (FIGS. 3A and 3B): (1) anti-AFP-CD8T-z-CAR (SEQ ID NO:1); (2) anti-AFP-CD8T-z-CAR+anti-GPC3-CD28-CSR (SEQ ID NO:1+SEQ ID NO:14); or (3) anti-AFP-CD8T-z-CAR+anti-GPC3-CD30-CSR (SEQ ID NO:1+SEQ ID NO:13), or 2^{nd} generation CAR constructs (FIGS. 3C and 3D): (1) anti-AFP-CD28z-CAR (SEQ ID NO:7); (2) anti-AFP-CD28z-CAR+anti-GPC3-CD28-CSR (SEQ ID NO:7+SEQ ID NO:14); or (3) anti-AFP-CD28z-CAR+anti-GPC3-CD30-CSR (SEQ ID NO:7+SEQ ID NO:13) for 7-9 days. The effector cells were normalized to 35% receptor⁺ based on AFP158 tetramer staining.

The target cells used were HepG2 (A2⁺/AFP⁺/GPC3⁺) cells. The effector to target ratio (E:T ratio) in this experiment was 1:1. Specifically, 50,000 receptor⁺ T cells and 50,000 HepG2 cells were incubated together in each well in RPMI+10% FBS with no cytokine. The cells were rechallenged with 100,000 HepG2 cells per well every 7 days. The numbers of remaining target cells and receptor⁺ T cells were quantified on selected days after each target cell engagement. The results of T cell survival (total T cell numbers, not just receptor⁺ ones) and the long-term killing (represented by remaining target cells' percentage relative to target cells incubated with mock-transduced T cells) are shown in FIGS. 3A-3D, with 1^{st}-generation CARs' results in FIGS. 3A and 3B and 2^{nd}-generation CARs' results in FIGS. 3C and 3D. FIG. 3B shows that T cells expressing the 1^{st}-generation anti-AFP-CAR co-expressed with anti-GPC3-CD30-CSR or anti-GPC3-CD28-CSR both killed many more target cells than T cell expressing the CAR alone. Surprisingly, T cells expressing the 1s-generation CAR co-expressed with CD30-CSR killed significantly more target cells than corresponding T cells with CD28-CSR.

FIG. 3D shows that T cells expressing the 2^{nd}-generation anti-AFP-CAR (anti-AFP-CD28z-CAR) co-expressed with anti-GPC3-CD30-CSR or anti-GPC3-CD28-CSR both effectively mediated the killing of almost all of the initially engaged and the rechallenged target cells, unlike T cells expressing the 2^{nd}-generation anti-AFP-CAR only which hardly killed any target cells relative to mock-transduced T cells. Surprisingly, FIGS. 3A and 3C show that T cells expressing anti-AFP-CD8-z-CAR+anti-GPC3-CD30-CSR and anti-AFP-CD28z-CAR+anti-GPC3-CD30-CSR, respectively, not only survived much better than mock-transduced T cells and T cells expressing only the corresponding CARs, but also survived and even multiplied significantly better than T cells expressing the corresponding CARs+CD28-CSR.

### Example 13—Expression of T Cell Exhaustion Markers in Anti-AFP/MHC CAR+Anti-GPC3-CSR T Cells T Cells after Co-Culture with Target Cells

To examine the level of exhaustion markers expressed on CAR-transduced cells upon antigen stimulation, CD3⁺ T cells were prepared from PBMC-enriched whole blood using EasySep Human T Cell Isolation Kit (StemCell Technologies) and activated with CD3/CD28 Dynabeads. The activated and expanded cell population was >99% CD3⁺ by flow cytometry. These cells were then transduced with lentiviral vectors encoding the construct described in Tables 2, 3, and 4 below for 7-9 days. The transduced cells (effector cells) were normalized to 35% receptor⁺ based on AFP158 tetramer staining. The effector cells were then co-cultured with HepG2 target cells at an E:T ratio of 1:1. Specifically, 50,000 receptor⁺ T cells and 50,000 HepG2 cells were incubated together in each well in RPMI+10% FBS with no cytokine. The cells were rechallenged with 100,000 HepG2 cells per well every 7 days. The MFI levels of exhaustion markers PD-1, LAG3, and TIGIT on the receptor⁺ T cells were analyzed by flow cytometry on selected days after each target cell engagement. PD-1, LAG3, and TIGIT are inhibitory receptors that accumulate on T cells as T cells lose function. Because of this phenomenon these molecules' expression is seen as a marker of exhausted T cells. The MFI levels of these exhaustion markers and ratios of some exhaustion marker levels of the CAR+CD30-CSR T cells over those of CAR+CD28-CSR or CAR alone T cells are shown in Tables 2, 3, and 4. Surprisingly, expressing CAR+CD30-CSR resulted in T cells with significantly less exhaustion marker accumulation than expressing CAR alone or CAR+CD28-CSR, indicative of significantly more functional and less exhausted T cells. Also significantly, the lower levels of T cell exhaustion markers resulted from CAR+CD30-CSR expression were seen in both CD8⁺ T cells (cytotoxic T cells which are more directly involved in target cell killing) and CD4⁺ T cells (T helper cells which help the function of other immune cells including the activation and growth of cytotoxic T cells).

TABLE 2
PDI Expression Levels on Receptor⁺ T Cells

| | | | | Ratio of PDI | Ratio |
|---|---|---|---|---|---|
| | | | | Expression | of PDI |
| | | | | Levels | Expression |
| | | | | CAR + | Levels |
| | | | | CD30-CSR/ | CAR + |
| | | | | CAR + | CD30- |
| Date | | | | CD28-CSR | CSR/CAR |
| | PDI Expression Level on 1^{st}-gen CAR⁺ | | | | |
| | CD8⁺ T Cells (MFI) | | | | |
| | | αAFP- | αAFP- | | |
| | | CD8T-Z- | CD8T-Z- | | |
| | αAFP- | CAR + | CAR + | | |
| | CD8T-Z- | αGPC3- | αGPC3- | | |
| | CAR | CD30-CSR | CD28-CSR | | |
| E1D5 | 403 | 66.1 | 92.9 | 0.71 | 0.16 |
| E2D5 | 607 | 102 | 235 | 0.43 | 0.17 |
| E3D5 | 541 | 61.8 | 392 | 0.16 | 0.11 |
| | PDI Expression Level on 2^{nd}-gen CAR⁺ | | | | |
| | CD8⁺ T Cells (MFI) | | | | |
| | | αAFP- | αAFP- | | |
| | | CD28z- | CD28z- | | |
| | αAFP- | CAR + | CAR + | | |
| | CD28z- | αGPC3- | αGPC3- | | |
| | CAR | CD30-CSR | CD28-CSR | | |
| E1D5 | 599 | 69.5 | 173 | 0.40 | 0.12 |
| E2D5 | 952 | 91.5 | 405 | 0.23 | 0.10 |
| E3D5 | 652 | 42.8 | 281 | 0.15 | 0.07 |
| | PD1 Expression Level on 1^{st}-gen CAR⁺ | | | | |
| | CD4⁺ T Cells (MFI) | | | | |
| | | αAFP- | αAFP- | | |
| | | CD8T-Z- | CD8T-Z- | | |
| | αAFP- | CAR + | CAR + | | |
| | CD8T-Z- | αGPC3- | αGPC3- | | |
| | CAR | CD30-CSR | CD28-CSR | | |
| E1D5 | 1114 | 193 | 434 | 0.44 | 0.17 |
| E2D5 | 1092 | 288 | 1084 | 0.27 | 0.26 |
| E3D5 | 855 | 168 | 1703 | 0.10 | 0.20 |
| | PD1 Expression Level on 2^{nd}-gen CAR⁺ | | | | |
| | CD4⁺ T Cells (MFI) | | | | |
| | | αAFP- | αAFP- | | |
| | | CD28z- | CD28z- | | |
| | αAFP- | CAR + | CAR + | | |
| | CD28z- | αGPC3- | αGPC3- | | |
| | CAR | CD30-CSR | CD28-CSR | | |
| E1D5 | 1764 | 203 | 748 | 0.27 | 0.12 |
| E2D5 | 2280 | 322 | 1516 | 0.21 | 0.14 |
| E3D5 | 2350 | 161 | 1236 | 0.13 | 0.07 |

TABLE 3A
LAG3 Expression Levels on Receptor⁺ T Cells

| | | | | Ratio of |
|---|---|---|---|---|
| | LAG3 Expression Level | | | LAG3 |
| | on 1^{st}-gen CAR⁺ CD8⁺ | | | Expression |
| | T Cells (MFI) | | | Levels |
| | | αAFP-CD8T- | αAFP-CD8T- | CAR + |
| | αAFP- | Z-CAR + | Z-CAR + | CD30-CSR/ |
| | CD8T- | αGPC3- | αGPC3- | CAR + |
| Date | z-CAR | CD30-CSR | CD28-CSR | CD28-CSR |
| E1D5 | 514 | 432 | 571 | 0.76 |
| E2D5 | 208 | 225.1 | 346.8 | 0.65 |
| E3D5 | 420 | 448 | 655 | 0.68 |

TABLE 3B
LAG3 Expression Levels on Receptor⁺ T Cells

| | LAG3 Expression Level | | | Ratio of | |
|---|---|---|---|---|---|
| | on 2^{nd}-gen CAR⁺ CD8⁺ | | | LAG3 | Ratio of |
| | T Cells (MFI) | | | Expression | LAG3 |
| | | αAFP- | αAFP- | Levels | Expression |
| | | CD28z- | CD28z- | CAR + | Levels |
| | αAFP- | CAR + | CAR + | CD30-CSR/ | CAR + |
| | CD28z- | αGPC3- | αGPC3- | CAR + | CD30- |
| Date | CAR | CD30-CSR | CD28-CSR | CD28-CSR | CSR/CAR |
| E1D5 | 1004 | 814 | 1601 | 0.51 | 0.81 |
| E2D5 | 868 | 356 | 691 | 0.52 | 0.41 |
| E3D5 | 1248 | 575 | 830 | 0.69 | 0.46 |

TABLE 3C
LAG3 Expression Levels on Receptor⁺ T Cells

| | | | | Ratio of |
|---|---|---|---|---|
| | LAG3 Expression Level | | | LAG3 |
| | on 1^{st}-gen CAR⁺ CD4⁺ | | | Expression |
| | T Cells (MFI) | | | Levels |
| | | αAFP-CD8T- | αAFP-CD8T- | CAR + |
| | αAFP- | Z-CAR + | Z-CAR + | CD30-CSR/ |
| | CD8T- | αGPC3- | αGPC3- | CAR + |
| Date | z-CAR | CD30-CSR | CD28-CSR | CD28-CSR |
| E1D5 | 210 | 215.4 | 327.5 | 0.66 |
| E2D5 | 68 | 3 | 44 | 0.07 |
| E3D5 | 222.5 | 272.7 | 323.6 | 0.84 |

TABLE 3D
LAG3 Expression Levels on Receptor⁺ T Cells

| | LAG3 Expression Level | | | Ratio of | |
|---|---|---|---|---|---|
| | on 2^{nd}-gen CAR⁺ CD4⁺ | | | LAG3 | Ratio of |
| | T Cells (MFI) | | | Expression | LAG3 |
| | | αAFP- | αAFP- | Levels | Expression |
| | | CD28z- | CD28z- | CAR + | Levels |
| | αAFP- | CAR + | CAR + | CD30-CSR/ | CAR + |
| | CD28z- | αGPC3- | αGPC3- | CAR + | CD30- |
| Date | CAR | CD30-CSR | CD28-CSR | CD28-CSR | CSR/CAR |
| E1D5 | 593 | 443 | 709 | 0.62 | 0.75 |
| E2D5 | 355.1 | 163 | 315.98 | 0.52 | 0.46 |
| E3D5 | 523 | 476 | 557 | 0.85 | 0.91 |

TABLE 4
TIGIT Expression Levels on Receptor⁺ T Cells

| | TIGIT Expression Level on 1^{st}- | | Ratio of TIGIT |
|---|---|---|---|
| | gen CAR⁺ Total T Cells | | Expression |
| | αAFP-CD8T- | αAFP-CD8T- | Levels CAR + |
| | z-CAR + | z-CAR + | CD30-CSR/ |
| | αGPC3- | αGPC3- | CAR + |
| Date | CD30-CSR | CD28-CSR | CD28-CSR |
| E1D3 | 1161 | 1394 | 0.83 |
| E1D5 | 1378 | 1441 | 0.96 |
| E2D3 | 1012 | 1754 | 0.58 |
| E2D5 | 1627 | 2272 | 0.72 |
| E3D3 | 990 | 1677 | 0.59 |
| E3D5 | 867 | 1012 | 0.86 |
| E4D2 | 2117 | 3275 | 0.65 |
| E4D5 | 751 | 1438 | 0.52 |
| E5D2 | 1277 | 2095 | 0.61 |
| E5D5 | 785 | 1178 | 0.67 |

### Example 14—Development and Maintenance of Memory Cells from Anti-AFP/MHC CAR+Anti-GPC3-CSR T Cells

This example shows that CAR+CD30-CSR T cells developed into and maintained a high memory T cell population after target stimulation, including central memory and effector memory T cells. To determine the effect of expressing CAR+CD30-CSR on T cells' ability to develop into and maintain memory T cells as compared to expressing CAR only or CAR+CD28-CSR, we measured the cell surface expression of memory T cell markers CCR7 and CD45RA. As known in the field, T cells with high CCR7 expression levels and low CD45RA expression levels are considered as central memory T cells, T cells with low CCR7 and low CD45RA expression levels are effector memory T cells, T cells with low CCR7 and high CD45RA expression levels are effector T cells, while T cells with high CCR7 and high CD45RA are naïve T cells which are the initial type of T cells before target/antigen challenge/recognition (Eur J Immunol. 2013 November; 43(11):2797-809. doi: 10.1002/eji.201343751. Epub 2013 Oct. 30. The who's who of T-cell differentiation: human memory T-cell subsets. Mahnke Y D1, Brodie T M, Sallusto F, Roederer M, Lugli E.). When in response to antigen encounter, naïve T cells proliferate and differentiate into effector cells, most of which carry out the job of destroying targets and then die, while a small pool of T cells ultimately develops into long-lived memory T cells which can store the T cell immunity against the specific target. Among the memory T cells, the central memory T cells were found to have longer lives than effector memory T cells and be capable of generating effector memory T cells, but not vice versa. Therefore, the ability to develop into and maintain memory T cells, especially central memory T cells, is an important and desired feature for potentially successful T cell therapies. Primary T cells were mocked transduced or transduced with vectors encoding various CAR constructs. The effector cells were transduced with vectors encoding: 1^{st} generation CAR constructs: (1) anti-AFP-CD8T-z-CAR (SEQ ID NO:1); (2) anti-AFP-CD8T-z-CAR+anti-GPC3-CD28-CSR (SEQ ID NO:1+SEQ ID NO:14); or (3) anti-AFP-CD8T-z-CAR+anti-GPC3-CD30-CSR (SEQ ID NO:1+SEQ ID NO:13); or 2^{nd} generation CAR constructs: (1) anti-AFP-CD28z-CAR (SEQ ID NO:7); (2) anti-AFP-CD28z-CAR+anti-GPC3-CD28-CSR (SEQ ID NO:7+SEQ ID NO:14); or (3) anti-AFP-CD28z-CAR+anti-GPC3-CD30-CSR (SEQ ID NO:7+SEQ ID NO:13) for 7-9 days. The effector cells were normalized to 35% receptor⁺ based on AFP158 tetramer staining.

The effector cells expressing 1^{st} generation or 2^{nd} generation CAR constructs alone (anti-AFP-CD8T-z-CAR or anti-AFP-CD28z-CAR) were incubated with HepG2 target cells at an E:T ratio of 2:1 (100,000 receptor⁺ T cells and 50,000 HepG2 cells in each well on a 96-well plate) for 7 days. The cells were then rechallenged with 100,000 HepG2 cells per well every 7 days.

The effector cells expressing CAR+CSR constructs were incubated with HepG2 target cells at an E:T ratio of 1:2 (25,000 receptor⁺ T cells and 50,000 HepG2 cells in each well) for 7 days. The cells were then rechallenged with 100,000 HepG2 cells per well every 7 days.

Each different T cell and target cell mixture sample was made in replicates to ensure at least one mixture to be available for quantification on each selected day. The CAR or CAR+CSR effector and target cell mixtures were diluted 1:6 before the fourth and fifth target cell engagement (E4 and E5) to avoid the overcrowdedness of T cells due to the significant T cell expansion, so that only one sixth of the previously remaining cells were rechallenged with 100,000 HepG2 cells.

On selected days after each target cell engagement, the entire cell mixture in a well from each sample was stained with antibodies against CCR7 and CD45RA and analyzed by flow cytometry. Receptor⁺ T cell numbers were counted, and cells were grouped into various T cell types based on their CCR7 and CD45RA expression levels: central memory T cells (CD45RA⁻ CCR7⁺), effector memory T cells (CD45RA⁻ CCR7⁻), effector T cells (CD45RA⁺ CCR7⁻), and naïve T cells (CD45RA⁺ CCR7⁺). Percentages of various types of T cells among the total number of receptor⁺ T cells were calculated. In some experiments, the cells were also stained with antibodies against CD8 or CD4 to determine the CD8-CD4 characteristics of the counted T cells.

The results of central memory T cell counts of total receptor⁺ T cells (including CD8⁺ and CD4⁺ T cells) as well as ratios of memory T cell counts of the CAR+CD30-CSR T cells over those of CAR+CD28-CSR or CAR alone T cells are shown in Tables 5-7. Tables 5 and 6 show central memory T cell counts of 1^{st}-generation CAR⁺ T cells expressing αAFP-CD8T-z-CAR alone or also expressing CSR (αGPC3-CD28-CSR or αGPC3-CD30-CSR). The CAR and CSR co-expressed in the CAR+CSR T cells of Table 5 were encoded on two separate vectors, while the CAR and CSR of Table 6 were encoded on one vector. Table 7 shows central memory T cell counts of 2^{nd}-generation CAR⁺ T cells expressing αAFP-CD28z-CAR alone or also expressing CSR (αGPC3-CD28-CSR or αGPC3-CD30-CSR). The 2^{nd}-gen CAR and CSR co-expressed in all the experiments disclosed in this Example, including that of Table 7, were encoded on two separate vectors. The results in Tables 5-7 show that, surprisingly, expressing CAR+CD30-CSR resulted in many more central memory T cells than expressing CAR alone or CAR+CD28-CSR at almost all timepoints, especially at extended times after engagement with target cells (e.g., 7 days after the 1^{st} engagement and starting from the 2^{nd} engagement).

TABLE 5
Central Memory T Cell Counts of 1^{st}-generation CAR⁺ T Cells with
CAR and CSR Encoded on Two Separate Vectors.

| | | αAFP- | αAFP- | Ratio of Central | Ratio of |
|---|---|---|---|---|---|
| | | CD8T- | CD8T-z- | Memory T Cell | Central |
| | | z-CAR + | CAR + | Counts CAR + | Memory T |
| | αAFP- | αGPC3- | αGPC3- | CD30-CSR/ | Cell Counts |
| | CD8T- | CD30- | CD28- | CAR + | CAR + CD30- |
| Date | z-CAR | CSR | CSR | CD28-CSR | CSR/CAR |
| E1D3 | 1,657 | 10,543 | 9,359 | 1.13 | 6.36 |
| E1D5 | 1,721 | 14,425 | 12,041 | 1.20 | 8.38 |
| E1D7 | 1,486 | 29,757 | 7,503 | 3.97 | 20.02 |
| E2D3 | 959 | 45,048 | 23,519 | 1.92 | 46.97 |
| E2D5 | 508 | 57,737 | 10,470 | 5.51 | 113.66 |
| E2D7 | 1,869 | 53,986 | 25,167 | 2.15 | 28.88 |
| E3D3 | 109 | 19,674 | 7,430 | 2.65 | 180.50 |
| E3D5 | 609 | 35,055 | 5,088 | 6.89 | 57.56 |
| E4D2^{a} | 14 | 2,075 | 376 | 5.52 | 148.21 |
| E4D5^{a} | 16 | 13,538 | 1,368 | 9.90 | 846.13 |
| E4D7^{a} | 15 | 8,059 | 2,759 | 2.92 | 537.27 |

^{a}The CAR or CAR + CSR effector and target cell mixtures were diluted 1:6 before the fourth target cell engagement (E4).

TABLE 6
Central Memory T Cell Counts of 1^{st}-generation CAR⁺ T Cells
with CAR and CSR Encoded on One Vector.

| | | | Ratio of Central |
|---|---|---|---|
| | | | Memory |
| | αAFP-CD8T-z- | αAFP-CD8T-z- | T Cell Counts |
| | CAR + αGPC3- | CAR + αGPC3- | CAR + CD30-CSR/ |
| Date | CD30-CSR | CD28-CSR | CAR + CD28-CSR |
| E1D3 | 13,558 | 9,882 | 1.37 |
| E1D5 | 22,374 | 14,575 | 1.54 |
| E1D7 | 11,741 | 6,942 | 1.69 |
| E2D3 | 49,009 | 26,084 | 1.88 |
| E2D5 | 38,242 | 14,120 | 2.71 |
| E2D7 | 61,612 | 28,202 | 2.18 |
| E3D3 | 23,571 | 6,150 | 3.83 |
| E3D5 | 26,626 | 10,014 | 2.66 |
| E4D2^{a} | 6,048 | 649 | 9.32 |
| E4D5^{a} | 22,747 | 2,061 | 11.04 |
| E4D7^{a} | 19,363 | 1,681 | 11.52 |

^{a}The CAR or CAR + CSR effector and target cell mixtures were diluted 1:6 before the fourth target cell engagement (E4).

TABLE 7
Central Memory T Cell Counts of 2^{nd}-generation CAR⁺ T Cells
with CAR and CSR Encoded on Two Separate Vectors.

| | | αAFP- | αAFP- | Ratio of Central | |
|---|---|---|---|---|---|
| | | CD28z- | CD28z- | Memory T Cell | |
| | | CAR + | CAR + | Counts | Ratio of Central |
| | αAFP- | αGPC3- | αGPC3- | CAR + CD30- | Memory T Cell |
| | CD28z- | CD30- | CD28- | CSR/CAR + | Counts CAR + |
| Date | CAR | CSR | CSR | CD28-CSR | CD30-CSR/CAR |
| E1D3 | 11684 | 7756 | 3684 | 2.11 | 0.66 |
| E1D5 | 1317 | 14032 | 15872 | 0.88 | 10.65 |
| E1D7 | 781 | 13859 | 7419 | 1.87 | 17.75 |
| E2D3 | 940 | 36436 | 22227 | 1.64 | 38.76 |
| E2D5 | 1153 | 49787 | 29493 | 1.69 | 43.18 |
| E2D7 | 600 | 49888 | 16122 | 3.09 | 83.15 |
| E3D3 | 20 | 24263 | 7132 | 3.40 | 1213.15 |
| E3D5 | 57 | 25743 | 11848 | 2.17 | 451.63 |
| E4D2^{a} | 6 | 8246 | 2353 | 3.50 | 1374.33 |
| E4D5^{a} | 30 | 25570 | 2445 | 10.46 | 852.33 |
| E5D2^{b} | 8 | 16350 | 727 | 22.49 | 2043.75 |
| E5D5^{b} | 7 | 23945 | 2994 | 8.00 | 3420.71 |
| E5D7^{b} | 10 | 4466 | 761 | 5.87 | 446.60 |

^{a}The CAR or CAR + CSR effector and target cell mixtures were diluted 1:6 before the fourth target cell engagement (E4).
^{b}The CAR or CAR + CSR effector and target cell mixtures were diluted 1:6 again before the fifth target cell engagement (E5).

In addition to T cell counts, percentages of central memory T cells among the total number of receptor T cells were calculated and shown in Tables 8-10, together with ratios of memory T cell percentages of the CAR+CD3-CSR T cells over those of CAR+CD28-CSR T cells. The T cells whose percentage data are shown in these tables were the same T cells whose cell count data are shown in Tables 5-7.

TABLE 8
Central Memory T Cell Percentages among 1^{st}-generation CAR⁺ T
Cells Expressing CAR and CSR Encoded on Two Separate Vectors.

| | | | Ratio of Central |
|---|---|---|---|
| | | | Memory T |
| | aAFP-CD8T- | aAFP-CD8T- | Cell Percentages |
| | z-CAR + aGPC3- | z-CAR + aGPC3- | CAR + CD30-CSR/ |
| Date | CD30-CSR | CD28-CSR | CAR + CD28-CSR |
| E1D3 | 22.6 | 16.4 | 1.38 |
| E1D5 | 43 | 31.3 | 1.37 |
| E1D7 | 38.7 | 37.4 | 1.03 |
| E2D3 | 34 | 27.8 | 1.22 |
| E2D5 | 53.6 | 40.6 | 1.32 |
| E2D7 | 64.7 | 39.3 | 1.65 |
| E3D3 | 25.4 | 9.37 | 2.71 |
| E3D5 | 35.4 | 21.9 | 1.62 |
| E4D2 | 16.5 | 6.63 | 2.49 |
| E4D5 | 17.6 | 10.1 | 1.74 |
| E4D7 | 39.7 | 28.8 | 1.38 |

TABLE 9
Central Memory T Cell Percentages among 1^{st}-generation CAR⁺ T
Cells Expressing CAR and CSR Encoded on One Vector.

| | | | Ratio of Central |
|---|---|---|---|
| | | | Memory T |
| | aAFP-CD8T- | aAFP-CD8T- | Cell Percentages |
| | z-CAR + aGPC3- | z-CAR + aGPC3- | CAR + CD30-CSR/ |
| Date | CD30-CSR | CD28-CSR | CAR + CD28-CSR |
| E1D3 | 38 | 18.8 | 2.02 |
| E1D5 | 48.5 | 32.9 | 1.47 |
| E1D7 | 55.4 | 31.3 | 1.77 |
| E2D3 | 37.4 | 22.8 | 1.64 |
| E2D5 | 57.2 | 31.2 | 1.83 |
| E2D7 | 63.5 | 38.4 | 1.65 |
| E3D3 | 27.8 | 11.3 | 2.46 |
| E3D5 | 38.4 | 37.1 | 1.04 |
| E4D2 | 15.7 | 5.01 | 3.13 |
| E4D5 | 26.7 | 9.77 | 2.73 |
| E4D7 | 53.3 | 25.6 | 2.08 |

TABLE 10
Central Memory T Cell Percentages among 2^{nd}-generation CAR⁺ T Cells
Expressing CAR and CSR Encoded on Two Separate Vectors.

| | | | Ratio of Central Memory T |
|---|---|---|---|
| | αAFP-CD28z- | αAFP-CD28z- | Cell Percentages |
| | CAR + αGPC3- | CAR + αGPC3- | CAR + CD30-CSR/ |
| Date | CD30-CSR | CD28-CSR | CAR + CD28-CSR |
| E1D3 | 18.3 | 24.1 | 0.76 |
| E1D5 | 42.4 | 24.1 | 1.76 |
| E1D7 | 47.4 | 33.4 | 1.42 |
| E2D3 | 34.4 | 18.1 | 1.90 |
| E2D5 | 59 | 24.3 | 2.43 |
| E2D7 | 60.7 | 23 | 2.64 |
| E3D3 | 32.1 | 13.9 | 2.31 |
| E3D5 | 39.3 | 16.3 | 2.41 |
| E4D2 | 17.7 | 5.12 | 3.46 |
| E4D5 | 23 | 7.42 | 3.10 |
| E5D2 | 30.5 | 9.03 | 3.38 |
| E5D5 | 40.4 | 7.27 | 5.56 |
| E5D7 | 20.8 | 8.72 | 2.39 |

These surprising results show that T cells expressing CAR+CD30-CSR were able to develop into and maintain high numbers and high percentages of central memory T cells upon engagement with target calls, higher than T cells expressing CAR alone or CAR+CD28-CSR, making the CAR+CD30-CSR T cell platform a potentially successful T cell therapy platform.

In addition to determining the total central memory receptor⁺ T cell counts and their percentages among all receptor⁺ T cells, the same T cell-target cell mixture samples were also stained with antibodies against CD8, in order to determine the numbers of CD8⁺ receptor⁺ central memory T cells and calculate the percentages of central memory T cells among CD8⁺ receptor⁺ T cells. The results are shown in Tables 11-16, together with ratios of memory T cell counts or percentages of the CAR+CD30-CSR T cells over those of CAR+CD28-CSR or CAR alone T cells.

TABLE 11
CD8⁺ Central Memory T Cell Counts of 1^{st}-generation CAR⁺ T Cells with CAR
and CSR Encoded on Two Separate Vectors.

| | | | | | Ratio of |
|---|---|---|---|---|---|
| | | | | Ratio of Central | Central |
| | | aAFP-CD8T- | aAFP-CD8T- | Memory T Cell | Memory T |
| | aAFP- | z-CAR + | Z-CAR + | Counts | Cell Counts |
| | CD8T-Z- | aGPC3- | aGPC3- | CAR + CD30-CSR/ | CAR + CD30- |
| Date | CAR | CD30-CSR | CD28-CSR | CAR + CD28-CSR | CSR/CAR |
| E1D3 | 762 | 2,395 | 2,376 | 1.01 | 3.14 |
| E1D5 | 1,297 | 7,707 | 4,559 | 1.69 | 5.94 |
| E1D7 | 394 | 15,043 | 4,049 | 3.72 | 38.18 |
| E2D3 | 114 | 17,183 | 9,397 | 1.83 | 150.73 |
| E2D5 | 231 | 34,106 | 5,350 | 6.37 | 147.65 |
| E2D7 | 299 | 47,880 | 18,983 | 2.52 | 160.13 |
| E3D3 | 14 | 8,937 | 2,655 | 3.37 | 638.36 |
| E3D5 | 46 | 16,453 | 1,986 | 8.28 | 357.67 |
| E4D2 | 0 | 666 | 91 | 7.32 | >666.00 |
| E4D5 | 0 | 6,624 | 651 | 10.18 | >6624.00 |
| E4D7 | 1 | 7,142 | 1,057 | 6.76 | 7142.00 |

TABLE 12
CD8⁺ Central Memory T Cell Counts of l^{st}-generation CAR⁺ T Cells
with CAR and CSR Encoded on One Vector.

| | | | Ratio of Central |
|---|---|---|---|
| | | | Memory T Cell |
| | aAFP-CD8T-z- | aAFP-CD8T-z- | Counts |
| | CAR + aGPC3- | CAR + aGPC3- | CAR + CD30-CSR/ |
| Date | CD30-CSR | CD28-CSR | CAR + CD28-CSR |
| E1D3 | 4,178 | 2,740 | 1.52 |
| E1D5 | 10,332 | 5,291 | 1.95 |
| E1D7 | 6,157 | 4,355 | 1.41 |
| E2D3 | 21,963 | 14,518 | 1.51 |
| E2D5 | 21,292 | 8,567 | 2.49 |
| E2D7 | 46,229 | 20,347 | 2.27 |
| E3D3 | 9,296 | 2,536 | 3.67 |
| E3D5 | 11,429 | 1,281 | 8.92 |
| E4D2 | 1,265 | 113 | 11.19 |
| E4D5 | 14,457 | 879 | 16.45 |
| E4D7 | 12,452 | 669 | 18.61 |

TABLE 13
CD8⁺ Central Memory T Cell Counts of 2^{nd}-generation CAR⁺ T Cells with CAR
and CSR Encoded on Two Separate Vectors.

| | | | | | Ratio of |
|---|---|---|---|---|---|
| | | | | | Central |
| | | | | Ratio of Central | Memory T |
| | | αAFP-CD28z- | αAFP-CD28z- | Memory T Cell | Cell |
| | αAFP- | CAR + | CAR + | Counts | Counts |
| | CD28z- | αGPC3-CD30- | αGPC3-CD28- | CAR + CD30-CSR/ | CAR + CD30- |
| Date | CAR | CSR | CSR | CAR + CD28-CSR | CSR/CAR |
| E1D3 | 2955 | 3782 | 1731 | 2.18 | 1.28 |
| E1D5 | 1155 | 7604 | 7107 | 1.07 | 6.58 |
| E1D7 | 361 | 7987 | 3047 | 2.62 | 22.12 |
| E2D3 | 587 | 17883 | 8676 | 2.06 | 30.47 |
| E2D5 | 853 | 23007 | 13476 | 1.71 | 26.97 |
| E2D7 | 409 | 33419 | 10986 | 3.04 | 81.71 |
| E3D3 | 10 | 12673 | 2968 | 4.27 | 1267.30 |
| E3D5 | 60 | 14446 | 4819 | 3.00 | 240.77 |
| E4D2 | 1 | 2676 | 758 | 3.53 | 2676.00 |
| E4D5 | 0 | 16709 | 1248 | 13.39 | >16709.00 |
| E5D2 | 0 | 10885 | 244 | 44.61 | >10885.00 |
| E5D5 | 0 | 17829 | 1045 | 17.06 | >17829.00 |
| E5D7 | 0 | 3390 | 214 | 15.84 | >3390.00 |

TABLE 14
Central Memory T Cell Percentages among l^{st}-generation CD8⁺ CAR⁺
T Cells Expressing CAR and CSR Encoded on Two Separate Vectors.

| | | | Ratio of Central |
|---|---|---|---|
| | | | Memory T Cell |
| | aAFP-CD8T-z- | aAFP-CD8T-z- | Percentages |
| | CAR + aGPC3- | CAR + aGPC3- | CAR + CD30-CSR/ |
| Date | CD30-CSR | CD28-CSR | CAR + CD28-CSR |
| E1D3 | 11 | 7.77 | 1.42 |
| E1D5 | 34.2 | 18.8 | 1.82 |
| E1D7 | 28.6 | 22.8 | 1.25 |
| E2D3 | 23.7 | 18.4 | 1.29 |
| E2D5 | 47.5 | 29.2 | 1.63 |
| E2D7 | 59.3 | 31.3 | 1.89 |
| E3D3 | 18.6 | 4.71 | 3.95 |
| E3D5 | 27.2 | 9.12 | 2.98 |
| E4D2 | 8.58 | 2.32 | 3.70 |
| E4D5 | 13.9 | 5.03 | 2.76 |
| E4D7 | 36.2 | 13.5 | 2.68 |

TABLE 15
Central Memory T Cell Percentages among regeneration CD8⁺ CAR⁺
T Cells Expressing CAR and CSR Encoded on One Vector.

| | | | Ratio of Central |
|---|---|---|---|
| | | | Memory T Cell |
| | aAFP-CD8T-z- | aAFP-CD8T-z- | Percentages |
| | CAR + aGPC3- | CAR + aGPC3- | CAR + CD30-CSR/ |
| Date | CD30-CSR | CD28-CSR | CAR + CD28-CSR |
| E1D3 | 27.5 | 11.4 | 2.41 |
| E1D5 | 42 | 20.9 | 2.01 |
| E1D7 | 44 | 23.6 | 1.86 |
| E2D3 | 30.2 | 18.9 | 1.60 |
| E2D5 | 50.3 | 24.6 | 2.04 |
| E2D7 | 58 | 32.3 | 1.80 |
| E3D3 | 20.9 | 7.33 | 2.85 |
| E3D5 | 31.7 | 9.11 | 3.48 |
| E4D2 | 8.13 | 1.56 | 5.21 |
| E4D5 | 24.9 | 4.96 | 5.02 |
| E4D7 | 49 | 14.8 | 3.31 |

TABLE 16
Central Memory T Cell Percentages among 2^{nd}-generation CD8⁺ CAR⁺
T Cells Expressing CAR and CSR Encoded on Two Separate Vectors.

| | | | Ratio of Central |
|---|---|---|---|
| | | | Memory T Cell |
| | αAFP-CD28z- | αAFP-CD28z- | Percentages |
| | CAR + αGPC3- | CAR + αGPC3- | CAR + CD30-CSR/ |
| Date | CD30-CSR | CD28-CSR | CAR + CD28-CSR |
| E1D3 | 13 | 17.3 | 0.75 |
| E1D5 | 38.5 | 18.9 | 2.04 |
| E1D7 | 40.5 | 21.7 | 1.87 |
| E2D3 | 27.2 | 11.1 | 2.45 |
| E2D5 | 50.6 | 18.2 | 2.78 |
| E2D7 | 54.7 | 19 | 2.88 |
| E3D3 | 25.8 | 8.4 | 3.07 |
| E3D5 | 31.3 | 7.41 | 4.22 |
| E4D2 | 12.5 | 3.24 | 3.86 |
| E4D5 | 20.3 | 3.76 | 5.40 |
| E5D2 | 26 | 4.23 | 6.15 |
| E5D5 | 35.9 | 2.85 | 12.60 |
| E5D7 | 16.4 | 2.04 | 8.04 |

These surprising results show that CD8⁺ cytotoxic T cells expressing CAR+CD30-CSR were able to develop into and maintain high numbers and high percentages of central memory T cells, higher than CD8⁺ T cells expressing CAR alone or CAR+CD28-CSR, making the CAR+CD30-CSR T cell platform a great T cell therapy platform especially for target cell (including cancer cell) killing and cancer treatment.

From this experiment, it was also found that, at least with the 1^{st}-generation CAR, co-expressing CD30-CSR also resulted in more effector memory T cells (in addition to more central memory T cells) than expressing CAR alone or co-expressing CD28-CSR (data not shown), which are also helpful in T cell therapies.

### Example 15—In Vivo Tumor Infiltration by Anti-AFP-CAR+Anti-GPC3-CSR T Cells

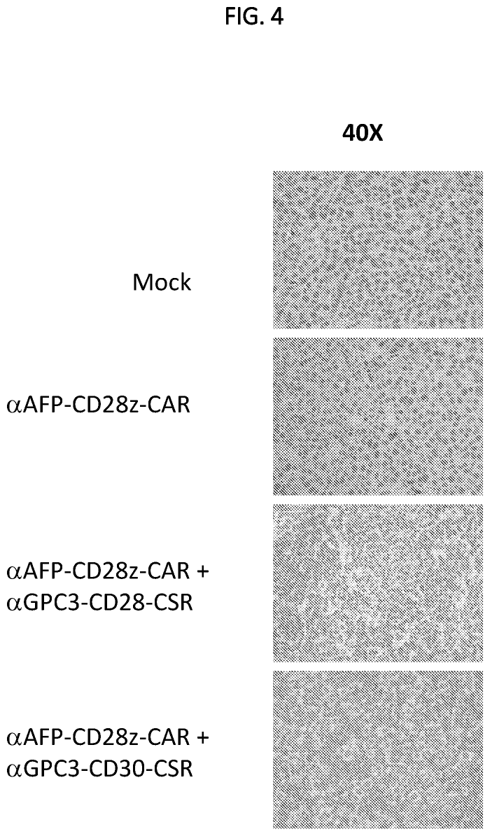

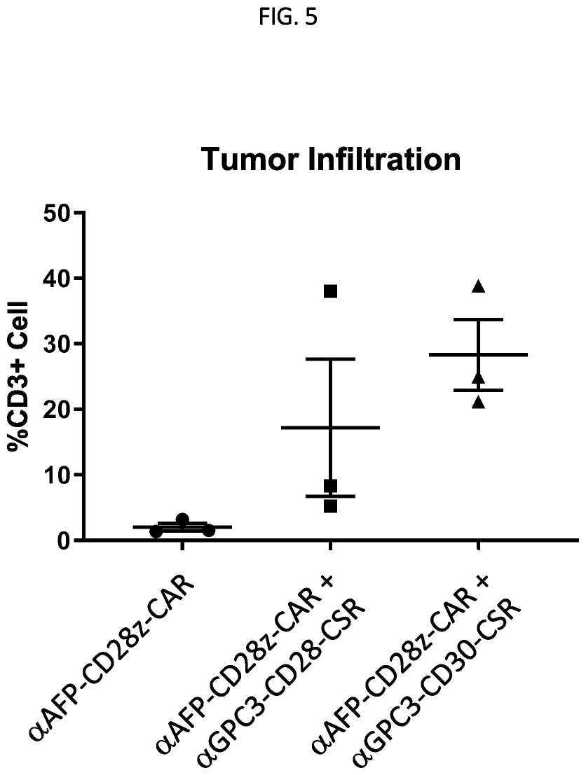

About 10⁷ HepG2 tumor cells were implanted subcutaneously in NSG mice and allowed to form a solid tumor mass 150 mm³. In one experiment, 5×10⁶ mock-transduced T cells or CAR⁺ T cells expressing (1) αAFP-CD28z-CAR (SEQ ID NO:7); (2) αAFP-CD28z-CAR+αGPC3-CD28-CSR (SEQ ID NO:7+SEQ ID NO:14); or (3) αAFP-CD28z-CAR+αGPC3-CD30-CSR (SEQ ID NO:7+SEQ ID NO:13) were injected i.v. into the tumor bearing mice, with three mice in each sample group. Three weeks after T-cell dosing, the mice were sacrificed and tumors removed, fixed, and sectioned onto slides. Tumor sections were stained with anti-CD3 antibody to visualize the T cells that were present within the solid tumor. Representative images of tumor sections from each sample group are shown in FIG. 4. Quantification of the number of CD3⁺ cells (T cells) as well as that of all cells was done on four representative sections of each mouse's tumors, and the mean T cell % (% of all cells that were CD3⁺ cells) for each CAR T sample group was calculated and shown in FIG. 5 and Table 17, as an indicator of tumor infiltration ability of the CAR T cells. FIGS. 4 and 5 as well as Table 17 show that, surprisingly, αAFP-CD28z-CAR+αGPC3-CD30-CSR T cells had significantly higher in vivo tumor infiltration/penetration rates/levels/capabilities (i.e., higher % CD3⁺ cells among all cells) than corresponding CAR T cells without CSR or corresponding CAR+CD28-CSR T cells. Table 17 further shows the ratio of % of CAR+CD30-CSR T cells (CD3⁺) among all cells over % of CAR+CD28-CSR or CAR alone T cells among all cells in the tumor samples.

TABLE 17
Tumor Infiltration by Anti-AFP CAR and Anti-AFP-CAR + Anti-GPC3-CSR T
Cells.

| Mean % CD3⁺ Cells among All Cells in | | | | |
|---|---|---|---|---|
| Tumor Samples | | | Ratio of CD3⁺ Cell | Ratio of CD3⁺ Cell |
| αAFP- | αAFP-CD28z- | αAFP-CD28z- | Percentages | Percentages |
| CD28z- | CAR + αGPC3- | CAR + αGPC3- | CAR + CD30-CSR/ | CAR + CD30-CSR/ |
| CAR | CD30-CSR | CD28-CSR | CAR + CD28-CSR | CAR |
| 2.0% | 28.3% | 17.2% | 1.65 | 14.10 |

### Example 16—In Vivo Tumor Infiltration by Anti-GPC3-CAR+Anti-GPC3-CSR T Cells

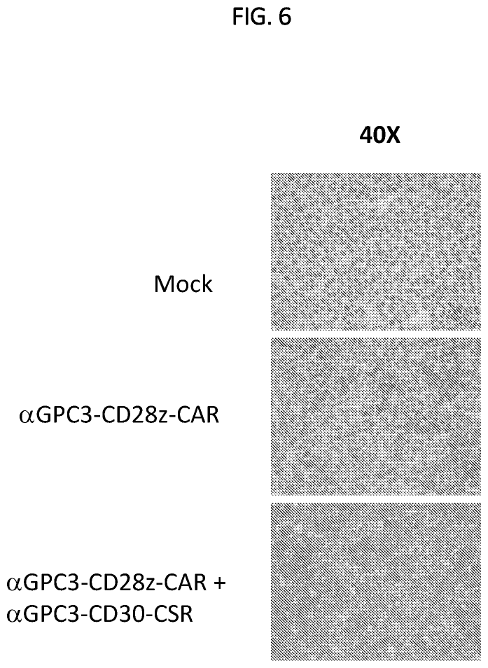

Following a similar protocol as described in Example 15, including using HepG2 tumor cells to implant NSG mice, the tumor infiltration abilities of αGPC3-CD28z-CAR and αGPC3-CD28z-CAR+αGPC3-CD30-CSR T cells were also tested in vivo. Such CAR T cells were generated by transducing primary T cells with lentiviral vectors encoding αGPC3-CD28z-CAR or αGPC3-CD28z-CAR+αGPC3-CD30-CSR. The αGPC3-CD30-CSR is identical to the αGPC3-CD30-CSR co-expressed with the αAFP-CD28z-CAR disclosed in the previous examples. The αGPC3 antibody moieties in the CAR and the CSR of these T cells comprise different GPC3-binding sequences as disclosed in the informal sequence listing. 10⁷ HepG2 tumor cells were implanted subcutaneously in NSG mice and allowed to form a solid tumor with a mass of about 250 mm³. 1×10⁷ CAR T cells (50% CAR receptor positive) or 5×10⁶ Mock T cells were injected i.v. into the tumor-bearing mice. Two weeks after T cell dosing, the mice were sacrificed and tumors removed, fixed and sectioned onto slides. Tumor sections were stained with anti-CD3 antibody to visualize the T cells that were present within the solid tumor. Quantification of the numbers of CD3⁺ cells (T cells) and total cells was done using the QuPath software to score the tumor infiltration capability of the T cells (combined tumor penetration and post-penetration T cell proliferation capabilities). Representative images of tumor sections from each sample group are shown in FIG. 6. Quantification of the numbers of T cells and total cells was done on four representative sections of each mouse's tumors, and the mean T cell % (% of all cells that were CD3⁺ cells) for each CAR T sample group was calculated and shown in Table 18. Table 18 further shows the ratio of % of CAR+CD30-CSR T cells among all cells over % of CAR alone T cells among all cells in the tumor samples. FIG. 6 as well as Table 18 show that αGPC3-CD28z-CAR+αGPC3-CD30-CSR T cells had significantly higher in vivo tumor infiltration capability (i.e., higher % CD3⁺ cells among all cells) than corresponding CAR T cells without CSR.

TABLE 18
Tumor Infiltration by Anti-GPC3 CAR and
Anti-GPC3-CAR + Anti-GPC3-CSR T Cells.

| Mean % CD3⁺ Cells among All Cells in | | |
|---|---|---|
| Tumor Samples | | |
| aGPC3- | αGPC3-CD28z-CAR + | Ratio of CD3⁺ Cell Percentages |
| CD28z-CAR | αGPC3-CD30-CSR | CAR + CD30-CSR/CAR |
| 14.8% | 32.3% | 2.18 |

### Example 17—Additional In Vitro and In Vivo Assays of Anti-GPC3-CAR T Cells Expressing Anti-GPC3-CD30-CSR vs. Anti-GPC3-CD30T-CD28-CSR In Vitro Tumor Cell Killing Assay

An LDH-based assay comparing the short-term killing ability of various anti-GPC3-CAR T cells was performed using the method described in Examples 1A and 1B. Effector cell groups used in this example include the following. These CAR T cells were generated by transducing primary T cells (from a different donor than the source of the primary T cells used in Example 16) with lentiviral vectors encoding the following CAR or CAR+CSR. Group 1) CAR T cells without CSR: anti-GPC3-CD28z-CAR; Group 2) CAR T cells with a CSR that comprises CD30 transmembrane and CD28 intracellular domains: anti-GPC3-CD28z-CAR+anti-GPC3-CD30T-CD28-CSR Group 3) CAR T cells with a CSR that comprises a CD30 transmembrane domain and the intracellular CD30 costimulatory domain (CD30 IC domain): anti-GPC3-CD28z-CAR+anti-GPC3-CD30-CSR.

Activated effector cells (anti-GPC3-CAR receptor positive T cells) and the target cells (HepG2 cells which are GPC3⁺), with SKHep1 (GPC3⁻) cells as the negative control, were co-cultured at an E:T ratio of 2:1 for 16 hours. Specific killing was determined by measuring LDH activity in culture supernatants. Tumor cytotoxicity was assayed by LDH Cytotoxicity Assay (Promega). The result showed that all three anti-GPC3 CAR T cell groups displayed significant and comparable GPC3-specific killing efficacies (all about 60% specific lysis).

In Vivo Tumor Infiltration Assay

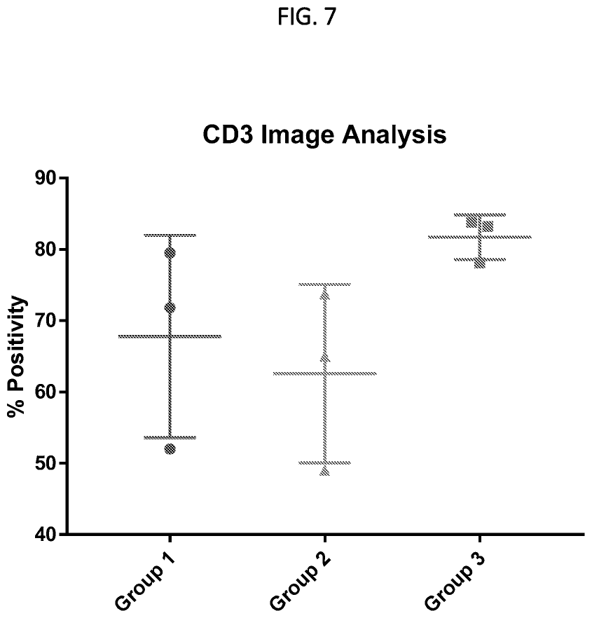

Further, following a similar protocol to those described in Examples 8A, 8B, 15, and 16, the three different CAR T effector cell groups described above in Example 17 (A) were tested for in vivo tumor infiltration capabilities. 5×10⁶ HepG2 tumor cells were implanted subcutaneously in each NSG mouse and allowed to form a solid tumor with a mass of about 150 mm³. When tumors reached the appropriate size, animals were assigned to experimental groups, with three mice tested per group. 1×10⁷ total T cells (50% CAR receptor positive for CAR T cell groups) were injected i.v. into the tumor bearing mice. Animals were sacrificed when their tumor growth plateaued 10 days after T-cell dosing. At this time the mice were sacrificed, and tumors were removed, fixed and sectioned onto slides. Immunohistochemistry was performed on tumor sections to stain for CD3. The CD3-positive and CD3-negative cells in these sections were quantified with an automated immunohistochemistry imager in order to determine the fraction of tumor mass infiltrated by T cells. Quantification of the number of CD3⁺ cells (T cells) as well as that of all cells was done on a representative section of each mouse's tumor with total cell numbers ranging from over 55,000 to almost 700,000 per section. The mean T cell % (% of all cells that were CD3⁺ cells) for each CAR T sample group was calculated and shown in FIG. 7 and Table 19. FIG. 7 as well as Table 19 show that αGPC3-CD28z-CAR+αGPC3-CD30-CSR T cells (“Group 3”) had significantly higher in vivo tumor infiltration capability (i.e., higher % CD3⁺ cells among all cells) than corresponding CAR T cells without CSR (“Group 1”) or with αGPC3-CD30T-CD28-CSR (“Group 2”).

TABLE 19
Tumor Infiltration by Anti-GPC3 CAR, Anti-GPC3-CAR + Anti-GPC3-CD30-CSR,
and Anti-GPC3-CAR + Anti-GPC3-CD30T-CD28-CSR T Cells.

| Mean % CD3⁺ Cells among All Cells in | | | | |
|---|---|---|---|---|
| Tumor Samples | | | | |
| | αGPC3- | αGPC3-CD28z- | Ratio of CD3 + | Ratio of CD3 + |
| αGPC3- | CD28z-CAR + | CAR + αGPC3- | Cell Percentages | Cell Percentages |
| CD28z- | αGPC3-CD30- | CD30T-CD28- | CAR + CD30-CSR/ | CAR + CD30-CSR/ |
| CAR | CSR | CSR | CAR + CD28-CSR | CAR |
| 66.7% | 81.7% | 61.7% | 1.32 | 1.22 |

The result shows that in vivo tumor infiltration ability (combined tumor penetration and post-penetration T cell proliferation capabilities) was the highest with αGPC3-CD28z-CAR+αGPC3-CD30-CSR T cells, which has CD30 TM and CD30 IC domains, surprisingly much higher than αGPC3-CD28z-CAR+αGPC3-CD30T-CD28-CSR T cells, which only differs from the CAR+CD30-CSR T cells in the intracellular region, suggesting that the CD30 IC costimulatory domain played an important role in the high in vivo tumor infiltration capability of the CAR+CD30-CSR T cells.

In Vivo T Cell Expansion/Proliferation in Terminal Blood Samples

In order to test the in vivo cell expansion/proliferation capability of αGPC3-CD28z-CAR+αGPC3-CD30T-CD28-CSR T cells, terminal blood samples were drawn from the mice used in the in vivo tumor infiltration assay disclosed in Example 17 (B) when the mice were sacrificed. The concentrations of CAR receptor⁺ CD3⁺ cells and total CD3⁺ cells (numbers of cells per mL blood) were determined, and the result is shown in Table 20. Table 20 further shows the ratios of peripheral blood concentration of CAR+CD30-CSR T cells (CD3⁺) over the concentration of CAR+CD28-CSR or CAR alone T cells.

TABLE 20
In Vivo Cell Expansion/Proliferation of Anti-GPC3 CAR, Anti-GPC3-CAR + Anti-
GPC3-CD30-CSR, and Anti-GPC3-CAR + Anti-GPC3-CD30T-CD28-CSR T Cells.

| | Mean Cell Concentration | | | | |
|---|---|---|---|---|---|
| | (Number of Cells per mL Blood) | | | Ratio of Cell | |
| | | | αGPC3- | Concentration | |
| | | αGPC3- | CD28z-CAR + | CAR + CD30- | Ratio of Cell |
| Type of | αGPC3- | CD28z- | αGPC3- | CSR/ | Concentration |
| Cells | CD28z- | CAR + αGPC3- | CD30T- | CAR + CD28- | CAR + CD30- |
| Quantified | CAR | CD30-CSR | CD28-CSR | CSR | CSR/CAR |
| CAR⁺ | 81.3 | 128.7 | 43.6 | 2.95 | 1.58 |
| CD3⁺ | | | | | |
| Cells | | | | | |
| Total | 755.3 | 1,724.2 | 381.8 | 4.52 | 2.28 |
| CD3⁺ | | | | | |
| Cells | | | | | |

The result shows that in vivo cell expansion/proliferation capability was the highest with αGPC3-CD28z-CAR+αGPC3-CD30-CSR T cells, which has CD30 TM and CD30 IC domains, surprisingly much higher than αGPC3-CD28z-CAR+αGPC3-CD30T-CD28-CSR T cells, which only differs from the CAR+CD30-CSR T cells in the intracellular region, suggesting that the CD30 IC costimulatory domain played an important role in the high in vivo cell expansion/proliferation capability of the CAR+CD30-CSR T cells.

In Vivo Memory T Cell Counts of Terminal Blood Samples

In order to test the in vivo memory T cell generation capability of αGPC3-CD28z-CAR+αGPC3-CD30T-CD28-CSR T cells, terminal blood samples were drawn from the mice used in the in vivo tumor infiltration assay disclosed in Example 17 (B) when the mice were sacrificed. The numbers of central memory T cells (CD45RA⁻ CCR7⁺ T cells, CD8⁺ or CD4⁺) in peripheral blood were determined as described in the examples above, and percentages of central memory T cells among the total T cells were calculated and shown in Table 21. Table 21 further shows the ratios of central memory T cell percentage of CAR+CD30-CSR T cells over that of CAR+CD28-CSR or CAR alone T cells.

TABLE 21
In Vivo Central Memory T Cell Percentages of Anti-GPC3 CAR, Anti-GPC3-
CAR + Anti-GPC3-CD30-CSR, and Anti-GPC3-CAR + Anti-GPC3-CD30T-
CD28-CSR T Cells.

| | Mean Central Memory T Cell Percentage | | | | |
|---|---|---|---|---|---|
| | among All Terminal Blood CAR⁺ CD3⁺ | | | Ratio of Central | |
| | Cells | | | Memory T Cell | Ratio of |
| | | | αGPC3- | Percentages | Central |
| | | αGPC3- | CD28z-CAR + | CAR + CD30- | Memory T Cell |
| Type of | αGPC3- | CD28z-CAR + | αGPC3- | CSR/ | Percentages |
| Cells | CD28z- | αGPC3- | CD30T- | CAR + CD28- | CAR + CD30- |
| Quantified | CAR | CD30-CSR | CD28-CSR | CSR | CSR/CAR |
| CD8⁺ T | 4.7 | 12.7 | 7.9 | 1.60 | 2.69 |
| cells | | | | | |
| CD4⁺ T | 22.6 | 31.3 | 26.8 | 1.17 | 1.38 |
| cells | | | | | |

The in vivo peripheral blood result shows that both CD8⁺ and CD4⁺ central memory T cell percentages were also the highest with anti-GPC3-CD28z-CAR+anti-GPC3-CD30-CSR T cells, also surprisingly much higher than αGPC3-CD28z-CAR+αGPC3-CD30T-CD28-CSR T cells, which only differs from the CAR+CD30-CSR T cells in the intracellular region, suggesting that the CD30 IC costimulatory domain played an important role in the high in vivo central memory T cell generation capability of the CAR+CD30-CSR T cells.

### Example 18—Multiple In Vitro Assays of Anti-CD19-CAR T Cells Expressing Anti-CD19-CD30-CSR vs. Anti-CD19-CD28-CSR or 41BB-CSR

Short-Term Killing and IFN-Gamma Production

Assays comparing the short-term killing ability of the various T cells were performed as in Example 1A using the following constructs:

1^{st} generation constructs used: □

### αCD19-CD8T-z-CAR+αCD19-CD28-CSR; and

### αCD19-CD8T-z-CAR+αCD19-CD30-CSR.

2^{nd} generation constructs used:

### αCD19-CD8T-41BBz-CAR+αCD19-CD28T-41BB-CSR; and

### αCD19-CD8T-41BBz-CAR+αCD19-CD28T-CD30-CSR.

Primary T cells were transduced with a vector encoding each construct. The transduction efficiency was determined and T cells were matched at 40 percent receptor positivity by mixing with mock transduced T-cells. Nalm6 or Raji cells were used at an effector-to-target ratio of 1:1. The release of IFNγ into the media was measured after 72 hours. The IFNγ levels in the culture medium were measured using the Magpix multiplex system (Luminex) with the Bio-plex Pro Human Cytokine 8-plex Assay (BioRad). Assay supernatants from Nalm6 or Raji target reactions were diluted 4-fold. Cytokine concentrations were determined with the standard curve supplied with the BioRad Bio-plex kit.

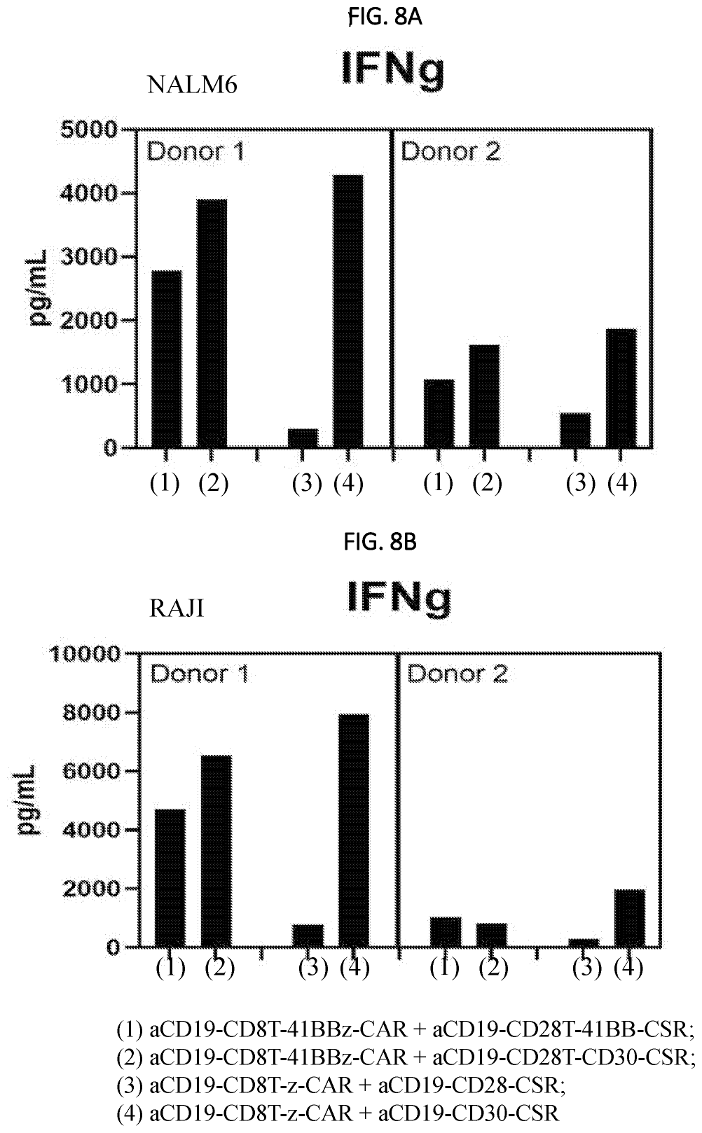

The in vitro killing and subsequent increase in cytokine levels was notably greater or comparable using the CAR+CD30-CSR as compared to the CAR+41BB-CSR, with the most dramatic increases observed in the 1^{st} generation CAR-expressing effector T cells using both Nalm6 and Raji cells (FIGS. 8A and 8B). The notable increase in IFNγ release seen with CAR+CD30-CSR when killing Nalm6 or Raji demonstrated that the CAR+CD30-CSR retained its increased cytotoxic signaling potential in an anti-CD19 model.

Long-Term Killing Assays and Central Memory T Cell Measurements Using 1^{st} Generation CAR+CSR Effector and Nalm6 Target Cells

Assays comparing the long-term killing ability of the various T cells were performed (see, e.g., Examples s 3A, 3B, and 12 for sample experimental protocol) using 1^{st} generation CAR+CSR-expressing effector cells:

1^{st}-gen CARs used:

### αCD19-CD8T-z-CAR+αCD19-CD28-CSR;

### αCD19-CD8T-z-CAR+αCD19-CD30-CSR.

The percentage of central memory T cells (Tcm) represents the % Tcm in the population of total receptor⁺ T cells (CD8⁺ T cells and CD4⁺ T cells). The % of central memory T cells was measured during the course of a multiweek assay and a ratio was calculated using the % memory T cells of the CAR+CD30-CSR expressing cells divided by the % Tcm of the CAR+CD28-CSR-expressing effector cells. Table 22 shows that the CAR-CD30-CSR expression induced considerably more central memory T cells to persist and expand over the course of the assay, consistently better than CAR+CD28-CSR cells. The target cells were Nalm6. Representative data is shown.

TABLE 22
Central Memory T Cell Percentages among l^{st}-generation CAR⁺ T Cells
Expressing CAR and CSR

| | % Central Memory T Cells | | Ratio of % Central |
|---|---|---|---|
| | l^{st}-gen αCD19-CD8T- | l^{st}-gen αCD19-CD8T- | Memory T cells |
| | z-CAR + αCD19- | z-CAR + αCD19- | CAR + CD30-CSR/ |
| Date | CD30-CSR | CD28-CSR | CAR + CD28-CSR |
| E1D3 | 30.5 | 7.14 | 4.27 |
| E1D5 | 44.2 | 18.3 | 2.42 |
| E1D7 | 44.3 | 22.1 | 2.00 |
| E2D3 | 36.8 | 16.8 | 1.95 |
| E2D5 | 33.3 | 18.9 | 1.76 |
| E2D7 | 27.3 | 13.6 | 2.01 |
| E3D3 | 29.1 | 9.57 | 3.04 |
| E3D5 | 18.7 | 6.26 | 2.99 |
| E3D7 | 17.3 | 5.62 | 3.08 |
| E4D3 | 26.3 | 6.55 | 4.02 |
| E4D5 | 16.5 | 6.1 | 2.70 |
| E4D7 | 13.7 | 6.04 | 2.27 |

Assays using Raji cells as the target also showed more Tcm cells were present in the CAR+CD30-CSR population than in the CAR+CD28-CSR population expressed as a percentage of receptor⁺ T cells (CD8⁺ CD4⁺). The % Central Memory T cells in assays with CAR+CD30-CSR-expressing effector cells was appreciably and consistently higher throughout the assay period (data not shown) than effector cells expressing CAR+CD28-CSR.

Long-Term Killing Assays and Central Memory T cell Measurements using 2^{nd} Generation CAR+CSR Effector Cells and Nalm6 Target Cells

Constructs used in this example include the following 2^{nd} generation CARs: αCD19-CD8T-41BBz-CAR+αCD19-CD28T-41BB-CSR; αCD19-CD8T-41BBz-CAR+αCD19-CD28T-CD30-CSR.

The expansion of the target antigen-specific memory T cell component of total T cells (CD4⁺ T cells and CD8⁺ T cells) following Nalm6 target cell engagement was assessed in a long-term assay using 2^{nd} generation CAR effector cells expressing the CAR+CD30-CSR and CAR+41BB-CSR. The ratio of central memory T cell percentages of CAR+CD3-CSR compared to CAR+41BB-CSR was dependably higher during the entire course of the long-term assay. The ratio of percentages of CD8R central memory T cells likewise was consistently greater in CAR+CD3D-CSR-expressing effector cells compared to CAR+41BB-CSR-expressing cells during the same period. All CAR+CSR assays showed robust target cell killing (not shown). Representative data are shown; see Tables 23A and Table 23B.

TABLE 23A
Central Memory T Cell Percentages among 2^{nd}-generation
CAR⁺ T Cells Expressing CAR and CSR

| | % Total Central Memory T Cells | | |
|---|---|---|---|
| | αCD19-CD8T- | αCD19-CD8T- | Ratio of % Central |
| | 41BBz-CAR + | 41BBz-CAR + | Memory T cells |
| | αCD19-CD28T- | αCD19-CD28T- | CAR + CD30-CSR/ |
| Date | CD30-CSR | 41BB-CSR | CAR + 41BB-CSR |
| E1D7 | 48.4 | 44.2 | 1.10 |
| E2D3 | 40 | 33.4 | 1.20 |
| E2D5 | 36.4 | 30.7 | 1.19 |
| E2D7 | 32.6 | 25.8 | 1.26 |
| E3D3 | 32.4 | 21.9 | 1.48 |
| E3D5 | 22.5 | 17.4 | 1.29 |
| E3D7 | 21.6 | 16.5 | 1.31 |
| E4D2 | 27.1 | 13 | 2.08 |
| E4D5 | 20.5 | 9.75 | 2.10 |
| E4D7 | 15.8 | 8.81 | 1.79 |

Table 23B
CD8⁺ Central Memory T Cell Percentages among 2^{nd}-generation
CAR⁺ T Cells Expressing CAR and CSR

| | % CD8⁺ Central Memory T Cells | | Ratio of % |
|---|---|---|---|
| | αCD19-CD8T- | αCD19-CD8T- | CD8⁺ Central |
| | 41BBz-CAR + | 41BBz-CAR + | Memory T cells |
| | αCD19-CD28T- | αCD19-CD28T- | CAR + CD30-CSR/ |
| Date | CD30-CSR | 41BB-CSR | CAR + 41BB-CSR |
| E1D7 | 41.1 | 34.9 | 1.19 |
| E2D3 | 29.3 | 20.8 | 1.41 |
| E2D5 | 23 | 20.6 | 1.12 |
| E2D7 | 24.5 | 19.1 | 1.28 |
| E3D3 | 21.2 | 13.2 | 1.61 |
| E3D5 | 15.4 | 12.7 | 1.21 |
| E3D7 | 17.4 | 13.8 | 1.26 |
| E4D2 | 19.6 | 8.81 | 2.22 |
| E4D5 | 16.4 | 5.78 | 2.84 |
| E4D7 | 11.3 | 5.78 | 1.96 |

The Raji cell data showed comparable central memory T cell percentages between the CAR+41BB-CSR and the CAR+CD30-CSR populations during the course of the assay.

Long-Term Killing Assays and Measurement of Total T Cell and Receptor⁺ T Cells Using 2nd-Generation CAR+CSR Effector Cells and Nalm6 as Target Cells

Constructs used in this example include the following:

2^{nd} generation CARs used:

### αCD19-CD8T-41BBz-CAR+αCD19-CD28T-41BB-CSR;

### αCD19-CD8T-41BBz-CAR+αCD19-CD28T-CD30-CSR.

The total T cell population, the receptor⁺ component and the target component of T cells was measured during the course of a long-term killing assay using 2^{nd} generation CAR T cells expressing 41BB-CSRs or CD30-CSRs with a Nalm6 target cell population. A comparison of total T cell, Receptor⁺ T cell and target cell numbers showed a consistently greater number of total T cells in the population expressing the CAR+CD30-CSR than in the population expressing the CAR+41BB-CSR. The receptor⁺ component of these T cells was greater for CAR+CD30-CSR-expressing effector population than for CAR+41BB-CSR expressing cells during the final weeks of the assay (E3D5 through E4D7), although in the beginning the levels were comparable in cultures with both CD30-CSR and 41BB-CSR effector cells. Low numbers of target cells were found in both populations during the duration of the assay. The ratio of total T cell counts and receptor⁺ T cell counts comparing CD30-CSR to 41BB-CSR cell numbers showed a consistently greater number of T cells and R⁺ T cells present in CD30-CSR expressing effector cell cultures. Representative data are shown. See Tables 24A and Table 24B.

TABLE 24A
Total T cell counts in using effector cells expressing
CAR + CSRs following Nalm6 target cell engagement

| | | | Ratio of total T |
|---|---|---|---|
| | Total T cell Count | | cell numbers |
| | αCD19-CD8T- | αCD19-CD8T- | CAR + CD28T- |
| | 41BBz-CAR + | 41BBz-CAR + | CD30-CSR/ |
| | αCD19-CD28T- | αCD19-CD28T- | CAR + CD8T- |
| Date | CD30-CSR | 41BB-CSR | 41BB-CSR |
| E3D3 | 143979 | 101433 | 1.42 |
| E3D5 | 124636 | 108033 | 1.15 |
| E3D7 | 130150 | 78410 | 1.66 |
| E4D3 | 95522 | 47447 | 2.01 |
| E4D5 | 70304 | 51440 | 1.37 |
| E4D7 | 63283 | 18642 | 3.39 |

TABLE 24B
Receptor⁺ T cell counts in using effector cells expressing
CAR + CSRs following Nalm6 target cell engagement.

| | | | Ratio of receptor⁺ |
|---|---|---|---|
| | Receptor⁺ T cell Count | | T cell numbers |
| | αCD19-CD8T- | αCD19-CD8T- | CAR + CD28T- |
| | 41BBz-CAR + | 41BBz-CAR + | CD30-CSR/ |
| | αCD19-CD28T- | αCD19-CD8T- | CAR + CD8T- |
| Date | CD30-CSR | 41BB-CSR | 41BB-CSR |
| E3D3 | 84826 | 60487 | 1.40 |
| E3D5 | 55021 | 51949 | 1.06 |
| E3D7 | 55305 | 26833 | 2.06 |
| E4D3 | 46331 | 14076 | 3.29 |
| E4D5 | 22383 | 15050 | 1.49 |
| E4D7 | 28597 | 5402 | 5.29 |

The data from the Raji target engagement showed similar T cell and receptor⁺ T cell counts during each assay time point, as well as low target cell numbers during the course of the assay (not shown).

Exhaustion Marker Expression in Long-Term Killing Assays using 1^{st} or 2^{nd} Generation CAR+CSR Effector Cells

Constructs used in this example include the following 2^{nd} generation CARs in assays measuring PD1 expression levels in Total T cells (CD4⁺ CD8⁺):

### αCD19-CD8T-41BBz-CAR+αCD19-CD28T-41BB-CSR;

### αCD19-CD8T-41BBz-CAR+αCD19-CD28T-CD30-CSR.

The PD1 exhaustion marker expression was analyzed in long term cultures of CAR+CSR expressing effector cells using both Nalm6 and Raji cells as the target. The PD1 exhaustion marker expression in total T cells and in CD8⁺ T cells following target engagement was lower in CD30-CSR-expressing populations compared to 41BB-CSR-expressing populations during the course of the assay (EID3-E4D7) using Nalm6 or Raji cells as targets. PD1 expression in T cells was measured by flow cytometry and calculating the Mean Fluorescent Intensity (MFI) of PD1. The reduced expression of PD1 in the T cells show that CAR+CD30-CSR expression reduces the deterioration of T cell function when compared to CAR+41BB-CSR expression in long-term assay cultures. See Tables 25A, 25B, 26A, and 26B.

TABLE 25A
PD1 Expression Levels in total T cells following
Nalm6 target cell engagement

| | PD1 Expression Level in 2^{nd}-gen | | Ratio of PD1 |
|---|---|---|---|
| | CAR + CSR Total T Cells (MFI) | | Expression |
| | αCD19-CD8T- | αCD19-CD8T- | Levels |
| | 41BBz-CAR + | 41BBz-CAR + | CAR-CD30- |
| | αCD19-CD28T- | αCD19-CD28T- | CSR/CAR- |
| Date | CD30-CSR | 41BB-CSR | 41BB-CSR |
| E1D3 | 320 | 379 | 0.84 |
| E2D3 | 254 | 304 | 0.84 |
| E3D3 | 298 | 307 | 0.97 |
| E3D5 | 209 | 248 | 0.84 |
| E4D3 | 271 | 333 | 0.81 |
| E4D5 | 187 | 238 | 0.79 |
| E4D7 | 250 | 292 | 0.86 |

TABLE 25B
PD1 Expression Levels in CD8⁺ T cells following
Nalm6 target cell engagement

| | PD1 Expression Level of ^{2nd}-gen | | Ratio of PD1 |
|---|---|---|---|
| | CAR + CSR CD8⁺ T Cells (MFI) | | Expression |
| | αCD19-CD8T- | αCD19-CD8T- | Levels |
| | 41BBz-CAR + | 41BBz-CAR + | CAR-CD30- |
| | αCD19-CD28T- | αCD19-CD28T- | CSR/CAR- |
| Date | CD30-CSR | 41BB-CSR | 41BB-CSR |
| E1D3 | 156 | 213 | 0.73 |
| E2D3 | 188 | 224 | 0.84 |
| E3D3 | 239 | 267 | 0.90 |
| E3D5 | 151 | 207 | 0.73 |
| E4D3 | 172 | 285 | 0.60 |
| E4D5 | 115 | 169 | 0.68 |
| E4D7 | 201 | 240 | 0.84 |

### Example 19—Multiple In Vitro Assays of Anti-ROR1-CAR T Cells Expressing Anti-ROR1-CD28T-CD30-CSR vs. Anti-ROR1-CD28T-41BB-CSR

This example shows that ROR1-targeting CAR T cells co-expressing an ROR1-targeting CSR comprising a CD30 costimulatory domain killed cancer cells effectively and out-performed corresponding CAR T cells co-expressing a CSR comprising a 4-1BB costimulatory domain in cell exhaustion marker level and central memory T cell measurement assays.

The two effector cell groups used in this example are the following.

Mock transduced T cells;
αROR1-CD8T-41BBz-CAR+αROR1-CD28T-CD30-CSR T cells (“tCD30”); and
αROR1-CD8T-41BBz-CAR+αROR1-CD28T-41BB-CSR T cells (“t41BB”).

The anti-ROR1 antigen-binding domains (antibody moieties) of these CAR and CSR comprise the same scFv sequence (SEQ ID NO: 50). These CAR T cells were generated by transducing primary T cells with lentiviral vectors with CAR and CSR encoded on a single vector.

The target cell lines used in this example are the following, all expressing ROR1 (ROR1⁺).

Jekol (a lymphoma cell line);
RPMI8226 (a multiple myeloma cell line);
MDA-MB-231 (a breast cancer cell line); and
A549, H1975, and H1703 (three different lung cancer cell lines).

A. Short-Term Killing and Cytokine Production Related to Cancer Cell Killing by Anti-ROR1 CAR+CSR T Cells

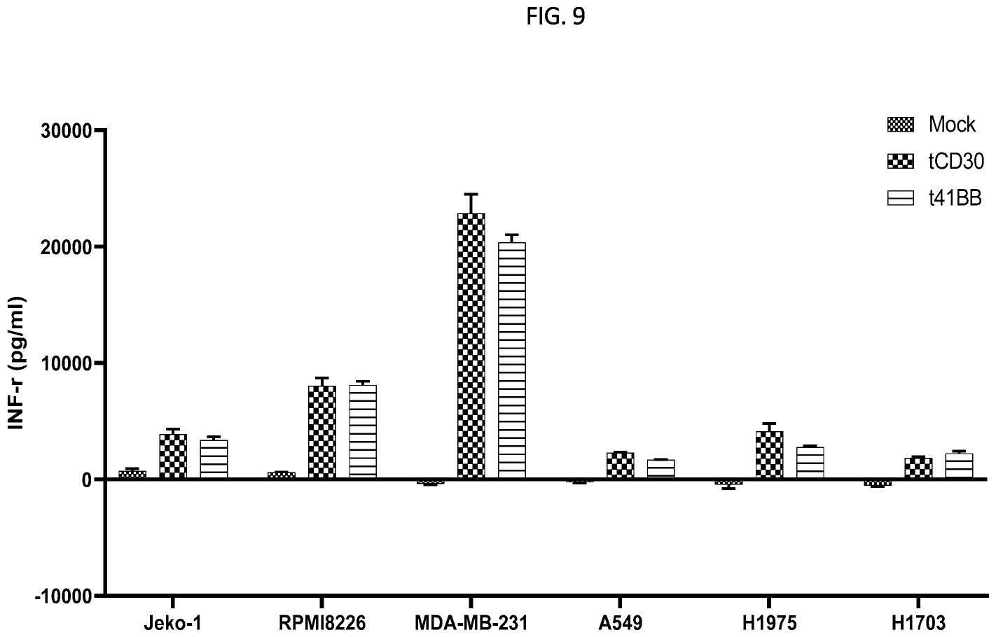

The short-term in vitro target cell killing ability of the two anti-ROR1 CAR+CSR T cell groups was determined as described in Example 1B by measuring the amounts/levels of cytokines released from T cells upon engagement with various target cells. 2×10⁵ CAR⁺ T cells were co-cultured with target cells at an ET ratio of 1:1 for about 16 h. The levels of IFNγ release in the supernatant after co-culture were quantified. The results are shown in FIG. 9, and they indicate that anti-ROR1-CD8T-41BBz-CAR+anti-ROR1-CD28T-CD30-CSR T cells (“tCD30”) had significant ROR1-specific cell killing capability against all six tested cancer cell lines (measured by IFNγ release level) as compared to the mock-transduced T cells, and their cell killing capability is comparable to or somewhat better than that of corresponding CAR T cells co-expressing a CSR comprising a 4-1BB costimulatory domain (“t41BB”).

B. Long-Term Anti-ROR1 CAR+CSR T Cell and Target Cell Counts After Multi-Week Engagements

This example shows that in a long-term killing assay, anti-ROR1 CAR+CD30-CSR T cells killed more target cells and mostly survived better than corresponding CAR+41BB-CSR T cells did.

10⁵ CAR⁺ (receptor⁺) T cells of the two anti-ROR1 CAR+CSR cell groups as described in this example were first co-cultured/engaged with various target cells at an ET ratio of 1:1 in multiple duplicates. The target cells used in this example (Example 19 B) were MDA-MB-231 (a breast cancer cell line), A549, H1975, and H1703 (lung cancer cell lines), which are all solid tumor cells and adherent cells. Every seven days after the first engagement, the remaining live target cells (adhered to plates) of one sample T cell-target cell mixture per sample group were lysed and stained with crystal violet for total target cell number/mass quantification, while the unlysed samples of each group, including the T cells in culture suspension and the adhered target cells, were re-challenged with 10⁵ fresh target cells every seven days.

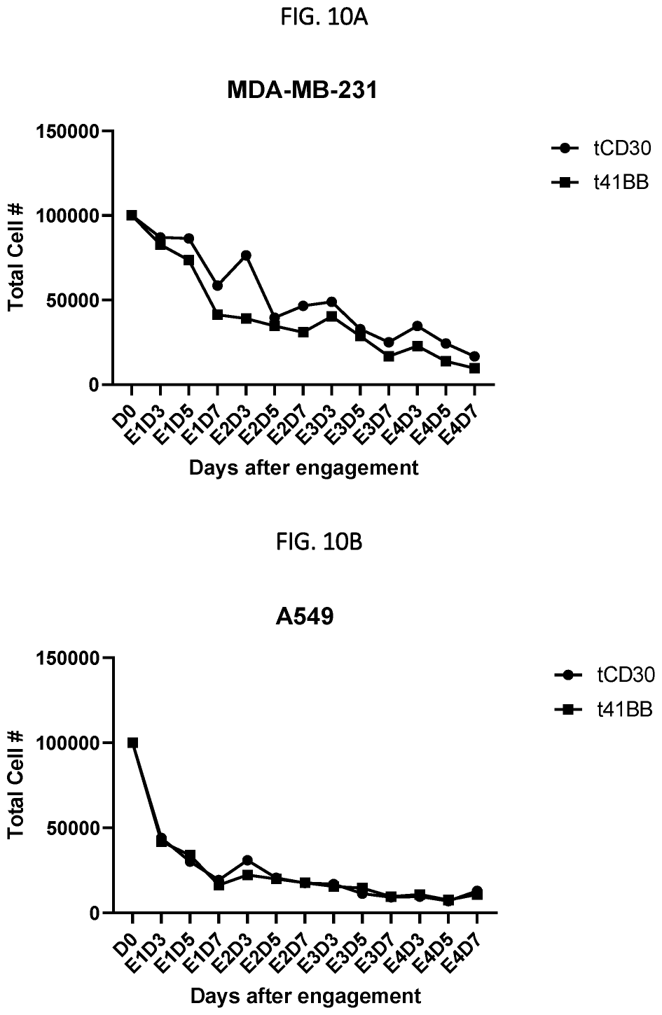

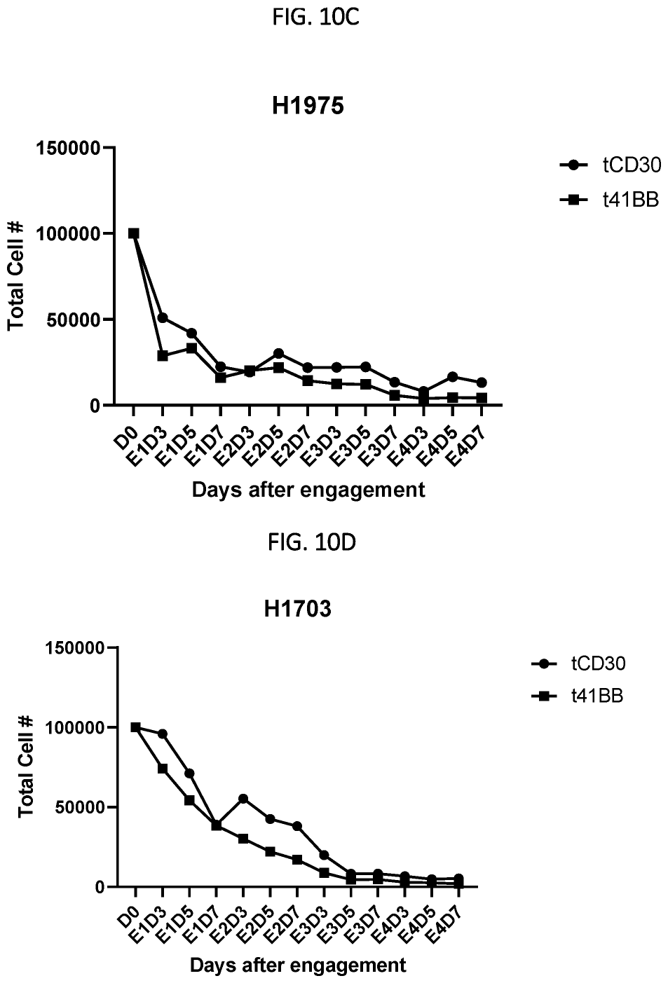

Table 26 shows ratios of remaining target cell number/mass after challenging αROR1-CD8T-41BBz-CAR+αROR1-CD28T-CD30-CSR T cells (“CAR+CD30-CSR”) vs. challenging αROR1-CD8T-41BBz-CAR+αROR1-CD28T-41BB-CSR T cells (“CAR+41BB-CSR”), using various cancer cell lines (H1975, MDA-MB-231, H1703, and A549). Live T cells remaining in each sample group (in culture suspension) were quantified on various days after each target cell engagement using FACS, and the results are shown in FIGS. 10A-10D.

TABLE 26

| | Ratio of Remaining Target Cell Number/Mass | | | |
|---|---|---|---|---|
| | CAR + CD30-CSR/CAR + 41BB-CSR | | | |
| Date | H1975 | MDA-MB-231 | H1703 | A549 |
| E1D7 | 0.99 | 0.83 | 0.30 | 1.10 |
| E2D7 | 0.97 | 0.73 | 0.78 | 0.91 |
| E3D7 | 0.55 | 0.76 | 0.78 | 0.88 |
| E4D7 | 0.44 | 0.85 | 0.93 | 0.93 |

As can be seen in Table 26 that, in the long-term killing assay, the anti-ROR1 CAR+CD30-CSR T cells surprisingly killed more target cells than CAR+41BB-CSR T cells did in each of the four target cell lines' challenges. FIGS. 10A-10D showed that, in the long-term killing assay, the anti-ROR1 CAR+CD30-CSR T cells mostly had higher cell survival than CAR+41BB-CSR T cells, which indicated that CAR+CD30-CSR T cells had better T cell persistence. Further, because the only difference between the two CAR+CSR constructs is the intracellular domain of CSR, the CAR+CD30-CSR T cells' higher long-term target cell killing and T cell survival capabilities are mainly due to the intracellular domain, or costimulatory domain, of CD30.

C. Expression of T Cell Exhaustion Markers in Anti-ROR1 CAR+CSR T Cells after Co-Culture with Target Cells

The expression levels of the T cell exhaustion marker PD1 of the two anti-ROR1 CAR+CSR T cell groups were measured according to the methods described in Examples 6 and 13, using some of the ROR1⁺ cancer cell lines disclosed above. 10⁵ CAR⁺ (receptor⁺) T cells of the two anti-ROR1 CAR+CSR cell groups as described in this example were first co-cultured/engaged with various target cells at an ET ratio of 1:1 in multiple duplicates on 96-well plates. The target cells used in this example (Example 19 C) are A549, H1975, MDA-MB-231, and a multiple myeloma cell line RPMI8226. PD1 expression levels of T cells of at least one sample per sample group were measured on selected days after target cell engagements. Every seven days after the first engagement, the unused duplicate cell mixture samples of each group were re-challenged with 10⁵ fresh target cells. Representative results of the PD1 expression level measurements (MFI values) and calculated ratios of MFI value of CAR+CD30-CSR vs. CAR+41BB-CSR T cells are shown below in Tables 26A to 29B.

TABLE 26A
Comparison of PD1 Expression Levels of Anti-ROR1 CAR + CSR
CD8⁺ T Cells after Engagements with A549 Target Cells.

| | | | Ratio of PD1 |
|---|---|---|---|
| | PD1 Expression Level on | | Expression |
| | CD8⁺ T Cells (MFI) | | Levels on |
| | αROR1-CD8T- | αROR1-CD8T- | CD8⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 130 | 138 | 0.94 |
| E2D5 | 123 | 177 | 0.69 |
| E3D5 | 180 | 219 | 0.82 |
| E4D5 | 197 | 306 | 0.64 |

TABLE 26B
Comparison of PD1 Expression Levels of Anti-ROR1 CAR + CSR
CD4⁺ T Cells after Engagements with A549 Target Cells.

| | | | Ratio of PD1 |
|---|---|---|---|
| | PD1 Expression Level on | | Expression |
| | CD4⁺ T Cells (MFI) | | Levels on |
| | αROR1-CD8T- | αROR1-CD8T- | CD4⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 462 | 702 | 0.66 |
| E2D5 | 922 | 2175 | 0.42 |
| E3D5 | 1184 | 2570 | 0.46 |
| E4D5 | 1080 | 2175 | 0.50 |

TABLE 27A
Comparison of PD1 Expression Levels of Anti-ROR1 CAR + CSR
CD8⁺ T Cells after Engagements with H1975 Target Cells.

| | | | Ratio of PD1 |
|---|---|---|---|
| | PD1 Expression Level on | | Expression |
| | CD8⁺ T Cells (MFI) | | Levels on |
| | αROR1-CD8T- | αROR1-CD8T- | CD8⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 171 | 166 | 1.03 |
| E2D5 | 184 | 211 | 0.87 |
| E3D5 | 179 | 262 | 0.68 |
| E4D5 | 163 | 214 | 0.76 |

TABLE 27B
Comparison of PD1 Expression Levels of Anti-ROR1 CAR + CSR
CD4⁺ T Cells after Engagements with H1975 Target Cells.

| | | | Ratio of PD1 |
|---|---|---|---|
| | PD1 Expression Level on | | Expression |
| | CD4⁺ T Cells (MFI) | | Levels on |
| | αROR1-CD8T- | αROR1-CD8T- | CD4⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 522 | 759 | 0.69 |
| E2D5 | 710 | 1316 | 0.54 |
| E3D5 | 772 | 1643 | 0.47 |
| E4D5 | 565 | 1305 | 0.43 |

TABLE 28A
Comparison of PD1 Expression Levels of Anti-ROR1 CAR + CSR
CD8⁺ T Cells after Engagements with MDA-MB-231 Target Cells.

| | | | Ratio of PD1 |
|---|---|---|---|
| | PD1 Expression Level on | | Expression |
| | CD8⁺ T Cells (MFI) | | Levels on |
| | αROR1-CD8T- | αROR1-CD8T- | CD8⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 114 | 124 | 0.92 |
| E2D5 | 163 | 203 | 0.80 |
| E3D5 | 194 | 232 | 0.84 |
| E4D5 | 269 | 292 | 0.92 |

TABLE 28B
Comparison of PD1 Expression Levels of Anti-ROR1 CAR + CSR
CD4⁺ T Cells after Engagements with MDA-MB-231 Target Cells.

| | | | Ratio of PD1 |
|---|---|---|---|
| | PD1 Expression Level on | | Expression |
| | CD4⁺ T Cells (MFI) | | Levels on |
| | αROR1-CD8T- | αROR1-CD8T- | CD4⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 360 | 630 | 0.57 |
| E2D5 | 666 | 1406 | 0.47 |
| E3D5 | 650 | 1241 | 0.52 |
| E4D5 | 687 | 904 | 0.76 |

TABLE 29A
Comparison of PD1 Expression Levels of Anti-ROR1 CAR + CSR
CD8⁺ T Cells after Engagements with RPMI8226 Target Cells.

| | | | Ratio of PD1 |
|---|---|---|---|
| | PD1 Expression Level on | | Expression |
| | CD8⁺ T Cells (MFI) | | Levels on |
| | αROR1-CD8T- | αROR1-CD8T- | CD8⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 121 | 138 | 0.88 |
| E2D5 | 119 | 201 | 0.59 |
| E3D5 | 167 | 180 | 0.93 |
| E4D5 | 222 | 266 | 0.83 |

TABLE 29B
Comparison of PD1 Expression Levels of Anti-ROR1 CAR + CSR
CD4⁺ T Cells after Engagements with RPMI8226 Target Cells.

| | | | Ratio of PD1 |
|---|---|---|---|
| | PD1 Expression Level on | | Expression |
| | CD4⁺ T Cells (MFI) | | Levels on |
| | αROR1-CD8T- | αROR1-CD8T- | CD4⁺ T Cells |
| | 41BBz-CAR + | 41BBz-CAR + | CAR + CD30- |
| | αROR1-CD28T- | αROR1-CD28T- | CSR/CAR + |
| Date | CD30-CSR | 41BB-CSR | 41BB-CSR |
| E1D5 | 258 | 320 | 0.81 |
| E2D5 | 265 | 438 | 0.61 |
| E3D5 | 334 | 476 | 0.70 |
| E4D5 | 428 | 547 | 0.78 |

The results show that, surprisingly, expressing CAR+CD30-CSR resulted in T cells with significantly less exhaustion marker PD1 accumulation than expressing CAR+41BB-CSR, indicative of significantly more functional and less exhausted T cells. Further, because the only difference between the two CAR+CSR constructs is the intracellular domain of CSR, the reduction of cell exhaustion capability is mainly due to the intracellular domain, or costimulatory domain, of CD30. Also significantly, the lower levels of T cell exhaustion markers resulted from CAR+CD30-CSR expression as compared to CAR+41BB-CSR were also seen in both CD8⁺ T cells (cytotoxic T cells) and CD4⁺ T cells (T helper cells). anti-ROR1-cd8TM-41BBz-CAR+anti-ROR1-cd28TM-CD30IC-CSR T cells also exhibited lower PD-1 expression, and displayed a higher percentage and total cell number of central memory T subset, indicating better persistence.

D. Development and Maintenance of Central Memory T Cells from Anti-ROR1 CAR+CSR T Cells after Co-Culture with Target Cells

This example shows that anti-ROR1-CAR+anti-ROR1-CD30-CSR T cells developed into and maintained a high central memory T cell population after target stimulation, including total CD3⁺ central memory T cells and CD8⁺ subset central memory T cells. The cell surface expression of memory T cell markers CCR7 and CD45RA according to the methods described in Examples 5A, 5B, and 14, using all six of the ROR1V cancer cell lines disclosed above. 10⁵ CAR⁺ (receptor⁺) T cells of the two anti-ROR1 CAR+CSR cell groups as described in this example were first co-cultured/engaged with various target cells at an ET ratio of 1:1 in multiple duplicates on 96-well plates. Numbers of central memory T cells (Tcm cell counts) of at least one sample per sample group were quantified on selected days after target cell engagements, which were also labeled for CD3 (for total T cells, including CD8⁺ T cells and CD4⁺ T cells) or CD8 (for CD8⁺ T cells). Every seven days after the first engagement, the unused duplicate cell mixture samples of each group were re-challenged with 10⁵ fresh target cells. Representative results of central memory T cell counts and Tcm percentages of CAR+CD30-CSR and CAR+41BB-CSR T cells are shown in Tables 30 to 44. Central memory T cell percentage among all T cells is % Tcm in the population of total T cells (CD3⁺ cells). Tcm percentage among CD8⁺ cells is % Tcm in the population of CD8⁺ T cells. Ratios of Tcm cell counts and Tcm percentages were calculated for CAR+CD30-CSR over CAR+41BB-CSR T cells.

TABLE 30
Total Central Memory T (Tcm) Cell Counts of Anti-ROR1 CAR + CSR T Cells
after Engagements with A549 Target Cells.

| | Total Central Memory T Cell Count | | Ratio of Total Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 6591 | 4569 | 1.44 |
| E1D5 | 4678 | 4185 | 1.12 |
| E1D7 | 3272 | 2216 | 1.48 |
| E2D3 | 5861 | 3285 | 1.78 |
| E2D5 | 3301 | 2197 | 1.50 |
| E2D7 | 2498 | 2311 | 1.08 |
| E3D3 | 3063 | 2099 | 1.46 |
| E3D7 | 1301 | 1063 | 1.22 |
| E4D3 | 1285 | 1180 | 1.09 |
| E4D7 | 1876 | 1318 | 1.42 |

TABLE 31
Total Central Memory T (Tcm) Cell Counts of Anti-ROR1 CAR + CSR T Cells
after Engagements with H1703 Target Cells.

| | Total Central Memory T Cell Count | | Ratio of Total Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 14114 | 9043 | 1.56 |
| E1D5 | 14710 | 8351 | 1.76 |
| E1D7 | 8367 | 5256 | 1.59 |
| E2D3 | 9152 | 4072 | 2.25 |
| E2D5 | 6078 | 2921 | 2.08 |
| E2D7 | 5166 | 2152 | 2.4 |
| E3D3 | 2894 | 1573 | 1.84 |
| E3D5 | 1552 | 870 | 1.78 |
| E3D7 | 1280 | 692 | 1.85 |
| E4D3 | 685 | 445 | 1.54 |
| E4D5 | 701 | 421 | 1.67 |
| E4D7 | 805 | 385 | 2.09 |

TABLE 32
Total Central Memory T (Tcm) Cell Counts of Anti-ROR1 CAR + CSR T Cells
after Engagements with H1975 Target Cells.

| | Total Central Memory T Cell Count | | Ratio of Total Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 8487 | 3919 | 2.17 |
| E1D5 | 8162 | 5441 | 1.50 |
| E1D7 | 4835 | 2743 | 1.76 |
| E2D3 | 4157 | 3597 | 1.16 |
| E2D5 | 6404 | 3973 | 1.61 |
| E2D7 | 3899 | 2246 | 1.74 |
| E3D3 | 4134 | 2059 | 2.01 |
| E3D5 | 4229 | 2037 | 2.08 |
| E3D7 | 2353 | 744 | 3.16 |
| E4D3 | 1143 | 366 | 3.12 |
| E4D5 | 1870 | 480 | 3.90 |
| E4D7 | 2307 | 588 | 3.92 |

TABLE 33
Total Central Memory T (Tcm) Cell Counts of Anti-ROR1 CAR + CSR T Cells
after Engagements with Jekol Target Cells.

| | Total Central Memory T Cell Count | | |
|---|---|---|---|
| | αROR1-CD8T-41BBz- | αROR1-CD8T-41BBz- | Ratio of Total Tcm Cell |
| | CAR + αROR1-CD28T- | CAR + αROR1-CD28T- | Count CAR + CD30- |
| Date | CD30-CSR | 41BB-CSR | CSR/CAR + 41BB-CSR |
| E1D3 | 11112 | 8020 | 1.39 |
| E1D5 | 15748 | 12297 | 1.28 |
| E1D7 | 7997 | 6201 | 1.29 |
| E2D3 | 18543 | 15587 | 1.19 |
| E2D5 | 21033 | 17589 | 1.20 |
| E2D7 | 15352 | 16600 | 0.92 |
| E3D3 | 26869 | 28181 | 0.95 |
| E3D5 | 21111 | 20372 | 1.04 |
| E3D7 | 15848 | 14179 | 1.12 |
| E4D3 | 16083 | 13652 | 1.18 |
| E4D5 | 12481 | 10937 | 1.14 |
| E4D7 | 12050 | 7060 | 1.71 |

TABLE 34
Total Central Memory T (Tcm) Cell Counts of Anti-ROR1 CAR + CSR T Cells
after Engagements with MDA-MB-231 Target Cells.

| | Total Central Memory T Cell Count | | Ratio of Total Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 13170 | 9671 | 1.36 |
| E1D5 | 22288 | 15219 | 1.46 |
| E1D7 | 16944 | 8804 | 1.92 |
| E2D3 | 21900 | 8974 | 2.44 |
| E2D5 | 9364 | 7484 | 1.25 |
| E2D7 | 11086 | 6450 | 1.72 |
| E3D3 | 9536 | 6533 | 1.46 |
| E3D5 | 7950 | 6506 | 1.22 |
| E3D7 | 5911 | 4035 | 1.46 |
| E4D3 | 6617 | 4580 | 1.44 |
| E4D5 | 4499 | 2859 | 1.57 |
| E4D7 | 3570 | 2550 | 1.40 |

TABLE 35
Total Central Memory T (Tcm) Cell Counts of Anti-ROR1 CAR + CSR T Cells
after Engagements with RPMI8226 Target Cells.

| | Total Central Memory T Cell Count | | Ratio of Total Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 12357 | 8688 | 1.42 |
| E1D5 | 18237 | 12118 | 1.50 |
| E1D7 | 10122 | 6213 | 1.63 |
| E2D3 | 21261 | 19582 | 1.09 |
| E2D5 | 21338 | 23301 | 0.92 |
| E2D7 | 21499 | 22798 | 0.94 |
| E3D3 | 36404 | 21430 | 1.70 |
| E3D5 | 27640 | 15685 | 1.76 |
| E3D7 | 29114 | 16323 | 1.78 |
| E4D3 | 18272 | 13113 | 1.39 |
| E4D5 | 14910 | 13129 | 1.14 |
| E4D7 | 17514 | 15699 | 1.12 |

TABLE 36
Central Memory T Cell (Tcm) Percentage among All T Cells from Anti-ROR1
CAR + CSR T Cells after Engagements with A549 Target Cells.

| | Central Memory T Cell Percentage among | | |
|---|---|---|---|
| | All T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 14.9 | 10.9 | 1.37 |
| E1D5 | 15.5 | 12.3 | 1.26 |
| E1D7 | 17.0 | 13.6 | 1.25 |
| E2D3 | 19.0 | 14.8 | 1.28 |
| E2D5 | 16.0 | 11.0 | 1.45 |
| E2D7 | 14.3 | 13.1 | 1.09 |
| E3D3 | 18.1 | 13.5 | 1.34 |
| E3D7 | 14.1 | 11.1 | 1.27 |
| E4D3 | 13.5 | 10.9 | 1.24 |
| E4D7 | 14.5 | 12.1 | 1.20 |

TABLE 37
Central Memory T Cell (Tcm) Percentage among All T Cells from Anti-ROR1
CAR + CSR T Cells after Engagements with H1975 Target Cells.

| | Central Memory T Cell Percentage among | | |
|---|---|---|---|
| | All T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 16.6 | 13.6 | 1.22 |
| E1D5 | 19.5 | 16.4 | 1.19 |
| E1D7 | 21.6 | 17.1 | 1.26 |
| E2D3 | 21.5 | 17.8 | 1.21 |
| E2D5 | 21.2 | 18.1 | 1.17 |
| E2D7 | 17.7 | 15.7 | 1.13 |
| E3D3 | 18.8 | 16.5 | 1.14 |
| E3D5 | 19.0 | 16.8 | 1.13 |
| E3D7 | 17.5 | 12.8 | 1.37 |
| E4D3 | 14.2 | 9.26 | 1.53 |
| E4D5 | 11.3 | 10.7 | 1.06 |
| E4D7 | 17.5 | 13.5 | 1.30 |

TABLE 38
Central Memory T Cell (Tcm) Percentage among All T Cells from Anti-ROR1
CAR + CSR T Cells after Engagements with Jekol Target Cells.

| | Central Memory T Cell Percentage among | | |
|---|---|---|---|
| | All T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 13.9 | 8.96 | 1.55 |
| E1D5 | 18.1 | 13.4 | 1.35 |
| E1D7 | 20.0 | 15.6 | 1.28 |
| E2D3 | 21.1 | 16.8 | 1.26 |
| E2D5 | 21.6 | 17.7 | 1.22 |
| E2D7 | 24.1 | 21.9 | 1.10 |
| E3D3 | 32.8 | 26.8 | 1.22 |
| E3D5 | 28.9 | 22.9 | 1.26 |
| E3D7 | 30.5 | 27.0 | 1.13 |
| E4D3 | 31.1 | 21.6 | 1.44 |
| E4D5 | 21.7 | 17.8 | 1.22 |
| E4D7 | 25.5 | 23.7 | 1.08 |

TABLE 39
Central Memory T Cell (Tcm) Percentage among All T Cells from Anti-ROR1
CAR + CSR T Cells after Engagements with RPMI8226 Target Cells.

| | Central Memory T Cell Percentage among | | |
|---|---|---|---|
| | All T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 13.2 | 10.0 | 1.32 |
| E1D5 | 18.6 | 16.2 | 1.15 |
| E1D7 | 22.8 | 20.0 | 1.14 |
| E2D3 | 22.7 | 19.5 | 1.16 |
| E2D5 | 19.8 | 19.2 | 1.03 |
| E2D7 | 25.0 | 22.9 | 1.09 |
| E3D3 | 32.9 | 18.7 | 1.76 |
| E3D5 | 26.5 | 18.0 | 1.47 |
| E3D7 | 35.6 | 24.4 | 1.46 |
| E4D3 | 22.5 | 15.5 | 1.45 |
| E4D5 | 19.9 | 14.9 | 1.34 |
| E4D7 | 30.2 | 22.9 | 1.32 |

TABLE 40
CD8⁺ Central Memory T (Tcm) Cell Counts of Anti-ROR1 CAR + CSR T Cells
after Engagements with H1975 Target Cells.

| | CD8⁺ Central Memory T Cell Count | | Ratio of CD8⁺ Tcm Cell |
|---|---|---|---|
| | αROR1-CD8T-41BBz- | αROR1-CD8T-41BBz- | Count |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 2554 | 1308 | 1.95 |
| E1D5 | 3315 | 2173 | 1.53 |
| E1D7 | 2081 | 1264 | 1.65 |
| E2D3 | 2083 | 1864 | 1.12 |
| E2D5 | 2927 | 2042 | 1.43 |
| E2D7 | 1962 | 1264 | 1.55 |
| E3D3 | 2500 | 832 | 3.00 |
| E3D5 | 2146 | 1077 | 1.99 |
| E3D7 | 1264 | 391 | 3.23 |
| E4D3 | 588 | 168 | 3.50 |
| E4D5 | 893 | 224 | 3.99 |
| E4D7 | 1446 | 288 | 5.02 |

TABLE 41
CD8⁺ Central Memory T (Tcm) Cell Counts of Anti-ROR1 CAR + CSR T Cells
after Engagements with MDA-MB-231 Target Cells.

| | CD8⁺ Central Memory T Cell Count | | Ratio of CD8⁺ Tcm Cell |
|---|---|---|---|
| | αROR1-CD8T-41BBz- | αROR1-CD8T-41BBz- | Count |
| | CAR + αRORl-CD28T- | CAR + αRORl-CD28T- | CAR + CD30-CSR/ |
| Date | CD30-CSR | 41BB-CSR | CAR + 41BB-CSR |
| E1D3 | 4275 | 3077 | 1.39 |
| E1D5 | 8674 | 6812 | 1.27 |
| E1D7 | 7494 | 4388 | 1.71 |
| E2D3 | 12155 | 4945 | 2.46 |
| E2D5 | 4826 | 4037 | 1.20 |
| E2D7 | 5925 | 3650 | 1.62 |
| E3D3 | 7391 | 4589 | 1.62 |
| E3D5 | 4108 | 3337 | 1.23 |
| E3D7 | 3624 | 2323 | 1.56 |
| E4D3 | 4249 | 2892 | 1.47 |
| E4D5 | 1711 | 1261 | 1.36 |
| E4D7 | 1627 | 1490 | 1.09 |

TABLE 42
Central Memory T Cell (Tcm) Percentage among CD8⁺ Cells from Anti-ROR1
CAR + CSR T Cells after Engagements with H1975 Target Cells.

| | Central Memory T Cell Percentage among | | |
|---|---|---|---|
| | CD8⁺ T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 8.9 | 7.7 | 1.15 |
| E1D5 | 13.4 | 10.9 | 1.23 |
| E1D7 | 16.2 | 13.1 | 1.24 |
| E2D3 | 17.9 | 14.5 | 1.23 |
| E2D5 | 16.1 | 14.5 | 1.11 |
| E2D7 | 13.7 | 13.0 | 1.05 |
| E3D3 | 17.0 | 13.5 | 1.26 |
| E3D5 | 15.5 | 14.1 | 1.10 |
| E3D7 | 14.0 | 9.8 | 1.44 |
| E4D3 | 11.3 | 6.5 | 1.73 |
| E4D5 | 8.0 | 7.5 | 1.07 |
| E4D7 | 15.4 | 9.7 | 1.59 |

TABLE 43
Central Memory T Cell (Tcm) Percentage among CD8⁺ Cells from Anti-ROR1
CAR + CSR T Cells after Engagements with Jekol Target Cells.

| | Central Memory T Cell Percentage among | | |
|---|---|---|---|
| | CD8⁺ T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 6.0 | 3.5 | 1.71 |
| E1D5 | 9.5 | 7.4 | 1.27 |
| E1D7 | 12.7 | 10.3 | 1.23 |
| E2D3 | 12.4 | 10.0 | 1.24 |
| E2D5 | 14.5 | 12.3 | 1.18 |
| E2D7 | 18.0 | 16.6 | 1.08 |
| E3D3 | 26.6 | 22.2 | 1.20 |
| E3D5 | 22.1 | 17.9 | 1.23 |
| E3D7 | 24.8 | 21.8 | 1.14 |
| E4D3 | 23.7 | 16.2 | 1.46 |
| E4D5 | 17.8 | 14.2 | 1.25 |
| E4D7 | 20.4 | 18.0 | 1.13 |

TABLE 44
Central Memory T Cell (Tcm) Percentage among CD8⁺ Cells from Anti-ROR1
CAR + CSR T Cells after Engagements with RPMI8226 Target Cells.

| | Central Memory T Cell Percentage among | | |
|---|---|---|---|
| | CD8⁺ T Cells (%) | | |
| | αROR1-CD8T-41BBz- | αROR1-CD8T-41BBz- | Ratio of Tcm Percentages |
| | CAR + αROR1-CD28T- | CAR + αROR1-CD28T- | CAR + CD30-CSR/ |
| Date | CD30-CSR | 41BB-CSR | CAR + 41BB-CSR |
| E1D3 | 6.8 | 5.0 | 1.36 |
| E1D5 | 11.2 | 10.4 | 1.08 |
| E1D7 | 16.3 | 14.2 | 1.15 |
| E2D3 | 11.9 | 10.2 | 1.17 |
| E2D5 | 14.6 | 15.1 | 0.97 |
| E2D7 | 20.0 | 18.5 | 1.08 |
| E3D3 | 24.4 | 15.9 | 1.53 |
| E3D5 | 22.1 | 15.4 | 1.44 |
| E3D7 | 30.7 | 19.5 | 1.57 |
| E4D3 | 18.0 | 12.0 | 1.50 |
| E4D5 | 17.2 | 13.0 | 1.32 |
| E4D7 | 26.4 | 19.8 | 1.33 |

The results show that, surprisingly, expressing CAR+CD30-CSR resulted in T cells with significantly higher central memory T cells than expressing CAR+41BB-CSR, indicative of more capability to “store” long-term T cell immune function against the specific targets Further, because the only difference between the two CAR+CSR constructs is the intracellular domain of CSR, the capability of developing and maintaining high numbers of central memory T cells is mainly due to the intracellular domain, or costimulatory domain, of CD30.

### EXEMPLARY EMBODIMENTS

Exemplary embodiments provided in accordance with the presently disclosed subject matter include, but are not limited to, the claims and the following embodiments:

1. An immune cell comprising:
(a) a chimeric antigen receptor (CAR) comprising:
(i) an extracellular target-binding domain comprising an antibody moiety (a CAR antibody moiety);
(ii) a transmembrane domain (a CAR transmembrane domain); and
(iii) a primary signaling domain, and
(b) a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain, wherein the CSR lacks a functional primary signaling domain.
2. The immune cell of embodiment 1, wherein the CD30 costimulatory domain comprises a sequence that can bind to an intracellular TRAF signaling protein.
3. The immune cell of embodiment 2, wherein the sequence that can bind to an intracellular TRAF signaling protein corresponds to residues 561-573 or 578-586 of a full-length CD30 having the sequence of SEQ ID NO:65.
4. The immune cell of any one of embodiments 1 to 3, wherein the CD30 costimulatory domain comprises a sequence that is at least 80%, 85%, 90%, 95%, or 100% identical to residues 561-573 or 578-586 of SEQ ID NO:65.
5. The immune cell of any one of embodiments 1 to 4, wherein the CD30 costimulatory domain comprises a sequence that is at least 70%, 75%, 80%, 85%, 90%, 95%, or 100% identical to the sequence of SEQ ID NO:75.
6. The immune cell of any one of embodiments 1 to 5, wherein the CSR comprises more than one CD30 costimulatory domain.
7. The immune cell of any one of embodiments 1 to 6, wherein the CSR further comprises at least one costimulatory domain which comprises the intracellular sequence of a costimulatory molecule that is different from CD30.
8. The immune cell of embodiment 7, wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.
9. The immune cell of any one of embodiments 1 to 8, wherein the CAR further comprises a costimulatory domain (a CAR costimulatory domain).
10. The immune cell of embodiment 9, wherein the CAR costimulatory domain is derived from the intracellular domain of a costimulatory receptor.
11. The immune cell of embodiment 10, wherein the costimulatory receptor is selected from the group consisting of CD30, CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.
12. The immune cell of any one of embodiments 1 to 11, wherein the ligand-binding module of the CSR is derived from the extracellular domain of a receptor.
13. The immune cell of any one of embodiments 1 to 11, the ligand-binding module of the CSR comprises an antibody moiety (a CSR antibody moiety).
14. The immune cell of embodiment 13, wherein the CSR antibody moiety is a single chain antibody fragment.
15. The immune cell of any one of embodiments 1 to 14, wherein the CAR antibody moiety is a single chain antibody fragment.
16. The immune cell of any one of embodiments 1 to 15, wherein the CAR antibody moiety and/or the CSR antibody moiety is a single chain Fv (scFv), a single chain Fab, a single chain Fab′, a single domain antibody fragment, a single domain multispecific antibody, an intrabody, a nanobody, or a single chain immunokine.
17. The immune cell of embodiment 16, wherein the CAR antibody moiety and/or the CSR antibody moiety is a single domain multispecific antibody.
18. The immune cell of embodiment 17, wherein the single domain multispecific antibody is a single domain bispecific antibody.
19. The immune cell of any one of embodiments 1 to 18, wherein the CAR antibody moiety and/or the CSR antibody moiety is a single chain Fv (scFv).
20. The immune cell of embodiment 19, wherein the scFv is a tandem scFv.
21. The immune cell of any one of embodiments 1 to 20, wherein the CAR antibody moiety and/or the CSR antibody moiety specifically binds to a disease-related antigen.
22. The immune cell of embodiment 21, wherein the disease-related antigen is a cancer-related antigen.
23. The immune cell of embodiment 21, wherein the disease-related antigen is a virus-related antigen.
24. The immune cell of any one of embodiments 1 to 23, wherein the CAR antibody moiety and/or the CSR antibody moiety specifically binds to a cell surface antigen.
25. The immune cell of embodiment 24, wherein the cell surface antigen is selected from the group consisting of protein, carbohydrate, and lipid.
26. The immune cell of embodiment 24 or 25, wherein the cell surface antigen is CD19, CD20, CD22, CD47, CD158e, GPC3, ROR1, ROR2, BCMA, GPRC5D, FcRL5, MUC16, MCT4, PSMA, or a variant or mutant thereof.
27. The immune cell of any one of embodiments 1 to 26, wherein the CAR antibody moiety and the CSR antibody moiety specifically bind to the same antigen.
28. The immune cell of embodiment 27, wherein the CAR antibody moiety and the CSR antibody moiety specifically bind to different epitopes on the same antigen.
29. The immune cell of any one of embodiments 1 to 26, wherein the CAR antibody moiety and/or the CSR antibody moiety specifically binds to a MHC-restricted antigen.
30. The immune cell of embodiment 29, wherein the MHC-restricted antigen is a complex comprising a peptide and an MHC protein, and wherein the peptide is derived from a protein selected from the group consisting of WT-1, AFP, GPC3, HPV16-E7, NY-ESO-1, PRAME, EBV-LMP2A, HIV-1, KRAS, FoxP3, Histone H3.3, PSA, ROR1, and a variant or mutant thereof.
31. The immune cell of any one of embodiments 1 to 28, wherein the CAR antibody moiety binds to CD19, and wherein the ligand-binding module of the CSR binds to CD19.
32. The immune cell of any one of embodiments 1 to 28, wherein the CAR antibody moiety binds to CD22, and wherein the ligand-binding module of the CSR binds to CD22.
33. The immune cell of any one of embodiments 1 to 28, wherein the CAR antibody moiety binds to CD20, and wherein the ligand-binding module of the CSR binds to CD20.
34. The immune cell of any one of embodiments 1 to 26, wherein the CAR antibody moiety binds to CD19, and wherein the ligand-binding module of the CSR binds to CD22.
35. The immune cell of any one of embodiments 1 to 26, wherein the CAR antibody moiety binds to CD19, and wherein the ligand-binding module of the CSR binds to CD20.
36. The immune cell of any one of embodiments 1 to 26, wherein the CAR antibody moiety binds to CD22, and wherein the ligand-binding module of the CSR binds to CD20.
37. The immune cell of any one of embodiments 1 to 26, wherein the CAR antibody moiety binds to CD22, and wherein the ligand-binding module of the CSR binds to CD19.
38. The immune cell of any one of embodiments 1 to 26, wherein the CAR antibody moiety binds to CD20, and wherein the ligand-binding module of the CSR binds to CD19.
39. The immune cell of any one of embodiments 1 to 26, wherein the CAR antibody moiety binds to CD20, and wherein the ligand-binding module of the CSR binds to CD22.
40. The CAR of any one of embodiments 1 to 26, wherein the CAR antibody moiety and/or the ligand-binding module of the CSR binds to both CD19 and CD22.
41. The CAR of any one of embodiments 1 to 26, wherein the CAR antibody moiety and/or the ligand-binding module of the CSR binds to both CD19 and CD20.
42. The CAR of any one of embodiments 1 to 26, wherein the CAR antibody moiety and/or the ligand-binding module of the CSR binds to both CD20 and CD22.
43. The CAR of any one of embodiments 1 to 26, wherein the CAR antibody moiety and/or the ligand-binding module of the CSR binds to CD19, CD20, and CD22.
44. The immune cell of any one of embodiments 1 to 30, wherein the CAR antibody moiety specifically binds to a complex comprising an alpha-fetoprotein (AFP) peptide and an MHC class I protein.
45. The immune cell of embodiment 44, wherein the AFP peptide comprises a sequence of any one of SEQ ID NOS:157-167.
46. The immune cell of embodiment 44 or 45, wherein antibody moiety comprises:
(a) sequences of HCDR1, HCDR2, and HCDR3 of SEQ ID NOS:168-170, respectively, and optionally a heavy chain variable region having the sequence of SEQ ID NO:171, and sequences LCDR1, LCDR2, and LCDR3 of SEQ ID NOS:172-174, respectively, and optionally a light chain variable region having the sequence of SEQ ID NO:175; or
(b) sequences of HCDR1, HCDR2, and HCDR3 of SEQ ID NOS:176-178, respectively, and optionally a heavy chain variable region having the sequence of SEQ ID NO:179, and sequences LCDR1, LCDR2, and LCDR3 of SEQ ID NOS:180-182, respectively, and optionally a light chain variable region having the sequence of SEQ ID NO:183; or
(c) sequences of HCDR1, HCDR2, and HCDR3 of SEQ ID NOS:184-186, respectively, and optionally a heavy chain variable region having the sequence of SEQ ID NO:187, and sequences LCDR1, LCDR2, and LCDR3 of SEQ ID NOS:188-190, respectively, and optionally a light chain variable region having the sequence of SEQ ID NO:191; or
(d) sequences of HCDR1, HCDR2, and HCDR3 of SEQ ID NOS:192-194, respectively, and optionally a heavy chain variable region having the sequence of SEQ ID NO:195, and sequences LCDR1, LCDR2, and LCDR3 of SEQ ID NOS:196-198, respectively, and optionally a light chain variable region having the sequence of SEQ ID NO:199; or
(e) sequences of HCDR1, HCDR2, and HCDR3 of SEQ ID NOS:200-202, respectively, and optionally a heavy chain variable region having the sequence of SEQ ID NO:203, and sequences LCDR1, LCDR2, and LCDR3 of SEQ ID NOS:204-206, respectively, and optionally a light chain variable region having the sequence of SEQ ID NO:207.
47. The immune cell of any one of embodiments 1 to 30, wherein the CAR antibody moiety specifically binds to glypican 3 (GPC3).
48. The immune cell of any one of embodiments 1 to 30 and 47, wherein the ligand-binding module of the CSR specifically binds to GPC3.
49. The immune cell of any one of embodiments 1 to 30, wherein the CAR antibody moiety binds to a complex comprising an AFP peptide and an MHC class I protein, and wherein the ligand-binding module of the CSR binds to GPC3.
50. The immune cell of any one of embodiments 1 to 30, wherein both the CAR antibody moiety and the ligand-binding module of the CSR bind to GPC3.
51. The immune cell of any one of embodiments 47, 48, and 50, wherein the CAR antibody moiety and the ligand-binding module of the CSR specifically bind to different epitopes on GPC3.
52. The immune cell of any one of embodiments 47, 48, 50, and 51, wherein the CAR antibody moiety and/or the ligand-binding module of the CSR comprises:
(a) sequences of HCDR1, HCDR2, and HCDR3 of SEQ ID NOS:208-210, respectively, and optionally a heavy chain variable region having the sequence of SEQ ID NO:211, and sequences LCDR1, LCDR2, and LCDR3 of SEQ ID NOS:212-214, respectively, and optionally a light chain variable region having the sequence of SEQ ID NO:215; or
(b) sequences of HCDR1, HCDR2, and HCDR3 of SEQ ID NOS:216-218, respectively, and optionally a heavy chain variable region having the sequence of SEQ ID NO:219, and sequences LCDR1, LCDR2, and LCDR3 of SEQ ID NOS:220-222, respectively, and optionally a light chain variable region having the sequence of SEQ ID NO:223; or
(c) sequences of HCDR1, HCDR2, and HCDR3 of SEQ ID NOS:224-226, respectively, and optionally a heavy chain variable region having the sequence of SEQ ID NO:227, and sequences LCDR1, LCDR2, and LCDR3 of SEQ ID NOS:228-230, respectively, and optionally a light chain variable region having the sequence of SEQ ID NO:231; or
(d) sequences of HCDR1, HCDR2, and HCDR3 of SEQ ID NOS:232-234, respectively, and optionally a heavy chain variable region having the sequence of SEQ ID NO:235, and sequences LCDR1, LCDR2, and LCDR3 of SEQ ID NOS:236-238, respectively, and optionally a light chain variable region having the sequence of SEQ ID NO:239; or
(e) sequences of HCDR1, HCDR2, and HCDR3 of SEQ ID NOS:240-242, respectively, and optionally a heavy chain variable region having the sequence of SEQ ID NO:243, and sequences LCDR1, LCDR2, and LCDR3 of SEQ ID NOS:244-246, respectively, and optionally a light chain variable region having the sequence of SEQ ID NO:247; or
(f) sequences of HCDR1, HCDR2, and HCDR3 of SEQ ID NOS:248-250, respectively, and optionally a heavy chain variable region having the sequence of SEQ ID NO:251, and sequences LCDR1, LCDR2, and LCDR3 of SEQ ID NOS:252-254, respectively, and optionally a light chain variable region having the sequence of SEQ ID NO:255; or
(g) sequences of HCDR1, HCDR2, and HCDR3 of SEQ ID NOS:256-258, respectively, and optionally a heavy chain variable region having the sequence of SEQ ID NO:259, and sequences LCDR1, LCDR2, and LCDR3 of SEQ ID NOS:260-262, respectively, and optionally a light chain variable region having the sequence of SEQ ID NO:263.
53. The immune cell of any one of embodiments 1 to 30, wherein the CAR antibody moiety specifically binds to a complex comprising a KRAS peptide and an MHC class I protein.
54. The immune cell of embodiment 53, wherein the KRAS peptide comprises a sequence of any one of SEQ ID NOS:264-272.
55. The immune cell of embodiment 53 or 54, wherein antibody moiety comprises:
(a) sequences of HCDR1, HCDR2, and HCDR3 of SEQ ID NOS:273-275, respectively, and optionally a heavy chain variable region having the sequence of SEQ ID NO:276, and sequences LCDR1, LCDR2, and LCDR3 of SEQ ID NOS:277-279, respectively, and optionally a light chain variable region having the sequence of SEQ ID NO:280, and optionally an scFv having the sequence of SEQ ID NO:281; or
(b) sequences of HCDR1, HCDR2, and HCDR3 of SEQ ID NOS:282-284, respectively, and optionally a heavy chain variable region having the sequence of SEQ ID NO:285, and sequences LCDR1, LCDR2, and LCDR3 of SEQ ID NOS:286-288, respectively, and optionally a light chain variable region having the sequence of SEQ ID NO:289, and optionally an scFv having the sequence of SEQ ID NO:290; or
(c) sequences of HCDR1, HCDR2, and HCDR3 of SEQ ID NOS:291-293, respectively, and optionally a heavy chain variable region having the sequence of SEQ ID NO:294, and sequences LCDR1, LCDR2, and LCDR3 of SEQ ID NOS:295-297, respectively, and optionally a light chain variable region having the sequence of SEQ ID NO:298, and optionally an scFv having the sequence of SEQ ID NO:299; or
(d) sequences of HCDR1, HCDR2, and HCDR3 of SEQ ID NOS:300-302, respectively, and optionally a heavy chain variable region having the sequence of SEQ ID NO:303, and sequences LCDR1, LCDR2, and LCDR3 of SEQ ID NOS:304-306, respectively, and optionally a light chain variable region having the sequence of SEQ ID NO:307, and optionally an scFv having the sequence of SEQ ID NO:308; or
(e) sequences of HCDR1, HCDR2, and HCDR3 of SEQ ID NOS:309-311, respectively, and optionally a heavy chain variable region having the sequence of SEQ ID NO:312, and sequences LCDR1, LCDR2, and LCDR3 of SEQ ID NOS:313-315, respectively, and optionally a light chain variable region having the sequence of SEQ ID NO:316, and optionally an scFv having the sequence of SEQ ID NO:317; or
(f) sequences of HCDR1, HCDR2, and HCDR3 of SEQ ID NOS:318-320, respectively, and optionally a heavy chain variable region having the sequence of SEQ ID NO:321, and sequences LCDR1, LCDR2, and LCDR3 of SEQ ID NOS:322-324, respectively, and optionally a light chain variable region having the sequence of SEQ ID NO:325, and optionally an scFv having the sequence of SEQ ID NO:326; or
(g) sequences of HCDR1, HCDR2, and HCDR3 of SEQ ID NOS:327-329, respectively, and optionally a heavy chain variable region having the sequence of SEQ ID NO:330, and sequences LCDR1, LCDR2, and LCDR3 of SEQ ID NOS:331-333, respectively, and optionally a light chain variable region having the sequence of SEQ ID NO:334, and optionally an scFv having the sequence of SEQ ID NO:335; or
(h) sequences of HCDR1, HCDR2, and HCDR3 of SEQ ID NOS:336-338, respectively, and optionally a heavy chain variable region having the sequence of SEQ ID NO:339, and sequences LCDR1, LCDR2, and LCDR3 of SEQ ID NOS:340-342, respectively, and optionally a light chain variable region having the sequence of SEQ ID NO:343, and optionally an scFv having the sequence of SEQ ID NO:344.
56. The immune cell of any one of embodiments 1 to 30, wherein the CAR antibody moiety specifically binds to a complex comprising a PSA peptide and an MHC class I protein.
57. The immune cell of embodiment 56, wherein the PSA peptide comprises a sequence of any one of SEQ ID NOS:345-355.
58. The immune cell of embodiment 56 or 57, wherein antibody moiety comprises:
(a) an HCDR1 having a sequence of any one of SEQ ID NOS:356-370, an HCDR2 having a sequence of any one of SEQ ID NOS:371-384, an HCDR3 having a sequence of any one of SEQ ID NOS:385-402, and optionally a heavy chain variable region having a sequence of any one of SEQ ID NOS:403-420; and/or
(b) an LCDR1 having a sequence of any one of SEQ ID NOS:421-437, an LCDR2 having a sequence of any one of SEQ ID NOS:438-450, an LCDR3 having a sequence of any one of SEQ ID NOS:451-468, and optionally a light chain variable region having a sequence of any one of SEQ ID NOS:469-486.
59. The immune cell of any one of embodiments 1 to 30, wherein the CAR antibody moiety specifically binds to a complex comprising a PSMA peptide and an MHC class I protein.
60. The immune cell of embodiment 59, wherein antibody moiety comprises an scFv having a sequence of SEQ ID NO:487-488.
61. The immune cell of any one of embodiments 1 to 30, wherein the CAR antibody moiety and/or the ligand-binding module of the CSR bind to ROR1.
62. The immune cell of embodiment 61, wherein the CAR antibody moiety and/or the ligand-binding module of the CSR binds to a ROR1 peptide having a sequence of any one of SEQ ID NOS:489-492.
63. The immune cell of embodiment 61 or 62, wherein the CAR antibody moiety and/or the ligand-binding module of the CSR comprises:
(a) sequences of HCDR1, HCDR2, and HCDR3 of SEQ ID NOS:493-495, respectively, and optionally a heavy chain variable region having the sequence of SEQ ID NO:496, and sequences LCDR1, LCDR2, and LCDR3 of SEQ ID NOS:497-499, respectively, and optionally a light chain variable region having the sequence of SEQ ID NO:500; or
(b) sequences of HCDR1, HCDR2, and HCDR3 of SEQ ID NOS:501-503, respectively, and optionally a heavy chain variable region having the sequence of SEQ ID NO:504, and sequences LCDR1, LCDR2, and LCDR3 of SEQ ID NOS:505-507, respectively, and optionally a light chain variable region having the sequence of SEQ ID NO:508.
64. The immune cell of any one of embodiments 1 to 30, wherein the CAR antibody moiety specifically binds to a complex comprising a NY-ESO-1 peptide and an MHC class I protein.
65. The immune cell of embodiment 64, wherein the NY-ESO-1 peptide comprises a sequence of any one of SEQ ID NOS:509-519.
66. The immune cell of embodiment 64 or 65, wherein the antibody moiety comprises:
(a) sequences of HCDR1, HCDR2, and HCDR3 of SEQ ID NOS:520-522, respectively, and optionally a heavy chain variable region having the sequence of SEQ ID NO:523, and sequences LCDR1, LCDR2, and LCDR3 of SEQ ID NOS:524-526, respectively, and optionally a light chain variable region having the sequence of SEQ ID NO:527; or
(b) sequences of HCDR1, HCDR2, and HCDR3 of SEQ ID NOS:528-530, respectively, and optionally a heavy chain variable region having the sequence of SEQ ID NO:531, and sequences LCDR1, LCDR2, and LCDR3 of SEQ ID NOS:532-534, respectively, and optionally a light chain variable region having the sequence of SEQ ID NO:535; or
(c) sequences of HCDR1, HCDR2, and HCDR3 of SEQ ID NOS:536-538, respectively, and optionally a heavy chain variable region having the sequence of SEQ ID NO:539, and sequences LCDR1, LCDR2, and LCDR3 of SEQ ID NOS:540-542, respectively, and optionally a light chain variable region having the sequence of SEQ ID NO:543; or
(d) sequences of HCDR1, HCDR2, and HCDR3 of SEQ ID NOS:544-546, respectively, and optionally a heavy chain variable region having the sequence of SEQ ID NO:547, and sequences LCDR1, LCDR2, and LCDR3 of SEQ ID NOS:548-550, respectively, and optionally a light chain variable region having the sequence of SEQ ID NO:551; or
(e) sequences of HCDR1, HCDR2, and HCDR3 of SEQ ID NOS:552-554, respectively, and optionally a heavy chain variable region having the sequence of SEQ ID NO:555, and sequences LCDR1, LCDR2, and LCDR3 of SEQ ID NOS:556-558, respectively, and optionally a light chain variable region having the sequence of SEQ ID NO:559; or
(f) sequences of HCDR1, HCDR2, and HCDR3 of SEQ ID NOS:560-562, respectively, and optionally a heavy chain variable region having the sequence of SEQ ID NO:563, and sequences LCDR1, LCDR2, and LCDR3 of SEQ ID NOS:564-566, respectively, and optionally a light chain variable region having the sequence of SEQ ID NO:567; or
(g) sequences of HCDR1, HCDR2, and HCDR3 of SEQ ID NOS:568-570, respectively, and optionally a heavy chain variable region having the sequence of SEQ ID NO:571, and sequences LCDR1, LCDR2, and LCDR3 of SEQ ID NOS:572-574, respectively, and optionally a light chain variable region having the sequence of SEQ ID NO:575.
67. The immune cell of any one of embodiments 1 to 30, wherein the CAR antibody moiety specifically binds to a complex comprising a PRAME peptide and an MHC class I protein.
68. The immune cell of embodiment 67, wherein the PRAME peptide comprises a sequence of any one of SEQ ID NOS:576-580.
69. The immune cell of embodiment 67 or 68, wherein the antibody moiety comprises:
(a) sequences of HCDR1, HCDR2, and HCDR3 of SEQ ID NOS:581-583, respectively, and optionally a heavy chain variable region having the sequence of SEQ ID NO:584, and sequences LCDR1, LCDR2, and LCDR3 of SEQ ID NOS:585-587, respectively, and optionally a light chain variable region having the sequence of SEQ ID NO:588; or
(b) sequences of HCDR1, HCDR2, and HCDR3 of SEQ ID NOS:589-591, respectively, and optionally a heavy chain variable region having the sequence of SEQ ID NO:592, and sequences LCDR1, LCDR2, and LCDR3 of SEQ ID NOS:593-595, respectively, and optionally a light chain variable region having the sequence of SEQ ID NO:596; or
(c) sequences of HCDR1, HCDR2, and HCDR3 of SEQ ID NOS:597-599, respectively, and optionally a heavy chain variable region having the sequence of SEQ ID NO:600, and sequences LCDR1, LCDR2, and LCDR3 of SEQ ID NOS:601-603, respectively, and optionally a light chain variable region having the sequence of SEQ ID NO:604; or
(d) sequences of HCDR1, HCDR2, and HCDR3 of SEQ ID NOS:605-607, respectively, and optionally a heavy chain variable region having the sequence of SEQ ID NO:608, and sequences LCDR1, LCDR2, and LCDR3 of SEQ ID NOS:609-611, respectively, and optionally a light chain variable region having the sequence of SEQ ID NO:612; or
(e) sequences of HCDR1, HCDR2, and HCDR3 of SEQ ID NOS:613-615, respectively, and optionally a heavy chain variable region having the sequence of SEQ ID NO:616, and sequences LCDR1, LCDR2, and LCDR3 of SEQ ID NOS:617-619, respectively, and optionally a light chain variable region having the sequence of SEQ ID NO:620; or
(a) sequences of HCDR1, HCDR2, and HCDR3 of SEQ ID NOS:621-623, respectively, and optionally a heavy chain variable region having the sequence of SEQ ID NO:624, and sequences LCDR1, LCDR2, and LCDR3 of SEQ ID NOS:625-627, respectively, and optionally a light chain variable region having the sequence of SEQ ID NO:628; or
(a) sequences of HCDR1, HCDR2, and HCDR3 of SEQ ID NOS:629-631, respectively, and optionally a heavy chain variable region having the sequence of SEQ ID NO:632, and sequences LCDR1, LCDR2, and LCDR3 of SEQ ID NOS:633-635, respectively, and optionally a light chain variable region having the sequence of SEQ ID NO:636.
70. The immune cell of any one of embodiments 1 to 30, wherein the CAR antibody moiety specifically binds to a complex comprising a WT1 peptide and an MHC class I protein.
71. The immune cell of embodiment 70, wherein the WT1 peptide comprises a sequence of SEQ ID NO:637.
72. The immune cell of embodiment 70 or 71, wherein antibody moiety comprises:
(a) sequences of HCDR1, HCDR2, and HCDR3 of SEQ ID NOS:638-640, respectively, and optionally a heavy chain variable region having the sequence of SEQ ID NO:641, and sequences LCDR1, LCDR2, and LCDR3 of SEQ ID NOS:642-644, respectively, and optionally a light chain variable region having the sequence of SEQ ID NO:645, and optionally an scFv having the sequence of SEQ ID NO:646; or
(b) sequences of HCDR1, HCDR2, and HCDR3 of SEQ ID NOS:647-649, respectively, and optionally a heavy chain variable region having the sequence of SEQ ID NO:650, and sequences LCDR1, LCDR2, and LCDR3 of SEQ ID NOS:651-653, respectively, and optionally a light chain variable region having the sequence of SEQ ID NO:654, and optionally an scFv having the sequence of SEQ ID NO:655; or
(c) sequences of HCDR1, HCDR2, and HCDR3 of SEQ ID NOS:656-658, respectively, and optionally a heavy chain variable region having the sequence of SEQ ID NO:659, and sequences LCDR1, LCDR2, and LCDR3 of SEQ ID NOS:660-662, respectively, and optionally a light chain variable region having the sequence of SEQ ID NO:663, and optionally an scFv having the sequence of SEQ ID NO:664; or
(d) sequences of HCDR1, HCDR2, and HCDR3 of SEQ ID NOS:665-667, respectively, and optionally a heavy chain variable region having the sequence of SEQ ID NO:668, and sequences LCDR1, LCDR2, and LCDR3 of SEQ ID NOS:669-671, respectively, and optionally a light chain variable region having the sequence of SEQ ID NO:672, and optionally an scFv having the sequence of SEQ ID NO:673; or
(e) sequences of HCDR1, HCDR2, and HCDR3 of SEQ ID NOS:674-676, respectively, and optionally a heavy chain variable region having the sequence of SEQ ID NO:677, and sequences LCDR1, LCDR2, and LCDR3 of SEQ ID NOS:678-680, respectively, and optionally a light chain variable region having the sequence of SEQ ID NO:681, and optionally an scFv having the sequence of SEQ ID NO:682; or
(f) sequences of HCDR1, HCDR2, and HCDR3 of SEQ ID NOS:683-685, respectively, and optionally a heavy chain variable region having the sequence of SEQ ID NO:686, and sequences LCDR1, LCDR2, and LCDR3 of SEQ ID NOS:687-689, respectively, and optionally a light chain variable region having the sequence of SEQ ID NO:690, and optionally an scFv having the sequence of SEQ ID NO:691.
73. The immune cell of any one of embodiments 1 to 30, wherein the CAR antibody moiety specifically binds to a complex comprising a histone H3.3 peptide and an MHC class I protein.
74. The immune cell of embodiment 73, wherein the histone H3.3 peptide comprises a sequence of any one of SEQ ID NOS:692-711.
75. The immune cell of embodiment 73 or 74, wherein antibody moiety comprises:
(a) sequences of HCDR1, HCDR2, and HCDR3 of SEQ ID NOS:712-714, respectively, and optionally a heavy chain variable region having the sequence of SEQ ID NO:715, and sequences LCDR1, LCDR2, and LCDR3 of SEQ ID NOS:716-718, respectively, and optionally a light chain variable region having the sequence of SEQ ID NO:719; or
(b) sequences of HCDR1, HCDR2, and HCDR3 of SEQ ID NOS:720-722, respectively, and optionally a heavy chain variable region having the sequence of SEQ ID NO:723, and sequences LCDR1, LCDR2, and LCDR3 of SEQ ID NOS:724-726, respectively, and optionally a light chain variable region having the sequence of SEQ ID NO:727; or
(c) sequences of HCDR1, HCDR2, and HCDR3 of SEQ ID NOS:728-730, respectively, and optionally a heavy chain variable region having the sequence of SEQ ID NO:731, and sequences LCDR1, LCDR2, and LCDR3 of SEQ ID NOS:732-734, respectively, and optionally a light chain variable region having the sequence of SEQ ID NO:735; or
(d) sequences of HCDR1, HCDR2, and HCDR3 of SEQ ID NOS:736-738, respectively, and optionally a heavy chain variable region having the sequence of SEQ ID NO:739, and sequences LCDR1, LCDR2, and LCDR3 of SEQ ID NOS:740-742, respectively, and optionally a light chain variable region having the sequence of SEQ ID NO:743; or
(e) sequences of HCDR1, HCDR2, and HCDR3 of SEQ ID NOS:744-746, respectively, and optionally a heavy chain variable region having the sequence of SEQ ID NO:747, and sequences LCDR1, LCDR2, and LCDR3 of SEQ ID NOS:748-750, respectively, and optionally a light chain variable region having the sequence of SEQ ID NO:751; or
(f) sequences of HCDR1, HCDR2, and HCDR3 of SEQ ID NOS:752-754, respectively, and optionally a heavy chain variable region having the sequence of SEQ ID NO:755, and sequences LCDR1, LCDR2, and LCDR3 of SEQ ID NOS:756-758, respectively, and optionally a light chain variable region having the sequence of SEQ ID NO:759; or
(g) sequences of HCDR1, HCDR2, and HCDR3 of SEQ ID NOS:760-762, respectively, and optionally a heavy chain variable region having the sequence of SEQ ID NO:763, and sequences LCDR1, LCDR2, and LCDR3 of SEQ ID NOS:764-766, respectively, and optionally a light chain variable region having the sequence of SEQ ID NO:767; or
(h) sequences of HCDR1, HCDR2, and HCDR3 of SEQ ID NOS:768-770, respectively, and optionally a heavy chain variable region having the sequence of SEQ ID NO:771, and sequences LCDR1, LCDR2, and LCDR3 of SEQ ID NOS:772-774, respectively, and optionally a light chain variable region having the sequence of SEQ ID NO:775; or
(i) sequences of HCDR1, HCDR2, and HCDR3 of SEQ ID NOS:776-778, respectively, and optionally a heavy chain variable region having the sequence of SEQ ID NO:779, and sequences LCDR1, LCDR2, and LCDR3 of SEQ ID NOS:780-782, respectively, and optionally a light chain variable region having the sequence of SEQ ID NO:783; or
(j) sequences of HCDR1, HCDR2, and HCDR3 of SEQ ID NOS:784-786, respectively, and optionally a heavy chain variable region having the sequence of SEQ ID NO:787, and sequences LCDR1, LCDR2, and LCDR3 of SEQ ID NOS:788-790, respectively, and optionally a light chain variable region having the sequence of SEQ ID NO:791; or
(k) sequences of HCDR1, HCDR2, and HCDR3 of SEQ ID NOS:792-794, respectively, and optionally a heavy chain variable region having the sequence of SEQ ID NO:795, and sequences LCDR1, LCDR2, and LCDR3 of SEQ ID NOS:796-798, respectively, and optionally a light chain variable region having the sequence of SEQ ID NO:799; or
(l) sequences of HCDR1, HCDR2, and HCDR3 of SEQ ID NOS:800-802, respectively, and optionally a heavy chain variable region having the sequence of SEQ ID NO:803, and sequences LCDR1, LCDR2, and LCDR3 of SEQ ID NOS:804-806, respectively, and optionally a light chain variable region having the sequence of SEQ ID NO:807.
76. The immune cell of any one of embodiments 1 to 30, wherein the ligand-binding module of the CSR binds to an MSLN peptide.
77. The immune cell of embodiment 76, wherein the ligand-binding module of the CSR comprises sequences of HCDR1, HCDR2, and HCDR3 of SEQ ID NOS:808-810, respectively, and optionally a heavy chain variable region having the sequence of SEQ ID NO:811, and sequences LCDR1, LCDR2, and LCDR3 of SEQ ID NOS:812-814, respectively, and optionally a light chain variable region having the sequence of SEQ ID NO:815.
78. The immune cell of any one of embodiments 1 to 30, wherein the ligand-binding module of the CSR binds to a ROR2 peptide.
79. The immune cell of embodiment 78, wherein the ROR2 peptide comprises the sequence of SEQ ID NO:816.
80. The immune cell of embodiment 78 or 79, wherein the ligand-binding module of the CSR comprises:
(a) sequences of HCDR1, HCDR2, and HCDR3 of SEQ ID NOS:817-819, respectively, and optionally a heavy chain variable region having the sequence of SEQ ID NO:820, and sequences LCDR1, LCDR2, and LCDR3 of SEQ ID NOS:821-823, respectively, and optionally a light chain variable region having the sequence of SEQ ID NO:824; or
(b) sequences of HCDR1, HCDR2, and HCDR3 of SEQ ID NOS:825-827, respectively, and optionally a heavy chain variable region having the sequence of SEQ ID NO:828, and sequences LCDR1, LCDR2, and LCDR3 of SEQ ID NOS:829-831, respectively, and optionally a light chain variable region having the sequence of SEQ ID NO:832; or
(c) sequences of HCDR1, HCDR2, and HCDR3 of SEQ ID NOS:833-835, respectively, and optionally a heavy chain variable region having the sequence of SEQ ID NO:836, and sequences LCDR1, LCDR2, and LCDR3 of SEQ ID NOS:837-839, respectively, and optionally a light chain variable region having the sequence of SEQ ID NO:840; or
(d) sequences of HCDR1, HCDR2, and HCDR3 of SEQ ID NOS:841-843, respectively, and optionally a heavy chain variable region having the sequence of SEQ ID NO:844, and sequences LCDR1, LCDR2, and LCDR3 of SEQ ID NOS:845-847, respectively, and optionally a light chain variable region having the sequence of SEQ ID NO:848; or
(e) sequences of HCDR1, HCDR2, and HCDR3 of SEQ ID NOS:849-851, respectively, and optionally a heavy chain variable region having the sequence of SEQ ID NO:852, and sequences LCDR1, LCDR2, and LCDR3 of SEQ ID NOS:853-855, respectively, and optionally a light chain variable region having the sequence of SEQ ID NO:856.
81. The immune cell of any one of embodiments 1 to 30, wherein the ligand-binding module of the CSR binds to a HER2 peptide.
82. The immune cell of embodiment 81, wherein the ligand-binding module of the CSR comprises sequences of HCDR1, HCDR2, and HCDR3 of SEQ ID NOS:857-859, respectively, and optionally a heavy chain variable region having the sequence of SEQ ID NO:860, and sequences LCDR1, LCDR2, and LCDR3 of SEQ ID NOS:861-863, respectively, and optionally a light chain variable region having the sequence of SEQ ID NO:864, and optionally an scFv having the sequence of SEQ ID NO:865.
83. The immune cell of any one of embodiments 1 to 30, wherein the ligand-binding module of the CSR binds to an EpCAM peptide.
84. The immune cell of embodiment 83, wherein the ligand-binding module of the CSR comprises sequences of HCDR1, HCDR2, and HCDR3 of SEQ ID NOS:866-868, respectively, and optionally a heavy chain variable region having the sequence of SEQ ID NO:869, and sequences LCDR1, LCDR2, and LCDR3 of SEQ ID NOS:870-872, respectively, and optionally a light chain variable region having the sequence of SEQ ID NO:873.
85. The immune cell of any one of embodiments 1 to 30, wherein the ligand-binding module of the CSR binds to a MUC1 peptide.
86. The immune cell of embodiment 85, wherein the ligand-binding module of the CSR comprises sequences of HCDR1, HCDR2, and HCDR3 of SEQ ID NOS:874-876, respectively, and optionally a heavy chain variable region having the sequence of SEQ ID NO:877, and sequences LCDR1, LCDR2, and LCDR3 of SEQ ID NOS:878-880, respectively, and optionally a light chain variable region having the sequence of SEQ ID NO:881.
87. The immune cell of any one of embodiments 1 to 30, wherein the ligand-binding module of the CSR binds to a MUC16 peptide.
88. The immune cell of embodiment 87, wherein the ligand-binding module of the CSR comprises:
(a) sequences of HCDR1, HCDR2, and HCDR3 of SEQ ID NOS:882-884, respectively, and optionally a heavy chain variable region having the sequence of SEQ ID NO:885, and sequences LCDR1, LCDR2, and LCDR3 of SEQ ID NOS:886-888, respectively, and optionally a light chain variable region having the sequence of SEQ ID NO:889; or
(b) sequences of HCDR1, HCDR2, and HCDR3 of SEQ ID NOS:890-892, respectively, and optionally a heavy chain variable region having the sequence of SEQ ID NO:893, and sequences LCDR1, LCDR2, and LCDR3 of SEQ ID NOS:894-896, respectively, and optionally a light chain variable region having the sequence of SEQ ID NO:897; or
(c) sequences of HCDR1, HCDR2, and HCDR3 of SEQ ID NOS:898-900, respectively, and optionally a heavy chain variable region having the sequence of SEQ ID NO:901 or 902, and sequences LCDR1, LCDR2, and LCDR3 of SEQ ID NOS:903-905, respectively, and optionally a light chain variable region having the sequence of SEQ ID NO:906 or 907; or
(d) sequences of HCDR1, HCDR2, and HCDR3 of SEQ ID NOS:908-910, respectively, and optionally a heavy chain variable region having the sequence of SEQ ID NO:911 or 912, and sequences LCDR1, LCDR2, and LCDR3 of SEQ ID NOS:913-915, respectively, and optionally a light chain variable region having the sequence of SEQ ID NO:916 or 917.
89. The immune cell of any one of embodiments 1 to 30, wherein the ligand-binding module of the CSR binds to an FRα peptide.
90. The immune cell of embodiment 89, wherein the ligand-binding module of the CSR comprises:
(a) sequences of HCDR1, HCDR2, and HCDR3 of SEQ ID NOS:918-920, respectively, and optionally a heavy chain variable region having the sequence of SEQ ID NO:921, and sequences LCDR1, LCDR2, and LCDR3 of SEQ ID NOS:923-925, respectively, and optionally a light chain variable region having the sequence of SEQ ID NO:926.
91. The immune cell of any one of embodiments 1 to 30, wherein the ligand-binding module of the CSR binds to an EGFR peptide.
92. The immune cell of embodiment 91, wherein the ligand-binding module of the CSR comprises sequences of HCDR1, HCDR2, and HCDR3 of SEQ ID NOS:928-930, respectively, and optionally a heavy chain variable region having the sequence of SEQ ID NO:931, and sequences LCDR1, LCDR2, and LCDR3 of SEQ ID NOS:932-934, respectively, and optionally a light chain variable region having the sequence of SEQ ID NO:935; and optionally an scFv having the sequence of SEQ ID NO:936.
93. The immune cell of any one of embodiments 1 to 30, wherein the ligand-binding module of the CSR binds to an EGFRVIII peptide.
94. The immune cell of embodiment 93, wherein the ligand-binding module of the CSR comprises sequences of HCDR1, HCDR2, and HCDR3 of SEQ ID NOS:937-939, respectively, and optionally a heavy chain variable region having the sequence of SEQ ID NO:940, and sequences LCDR1, LCDR2, and LCDR3 of SEQ ID NOS:941-943, respectively, and optionally a light chain variable region having the sequence of SEQ ID NO:944.
95. The immune cell of any one of embodiments 1 to 30, wherein the ligand-binding module of the CSR binds to an HER3 peptide.
96. The immune cell of embodiment 95, wherein the ligand-binding module of the CSR comprises sequences of HCDR1, HCDR2, and HCDR3 of SEQ ID NOS:945-947, respectively, and optionally a heavy chain variable region having the sequence of SEQ ID NO:948, and sequences LCDR1, LCDR2, and LCDR3 of SEQ ID NOS:949-951, respectively, and optionally a light chain variable region having the sequence of SEQ ID NO:952, and optionally an scFv having the sequence of SEQ ID NO:953.
97. The immune cell of any one of embodiments 1 to 30, wherein the ligand-binding module of the CSR binds to a DLL3 peptide.
98. The immune cell of embodiment 97, wherein the ligand-binding module of the CSR comprises sequences of HCDR1, HCDR2, and HCDR3 of SEQ ID NOS:954-956, respectively, and optionally a heavy chain having the sequence of SEQ ID NO:957, and sequences LCDR1, LCDR2, and LCDR3 of SEQ ID NOS:958-960, respectively, and optionally a light chain having the sequence of SEQ ID NO:961.
99. The immune cell of any one of embodiments 1 to 30, wherein the ligand-binding module of the CSR binds to a c-Met peptide.
100. The immune cell of embodiment 99, wherein the ligand-binding module of the CSR comprises sequences of HCDR1, HCDR2, and HCDR3 of SEQ ID NOS:962-964, respectively, and optionally a heavy chain having the sequence of SEQ ID NO:965, and sequences LCDR1, LCDR2, and LCDR3 of SEQ ID NOS:966-968, respectively, and optionally a light chain having the sequence of SEQ ID NO: 969.
101. The immune cell of any one of embodiments 1 to 30, wherein the ligand-binding module of the CSR binds to a CD70 peptide.
102. The immune cell of embodiment 101, wherein the ligand-binding module of the CSR comprises sequences of HCDR1, HCDR2, and HCDR3 of SEQ ID NOS:970-972, respectively, and optionally a heavy chain variable region having the sequence of SEQ ID NO:973, and sequences LCDR1, LCDR2, and LCDR3 of SEQ ID NOS:974-976, respectively, and optionally a light chain having the sequence of SEQ ID NO:977.
103. The immune cell of any one of embodiments 1 to 102, wherein the CAR transmembrane domain is the transmembrane domain of CD30.
104. The immune cell of any one of embodiments 1 to 102, wherein the CAR transmembrane domain is the transmembrane domain of CD8.
105. The immune cell of any one of embodiments 1 to 104, wherein the CAR transmembrane domain and/or the CSR transmembrane domain is derived from the transmembrane domain of a TCR co-receptor or a T cell co-stimulatory molecule.
106. The immune cell of embodiment 105, wherein the TCR co-receptor or T cell co-stimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154.
107. The immune cell of embodiment 105 or 106, wherein the TCR co-receptor or T cell co-stimulatory molecule is CD30 or CD8.
108. The immune cell of embodiment 107, wherein the T cell co-stimulatory molecule is CD30.
109. The immune cell of embodiment 107, wherein the TCR co-receptor is CD8.
110. The immune cell of any one of embodiments 1 to 104, wherein the CAR transmembrane domain and/or the CSR transmembrane domain is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154.
111. The immune cell of embodiment 110, wherein the CAR transmembrane domain and/or the CSR transmembrane domain is the transmembrane domain of CD30 or CD8.
112. The immune cell of embodiment 111, wherein the CAR transmembrane domain and/or the CSR transmembrane domain is the transmembrane domain of CD30.
113. The immune cell of embodiment 112, wherein the CSR transmembrane domain is the transmembrane domain of CD30.
114. The immune cell of embodiment 112, wherein the CAR transmembrane domain and/or the CSR transmembrane domain is the transmembrane domain of CD8.
115. The immune cell of any one of embodiments 1 to 114, wherein the CAR transmembrane domain and/or the CSR transmembrane domain comprises an amino acid sequence selected from the group consisting of SEQ ID NOS:66-71.
116. The immune cell of any one of embodiments 1 to 115, wherein the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d.
117. The immune cell of embodiment 116, wherein the primary signaling domain comprises a sequence derived from the intracellular signaling sequence of CD3ζ.
118. The immune cell of embodiment 116, wherein the primary signaling domain comprises the intracellular signaling sequence of CD3ζ.
119. The immune cell of any one of embodiments 1 to 118, wherein the primary signaling domain comprises a sequence that is at least 80%, 85%, 90%, 95%, or 100% identical to the sequence of SEQ ID NO:77.
120. The immune cell of any one of embodiments 1 to 119, further comprises a peptide linker between the extracellular target-binding domain and the transmembrane domain of the CAR.
121. The immune cell of any one of embodiments 1 to 120, further comprises a peptide linker between the transmembrane domain and the costimulatory domain of the CAR.
122. The immune cell of any one of embodiments 1 to 121, further comprises a peptide linker between the costimulatory domain and the primary signaling domain of the CAR.
123. The immune cell of any one of embodiments 1 to 122, further comprises a peptide linker between the ligand-binding module and the transmembrane domain of the CSR.
124. The immune cell of any one of embodiments 1 to 123, further comprises a peptide linker between the transmembrane domain and the CD30 costimulatory domain of the CSR.
125. The immune cell of any one of embodiments 1 to 124, wherein the expression of the CSR is inducible.
126. The immune cell of embodiment 125, wherein the expression of the CSR is inducible upon activation of the immune cell.
127. The immune cell of any one of embodiments 1 to 126, wherein the immune cell is selected from the group consisting of a cytotoxic T cell, a helper T cell, a natural killer T cell, and a suppressor T cell.
128. One or more nucleic acids encoding the CAR and CSR comprised by the immune cell of any one of embodiments 1 to 127, wherein the CAR and CSR each consist of one or more polypeptide chains encoded by the one or more nucleic acids.
129. One or more vectors comprising the one or more nucleic acids of embodiment 128.
130. A pharmaceutical composition comprising: (a) the immune cell of any one of embodiments 1 to 127, the nucleic acid(s) of embodiment 128, or the vector(s) of embodiment 129, and (b) a pharmaceutically acceptable carrier or diluent.
131. A method of killing target cells, comprising:
contacting one or more target cells with the immune cell of any one of embodiments 1 to 127 under conditions and for a time sufficient so that the immune cells mediate killing of the target cells,
   wherein the target cells express an antigen specific to the immune cell, and
   wherein the immune cell expresses a low cell exhaustion level upon contacting the target cells.
   132. The method of embodiment 131, wherein the immune cell expresses a low cell exhaustion level of an exhaustion marker selected from the group consisting of PD-1, TIM-3, TIGIT, and LAG-3.
   133. The method of embodiment 131 or 132, wherein the immune cell is a T cell.
   134. The method of any one of embodiments 131 to 133, wherein the immune cell expresses a low cell exhaustion level of PD-1.
   135. The method of any one of embodiments 131 to 133, wherein the immune cell expresses a low cell exhaustion level of TIM-3.
   136. The method of any one of embodiments 131 to 133, wherein the immune cell expresses a low cell exhaustion level of LAG-3.
   137. The method of any one of embodiments 131 to 133, wherein the immune cell expresses a low cell exhaustion level of TIGIT.
   138. The method of any one of embodiments 131 to 137, wherein the immune cell expresses a lower level of PD-1, TIM-3, TIGIT, or LAG-3 than corresponding immune cell expressing a CSR comprising a CD28 costimulatory domain.
   139. The method of embodiment 138, wherein the immune cell expresses a lower level of PD-1 than the corresponding CD28 CSR immune cell, and wherein the ratio of PD-1 expression level of the immune cell to the corresponding CD28 CSR immune cell is 0.9, 0.8, 0.7, 0.6, 0.5, 0.4, 0.3, 0.2, 0.1 or lower.
   140. The method of embodiment 138, wherein the immune cell expresses a lower level of TIM-3 than the corresponding CD28 CSR immune cell, and wherein the ratio of TIM-3 expression level of the immune cell to the corresponding CD28 CSR immune cell is 0.9, 0.8, 0.7, 0.6, 0.5, 0.4, 0.3, 0.2, 0.1 or lower.
   141. The method of embodiment 138, wherein the immune cell expresses a lower level of LAG-3 than the corresponding CD28 CSR immune cell, and wherein the ratio of LAG-3 expression level of the immune cell to the corresponding CD28 CSR immune cell is 0.9, 0.8, 0.7, 0.6, 0.5, 0.4, 0.3, 0.2, 0.1 or lower.
   142. The method of embodiment 138, wherein the immune cell expresses a lower level of TIGIT than the corresponding CD28 CSR immune cell, and wherein the ratio of TIGIT expression level of the immune cell to the corresponding CD28 CSR immune cell is 0.9, 0.8, 0.7, 0.6, 0.5, 0.4, 0.3, 0.2, 0.1 or lower.
   143. The method of any one of embodiments 131 to 137, wherein the immune cell expresses a lower level of PD-1, TIM-3, TIGIT, or LAG-3 than corresponding immune cell expressing a CSR comprising a 4-1BB costimulatory domain.
   144. The method of embodiment 143, wherein the immune cell expresses a lower level of PD-1 than the corresponding 4-1BB CSR immune cell, and wherein the ratio of PD-1 expression level of the immune cell to the corresponding 4-1BB CSR immune cell is 0.9, 0.8, 0.7, 0.6, 0.5, 0.4, 0.3, 0.2, 0.1 or lower.
   145. The method of embodiment 143, wherein the immune cell expresses a lower level of TIM-3 than the corresponding 4-1BB CSR immune cell, and wherein the ratio of TIM-3 expression level of the immune cell to the corresponding 4-1BB CSR immune cell is 0.9, 0.8, 0.7, 0.6, 0.5, 0.4, 0.3, 0.2, 0.1 or lower.
   146. The method of embodiment 143, wherein the immune cell expresses a lower level of LAG-3 than the corresponding 4-1BB CSR immune cell, and wherein the ratio of LAG-3 expression level of the immune cell to the corresponding 4-1BB CSR immune cell is 0.9, 0.8, 0.7, 0.6, 0.5, 0.4, 0.3, 0.2, 0.1 or lower.
   147. The method of embodiment 143, wherein the immune cell expresses a lower level of TIGIT than the corresponding 4-1BB CSR immune cell, and wherein the ratio of TIGIT expression level of the immune cell to the corresponding 4-1BB CSR immune cell is 0.9, 0.8, 0.7, 0.6, 0.5, 0.4, 0.3, 0.2, 0.1 or lower.
   148. The method of any one of embodiments 131 to 147, wherein the target cells are cancer cells.
   149. The method of embodiment 148, wherein the cancer cells are from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.
   150. The method of embodiment 147 or 148, wherein the cancer cells are hematological cancer cells.
   151. The method of embodiment 147 or 148, wherein the cancer cells are solid tumor cells.
   152. The method of any one of embodiments 131 to 147, wherein the target cells are virus-infected cells.
   153. The method of embodiment 152, wherein the virus-infected cells are from a viral infection caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).
   154. A method of treating a disease, the method comprising a step of administering to a subject the immune cell of any one of embodiments 1 to 127, the nucleic acid(s) of embodiment 128, or the vector(s) of embodiment 129, or the pharmaceutical composition of embodiment 130 to the subject.
   155. The method of embodiment 154, wherein the disease is a viral infection.
   156. The method of embodiment 154, wherein the disease is cancer.
   157. The method of embodiment 156, wherein the cancer is a hematological cancer.
   158. The method of embodiment 156, wherein the cancer is a solid tumor cancer.
   159. The method of embodiment 158, wherein the subject has a higher density of the immune cell of any one of embodiments 1 to 127 in the solid tumor cancer than in the rest of the subject's body.
   160. The method of any one of embodiments 154 to 159, wherein the subject has a higher density of the immune cell of any one of embodiments 1 to 127 in the peripheral blood of the subject as compared to treating the same type of disease with same number of immune cells expressing the same CAR and a corresponding CSR comprising a CD28 or 4-1BB costimulatory domain.
   161. The method of any one of embodiments 154 to 159, wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.
   162. A method for preventing and/or reversing T cell exhaustion in a subject, comprising administering to the subject the nucleic acid(s) of embodiment 128, or the vector(s) of embodiment 129, or the pharmaceutical composition of embodiment 130 comprising the nucleic acid(s) or the vector(s) to the subject.
   163. The method of embodiment 162, wherein the method decreases the expression of an exhaustion marker in a T cell.
   164. The method of embodiment 162 or 163, wherein the exhaustion marker is selected from the group consisting of PD-1, TIM-3, TIGIT, and LAG-3.
   165. A method of treating a solid tumor cancer in a subject with increased tumor infiltration as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell of any one of embodiments 1 to 127.
   166. A method of treating a solid tumor cancer in a subject with increased tumor regression as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell of any one of embodiments 1 to 127.
   167. A method for generating central memory T cells in a subject, comprising administering to the subject the nucleic acid(s) of embodiment 128, or the vector(s) of embodiment 129, or the pharmaceutical composition of embodiment 130 comprising the nucleic acid(s) or the vector(s) to the subject.
   168. The method of embodiment 167, wherein the method increases the number of central memory T cells and/or the percentage of central memory T cells among all T cells in the subject.
   169. A method for generating central memory T cells in vitro comprising: contacting one or more target cells with the immune cell of any one of embodiments 1 to 127 under conditions and for a time sufficient so that the immune cell develops into central memory T cells, wherein the target cells express an antigen specific to the immune cell.
   170. The method of embodiment 169, wherein the method increases the number of central memory T cells and/or the percentage of central memory T cells among all T cells descended from the immune cell.
   171. The method of embodiment 169 or 170, wherein the method generates higher number of central memory T cells and/or higher percentage of central memory T cells than corresponding immune cell expressing a CSR comprising a CD28 costimulatory domain.
   172. The method of embodiment 171, wherein the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 costimulatory domain.
   173. The method of any one of embodiments 169 to 172, wherein the central memory T cells express high levels of CCR7 and low levels of CD45RA.
   174. The method of any one of embodiments 169 to 173, wherein the central memory T cells are CD8+ T cells.

INFORMAL SEQUENCE LISTING

| SEQ | | |
|---|---|---|
| ID | | |
| NO. | Sequence | Notes |
| 1 | METDTLLLWVLLLWVPGSTGQSVLTQPASVSGSPGQSITISCTGTSSDVGGYNYVSW | 1st -gen αAFP-CD8T- |
| | YQQHPGKAPKLMIYDVNNRPSEVSNRFSGSKSGNTASLTISGLQAEDEADYYCSSYTT | Z-CAR |
| | GSRAVFGGGTKLTVLGSRGGGGSGGGGSGGGGSLEMAEVQLVQSGAEVKKPGESLTI | Signal peptide- AFP |
| | SCKASGYSFPNYWITWVRQMSGGGLEWMGRIDPGDSYTTYNPSFQGHVTISIDKSTN | scFv-myc tag-linker- |
| | TAYLHWNSLKASDTAMYYCARYYVSLVDIWGQGTLVTVSSEQKLISEEDLAAATGT | CD8T-CD3z |
| | TTPAPRPPTPAPTIASQPLSLRPEACRPAAGGAVHTRGLDFACDIYIWAPLAGTCGVLL | |
| | LSLVITLYCRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKDTYD | |
| | ALHMQALPPR | |
| 2 | METDTLLLWVLLLWVPGSTGLPVLTQPPSVSVAPGKTARITCGGNNIGSKSVHWYQQ | 1st -gen αCD19- |
| | KPGQAPVLVVYDDSDRPSGIPERFSGSNSGNTATLTISRVEAGDEADYYCQVWDSSSD | CD8T-Z-CAR |
| | YVVFGGGTKLTVLGSRGGGGSGGGGSGGGGSLEMAEVQLVQSGAEVKKPGESLKIS | Signal peptide- CD19 |
| | CKGSGYSFTSYWIGWVRQMPGKGLEWMGIIYPGDSDTRYSPSFQGQVTISADKSISTA | scFv-myc tag-linker- |
| | YLQWSSLKASDTAMYYCARQVWGWQGGMYPRSNWWYNLDSWGQGTLVTVSSEQ | CD8T-CD3z |
| | KLISEEDLAAATGTTTPAPRPPTPAPTIASQPLSLRPEACRPAAGGAVHTRGLDFACDIY | |
| | IWAPLAGTCGVLLLSLVITLYCRVKFSRSADAPAYQQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 3 | METDTLLLWVLLLWVPGSTGLPVLTQPPSVSVAPGKTARITCGGNNIGSKSVHWYQQ | 2^{nd}-gen |
| | KPGQAPVLVVYDDSDRPSGIPERFSGSNSGNTATLTISRVEAGDEADYYCQVWDSSSD | Anti-CD19-CD28z- |
| | YVVFGGGTKLTVLGSRGGGGSGGGGSGGGGSLEMAEVQLVQSGAEVKKPGESLKIS | CAR |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAAIEVMYPPPYLDNEKSNGTIIHVKGKHLCPSPLFPGPSKPFWVLVVVGG | |
| | VLACYSLLVTVAFIIFWVRSKRSRLLHSDYMNMTPRRPGPTRKHYQPYAPPRDFAAY | |
| | RSRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 4 | METDTLLLWVLLLWVPGSTGLPVLTQPPSVSVAPGKTARITCGGNNIGSKSVHWYQQ | 2^{nd}-gen |
| | KPGQAPVLVVYDDSDRPSGIPERFSGSNSGNTATLTISRVEAGDEADYYCQVWDSSSD | Anti-CD19-CD30z- |
| | YVVFGGGTKLTVLGSRGGGGSGGGGSGGGGSLEMAEVQLVQSGAEVKKPGESLKIS | CAR |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAATGAPPLGTQPDCNPTPENGEAPASTSPTQSLLVDSQASKTLPIPTSAPV | |
| | ALSSTGKPVLDAGPVLFWVILVLVVVVGSSAFLLCHRRACRKRIRQKLHLCYPVQTSQ | |
| | PKLELVDSRPRRSSTQLRSGASVTEPVAEERGLMSQPLMETCHSVGAAYLESLPLQDA | |
| | SPAGGPSSPRDLPEPRVSTEHTNNKIEKIVEVIKADTVIVGTVKAELPEGRGLAGPAEPE | |
| | LEEELEADHTPHYPEQETEPPLGSCSDVMLSVEEEGKEDPLPTAASGKRVKFSRSADA | |
| | PAYQQGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| 5 | METDTLLLWVLLLWVPGSTGLPVLTQPPSVSVAPGKTARITCGGNNIGSKSVHWYQQ | 2^{nd}-gen |
| | KPGQAPVLVVYDDSDRPSGIPERFSGSNSGNTATLTISRVEAGDEADYYCQVWDSSSD | Anti-CD19-CD8T- |
| | YVVFGGGTKLTVLGSRGGGGSGGGGSGGGGSLEMAEVQLVQSGAEVKKPGESLKIS | CD30z-CAR |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLAAATGTTTPAPRPPTPAPTIASQPLSLRPEACRPAAGGAVHTRGLDFACDIY | |
| | IWAPLAGTCGVLLLSLVITLYCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYIMKADTVIVGTVKAELPEGRGLAGPAEPELEEELEADHTPHYP | |
| | EQETEPPLGSCSDVMLSVEEEGKEDPLPTAASGKRVKFSRSADAPAYQQGQNQLYNE | |
| | LNLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| 6 | METDTLLLWVLLLWVPGSTGLPVLTQPPSVSVAPGKTARITCGGNNIGSKSVHWYQQ | 2^{nd}-gen |
| | KPGQAPVLVVYDDSDRPSGIPERFSGSNSGNTATLTISRVEAGDEADYYCQVWDSSSD | Anti-CD19-CD8T- |
| | YVVFGGGTKLTVLGSRGGGGSGGGGSGGGGSLEMAEVQLVQSGAEVKKPGESLKIS | 41BBz-CAR |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLTGTTTPAPRPPTPAPTIASQPLSLRPEACRPAAGGAVHTRGLDFACDIYIWA | |
| | PLAGTCGVLLLSLVITLYCKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 7 | METDTLLLWVLLLWVPGSTGQSVLTQPASVSGSPGQSITISCTGTSSDVGGYNYVSW | 2^{nd}-gen |
| | YQQHPGKAPKLMIYDVNNRPSEVSNRFSGSKSGNTASLTISGLQAEDEADYYCSSYTT | Anti-AFP-CD28z- |
| | GSRAVFGGGTKLTVLGSRGGGGSGGGGSGGGGSLEMAEVQLVQSGAEVKKPGESLTI | CAR |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAAIEV | |
| | MYPPPYLDNEKSNGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 8 | METDTLLLWVLLLWVPGSTGQSVLTQPASVSGSPGQSITISCTGTSSDVGGYNYVSW | 2^{nd}-gen |
| | YQQHPGKAPKLMIYDVNNRPSEVSNRFSGSKSGNTASLTISGLQAEDEADYYCSSYTT | Anti-AFP-CD30z- |
| | GSRAVFGGGTKLTVLGSRGGGGSGGGGSGGGGSLEMAEVQLVQSGAEVKKPGESLTI | CAR |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGA | |
| | PPLGTQPDCNPTPENGEAPASTSPTQSLLVDSQASKTLPIPTSAPVALSSTGKPVLDAGP | |
| | VLFWVILVLVVVVGSSAFLLCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYEVIKADTVIVGTVKAELPEGRGLAGPAEPELEEELEADHTPHYP | |
| | EQETEPPLGSCSDVMLSVEEEGKEDPLPTAASGKRVKFSRSADAPAYQQGQNQLYNE | |
| | LNLGRREEYDVLDKRRGRDPEMGGKPRRKNPQEGLYNELQKDKMAEAYSEIGMKG | |
| | ERRRGKGHDGLYQGLSTATKDTYDALHMQALPPR | |
| 9 | METDTLLLWVLLLWVPGSTGQSVLTQPASVSGSPGQSITISCTGTSSDVGGYNYVSW | 2^{nd}-gen |
| | YQQHPGKAPKLMIYDVNNRPSEVSNRFSGSKSGNTASLTISGLQAEDEADYYCSSYTT | AFP-CD8T-CD30z- |
| | GSRAVFGGGTKLTVLGSRGGGGSGGGGSGGGGSLEMAEVQLVQSGAEVKKPGESLTI | CAR |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGT | |
| | TTPAPRPPTPAPTIASQPLSLRPEACRPAAGGAVHTRGLDFACDIYIWAPLAGTCGVLL | |
| | LSLVITLYCHRRACRKRIRQKLHLCYPVQTSQPKLELVDSRPRRSSTQLRSGASVTEPV | |
| | AEERGLMSQPLMETCHSVGAAYLESLPLQDASPAGGPSSPRDLPEPRVSTEHTNNKIE | |
| | KIYIMKADTVIVGTVKAELPEGRGLAGPAEPELEEELEADHTPHYPEQETEPPLGSCSD | |
| | VMLSVEEEGKEDPLPTAASGKRVKFSRSADAPAYQQGQNQLYNELNLGRREEYDVL | |
| | DKRRGRDPEMGGKPRRKNPQEGLYNELQKDKMAEAYSEIGMKGERRRGKGHDGLY | |
| | QGLSTATKDTYDALHMQALPPR | |
| 10 | METDTLLLWVLLLWVPGSTGQSVLTQPASVSGSPGQSITISCTGTSSDVGGYNYVSW | 2^{nd}-gen |
| | YQQHPGKAPKLMIYDVNNRPSEVSNRFSGSKSGNTASLTISGLQAEDEADYYCSSYTT | Anti-AFP-CD8T- |
| | GSRAVFGGGTKLTVLGSRGGGGSGGGGSGGGGSLEMAEVQLVQSGAEVKKPGESLTI | 41BBz-CAR |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLTGTTTPA | |
| | PRPPTPAPTIASQPLSLRPEACRPAAGGAVHTRGLDFACDIYIWAPLAGTCGVLLLSLVI | |
| | TLYCKRGRKKLLYIFKQPFMRPVQTTQEEDGCSCRFPEEEEGGCELRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 11 | METDTLLLWVLLLWVPGSTGLPVLTQPPSVSVAPGKTARITCGGNNIGSKSVHWYQQ | 2^{nd}-gen |
| | KPGQAPVLVVYDDSDRPSGIPERFSGSNSGNTATLTISRVEAGDEADYYCQVWDSSSD | Anti-CD19-CD8T- |
| | YVVFGGGTKLTVLGSRGGGGSGGGGSGGGGSLEMAEVQLVQSGAEVKKPGESLKIS | CD28z-CAR |
| | CKGSGYSFTSYWIGWVRQMPGKGLEWMGIIYPGDSDTRYSPSFQGQVTISADKSISTA | |
| | YLQWSSLKASDTAMYYCARQVWGWQGGMYPRSNWWYNLDSWGQGTLVTVSSEQ | |
| | KLISEEDLTGTTTPAPRPPTPAPTIASQPLSLRPEACRPAAGGAVHTRGLDFACDIYIWA | |
| | PLAGTCGVLLLSLVITLYCRSKRSRLLHSDYMNMTPRRPGPTRKHYQPYAPPRDFAAY | |
| | RSRVKFSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALHMQAL | |
| | PPR | |
| 12 | METDTLLLWVLLLWVPGSTGQSVLTQPASVSGSPGQSITISCTGTSSDVGGYNYVSW | 2^{nd}-gen |
| | YQQHPGKAPKLMIYDVNNRPSEVSNRFSGSKSGNTASLTISGLQAEDEADYYCSSYTT | Anti-AFP-CD8T- |
| | GSRAVFGGGTKLTVLGSRGGGGSGGGGSGGGGSLEMAEVQLVQSGAEVKKPGESLTI | CD28z-CAR |
| | SCKASGYSFPNYWITWVRQMSGGGLEWMGRIDPGDSYTTYNPSFQGHVTISIDKSTN | |
| | TAYLHWNSLKASDTAMYYCARYYVSLVDIWGQGTLVTVSSEQKLISEEDLAAATGT | |
| | TTPAPRPPTPAPTIASQPLSLRPEACRPAAGGAVHTRGLDFACDIYIWAPLAGTCGVLL | |
| | LSLVITLYCAAARSKRSRLLHSDYMNMTPRRPGPTRKHYQPYAPPRDFAAYRSRVKF | |
| | SRSADAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALPPR | |
| 13 | METDTLLLWVLLLWVPGSTGQSVLTQPPSVSAAPGQRVTISCSGTRSNIGSDYVSWY | αGPC3-CD30-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated CD30 |
| | NTLYLQMNSLRAEDTAVYYCARTSYLNHGDYWGQGTLVTVSSEQKLISEEDLAAAT | (clone 37 anti-GPC3- |
| | GAPPLGTQPDCNPTPENGEAPASTSPTQSLLVDSQASKTLPIPTSAPVALSSTGKPVLD | CD30-CSR with SP |
| | AGPVLFWVILVLVVVVGSSAFLLCHRRACRKRIRQKLHLCYPVQTSQPKLELVDSRPR | and myc tag) |
| | RSSTQLRSGASVTEPVAEERGLMSQPLMETCHSVGAAYLESLPLQDASPAGGPSSPRD | |
| | LPEPRVSTEHTNNKIEKIYEMKADTVIVGTVKAELPEGRGLAGPAEPELEEELEADHTP | |
| | HYPEQETEPPLGSCSDVMLSVEEEGKEDPLPTAASGK | |
| 14 | METDTLLLWVLLLWVPGSTGQSVLTQPPSVSAAPGQRVTISCSGTRSNIGSDYVSWY | αGPC3-CD28-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated CD28 |
| | NTLYLQMNSLRAEDTAVYYCARTSYLNHGDYWGQGTLVTVSSEQKLISEEDLAAAIE | |
| | VMYPPPYLDNEKSNGTIIHVKGKHLCPSPLFPGPSKPFWVLVVVGGVLACYSLLVTVA | |
| | FIIFWVRSKRSRLLHSDYMNMTPRRPGPTRKHYQPYAPPRDFAAYRS | |
| 15 | METDTLLLWVLLLWVPGSTGQSVLTQPPSVSAAPGQRVTISCSGTRSNIGSDYVSWY | αGPC3-41BB-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated 41BB |
| | NTLYLQMNSLRAEDTAVYYCARTSYLNHGDYWGQGTLVTVSSEQKLISEEDLAAAT | |
| | GPADLSPGASSVTPPAPAREPGHSPQIISFFLALTSTALLFLLFFLTLRFSVVKRGRKKLL | |
| | YIFKQPFMRPVQTTQEEDGCSCRFPEEEEGGCEL | |
| 16 | METDTLLLWVLLLWVPGSTGQSVLTQPPSVSAAPGQRVTISCSGTRSNIGSDYVSWY | αGPC3-OX40-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-truncated OX40 |
| | NTLYLQMNSLRAEDTAVYYCARTSYLNHGDYWGQGTLVTVSSEQKLISEEDLAAAT | |
| | GDRDPPATQPQETQGPPARPITVQPTEAWPRTSQGPSTRPVEVPGGRAVAAILGLGLV | |
| | LGLLGPLAILLALYLLRRDQRLPPDAHKPPGGGSFRTPIQEEQADAHSTLAKI | |
| 17 | METDTLLLWVLLLWVPGSTGQSVLTQPPSVSAAPGQRVTISCSGTRSNIGSDYVSWY | αGPC3-CD27-CSR |
| | QHLPGTAPKLLVYGDNLRPSGIPDRFSASKSGTSATLGITGLQTGDEADYYCGTWDYT | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker-trucated CD27 |
| | NTLYLQMNSLRAEDTAVYYCARTSYLNHGDYWGQGTLVTVSSEQKLISEEDLAAAT | |
| | GPTHLPYVSEMLEARTAGHMQTLADFRQLPARTLSTHWPPQRSLCSSDFIRILVIFSGM | |
| | FLVFTLAGALFLHQRRKYRSNKGESPVEPAEPCRYSCPREEEGSTIPIQEDYRKPEPACS | |
| | P | |
| 18 | METDTLLLWVLLLWVPGSTGQSVLTQPPSVSAAPGQRVTISCSGTRSNIGSDYVSWY | αGPC3-CD30T-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD30T-CD28 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCRSKRSRLLHSDYMNMTPRRPGPTRKHYQPYAPP | |
| | RDFAAYRS | |
| 19 | METDTLLLWVLLLWVPGSTGQSVLTQPPSVSAAPGQRVTISCSGTRSNIGSDYVSWY | αGPC3-CD30T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD30T-41BB |
| | GAPPLGTQPDCNPTPENGEAPASTSPTQSLLVDSQASKTLPIPTSAPVALSSTGKPVLD | IC |
| | AGPVLFWVILVLVVVVGSSAFLLCKRGRKKLLYIFKQPFMRPVQTTQEEDGCSCRFPE | |
| | EEEGGCEL | |
| 20 | METDTLLLWVLLLWVPGSTGQSVLTQPPSVSAAPGQRVTISCSGTRSNIGSDYVSWY | αGPC3-CD30T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD30T-OX40 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCALYLLRRDQRLPPDAHKPPGGGSFRTPIQEEQAD | |
| | AHSTLAKI | |
| 21 | METDTLLLWVLLLWVPGSTGQSVLTQPPSVSAAPGQRVTISCSGTRSNIGSDYVSWY | αGPC3-CD30T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD30T-CD27 |
| | GAPPLGTQPDCNPTPENGEAPASTSPTQSLLVDSQASKTLPIPTSAPVALSSTGKPVLD | IC |
| | AGPVLFWVILVLVVVVGSSAFLLCQRRKYRSNKGESPVEPAEPCRYSCPREEEGSTIPI | |
| | QEDYRKPEPACSP | |
| 22 | METDTLLLWVLLLWVPGSTGQSVLTQPPSVSAAPGQRVTISCSGTRSNIGSDYVSWY | αGPC3-CD28T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD28T-CD30 |
| | GIEVMYPPPYLDNEKSNGTIIHVKGKHLCPSPLFPGPSKPFWVLVVVGGVLACYSLLV | IC |
| | TVAFIIFWVHRRACRKRIRQKLHLCYPVQTSQPKLELVDSRPRRSSTQLRSGASVTEPV | |
| | AEERGLMSQPLMETCHSVGAAYLESLPLQDASPAGGPSSPRDLPEPRVSTEHTNNKIE | |
| | KIYIMKADTVIVGTVKAELPEGRGLAGPAEPELEEELEADHTPHYPEQETEPPLGSCSD | |
| | VMLSVEEEGKEDPLPTAASGK | |
| 23 | METDTLLLWVLLLWVPGSTGQSVLTQPPSVSAAPGQRVTISCSGTRSNIGSDYVSWY | αGPC3-CD28T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD28T-41BB |
| | GIEVMYPPPYLDNEKSNGTIIHVKGKHLCPSPLFPGPSKPFWVLVVVGGVLACYSLLV | IC |
| | TVAFIIFWVKRGRKKLLYIFKQPFMRPVQTTQEEDGCSCRFPEEEEGGCEL | |
| 24 | METDTLLLWVLLLWVPGSTGQSVLTQPPSVSAAPGQRVTISCSGTRSNIGSDYVSWY | αGPC3-CD28T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD28T-OX40 |
| | GIEVMYPPPYLDNEKSNGTIIHVKGKHLCPSPLFPGPSKPFWVLVVVGGVLACYSLLV | IC |
| | TVAFIIFWVALYLLRRDQRLPPDAHKPPGGGSFRTPIQEEQADAHSTLAKI | |
| 25 | METDTLLLWVLLLWVPGSTGQSVLTQPPSVSAAPGQRVTISCSGTRSNIGSDYVSWY | αGPC3-CD28T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD28T-CD27 |
| | GIEVMYPPPYLDNEKSNGTIIHVKGKHLCPSPLFPGPSKPFWVLVVVGGVLACYSLLV | IC |
| | TVAFIIFWVQRRKYRSNKGESPVEPAEPCRYSCPREEEGSTIPIQEDYRKPEPACSP | |
| 26 | METDTLLLWVLLLWVPGSTGQSVLTQPPSVSAAPGQRVTISCSGTRSNIGSDYVSWY | αGPC3-41BBT-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- 41BBT-CD30 |
| | GPADLSPGASSVTPPAPAREPGHSPQIISFFLALTSTALLFLLFFLTLRFSVVHRRACRKR | IC |
| | IRQKLHLCYPVQTSQPKLELVDSRPRRSSTQLRSGASVTEPVAEERGLMSQPLMETCH | |
| | SVGAAYLESLPLQDASPAGGPSSPRDLPEPRVSTEHTNNKIEKIYIMKADTVIVGTVKA | |
| | ELPEGRGLAGPAEPELEEELEADHTPHYPEQETEPPLGSCSDVMLSVEEEGKEDPLPTA | |
| | ASGK | |
| 27 | METDTLLLWVLLLWVPGSTGQSVLTQPPSVSAAPGQRVTISCSGTRSNIGSDYVSWY | αGPC3-41BBT-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- 41BBT-CD28 |
| | GPADLSPGASSVTPPAPAREPGHSPQIISFFLALTSTALLFLLFFLTLRFSVVRSKRSRLL | IC |
| | HSDYMNMTPRRPGPTRKHYQPYAPPRDFAAYRS | |
| 28 | METDTLLLWVLLLWVPGSTGQSVLTQPPSVSAAPGQRVTISCSGTRSNIGSDYVSWY | αGPC3-41BBT-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- 41BBT-OX40 |
| | GPADLSPGASSVTPPAPAREPGHSPQIISFFLALTSTALLFLLFFLTLRFSVVALYLLRRD | IC |
| | QRLPPDAHKPPGGGSFRTPIQEEQADAHSTLAKI | |
| 29 | METDTLLLWVLLLWVPGSTGQSVLTQPPSVSAAPGQRVTISCSGTRSNIGSDYVSWY | αGPC3-41BBT-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- 41BBT-CD27 |
| | GPADLSPGASSVTPPAPAREPGHSPQIISFFLALTSTALLFLLFFLTLRFSVVQRRKYRSN | IC |
| | KGESPVEPAEPCRYSCPREEEGSTIPIQEDYRKPEPACSP | |
| 30 | METDTLLLWVLLLWVPGSTGQSVLTQPPSVSAAPGQRVTISCSGTRSNIGSDYVSWY | αGPC3-OX40T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- OX40T-CD30 |
| | GDRDPPATQPQETQGPPARPITVQPTEAWPRTSQGPSTRPVEVPGGRAVAAILGLGLV | IC |
| | LGLLGPLAILLHRRACRKRIRQKLHLCYPVQTSQPKLELVDSRPRRSSTQLRSGASVTE | |
| | PVAEERGLMSQPLMETCHSVGAAYLESLPLQDASPAGGPSSPRDLPEPRVSTEHTNNK | |
| | IEKIYEVIKADTVIVGTVKAELPEGRGLAGPAEPELEEELEADHTPHYPEQETEPPLGSCS | |
| | DVMLSVEEEGKEDPLPTAASGK | |
| 31 | METDTLLLWVLLLWVPGSTGQSVLTQPPSVSAAPGQRVTISCSGTRSNIGSDYVSWY | αGPC3- OX40T- |
| | QHLPGTAPKLLVYGDNLRPSGIPDRFSASKSGTSATLGITGLQTGDEADYYCGTWDYT | CD28-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- OX40T-CD28 |
| | GDRDPPATQPQETQGPPARPITVQPTEAWPRTSQGPSTRPVEVPGGRAVAAILGLGLV | IC |
| | LGLLGPLAILLRSKRSRLLHSDYMNMTPRRPGPTRKHYQPYAPPRDFAAYRS | |
| 32 | METDTLLLWVLLLWVPGSTGQSVLTQPPSVSAAPGQRVTISCSGTRSNIGSDYVSWY | αGPC3-OX40T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LNGVVFGGGTKLTVLGSRGGGGSGGGGSGGGGSLEMAQVQLVESGGGLVQPGGSLR | 37 scFv-myc tag- |
| | LSCAASGFTFSSYAMSWVRQAPGKGLEWVSVIYSGGSSTYYADSVKGRFTISRDNSK | linker- OX40T-41BB |
| | NTLYLQMNSLRAEDTAVYYCARTSYLNHGDYWGQGTLVTVSSEQKLISEEDLAAAT | IC |
| | GDRDPPATQPQETQGPPARPITVQPTEAWPRTSQGPSTRPVEVPGGRAVAAILGLGLV | |
| | LGLLGPLAILLKRGRKKLLYIFKQPFMRPVQTTQEEDGCSCRFPEEEEGGCEL | |
| 33 | METDTLLLWVLLLWVPGSTGQSVLTQPPSVSAAPGQRVTISCSGTRSNIGSDYVSWY | αGPC3- OX40T- |
| | QHLPGTAPKLLVYGDNLRPSGIPDRFSASKSGTSATLGITGLQTGDEADYYCGTWDYT | CD27-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- OX40T-CD27 |
| | GDRDPPATQPQETQGPPARPITVQPTEAWPRTSQGPSTRPVEVPGGRAVAAILGLGLV | IC |
| | LGLLGPLAILLQRRKYRSNKGESPVEPAEPCRYSCPREEEGSTIPIQEDYRKPEPACSP | |
| 34 | METDTLLLWVLLLWVPGSTGQSVLTQPPSVSAAPGQRVTISCSGTRSNIGSDYVSWY | αGPC3-CD27T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD27T-CD30 |
| | GPTHLPYVSEMLEARTAGHMQTLADFRQLPARTLSTHWPPQRSLCSSDFIRILVIFSGM | IC |
| | FLVFTLAGALFLHHRRACRKRIRQKLHLCYPVQTSQPKLELVDSRPRRSSTQLRSGAS | |
| | VTEPVAEERGLMSQPLMETCHSVGAAYLESLPLQDASPAGGPSSPRDLPEPRVSTEHT | |
| | NNKIEKIYEVIKADTVIVGTVKAELPEGRGLAGPAEPELEEELEADHTPHYPEQETEPPL | |
| | GSCSDVMLSVEEEGKEDPLPTAASGK | |
| 35 | METDTLLLWVLLLWVPGSTGQSVLTQPPSVSAAPGQRVTISCSGTRSNIGSDYVSWY | αGPC3- CD27T- |
| | QHLPGTAPKLLVYGDNLRPSGIPDRFSASKSGTSATLGITGLQTGDEADYYCGTWDYT | CD28-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD27T-CD28 |
| | GPTHLPYVSEMLEARTAGHMQTLADFRQLPARTLSTHWPPQRSLCSSDFIRILVIFSGM | IC |
| | FLVFTLAGALFLHRSKRSRLLHSDYMNMTPRRPGPTRKHYQPYAPPRDFAAYRS | |
| 36 | METDTLLLWVLLLWVPGSTGQSVLTQPPSVSAAPGQRVTISCSGTRSNIGSDYVSWY | αGPC3- CD27T- |
| | QHLPGTAPKLLVYGDNLRPSGIPDRFSASKSGTSATLGITGLQTGDEADYYCGTWDYT | 41BB-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD27T-41BB |
| | GPTHLPYVSEMLEARTAGHMQTLADFRQLPARTLSTHWPPQRSLCSSDFIRILVIFSGM | IC |
| | FLVFTLAGALFLHKRGRKKLLYIFKQPFMRPVQTTQEEDGCSCRFPEEEEGGCEL | |
| 37 | METDTLLLWVLLLWVPGSTGQSVLTQPPSVSAAPGQRVTISCSGTRSNIGSDYVSWY | αGPC3- CD27T- |
| | QHLPGTAPKLLVYGDNLRPSGIPDRFSASKSGTSATLGITGLQTGDEADYYCGTWDYT | OX40-CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD27T-OX40 |
| | GPTHLPYVSEMLEARTAGHMQTLADFRQLPARTLSTHWPPQRSLCSSDFIRILVIFSGM | IC |
| | FLVFTLAGALFLHALYLLRRDQRLPPDAHKPPGGGSFRTPIQEEQADAHSTLAKI | |
| 38 | METDTLLLWVLLLWVPGSTGQSVLTQPPSVSAAPGQRVTISCSGTRSNIGSDYVSWY | αGPC3-CD8T-CD30- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker-CD8T-CD30 IC |
| | GTTTPAPRPPTPAPTIASQPLSLRPEACRPAAGGAVHTRGLDFACDIYIWAPLAGTCGV | |
| | LLLSLVITLYCHRRACRKRIRQKLHLCYPVQTSQPKLELVDSRPRRSSTQLRSGASVTE | |
| | PVAEERGLMSQPLMETCHSVGAAYLESLPLQDASPAGGPSSPRDLPEPRVSTEHTNNK | |
| | IEKIYIMKADTVIVGTVKAELPEGRGLAGPAEPELEEELEADHTPHYPEQETEPPLGSCS | |
| | DVMLSVEEEGKEDPLPTAASGK | |
| 39 | METDTLLLWVLLLWVPGSTGQSVLTQPPSVSAAPGQRVTISCSGTRSNIGSDYVSWY | αGPC3-CD8T-CD28- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD8T-CD28 |
| | GTTTPAPRPPTPAPTIASQPLSLRPEACRPAAGGAVHTRGLDFACDIYIWAPLAGTCGV | IC |
| | LLLSLVITLYCRSKRSRLLHSDYMNMTPRRPGPTRKHYQPYAPPRDFAAYRS | |
| 40 | METDTLLLWVLLLWVPGSTGQSVLTQPPSVSAAPGQRVTISCSGTRSNIGSDYVSWY | αGPC3-CD8T-41BB- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD8T-41BB |
| | GTTTPAPRPPTPAPTIASQPLSLRPEACRPAAGGAVHTRGLDFACDIYIWAPLAGTCGV | IC |
| | LLLSLVITLYCKRGRKKLLYIFKQPFMRPVQTTQEEDGCSCRFPEEEEGGCEL | |
| 41 | METDTLLLWVLLLWVPGSTGQSVLTQPPSVSAAPGQRVTISCSGTRSNIGSDYVSWY | αGPC3-CD8T-OX40- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD8T-OX40 |
| | GTTTPAPRPPTPAPTIASQPLSLRPEACRPAAGGAVHTRGLDFACDIYIWAPLAGTCGV | IC |
| | LLLSLVITLYCALYLLRRDQRLPPDAHKPPGGGSFRTPIQEEQADAHSTLAKI | |
| 42 | METDTLLLWVLLLWVPGSTGQSVLTQPPSVSAAPGQRVTISCSGTRSNIGSDYVSWY | αGPC3-CD8T-CD27- |
| | QHLPGTAPKLLVYGDNLRPSGIPDRFSASKSGTSATLGITGLQTGDEADYYCGTWDYT | CSR |
| | LNGVVFGGGTKLTVLGSRGGGGSGGGGSGGGGSLEMAQVQLVESGGGLVQPGGSLR | Signal peptide-GPC3- |
| | LSCAASGFTFSSYAMSWVRQAPGKGLEWVSVIYSGGSSTYYADSVKGRFTISRDNSK | 37 scFv-myc tag- |
| | NTLYLQMNSLRAEDTAVYYCARTSYLNHGDYWGQGTLVTVSSEQKLISEEDLAAAT | linker- CD8T-CD27 |
| | GTTTPAPRPPTPAPTIASQPLSLRPEACRPAAGGAVHTRGLDFACDIYIWAPLAGTCGV | IC |
| | LLLSLVITLYCQRRKYRSNKGESPVEPAEPCRYSCPREEEGSTIPIQEDYRKPEPACSP | |
| 43 | QPVLTQPPSVSVAPGKTARITCGGNNIGSKSVHWYQQKPGQAPVLVIYYDSDRPSGIP | αGPC3-55_scFv |
| | ERFSGSNSGNTATLTISRVEAGDEADYYCQVWDSSSDHYVFGTGTKVTVLGSRGGGG | (αGPC3 scFv_clone |
| | SGGGGSGGGGSLEMAQVQLVQSGAEVKKPGASVKVSCKASGYTFTSYYMHWVRQA | 55) |
| | PGQGLEWMGIINPSGGSTSYAQKFQGRVTMTRDTSTSTVYMELSSLRSEDTAVYYCA | |
| | RWHGGPYDYWGQGTLVTVSS | |
| 44 | QSVLTQPPSVSVAPGKTARITCGGNNIGSKSVHWYQQKPGQAPVLVVYDDSDRPSGIP | αGPC3-58 scFv |
| | ERFSGSNSGNTATLTISRVEAGDEADYYCQVWDSSSDHVFGTGTKVTVLGSRGGGGS | (αGPC3 scFv_clone |
| | GGGGSGGGGSLEMAQVQLVQSGADVRKPGASVKVSCKASGYTFASHGISWVRQAPG | 58) |
| | QGLEWLGWISPYTGNTNYAQKFQGRVTMATDTSTSTAYMELRSLRSDDTAIYYCAR | |
| | GKRTLASCFDYWGQGTLVTVSS | |
| 45 | LPVLTQPPSVSVAPGKTARITCGGNNIGSKSVHWYQQKPGQAPVLVVYDDSDRPSGIP | αCD19 scFv |
| | ERFSGSNSGNTATLTISRVEAGDEADYYCQVWDSSSDYVVFGGGTKLTVLGSRGGGG | |
| | SGGGGSGGGGSLEMAEVQLVQSGAEVKKPGESLKISCKGSGYSFTSYWIGWVRQMP | |
| | GKGLEWMGIIYPGDSDTRYSPSFQGQVTISADKSISTAYLQWSSLKASDTAMYYCAR | |
| | QVWGWQGGMYPRSNWWYNLDSWGQGTLVTVSS | |
| 46 | QIVLSQSPAILSASPGEKVTMTCRASSSVSYIHWFQQKPGSSPKPWIYATSNLASGVPV | αCD20 scFv |
| | RFSGSGSGTSYSLTISRVEAEDAATYYCQQWTSNPPTFGGGTKLEIKRSRGGGGSGGG | |
| | GSGGGGSLEQVQLQQPGAELVKPGASVKMSCKASGYTFTSYNMHWVKQTPGRGLE | |
| | WIGAIYPGNGDTSYNQKFKGKATLTADKSSSTAYMQLSSLTSEDSAVYYCARSTYYG | |
| | GDWYFNVWGAGTTVTVSS | |
| 47 | DIQLTQSPSSLSTSVGDRVTITCQASHDIRNYLNVVYQQKPGKAPNLLIYAASNLQTGV | αCD22-8 scFv |
| | PSRFSGRGSGTDFTLTISSLQPEDIATYYCQQYDGLPLTFGQGTRLEIKRSRGGGGSGG | |
| | GGSGGGGSLEMAQVQLVESGGGLVQPGGSLRLSCAASGFTFSNYAMSWVRQAPGKG | |
| | LEWVSSISGSGGSTYYADSVKGRFTISRDTSKNTLYLQMNSLRAEDTAVYYCARYGS | |
| | AAWMDSWGQGTLVTVSS | |
| 48 | DIQLTQSPSSLSTSVGDRVTITCQASHDIRNYLNWYQQKPGKAPNLLIYAASNLQTGV | αCD22-αCD19 |
| | PSRFSGRGSGTDFTLTISSLQPEDIATYYCQQYDGLPLTFGQGTRLEIKRSRGGGGSGG | anti-CD22-clone 8 |
| | GGSGGGGSLEMAQVQLVESGGGLVQPGGSLRLSCAASGFTFSNYAMSWVRQAPGKG | scFv and anti-CD19 |
| | LEWVSSISGSGGSTYYADSVKGRFTISRDTSKNTLYLQMNSLRAEDTAVYYCARYGS | scFv - linker -anti- |
| | AAWMDSWGQGTLVTVSSGGGGSGGGGSLPVLTQPPSVSVAPGKTARITCGGNNIGSK | CD19 scFv |
| | SVHWYQQKPGQAPVLVVYDDSDRPSGIPERFSGSNSGNTATLTISRVEAGDEADYYC | |
| | QVWDSSSDYVVFGGGTKLTVLGSRGGGGSGGGGSGGGGSLEMAEVQLVQSGAEVK | |
| | KPGESLKISCKGSGYSFTSYWIGWVRQMPGKGLEWMGIIYPGDSDTRYSPSFQGQVTI | |
| | SADKSISTAYLQWSSLKASDTAMYYCARQVWGWQGGMYPRSNWWYNLDSWGQGT | |
| | LVTVSS | |
| 49 | LPVLTQPPSVSVAPGKTARITCGGNNIGSKSVHWYQQKPGQAPVLVVYDDSDRPSGIP | αCD19-αCD22-αCD20 |
| | ERFSGSNSGNTATLTISRVEAGDEADYYCQVWDSSSDYVVFGGGTKLTVLGSRGGGG | CD19 scFv-linker- |
| | SGGGGSGGGGSLEMAEVQLVQSGAEVKKPGESLKISCKGSGYSFTSYWIGWVRQMP | CD22 scFv-linker- |
| | GKGLEWMGIIYPGDSDTRYSPSFQGQVTISADKSISTAYLQWSSLKASDTAMYYCAR | CD20 scFv |
| | QVWGWQGGMYPRSNWWYNLDSWGQGTLVTVSSGGGGSGGGGSDIQLTQSPSSLST | |
| | SVGDRVTITCQASHDIRNYLNWYQQKPGKAPNLLIYAASNLQTGVPSRFSGRGSGTDF | |
| | TLTISSLQPEDIATYYCQQYDGLPLTFGQGTRLEIKRSRGGGGSGGGGSGGGGSLEMA | |
| | QVQLVESGGGLVQPGGSLRLSCAASGFTFSNYAMSWVRQAPGKGLEWVSSISGSGGS | |
| | TYYADSVKGRFTISRDTSKNTLYLQMNSLRAEDTAVYYCARYGSAAWMDSWGQGT | |
| | LVTVSSGGGGSGGGGSQIVLSQSPAILSASPGEKVTMTCRASSSVSYIHWFQQKPGSSP | |
| | KPWIYATSNLASGVPVRFSGSGSGTSYSLTISRVEAEDAATYYCQQWTSNPPTFGGGT | |
| | KLEIKRSRGGGGSGGGGSGGGGSLEQVQLQQPGAELVKPGASVKMSCKASGYTFTSY | |
| | NMHWVKQTPGRGLEWIGAIYPGNGDTSYNQKFKGKATLTADKSSSTAYMQLSSLTS | |
| | EDSAVYYCARSTYYGGDWYFNVWGAGTTVTVSS | |
| 50 | EIVLTQSPGTLSLSPGERATLSCRASQSVSSSYLAWYQQKPGQAPRLLIYGASSRATGIP | ROR1-18 scFv |
| | DRFSGSGSGTDFTLTISRLEPEDFAVYYCQQYGSSFGPGTKVDIKRSRGGGGSGGGGS | |
| | GGGGSLEMAQVQLVQSGTEVKKPGSSVKVSCQASGGSLSSHGVSWLRQAPGQGLEW | |
| | VGRIIPMFGVTDYAQKFQDRVTITADKSTSTVYMELISLGSDDTAVYFCARESRGATF | |
| | EYWGQGTLVTVSS | |
| 51 | QSVLTQPASVSGSPGQSITISCTGTSSDFGDYDYVSWYQQHPGKAPKLMIYDVSDRPS | ROR1-56 scFv |
| | GVSNRFSGSKSGNTASLTISGLQAEDEADYFCSSLTTSSTLVFGGGTKLTVLGSRGGG | |
| | GSGGGGSGGGGSLEMAQLQLQESGPGLVKPSETLSLTCTVSGGSISSSSYYWGWIRQP | |
| | PGKGLEWIGSIYYSGSTYYNPSLKSRVTISVDTSKNQFSLKLGSVTAADTAVYYCARH | |
| | DGTDAFDIWGQGTTVTVSS | |
| 52 | QSVLTQPPSVSGAPGQRVTISCTGSSSNIGAGYDVHWYQQLPGTAPKLLIYGNSNRPS | PSMA-A scFv |
| | GVPDRFSGSKSGTSASLAITGLQAEDEADYYCQSYDSSLSGYVFGTGTKVTVLGSRGG | |
| | GGSGGGGSGGGGSLEMAEVQLVQSGAEVKKPGESLKISCKGSGYSFTSYWIGWVRQ | |
| | MPGKGLEWMGIIYPGDSDTRYSPSFQGQVTISADKSISTAYLQWSSLKASDTAMYYC | |
| | ARSMGSSLYASSDVWGQGTLVTVSS | |
| 53 | QAVLTQPPSASGTPGQRVTISCSGSSSNIGSNTVNWYQQLPGTAPKLLMYSNNQRPSG | PSMA-B scFv |
| | VPDRFSGSKSGTSASLAISGLQSEDEADYYCAAWDDSLNGYVFGTGTKVTVLGSRGG | |
| | GGSGGGGSGGGGSLEMAEVQLVQSGAEMKKPGESLKISCKGSGYNFASYWVGWVR | |
| | QMPGKGLEWMGTIYPDDSDTRYGPAFQGQVTISADKSISTAYLQWSSLKASDTAMY | |
| | YCARDSYYGIDVWGQGTLVTVSS | |
| 54 | DILLTQSPVILSVSPGERVSFSCRASQSIGTNIHWYQQRTNGSPRLLIKYASESISGIPSRF | EGFR scFv |
| | SGSGSGTDFTLSINSVESEDIADYYCQQNNNWPTTFGAGTKLELKRSRGGGGSGGGGS | |
| | GGGGSLEMAQVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCARALTYYDYEF | |
| | AYWGQGTLVTVSS | |
| 55 | APPLGTQPDCNPTPENGEAPASTSPTQSLLVDSQASKTLPIPTSAPVALSSTGKPVLDA | CD30TM + IC |
| | GPVLFWVILVLVVVVGSSAFLLCHRRACRKRIRQKLHLCYPVQTSQPKLELVDSRPRR | |
| | SSTQLRSGASVTEPVAEERGLMSQPLMETCHSVGAAYLESLPLQDASPAGGPSSPRDL | |
| | PEPRVSTEHTNNKIEKIYIMKADTVIVGTVKAELPEGRGLAGPAEPELEEELEADHTPH | |
| | YPEQETEPPLGSCSDVMLSVEEEGKEDPLPTAASGK | |
| 56 | IEVMYPPPYLDNEKSNGTIIHVKGKHLCPSPLFPGPSKPFWVLVVVGGVLACYSLLVT | CD28TM + IC |
| | VAFIIFWVRSKRSRLLHSDYMNMTPRRPGPTRKHYQPYAPPRDFAAYRS | |
| 57 | PADLSPGASSVTPPAPAREPGHSPQIISFFLALTSTALLFLLFFLTLRFSVVKRGRKKLLY | 41BB TM + IC |
| | IFKQPFMRPVQTTQEEDGCSCRFPEEEEGGCEL | |
| 58 | DRDPPATQPQETQGPPARPITVQPTEAWPRTSQGPSTRPVEVPGGRAVAAILGLGLVL | OX40 TM + IC |
| | GLLGPLAILLALYLLRRDQRLPPDAHKPPGGGSFRTPIQEEQADAHSTLAKI | |
| 59 | PTHLPYVSEMLEARTAGHMQTLADFRQLPARTLSTHWPPQRSLCSSDFIRILVIFSGMF | CD27TM + IC |
| | LVFTLAGALFLHQRRKYRSNKGESPVEPAEPCRYSCPREEEGSTIPIQEDYRKPEPACSP | |
| 60 | IEVMYPPPYLDNEKSNGTIIHVKGKHLCPSPLFPGPSKPFWVLVVVGGVLACYSLLVT | CD28T-CD30 IC |
| | VAFIIFWVHRRACRKRIRQKLHLCYPVQTSQPKLELVDSRPRRSSTQLRSGASVTEPVA | |
| | EERGLMSQPLMETCHSVGAAYLESLPLQDASPAGGPSSPRDLPEPRVSTEHTNNKIEKI | |
| | YIMKADTVIVGTVKAELPEGRGLAGPAEPELEEELEADHTPHYPEQETEPPLGSCSDV | |
| | MLSVEEEGKEDPLPTAASGK | |
| 61 | PADLSPGASSVTPPAPAREPGHSPQIISFFLALTSTALLFLLFFLTLRFSVVHRRACRKRI | 41BBT-CD30 IC |
| | RQKLHLCYPVQTSQPKLELVDSRPRRSSTQLRSGASVTEPVAEERGLMSQPLMETCHS | |
| | VGAAYLESLPLQDASPAGGPSSPRDLPEPRVSTEHTNNKIEKIYIMKADTVIVGTVKAE | |
| | LPEGRGLAGPAEPELEEELEADHTPHYPEQETEPPLGSCSDVMLSVEEEGKEDPLPTAA | |
| | SGK | |
| 62 | DRDPPATQPQETQGPPARPITVQPTEAWPRTSQGPSTRPVEVPGGRAVAAILGLGLVL | OX40T-CD30 IC |
| | GLLGPLAILLHRRACRKRIRQKLHLCYPVQTSQPKLELVDSRPRRSSTQLRSGASVTEP | |
| | VAEERGLMSQPLMETCHSVGAAYLESLPLQDASPAGGPSSPRDLPEPRVSTEHTNNKI | |
| | EKIYIMKADTVIVGTVKAELPEGRGLAGPAEPELEEELEADHTPHYPEQETEPPLGSCS | |
| | DVMLSVEEEGKEDPLPTAASGK | |
| 63 | PTHLPYVSEMLEARTAGHMQTLADFRQLPARTLSTHWPPQRSLCSSDFIRILVIFSGMF | CD27T-CD30 IC |
| | LVFTLAGALFLHHRRACRKRIRQKLHLCYPVQTSQPKLELVDSRPRRSSTQLRSGASV | |
| | TEPVAEERGLMSQPLMETCHSVGAAYLESLPLQDASPAGGPSSPRDLPEPRVSTEHTN | |
| | NKIEKIYIMKADTVIVGTVKAELPEGRGLAGPAEPELEEELEADHTPHYPEQETEPPLG | |
| | SCSDVMLSVEEEGKEDPLPTAASGK | |
| 64 | TTTPAPRPPTPAPTIASQPLSLRPEACRPAAGGAVHTRGLDFACDIYIWAPLAGTCGVL | CD8T-CD30 IC |
| | LLSLVITLYCHRRACRKRIRQKLHLCYPVQTSQPKLELVDSRPRRSSTQLRSGASVTEP | |
| | VAEERGLMSQPLMETCHSVGAAYLESLPLQDASPAGGPSSPRDLPEPRVSTEHTNNKI | |
| | EKIYIMKADTVIVGTVKAELPEGRGLAGPAEPELEEELEADHTPHYPEQETEPPLGSCS | |
| | DVMLSVEEEGKEDPLPTAASGK | |
| 65 | MRVLLAALGLLFLGALRAFPQDRPFEDTCHGNPSHYYDKAVRRCCYRCPMGLFPTQ | Full length CD30 |
| | QCPQRPTDCRKQCEPDYYLDEADRCTACVTCSRDDLVEKTPCAWNSSRVCECRPGM | (NP_001234.3) |
| | FCSTSAVNSCARCFFHSVCPAGMIVKFPGTAQKNTVCEPASPGVSPACASPENCKEPSS | |
| | GTIPQAKPTPVSPATSSASTMPVRGGTRLAQEAASKLTRAPDSPSSVGRPSSDPGLSPT | |
| | QPCPEGSGDCRKQCEPDYYLDEAGRCTACVSCSRDDLVEKTPCAWNSSRTCECRPGM | |
| | ICATSATNSCARCVPYPICAAETVTKPQDMAEKDTTFEAPPLGTQPDCNPTPENGEAP | |
| | ASTSPTQSLLVDSQASKTLPIPTSAPVALSSTGKPVLDAGPVLFWVILVLVVVVGSSAF | |
| | LLCHRRACRKRIRQKLHLCYPVQTSQPKLELVDSRPRRSSTQLRSGASVTEPVAEERG | |
| | LMSQPLMETCHSVGAAYLESLPLQDASPAGGPSSPRDLPEPRVSTEHTNNKIEKIYIMK | |
| | ADTVIVGTVKAELPEGRGLAGPAEPELEEELEADHTPHYPEQETEPPLGSCSDVMLSV | |
| | EEEGKEDPLPTAASGK | |
| 66 | IYIWAPLAGTCGVLLLSLVIT | CD8 transmembrane |
| | | (TM) sequence |
| 67 | IISFFLALTSTALLFLLFFLTLRFSVV | 4-1BB TMsequence |
| 68 | ILVIFSGMFLVFTLAGALFLH | CD27 TM sequence |
| 69 | FWVLVVVGGVLACYSLLVTVAFIIFWV | CD28 TM sequence |
| 70 | PVLDAGPVLFWVILVLVVVVGSSAFLLC | CD30 TM sequence |
| 71 | VAAILGLGLVLGLLGPLAILL | OX40 TM sequence |
| 72 | KRGRKKLLYIFKQPFMRPVQTTQEEDGCSCRFPEEEEGGCEL | 4-1BB IC signaling |
| | | sequence |
| 73 | QRRKYRSNKGESPVEPAEPCRYSCPREEEGSTIPIQEDYRKPEPACSP | CD27 IC signaling |
| | | sequence |
| 74 | RSKRSRLLHSDYMNMTPRRPGPTRKHYQPYAPPRDFAAYRS | CD28 IC signaling |
| | | sequence |
| 75 | HRRACRKRIRQKLHLCYPVQTSQPKLELVDSRPRRSSTQLRSGASVTEPVAEERGLMS | CD30 IC signaling |
| | QPLMETCHSVGAAYLESLPLQDASPAGGPSSPRDLPEPRVSTEHTNNKIEKIYIMKADT | sequence |
| | VIVGTVKAELPEGRGLAGPAEPELEEELEADHTPHYPEQETEPPLGSCSDVMLSVEEE | |
| | GKEDPLPTAASGK | |
| 76 | ALYLLRRDQRLPPDAHKPPGGGSFRTPIQEEQADAHSTLAKI | OX40 IC signaling |
| | | sequence |
| 77 | RVKFSRSADAPAYQQGQNQLYNELNLGRREEYDVLDKRRGRDPEMGGKPRRKNPQE | CD3ζ IC signaling |
| | GLYNELQKDKMAEAYSEIGMKGERRRGKGHDGLYQGLSTATKDTYDALHMQALPP | sequence |
| | R | |
| 78 | SRGGGGSGGGGSGGGGSLEMA | Peptide linker |
| 79 | GGGGS | Peptide linker |
| 80 | GGSG | Peptide linker |
| 81 | SGGG | Peptide linker |
| 82 | GSGS | Peptide linker |
| 83 | GSGSGS | Peptide linker |
| 84 | GSGSGSGS | Peptide linker |
| 85 | GSGSGSGSGS | Peptide linker |
| 86 | GGSGGS | Peptide linker |
| 87 | GGSGGSGGS | Peptide linker |
| 88 | GGSGGSGGSGGS | Peptide linker |
| 89 | GGSG | Peptide linker |
| 90 | GGSGGGSG | Peptide linker |
| 91 | GGSGGGSGGGSG | Peptide linker |
| 92 | SRGGGGSGGGGSGGGGSLEMA | Peptide linker |
| 93 | HHHHHH | 6xHis Tag |
| 94 | YPYDVPDYA | HA peptide |
| 95 | YPYDVPDYAS | HA peptide |
| 96 | DYKDDDDK | FLAG peptide |
| 97 | EQKLISEEDL | Myc peptide |
| 98 | EVQLVESGGGLVQPGGSLRLSCAASGFTFSNYAMSWVRQAPGKGLEWVSAISGSGGS | Anti-CD22 V_{H} region |
| | TYYADSVKGRFTISRDNSKNTLYLQMNSLRAEDTAVYYCARPYYDDWGQGTLVTVS | |
| | S | |
| 99 | QSVVTQPPSVSAAPGQKVTISCSGSSSNIGNNYVSWYQQLPGTAPKLLIYENNKRPSGI | Anti-CD22 V_{L} region |
| | PDRFSGSKSGTSATLGITGLQTGDEADYYCGTWDSSLSAGAVFGGGTKLTVLG | |
| 100 | QVQLVESGGGLVQPGGSLRLSCAASGFTFSNYAMSWVRQAPGKGLEWVSSISGSGGS | Anti-CD22 V_{H} region |
| | TYYADSVKGRFTISRDTSKNTLYLQMNSLRAEDTAVYYCARYGSAAWMDSWGQGT | |
| | LVTVSS | |
| 101 | DIQLTQSPSSLSTSVGDRVTITCQASHDIRNYLNWYQQKPGKAPNLLIYAASNLQTGV | Anti-CD22 V_{L} region |
| | PSRFSGRGSGTDFTLTISSLQPEDIATYYCQQYDGLPLTFGQGTRLEIKR | |
| 102 | EVQLVQSGAEVKKPGESLKISCKGSGYSFTSYWIGWVRQMPGKGLEWMGIIYPGDSD | Anti-CD19 V_{H} region |
| | TRYSPSFQGQVTISADKSISTAYLQWSSLKASDTAMYYCARQVWGWQGGMYPRSNW | |
| | WYNLDSWGQGTLVTVSS | |
| 103 | LPVLTQPPSVSVAPGKTARITCGGNNIGSKSVHWYQQKPGQAPVLVVYDDSDRPSGIP | Anti-CD19 V_{L} region |
| | ERFSGSNSGNTATLTISRVEAGDEADYYCQVWDSSSDYVVFGGGTKLTVLG | |
| 104 | QVQLQQPGAELVKPGASVKMSCKASGYTFTSYNMHWVKQTPGRGLEWIGAIYPGNG | Anti-CD20 V_{H} region |
| | DTSYNQKFKGKATLTADKSSSTAYMQLSSLTSEDSAVYYCARSTYYGGDWYFNVWG | |
| | AGTTVTVSS | |
| 105 | QIVLSQSPAILSASPGEKVTMTCRASSSVSYIHWFQQKPGSSPKPWIYATSNLASGVPV | Anti-CD20 V_{L} region |
| | RFSGSGSGTSYSLTISRVEAEDAATYYCQQWTSNPPTFGGGTKLEIKR | |
| 106 | QVQLQESGPGLVKPSQTLSLTCTVSGYTFTNYYVFWVRQARGQRLEWIGDINPVNGD | Anti-CD47 V_{H} region |
| | TNFNEKFKNRVTISADKSISTAYLQWSSLKASDTAMYYCARGGYTMDYWGQGTLVT | |
| | VS | |
| 107 | DIVMTQTPLSLPVTPGEPASISCRSSQSLVHSNGNTYLHWYQQKPGKAPKLLIYKVSY | Anti-CD47 V_{L} region |
| | RFSGVPDRFSGSGSGTDFTLKISRVEAEDVGVYYCSQNTHVPRTFGQGTKVEIKR | |
| 108 | QVQLVQSGAEVKKPGASVKVSCKASGYTFTSYYMHWVRQAPGQGLEWMGIINPSGG | Anti-GPC3 V_{H} region |
| | STSYAQKFQGRVTMTRDTSTSTVYMELSSLRSEDTAVYYCARWHGGPYDYWGQGTL | |
| | VTVSS | |
| 109 | QPVLTQPPSVSVAPGKTARITCGGNNIGSKSVHWYQQKPGQAPVLVIYYDSDRPSGIP | Anti-GPC3 V_{L} region |
| | ERFSGSNSGNTATLTISRVEAGDEADYYCQVWDSSSDHYVFGTGTKVTVLG | |
| 110 | QVQLVQSGADVRKPGASVKVSCKASGYTFASHGISWVRQAPGQGLEWLGWISPYTG | Anti-GPC3 V_{H} region |
| | NTNYAQKFQGRVTMATDTSTSTAYMELRSLRSDDTAIYYCARGKRTLASCFDYWGQ | |
| | GTLVTVSS | |
| 111 | QSVLTQPPSVSVAPGKTARITCGGNNIGSKSVHWYQQKPGQAPVLVVYDDSDRPSGIP | Anti-GPC3 V_{L} region |
| | ERFSGSNSGNTATLTISRVEAGDEADYYCQVWDSSSDHVFGTGTKVTVLG | |
| 112 | GGAGGAAAAACTGTTTCATACAGAAGGCGTGGAGGAAAAACTGTTTCATACAGA | 6NFAT response |
| | AGGCGTGGAGGAAAAACTGTTTCATACAGAAGGCGTGGAGGAAAAACTGTTTCAT | element |
| | ACAGAAGGCGTGGAGGAAAAACTGTTTCATACAGAAGGCGTGGAGGAAAAACTG | |
| | TTTCATACAGAAGGCGT | |
| 113 | GCCGCCCCGACTGCATCTGCGTGTTCCAATTCGCCAATGACAAGACGCTGGGCGG | TA promoter |
| | GGTTTGTGTCATCATAGAACTAAAGACATGCAAATATATTTCTTCCGGGGACACC | |
| | GCCAGCAAACGCGAGCAACGGGCCACGGGGATGAAGCAG | |
| 114 | GGAGGAAAAACTGTTTCATACAGAAGGCGTGGAGGAAAAACTGTTTCATACAGA | NFAT-derived |
| | AGGCGTGGAGGAAAAACTGTTTCATACAGAAGGCGTGGAGGAAAAACTGTTTCAT | promoter |
| | ACAGAAGGCGTGGAGGAAAAACTGTTTCATACAGAAGGCGTGGAGGAAAAACTG | |
| | TTTCATACAGAAGGCGTCTCGAGGCCGCCCCGACTGCATCTGCGTGTTCCAATTCG | |
| | CCAATGACAAGACGCTGGGCGGGGTTTGTGTCATCATAGAACTAAAGACATGCAA | |
| | ATATATTTCTTCCGGGGACACCGCCAGCAAACGCGAGCAACGGGCCACGGGGATG | |
| | AAGCAG | |
| 115 | QPVLTQPPSASGTPGQRVTISCSGSSSNIGSNNVIWYQQLPGAAPKLLIYSNHRRPSGVP | anti-GPC3 scFv_clone |
| | DRFSGSRSGTSASLAISGLQSEDEADYYCAAWDDSLDGYLFGTGTKVTVLGSRGGGG | 34 |
| | SGGGGSGGGGSLEMAQVQLQQWGAGLLKPSETLSLTCAVYGGSFSGYYWSWIRQPP | |
| | GKGLEWIGEINHSGSTNYNPSLKSRVTISVDTSKNQFSLELSSVTAADTAVYYCARGY | |
| | GGRFDYWGQGTLVTVSS | |
| 116 | QSVLTQPPSVSAAPGQRVTISCSGTRSNIGSDYVSWYQHLPGTAPKLLVYGDNLRPSGI | anti-GPC3 scFv_clone |
| | PDRFSASKSGTSATLGITGLQTGDEADYYCGTWDYTLNGVVFGGGTKLTVLGSRGGG | 37 |
| | GSGGGGSGGGGSLEMAQVQLVESGGGLVQPGGSLRLSCAASGFTFSSYAMSWVRQA | |
| | PGKGLEWVSVIYSGGSSTYYADSVKGRFTISRDNSKNTLYLQMNSLRAEDTAVYYCA | |
| | RTSYLNHGDYWGQGTLVTVSS | |
| 117 | QSVLTQPPSVSGTPGQRVIISCPGSTSNIGTNTVNWYQQFPGTAPKLLIYSNNQRPSGVP | anti-GPC3 scFv_clone |
| | DRFSGSKSGTSASLAISGLQSEDEADYYCAAWDDSLNGVVFGGGTKLTVLGSRGGGG | 45 |
| | SGGGGSGGGGSLEMAQMQLVQSGGGLVKPGGSLRLSCAASGFTFSDYYMSWIRQAP | |
| | GKGLEWVSYISSSGSTIYYADSVKGRFTISRDNAKNSLYLQMNSLRAEDTAVYYCAR | |
| | ASDLYGDWGQGTLVTVSS | |
| 118 | QAVLTQPPSVSTPGQRVTISCSGSSSNFGSNTVHWYQQVPGTAPKLLIFSNTQRPSEIPD | anti-GPC3 scFv_clone |
| | RFSGSKSGTSASLAISGLQSEDEADYYCAAWDDSLTGVVFGGGTKLTVLGSRGGGGS | 46 |
| | GGGGSGGGGSLEMAQVQLVQSGAEVKKPGASVTVSCKASGYRFSNYGVSWVRQAP | |
| | GQGLEWMGWISGSNGNTNYAQKFLGRVTMTTDTSTTTAYMELSSLRSDDTAVYYCA | |
| | RGNRRYYSPIIDPWGQGTLVTVSS | |
| 119 | DVVMTQSPLSLPVTPGEPASVSCRSSQSLLHSNGYNYLDWYLQKPGQSPQLLIYLGSN | anti-GPC3 scFv_clone |
| | RASGVPDRFSGSGSGTDFTLKISRVEAEDVGVYYCMQALQTPWTFGQGTKVEIKRSR | 87 |
| | GGGGSGGGGSGGGGSLEMAEVQLVQSGAEVRKPGSSVKVSCQASGGTFGSYAISWV | |
| | RQAPGQGLEWMGRIIPVLGRTKYAQKFQGRVTVTADTSTSTVYMELTSLTSEDTAVY | |
| | YCARTNDSWGQGTLVTVSS | |
| 120 | QVQLQQWGAGLLKPSETLSLTCAVYGGSFSGYYWSWIRQPPGKGLEWIGEINHSGST | anti-GPC3 V_{H}_clone |
| | NYNPSLKSRVTISVDTSKNQFSLELSSVTAADTAVYYCARGYGGRFDYWGQGTLVTV | 34 |
| | SS | |
| 121 | QPVLTQPPSASGTPGQRVTISCSGSSSNIGSNNVIWYQQLPGAAPKLLIYSNHRRPSGVP | anti-GPC3 V_{L}_clone |
| | DRFSGSRSGTSASLAISGLQSEDEADYYCAAWDDSLDGYLFGTGTKVTVLG | 34 |
| 122 | QVQLVESGGGLVQPGGSLRLSCAASGFTFSSYAMSWVRQAPGKGLEWVSVIYSGGSS | anti-GPC3 V_{H}_clone |
| | TYYADSVKGRFTISRDNSKNTLYLQMNSLRAEDTAVYYCARTSYLNHGDYWGQGTL | 37 |
| | VTVSS | |
| 123 | QSVLTQPPSVSAAPGQRVTISCSGTRSNIGSDYVSWYQHLPGTAPKLLVYGDNLRPSGI | anti-GPC3 V_{L}_clone |
| | PDRFSASKSGTSATLGITGLQTGDEADYYCGTWDYTLNGVVFGGGTKLTVLG | 37 |
| 124 | QMQLVQSGGGLVKPGGSLRLSCAASGFTFSDYYMSWIRQAPGKGLEWVSYISSSGSTI | anti-GPC3 V_{H}_clone |
| | YYADSVKGRFTISRDNAKNSLYLQMNSLRAEDTAVYYCARASDLYGDWGQGTLVT | 45 |
| | VSS | |
| 125 | QSVLTQPPSVSGTPGQRVIISCPGSTSNIGTNTVNWYQQFPGTAPKLLIYSNNQRPSGVP | anti-GPC3 V_{L}_clone |
| | DRFSGSKSGTSASLAISGLQSEDEADYYCAAWDDSLNGVVFGGGTKLTVLG | 45 |
| 126 | QVQLVQSGAEVKKPGASVTVSCKASGYRFSNYGVSWVRQAPGQGLEWMGWISGSN | anti-GPC3 V_{H}_clone |
| | GNTNYAQKFLGRVTMTTDTSTTTAYMELSSLRSDDTAVYYCARGNRRYYSPIIDPWG | 46 |
| | QGTLVTVSS | |
| 127 | QAVLTQPPSVSGTPGQRVTISCSGSSSNFGSNTVHWYQQVPGTAPKLLIFSNTQRPSEIP | anti-GPC3 V_{L}_clone |
| | DRFSGSKSGTSASLAISGLQSEDEADYYCAAWDDSLTGVVFGGGTKLTVLG | 46 |
| 128 | QVQLVQSGAEVKKPGASVKVSCKASGYTFTSYYMHWVRQAPGQGLEWMGIINPSGG | anti-GPC3 V_{H}_clone |
| | STSYAQKFQGRVTMTRDTSTSTVYMELSSLRSEDTAVYYCARWHGGPYDYWGQGTL | 55 |
| | VTVSS | |
| 129 | QPVLTQPPSVSVAPGKTARITCGGNNIGSKSVHWYQQKPGQAPVLVIYYDSDRPSGIP | anti-GPC3 V_{L}_clone |
| | ERFSGSNSGNTATLTISRVEAGDEADYYCQVWDSSSDHYVFGTGTKVTVLG | 55 |
| 130 | QVQLVQSGADVRKPGASVKVSCKASGYTFASHGISWVRQAPGQGLEWLGWISPYTG | anti-GPC3 V_{H}_clone |
| | NTNYAQKFQGRVTMATDTSTSTAYMELRSLRSDDTAIYYCARGKRTLASCFDYWGQ | 58 |
| | GTLVTVSS | |
| 131 | QSVLTQPPSVSVAPGKTARITCGGNNIGSKSVHWYQQKPGQAPVLVVYDDSDRPSGIP | anti-GPC3 V_{L}_clone |
| | ERFSGSNSGNTATLTISRVEAGDEADYYCQVWDSSSDHVFGTGTKVTVLG | 58 |
| 132 | EVQLVQSGAEVRKPGSSVKVSCQASGGTFGSYAISWVRQAPGQGLEWMGRIIPVLGR | anti-GPC3 V_{H}_clone |
| | TKYAQKFQGRVTVTADTSTSTVYMELTSLTSEDTAVYYCARTNDSWGQGTLVTVSS | 87 |
| 133 | DVVMTQSPLSLPVTPGEPASVSCRSSQSLLHSNGYNYLDWYLQKPGQSPQLLIYLGSN | anti-GPC3 V_{L}_clone |
| | RASGVPDRFSGSGSGTDFTLKISRVEAEDVGVYYCMQALQTPWTFGQGTKVEIKR | 87 |
| 134 | METDTLLLWVLLLWVPGSTGQPVLTQPPSASGTPGQRVTISCSGSSSNIGSNNVIWYQ | anti-GPC3-CD8T-Z- |
| | QLPGAAPKLLIYSNHRRPSGVPDRFSGSRSGTSASLAISGLQSEDEADYYCAAWDDSL | CAR with signal |
| | DGYLFGTGTKVTVLGSRGGGGSGGGGSGGGGSLEMAQVQLQQWGAGLLKPSETLSL | peptide (SP) and myc |
| | TCAVYGGSFSGYYWSWIRQPPGKGLEWIGEINHSGSTNYNPSLKSRVTISVDTSKNQF | tag_clone 34 |
| | SLELSSVTAADTAVYYCARGYGGRFDYWGQGTLVTVSSEQKLISEEDLAAATGTTTP | |
| | APRPPTPAPTIASQPLSLRPEACRPAAGGAVHTRGLDFACDIYIWAPLAGTCGVLLLSL | |
| | VITLYCRVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDTYDALH | |
| | MQALPPR | |
| 135 | METDTLLLWVLLLWVPGSTGQSVLTQPPSVSAAPGQRVTISCSGTRSNIGSDYVSWY | anti-GPC3-CD8T-Z- |
| | QHLPGTAPKLLVYGDNLRPSGIPDRFSASKSGTSATLGITGLQTGDEADYYCGTWDYT | CAR with SP and myc |
| | LNGVVFGGGTKLTVLGSRGGGGSGGGGSGGGGSLEMAQVQLVESGGGLVQPGGSLR | tag_clone 37 |
| | LSCAASGFTFSSYAMSWVRQAPGKGLEWVSVIYSGGSSTYYADSVKGRFTISRDNSK | |
| | NTLYLQMNSLRAEDTAVYYCARTSYLNHGDYWGQGTLVTVSSEQKLISEEDLAAAT | |
| | GTTTPAPRPPTPAPTIASQPLSLRPEACRPAAGGAVHTRGLDFACDIYIWAPLAGTCGV | |
| | LLLSLVITLYCRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDTY | |
| | DALHMQALPPR | |
| 136 | METDTLLLWVLLLWVPGSTGQSVLTQPPSVSGTPGQRVIISCPGSTSNIGTNTVNWYQ | anti-GPC3-CD8T-Z- |
| | QFPGTAPKLLIYSNNQRPSGVPDRFSGSKSGTSASLAISGLQSEDEADYYCAAWDDSL | CAR with SP and myc |
| | NGVVFGGGTKLTVLGSRGGGGSGGGGSGGGGSLEMAQMQLVQSGGGLVKPGGSLR | tag_clone 45 |
| | LSCAASGFTFSDYYMSWIRQAPGKGLEWVSYISSSGSTIYYADSVKGRFTISRDNAKN | |
| | SLYLQMNSLRAEDTAVYYCARASDLYGDWGQGTLVTVSSEQKLISEEDLAAATGTTT | |
| | PAPRPPTPAPTIASQPLSLRPEACRPAAGGAVHTRGLDFACDIYIWAPLAGTCGVLLLS | |
| | LVITLYCRVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDTYDAL | |
| | HMQALPPR | |
| 137 | METDTLLLWVLLLWVPGSTGQAVLTQPPSVSTPGQRVTISCSGSSSNFGSNTVHWYQ | anti-GPC3-CD8T-Z- |
| | QVPGTAPKLLIFSNTQRPSEIPDRFSGSKSGTSASLAISGLQSEDEADYYCAAWDDSLT | CAR with SP and myc |
| | GVVFGGGTKLTVLGSRGGGGSGGGGSGGGGSLEMAQVQLVQSGAEVKKPGASVTV | tag_clone 46 |
| | SCKASGYRFSNYGVSWVRQAPGQGLEWMGWISGSNGNTNYAQKFLGRVTMTTDTS | |
| | TTTAYMELSSLRSDDTAVYYCARGNRRYYSPIIDPWGQGTLVTVSSEQKLISEEDLAA | |
| | ATGTTTPAPRPPTPAPTIASQPLSLRPEACRPAAGGAVHTRGLDFACDIYIWAPLAGTC | |
| | GVLLLSLVITLYCRVKFSRSADAPAYQQGQNQLYNELNLGRREEYDVLDKRRGRDPE | |
| | MGGKPRRKNPQEGLYNELQKDKMAEAYSEIGIMKGERRRGKGHDGLYQGLSTATKD | |
| | TYDALHMQALPPR | |
| 138 | METDTLLLWVLLLWVPGSTGQPVLTQPPSVSVAPGKTARITCGGNNIGSKSVHWYQQ | anti-GPC3-CD8T-Z |
| | KPGQAPVLVIYYDSDRPSGIPERFSGSNSGNTATLTISRVEAGDEADYYCQVWDSSSD | CAR with SP and myc |
| | HYVFGTGTKVTVLGSRGGGGSGGGGSGGGGSLEMAQVQLVQSGAEVKKPGASVKV | tag_clone 55 |
| | SCKASGYTFTSYYMHWVRQAPGQGLEWMGIINPSGGSTSYAQKFQGRVTMTRDTST | |
| | STVYMELSSLRSEDTAVYYCARWHGGPYDYWGQGTLVTVSSEQKLISEEDLAAATG | |
| | TTTPAPRPPTPAPTIASQPLSLRPEACRPAAGGAVHTRGLDFACDIYIWAPLAGTCGVL | |
| | LLSLVITLYCRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDTY | |
| | DALHMQALPPR | |
| 139 | METDTLLLWVLLLWVPGSTGQSVLTQPPSVSVAPGKTARITCGGNNIGSKSVHWYQQ | anti-GPC3-CD8T-Z- |
| | KPGQAPVLVVYDDSDRPSGIPERFSGSNSGNTATLTISRVEAGDEADYYCQVWDSSSD | CAR with SP and myc |
| | HVFGTGTKVTVLGSRGGGGSGGGGSGGGGSLEMAQVQLVQSGADVRKPGASVKVS | tag_clone 58 |
| | CKASGYTFASHGISWVRQAPGQGLEWLGWISPYTGNTNYAQKFQGRVTMATDTSTS | |
| | TAYMELRSLRSDDTAIYYCARGKRTLASCFDYWGQGTLVTVSSEQKLISEEDLAAAT | |
| | GTTTPAPRPPTPAPTIASQPLSLRPEACRPAAGGAVHTRGLDFACDIYIWAPLAGTCGV | |
| | LLLSLVITLYCRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDTY | |
| | DALHMQALPPR | |
| 140 | METDTLLLWVLLLWVPGSTGDVVMTQSPLSLPVTPGEPASVSCRSSQSLLHSNGYNY | anti-GPC3-CD8T-Z- |
| | LDWYLQKPGQSPQLLIYLGSNRASGVPDRFSGSGSGTDFTLKISRVEAEDVGVYYCM | CAR with SP and myc |
| | QALQTPWTFGQGTKVEIKRSRGGGGSGGGGSGGGGSLEMAEVQLVQSGAEVRKPGS | tag_clone 87 |
| | SVKVSCQASGGTFGSYAISWVRQAPGQGLEWMGRIIPVLGRTKYAQKFQGRVTVTA | |
| | DTSTSTVYMELTSLTSEDTAVYYCARTNDSWGQGTLVTVSSEQKLISEEDLAAATGTT | |
| | TPAPRPPTPAPTIASQPLSLRPEACRPAAGGAVHTRGLDFACDIYIWAPLAGTCGVLLL | |
| | SLVITLYCRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKDTYDAL | |
| | HMQALPPR | |
| 141 | METDTLLLWVLLLWVPGSTGQPVLTQPPSASGTPGQRVTISCSGSSSNIGSNNVIWYQ | anti-GPC3-CD28z- |
| | QLPGAAPKLLIYSNHRRPSGVPDRFSGSRSGTSASLAISGLQSEDEADYYCAAWDDSL | CAR with SP and myc |
| | DGYLFGTGTKVTVLGSRGGGGSGGGGSGGGGSLEMAQVQLQQWGAGLLKPSETLSL | tag_clone 34 |
| | TCAVYGGSFSGYYWSWIRQPPGKGLEWIGEINHSGSTNYNPSLKSRVTISVDTSKNQF | |
| | SLELSSVTAADTAVYYCARGYGGRFDYWGQGTLVTVSSEQKLISEEDLAAAIEVMYP | |
| | PPYLDNEKSNGTIIHVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRVKFSRSADAPAYQQ | |
| | GQNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 142 | METDTLLLWVLLLWVPGSTGQSVLTQPPSVSAAPGQRVTISCSGTRSNIGSDYVSWY | anti-GPC3-CD28z- |
| | QHLPGTAPKLLVYGDNLRPSGIPDRFSASKSGTSATLGITGLQTGDEADYYCGTWDYT | CAR with SP and myc |
| | LNGVVFGGGTKLTVLGSRGGGGSGGGGSGGGGSLEMAQVQLVESGGGLVQPGGSLR | tag_clone 37 |
| | LSCAASGFTFSSYAMSWVRQAPGKGLEWVSVIYSGGSSTYYADSVKGRFTISRDNSK | |
| | NTLYLQMNSLRAEDTAVYYCARTSYLNHGDYWGQGTLVTVSSEQKLISEEDLAAAIE | |
| | VMYPPPYLDNEKSNGTIIHVKGKHLCPSPLFPGPSKPFWVLVVVGGVLACYSLLVTVA | |
| | FIIFWVRSKRSRLLHSDYMNMTPRRPGPTRKHYQPYAPPRDFAAYRSRVKFSRSADAP | |
| | AYQQGQNQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPR | |
| 143 | METDTLLLWVLLLWVPGSTGQSVLTQPPSVSGTPGQRVIISCPGSTSNIGTNTVNWYQ | anti-GPC3-CD28z- |
| | QFPGTAPKLLIYSNNQRPSGVPDRFSGSKSGTSASLAISGLQSEDEADYYCAAWDDSL | CAR with SP and myc |
| | NGVVFGGGTKLTVLGSRGGGGSGGGGSGGGGSLEMAQMQLVQSGGGLVKPGGSLR | tag_clone 45 |
| | LSCAASGFTFSDYYMSWIRQAPGKGLEWVSYISSSGSTIYYADSVKGRFTISRDNAKN | |
| | SLYLQMNSLRAEDTAVYYCARASDLYGDWGQGTLVTVSSEQKLISEEDLAAAIEVM | |
| | YPPPYLDNEKSNGTIIHVKGKHLCPSPLFPGPSKPFWVLVVVGGVLACYSLLVTVAFII | |
| | FWVRSKRSRLLHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGLYNELQKDKM | |
| | AEAYSEIG1MKGERRRGKGHDGLYQGLSTATKDTYDALHMQALPPR | |
| 144 | METDTLLLWVLLLWVPGSTGQAVLTQPPSVSTPGQRVTISCSGSSSNFGSNTVHWYQ | anti-GPC3-CD28z- |
| | QVPGTAPKLLIFSNTQRPSEIPDRFSGSKSGTSASLAISGLQSEDEADYYCAAWDDSLT | CAR with SP and myc |
| | GVVFGGGTKLTVLGSRGGGGSGGGGSGGGGSLEMAQVQLVQSGAEVKKPGASVTV | tag_clone 46 |
| | SCKASGYRFSNYGVSWVRQAPGQGLEWMGWISGSNGNTNYAQKFLGRVTMTTDTS | |
| | TTTAYMELSSLRSDDTAVYYCARGNRRYYSPIIDPWGQGTLVTVSSEQKLISEEDLAA | |
| | AIEVMYPPPYLDNEKSNGTIIHVKGKHLCPSPLFPGPSKPFWVLVVVGGVLACYSLLV | |
| | TVAFIIFWVRSKRSRLLHSDYMNMTPRRPGPTRKHYQPYAPPRDFAAYRSRVKFSRSA | |
| | DAPAYQQGQNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALPPR | |
| 145 | METDTLLLWVLLLWVPGSTGQPVLTQPPSVSVAPGKTARITCGGNNIGSKSVHWYQQ | anti-GPC3-CD28z- |
| | KPGQAPVLVIYYDSDRPSGIPERFSGSNSGNTATLTISRVEAGDEADYYCQVWDSSSD | CAR with SP and myc |
| | HYVFGTGTKVTVLGSRGGGGSGGGGSGGGGSLEMAQVQLVQSGAEVKKPGASVKV | tag_clone 55 |
| | SCKASGYTFTSYYMHWVRQAPGQGLEWMGIINPSGGSTSYAQKFQGRVTMTRDTST | |
| | STVYMELSSLRSEDTAVYYCARWHGGPYDYWGQGTLVTVSSEQKLISEEDLAAAIEV | |
| | MYPPPYLDNEKSNGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 146 | METDTLLLWVLLLWVPGSTGQSVLTQPPSVSVAPGKTARITCGGNNIGSKSVHWYQQ | anti-GPC3-CD28z- |
| | KPGQAPVLVVYDDSDRPSGIPERFSGSNSGNTATLTISRVEAGDEADYYCQVWDSSSD | CAR with SP and myc |
| | HVFGTGTKVTVLGSRGGGGSGGGGSGGGGSLEMAQVQLVQSGADVRKPGASVKVS | tag_clone 58 |
| | CKASGYTFASHGISWVRQAPGQGLEWLGWISPYTGNTNYAQKFQGRVTMATDTSTS | |
| | TAYMELRSLRSDDTAIYYCARGKRTLASCFDYWGQGTLVTVSSEQKLISEEDLAAAIE | |
| | VMYPPPYLDNEKSNGTIIHVKGKHLCPSPLFPGPSKPFWVLVVVGGVLACYSLLVTVA | |
| | FIIFWVRSKRSRLLHSDYMNMTPRRPGPTRKHYQPYAPPRDFAAYRSRVKFSRSADAP | |
| | AYQQGQNQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPR | |
| 147 | METDTLLLWVLLLWVPGSTGDVVMTQSPLSLPVTPGEPASVSCRSSQSLLHSNGYNY | anti-GPC3-CD28z- |
| | LDWYLQKPGQSPQLLIYLGSNRASGVPDRFSGSGSGTDFTLKISRVEAEDVGVYYCM | CAR with SP and myc |
| | QALQTPWTFGQGTKVEIKRSRGGGGSGGGGSGGGGSLEMAEVQLVQSGAEVRKPGS | tag_clone 87 |
| | SVKVSCQASGGTFGSYAISWVRQAPGQGLEWMGRIIPVLGRTKYAQKFQGRVTVTA | |
| | DTSTSTVYMELTSLTSEDTAVYYCARTNDSWGQGTLVTVSSEQKLISEEDLAAAIEVM | |
| | YPPPYLDNEKSNGTIIHVKGKHLCPSPLFPGPSKPFWVLVVVGGVLACYSLLVTVAFII | |
| | FWVRSKRSRLLHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| 148 | METDTLLLWVLLLWVPGSTGQPVLTQPPSASGTPGQRVTISCSGSSSNIGSNNVIWYQ | anti-GPC3-CD30-CSR |
| | QLPGAAPKLLIYSNHRRPSGVPDRFSGSRSGTSASLAISGLQSEDEADYYCAAWDDSL | with SP and myc |
| | DGYLFGTGTKVTVLGSRGGGGSGGGGSGGGGSLEMAQVQLQQWGAGLLKPSETLSL | tag_clone 34 |
| | TCAVYGGSFSGYYWSWIRQPPGKGLEWIGEINHSGSTNYNPSLKSRVTISVDTSKNQF | |
| | SLELSSVTAADTAVYYCARGYGGRFDYWGQGTLVTVSSEQKLISEEDLAAATGAPPL | |
| | GTQPDCNPTPENGEAPASTSPTQSLLVDSQASKTLPIPTSAPVALSSTGKPVLDAGPVL | |
| | FWVILVLVVVVGSSAFLLCHRRACRKRIRQKLHLCYPVQTSQPKLELVDSRPRRSSTQ | |
| | LRSGASVTEPVAEERGLMSQPLMETCHSVGAAYLESLPLQDASPAGGPSSPRDLPEPR | |
| | VSTEHTNNKIEKIYEVIKADTVIVGTVKAELPEGRGLAGPAEPELEEELEADHTPHYPEQ | |
| | ETEPPLGSCSDVMLSVEEEGKEDPLPTAASGK | |
| 149 | METDTLLLWVLLLWVPGSTGQSVLTQPPSVSGTPGQRVIISCPGSTSNIGTNTVNWYQ | anti-GPC3-CD30-CSR |
| | QFPGTAPKLLIYSNNQRPSGVPDRFSGSKSGTSASLAISGLQSEDEADYYCAAWDDSL | with SP and myc |
| | NGVVFGGGTKLTVLGSRGGGGSGGGGSGGGGSLEMAQMQLVQSGGGLVKPGGSLR | tag_clone 45 |
| | LSCAASGFTFSDYYMSWIRQAPGKGLEWVSYISSSGSTIYYADSVKGRFTISRDNAKN | |
| | SLYLQMNSLRAEDTAVYYCARASDLYGDWGQGTLVTVSSEQKLISEEDLAAATGAPP | |
| | LGTQPDCNPTPENGEAPASTSPTQSLLVDSQASKTLPIPTSAPVALSSTGKPVLDAGPV | |
| | LFWVILVLVVVVGSSAFLLCHRRACRKRIRQKLHLCYPVQTSQPKLELVDSRPRRSST | |
| | QLRSGASVTEPVAEERGLMSQPLMETCHSVGAAYLESLPLQDASPAGGPSSPRDLPEP | |
| | RVSTEHTNNKIEKIYIMKADTVIVGTVKAELPEGRGLAGPAEPELEEELEADHTPHYPE | |
| | QETEPPLGSCSDVMLSVEEEGKEDPLPTAASGK | |
| 150 | METDTLLLWVLLLWVPGSTGQAVLTQPPSVSTPGQRVTISCSGSSSNFGSNTVHWYQ | anti-GPC3-CD30-CSR |
| | QVPGTAPKLLIFSNTQRPSEIPDRFSGSKSGTSASLAISGLQSEDEADYYCAAWDDSLT | with SP and myc |
| | GVVFGGGTKLTVLGSRGGGGSGGGGSGGGGSLEMAQVQLVQSGAEVKKPGASVTV | tag_clone 46 |
| | SCKASGYRFSNYGVSWVRQAPGQGLEWMGWISGSNGNTNYAQKFLGRVTMTTDTS | |
| | TTTAYMELSSLRSDDTAVYYCARGNRRYYSPIIDPWGQGTLVTVSSEQKLISEEDLAA | |
| | ATGAPPLGTQPDCNPTPENGEAPASTSPTQSLLVDSQASKTLPIPTSAPVALSSTGKPVL | |
| | DAGPVLFWVILVLVVVVGSSAFLLCHRRACRKRIRQKLHLCYPVQTSQPKLELVDSRP | |
| | RRSSTQLRSGASVTEPVAEERGLMSQPLMETCHSVGAAYLESLPLQDASPAGGPSSPR | |
| | DLPEPRVSTEHTNNKIEKIYIMKADTVIVGTVKAELPEGRGLAGPAEPELEEELEADHT | |
| | PHYPEQETEPPLGSCSDVMLSVEEEGKEDPLPTAASGK | |
| 151 | METDTLLLWVLLLWVPGSTGQPVLTQPPSVSVAPGKTARITCGGNNIGSKSVHWYQQ | anti-GPC3-CD30-CSR |
| | KPGQAPVLVIYYDSDRPSGIPERFSGSNSGNTATLTISRVEAGDEADYYCQVWDSSSD | with SP and myc |
| | HYVFGTGTKVTVLGSRGGGGSGGGGSGGGGSLEMAQVQLVQSGAEVKKPGASVKV | tag_clone 55 |
| | SCKASGYTFTSYYMHWVRQAPGQGLEWMGIINPSGGSTSYAQKFQGRVTMTRDTST | |
| | STVYMELSSLRSEDTAVYYCARWHGGPYDYWGQGTLVTVSSEQKLISEEDLAAATG | |
| | APPLGTQPDCNPTPENGEAPASTSPTQSLLVDSQASKTLPIPTSAPVALSSTGKPVLDA | |
| | GPVLFWVILVLVVVVGSSAFLLCHRRACRKRIRQKLHLCYPVQTSQPKLELVDSRPRR | |
| | SSTQLRSGASVTEPVAEERGLMSQPLMETCHSVGAAYLESLPLQDASPAGGPSSPRDL | |
| | PEPRVSTEHTNNKIEKIYIMKADTVIVGTVKAELPEGRGLAGPAEPELEEELEADHTPH | |
| | YPEQETEPPLGSCSDVMLSVEEEGKEDPLPTAASGK | |
| 152 | METDTLLLWVLLLWVPGSTGQSVLTQPPSVSVAPGKTARITCGGNNIGSKSVHWYQQ | anti-GPC3-CD30-CSR |
| | KPGQAPVLVVYDDSDRPSGIPERFSGSNSGNTATLTISRVEAGDEADYYCQVWDSSSD | with SP and myc |
| | HVFGTGTKVTVLGSRGGGGSGGGGSGGGGSLEMAQVQLVQSGADVRKPGASVKVS | tag_clone 58 |
| | CKASGYTFASHGISWVRQAPGQGLEWLGWISPYTGNTNYAQKFQGRVTMATDTSTS | |
| | TAYMELRSLRSDDTAIYYCARGKRTLASCFDYWGQGTLVTVSSEQKLISEEDLAAAT | |
| | GAPPLGTQPDCNPTPENGEAPASTSPTQSLLVDSQASKTLPIPTSAPVALSSTGKPVLD | |
| | AGPVLFWVILVLVVVVGSSAFLLCHRRACRKRIRQKLHLCYPVQTSQPKLELVDSRPR | |
| | RSSTQLRSGASVTEPVAEERGLMSQPLMETCHSVGAAYLESLPLQDASPAGGPSSPRD | |
| | LPEPRVSTEHTNNKIEKIYIMKADTVIVGTVKAELPEGRGLAGPAEPELEEELEADHTP | |
| | HYPEQETEPPLGSCSDVMLSVEEEGKEDPLPTAASGK | |
| 153 | METDTLLLWVLLLWVPGSTGDVVMTQSPLSLPVTPGEPASVSCRSSQSLLHSNGYNY | anti-GPC3-CD30-CSR |
| | LDWYLQKPGQSPQLLIYLGSNRASGVPDRFSGSGSGTDFTLKISRVEAEDVGVYYCM | with SP and myc |
| | QALQTPWTFGQGTKVEIKRSRGGGGSGGGGSGGGGSLEMAEVQLVQSGAEVRKPGS | tag_clone 87 |
| | SVKVSCQASGGTFGSYAISWVRQAPGQGLEWMGRIIPVLGRTKYAQKFQGRVTVTA | |
| | DTSTSTVYMELTSLTSEDTAVYYCARTNDSWGQGTLVTVSSEQKLISEEDLAAATGA | |
| | PPLGTQPDCNPTPENGEAPASTSPTQSLLVDSQASKTLPIPTSAPVALSSTGKPVLDAGP | |
| | VLFWVILVLVVVVGSSAFLLCHRRACRKRIRQKLHLCYPVQTSQPKLELVDSRPRRSS | |
| | TQLRSGASVTEPVAEERGLMSQPLMETCHSVGAAYLESLPLQDASPAGGPSSPRDLPE | |
| | PRVSTEHTNNKIEKIYEVIKADTVIVGTVKAELPEGRGLAGPAEPELEEELEADHTPHYP | |
| | EQETEPPLGSCSDVMLSVEEEGKEDPLPTAASGK | |
| 154 | METDTLLLWVLLLWVPGSTG | Signal peptide |
| 155 | LLAGLVAADAVASLLIVGAVFLCARPRRSPAQEDGKVYINMPGRG | Dap10 TM + IC |
| 156 | CARPRRSPAQEDGKVYINMPGRG | Dap10 IC signaling |
| | | sequence |
| 157 | PLFQVPEPV | AFP peptide can be |
| | | targeted by CAR; |
| | | hAFP137-145 |
| 158 | FMNKFIYEI | AFP peptide can be |
| | | targeted by CAR; |
| | | hAFP158-166 |
| 159 | GLSPNLNRFL | AFP peptide can be |
| | | targeted by CAR; |
| | | hAFP325-334 |
| 160 | GVALQTMKQ | AFP peptide can be |
| | | targeted by CAR; |
| | | hAFP542-550 |
| 161 | AMNKFIYEI | AFP peptide can be |
| | | targeted by CAR; |
| | | hAFP158 A1 |
| 162 | FMAKFIYEI | AFP peptide can be |
| | | targeted by CAR; |
| | | hAFP158 A3 |
| 163 | FMNAFIYEI | AFP peptide can be |
| | | targeted by CAR; |
| | | hAFP158 A4 |
| 164 | FMNKAIYEI | AFP peptide can be |
| | | targeted by CAR; |
| | | hAFP158 A5 |
| 165 | FMNKFAYEI | AFP peptide can be |
| | | targeted by CAR; |
| | | hAFP158 A6 |
| 166 | FMNKFIAEI | hAFP158 A7 |
| | | AFP peptide can be |
| 167 | FMNKFIYAI | targeted by CAR; |
| | | hAFP158 A8 |
| 168 | GYTFTSYG | Anti-AFP_HCDR1 |
| 169 | ISAYNGNT | Anti-AFP_HCDR2 |
| 170 | ARDSYYYYYGMDV | Anti-AFP_HCDR3 |
| 171 | EVQLVQSGAEVKKPGASVKVSCKASGYTFTSYGISWVRQAPGQGLEWMGWISAYNG | anti-AFP V_{H} |
| | NTNYAQKLQGRVTMTTDTSTSTAYMELRSLRSDDTAVYYCARDSYYYYYGMDVWG | |
| | QGTTVTVSS | |
| 172 | TGAVTSGHY | Anti-AFP_LCDR1 |
| 173 | DAS | Anti-AFP_LCDR2 |
| 174 | LLSYSDALV | Anti-AFP_LCDR3 |
| 175 | QAVVTQEPSLTVSPGGTVTLTCGSSTGAVTSGHYPYWFQQKPGQAPRTLIYDASDKH | anti-AFP V_{L} |
| | SWTPARFSGSLLGGKAALTLSGAQPEDEAEYYCLLSYSDALVFGGGTKLTVLG | |
| 176 | GYSFPNYW | Anti-AFP_HCDR1 |
| 177 | IDPGDSYT | Anti-AFP_HCDR2 |
| 178 | ARYYVSLVDI | Anti-AFP_HCDR3 |
| 179 | EVQLVQSGAEVKKPGESLTISCKASGYSFPNYWITWVRQMSGGGLEWMGRIDPGDSY | anti-AFP V_{H} |
| | TTYNPSFQGHVTISIDKSTNTAYLHWNSLKASDTAMYYCARYYVSLVDIWGQGTLVT | |
| | VSS | |
| 180 | SSDVGGYNY | Anti-AFP_LCDR1 |
| 181 | DVN | Anti-AFP_LCDR2 |
| 182 | SSYTTGSRAV | Anti-AFP_LCDR3 |
| 183 | QSVLTQPASVSGSPGQSITISCTGTSSDVGGYNYVSWYQQHPGKAPKLMIYDVNNRPS | anti-AFP V_{L} |
| | EVSNRFSGSKSGNTASLTISGLQAEDEADYYCSSYTTGSRAVFGGGTKLTVLG | |
| 184 | GFTFSNAW | Anti-AFP_HCDR1 |
| 185 | IRSKAYGGTT | Anti-AFP_HCDR2 |
| 186 | ARDGLYSSSWYDSDY | Anti-AFP_HCDR3 |
| 187 | EVQLVESGGGLVKPGGSLRLSCAASGFTFSNAWMSWVRQAPGKGLEWVGFIRSKAY | anti-AFP V_{H} |
| | GGTTEYAASVKGRFTISRDDSKSIAYLQMNNLKTEDTAVYYCARDGLYSSSWYDSDY | |
| | WGQGTLVTVSS | |
| 188 | SSNIGNNY | Anti-AFP_LCDR1 |
| 189 | DNN | Anti-AFP_LCDR2 |
| 190 | GTWDGSLYTML | Anti-AFP_LCDR3 |
| 191 | QSVVTQPPSVSAAPGQKVTISCSGSSSNIGNNYVSWYQQLPGTAPKLLIYDNNKRPSGI | anti-AFP V_{L} |
| | PDRFSGSKSGTSATLGITGLQTGDEADYYCGTWDGSLYTMLFGGGTKLTVLG | |
| 192 | GFTFDDYA | Anti-AFP_HCDR1 |
| 193 | ISWNSGSI | Anti-AFP_HCDR2 |
| 194 | AKDIHSGSYYGLLYYAMDV | Anti-AFP_HCDR3 |
| 195 | QMQLVQSGGGLVQPGRSLRLSCAASGFTFDDYAMHWVRQAPGKGLEWVSGISWNS | anti-AFP V_{H} |
| | GSIGYADSVKGRFTISRDNAKNSLYLQMNSLRAEDTALYYCAKDIHSGSYYGLLYYA | |
| | MDVWGQGTTVTVSS | |
| 196 | SSNIGAGYD | Anti-AFP_LCDR1 |
| 197 | GNS | Anti-AFP_LCDR2 |
| 198 | QSYDSSLSGSGV | Anti-AFP_LCDR3 |
| 199 | QSVLTQPPSVSGAPGQRVTISCTGSSSNIGAGYDVHWYQQLPGTAPKLLIFGNSNRPSG | anti-AFP VL |
| | VPDRFSGFKSGTSASLAITGLQAEDEADYFCQSYDSSLSGSGVFGTGTKVTVLG | |
| 200 | GYTFTSYG | Anti-AFP_HCDR1 |
| 201 | ISAYNGNT | Anti-AFP_HCDR2 |
| 202 | ARFQDWWYLGQFDQ | Anti-AFP_HCDR3 |
| 203 | EVQLVQSGAEVKKPGASVKVSCKASGYTFTSYGISWVRQAPGQGLEWMGWISAYNG | anti-AFP V_{H} |
| | NTNYAQKLQGRVTMTTDTSTSTAYMELRSLRSDDTAVYYCARFQDWWYLGQFDQW | |
| | GQGTLVTVSS | |
| 204 | GSDVGVYYY | Anti-AFP_LCDR1 |
| 205 | DVD | Anti-AFP_LCDR2 |
| 206 | ASYTNRNSLGYV | Anti-AFP_LCDR3 |
| 207 | QSALTQPASVSGSPGQSITISCTATGSDVGVYYYVSWYQQHPGKAPKVMIYDVDNRP | anti-AFP V_{L} |
| | PGVSNRFSGSKSGNTASLTISGLQAEDEADYYCASYTNRNSLGYVFGTGTKVTVLG | |
| 208 | GGSFSGYY | Anti-GPC3_HCDR1 |
| 209 | INHSGST | Anti-GPC3_HCDR2 |
| 210 | ARGYGGRFDY | Anti-GPC3_HCDR3 |
| 211 | QVQLQQWGAGLLKPSETLSLTCAVYGGSFSGYYWSWIRQPPGKGLEWIGEINHSGST | anti-GPC3 V_{H}_clone |
| | NYNPSLKSRVTISVDTSKNQFSLELSSVTAADTAVYYCARGYGGRFDYWGQGTLVTV | 34 |
| | SS | |
| 212 | SSNIGSNN | Anti-GPC3_LCDR1 |
| 213 | SNH | Anti-GPC3_LCDR2 |
| 214 | AAWDDSLDGYL | Anti-GPC3_LCDR3 |
| 215 | QPVLTQPPSASGTPGQRVTISCSGSSSNIGSNNVIWYQQLPGAAPKLLIYSNHRRPSGVP | anti-GPC3 V_{L}_clone |
| | DRFSGSRSGTSASLAISGLQSEDEADYYCAAWDDSLDGYLFGTGTKVTVLG | 34 |
| 216 | GFTFSSYA | Anti-GPC3_HCDR1 |
| 217 | IYSGGSST | Anti-GPC3_HCDR2 |
| 218 | ARTSYLNHGDY | Anti-GPC3_HCDR3 |
| 219 | QVQLVESGGGLVQPGGSLRLSCAASGFTFSSYAMSWVRQAPGKGLEWVSVIYSGGSS | anti-GPC3 V_{H}_clone |
| | TYYADSVKGRFTISRDNSKNTLYLQMNSLRAEDTAVYYCARTSYLNHGDYWGQGTL | 37 |
| | VTVSS | |
| 220 | RSNIGSDY | Anti-GPC3_LCDR1 |
| 221 | GDN | Anti-GPC3_LCDR2 |
| 222 | GTWDYTLNGVV | Anti-GPC3_LCDR3 |
| 223 | QSVLTQPPSVSAAPGQRVTISCSGTRSNIGSDYVSWYQHLPGTAPKLLVYGDNLRPSGI | anti-GPC3 V_{L}_clone |
| | PDRFSASKSGTSATLGITGLQTGDEADYYCGTWDYTLNGVVFGGGTKLTVLG | 37 |
| 224 | GFTFSDYY | Anti-GPC3_HCDR1 |
| 225 | ISSSGSTI | Anti-GPC3_HCDR2 |
| 226 | ARASDLYGD | Anti-GPC3_HCDR3 |
| 227 | QMQLVQSGGGLVKPGGSLRLSCAASGFTFSDYYMSWIRQAPGKGLEWVSYISSSGSTI | anti-GPC3 V_{H}_clone |
| | YYADSVKGRFTISRDNAKNSLYLQMNSLRAEDTAVYYCARASDLYGDWGQGTLVT | 45 |
| | VSS | |
| 228 | TSNIGTNT | Anti-GPC3_LCDR1 |
| 229 | SNN | Anti-GPC3_LCDR2 |
| 230 | AAWDDSLNGVV | Anti-GPC3_LCDR3 |
| 231 | QSVLTQPPSVSGTPGQRVIISCPGSTSNIGTNTVNWYQQFPGTAPKLLIYSNNQRPSGVP | anti-GPC3 V_{L}_clone |
| | DRFSGSKSGTSASLAISGLQSEDEADYYCAAWDDSLNGVVFGGGTKLTVLG | 45 |
| 232 | GYRFSNYG | Anti-GPC3_HCDR1 |
| 233 | ISGSNGNT | Anti-GPC3_HCDR2 |
| 234 | ARGNRRYYSPIIDP | Anti-GPC3_HCDR3 |
| 235 | QVQLVQSGAEVKKPGASVTVSCKASGYRFSNYGVSWVRQAPGQGLEWMGWISGSN | anti-GPC3 V_{H}_clone |
| | GNTNYAQKFLGRVTMTTDTSTTTAYMELSSLRSDDTAVYYCARGNRRYYSPIIDPWG | 46 |
| | QGTLVTVSS | |
| 236 | SSNFGSNT | Anti-GPC3_LCDR1 |
| 237 | SNT | Anti-GPC3_LCDR2 |
| 238 | AAWDDSLTGVV | Anti-GPC3_LCDR3 |
| 239 | QAVLTQPPSVSGTPGQRVTISCSGSSSNFGSNTVHWYQQVPGTAPKLLIFSNTQRPSEIP | anti-GPC3 V_{L}_clone |
| | DRFSGSKSGTSASLAISGLQSEDEADYYCAAWDDSLTGVVFGGGTKLTVLG | 46 |
| 240 | GGTFGSYA | Anti-GPC3_HCDR1 |
| 241 | IIPVLGRT | Anti-GPC3_HCDR2 |
| 242 | ARTNDS | Anti-GPC3_HCDR3 |
| 243 | EVQLVQSGAEVRKPGSSVKVSCQASGGTFGSYAISWVRQAPGQGLEWMGRIIPVLGR | anti-GPC3 V_{H}_clone |
| | TKYAQKFQGRVTVTADTSTSTVYMELTSLTSEDTAVYYCARTNDSWGQGTLVTVSS | 87 |
| 244 | QSLLHSNGYNY | Anti-GPC3_LCDR1 |
| 245 | LGS | Anti-GPC3_LCDR2 |
| 246 | MQALQTPWT | Anti-GPC3_LCDR3 |
| 247 | DVVMTQSPLSLPVTPGEPASVSCRSSQSLLHSNGYNYLDWYLQKPGQSPQLLIYLGSN | anti-GPC3 V_{L}_clone |
| | RASGVPDRFSGSGSGTDFTLKISRVEAEDVGVYYCMQALQTPWTFGQGTKVEIKR | 87 |
| 248 | GYTFTSYY | Anti-GPC3_HCDR1 |
| 249 | INPSGGST | Anti-GPC3_HCDR2 |
| 250 | ARWHGGPYDY | Anti-GPC3_HCDR3 |
| 251 | QVQLVQSGAEVKKPGASVKVSCKASGYTFTSYYMHWVRQAPGQGLEWMGIINPSGG | Anti-GPC3 V_{H} |
| | STSYAQKFQGRVTMTRDTSTSTVYMELSSLRSEDTAVYYCARWHGGPYDYWGQGTL | |
| | VTVSS | |
| 252 | NIGSKS | Anti-GPC3_LCDR1 |
| 253 | YDS | Anti-GPC3_LCDR2 |
| 254 | QVWDSSSDHYV | Anti-GPC3_LCDR3 |
| 255 | QPVLTQPPSVSVAPGKTARITCGGNNIGSKSWYQQKPGQAPVLVIYYDSDRPSGIP | Anti-GPC3 V_{L} |
| | ERFSGSNSGNTATLTISRVEAGDEADYYCQVWDSSSDHYVFGTGTKVTVLG | |
| 256 | GYTFASHG | Anti-GPC3_HCDR1 |
| 257 | ISPYTGNT | Anti-GPC3_HCDR2 |
| 258 | ARGKRTLASCFDY | Anti-GPC3_HCDR3 |
| 259 | QVQLVQSGADVRKPGASVKVSCKASGYTFASHGISWVRQAPGQGLEWLGWISPYTG | Anti-GPC3 V_{H} |
| | NTNYAQKFQGRVTMATDTSTSTAYMELRSLRSDDTAIYYCARGKRTLASCFDYWGQ | |
| | GTLVTVSS | |
| 260 | NIGSKS | Anti-GPC3_LCDR1 |
| 261 | DDS | Anti-GPC3_LCDR2 |
| 262 | QVWDSSSDHV | Anti-GPC3_LCDR3 |
| 263 | QSVLTQPPSVSVAPGKTARITCGGNNIGSKSVHWYQQKPGQAPVLVVYDDSDRPSGIP | Anti-GPC3 V_{L} |
| | ERFSGSNSGNTATLTISRVEAGDEADYYCQVWDSSSDHVFGTGTKVTVLG | |
| 264 | KLVVVGAGGV | KRAS peptide can be |
| | | targeted by CAR |
| 265 | KLVVVGAVGV | KRAS peptide can be |
| | | targeted by CAR |
| 266 | KLVVVGACGV | KRAS peptide can be |
| | | targeted by CAR |
| 267 | KLVVVGADGV | KRAS peptide can be |
| | | targeted by CAR |
| 268 | KLVVVGASGV | KRAS peptide can be |
| | | targeted by CAR |
| 269 | LVVVGAGGV | KRAS peptide can be |
| | | targeted by CAR |
| 270 | LVVVGAVGV | KRAS peptide can be |
| | | targeted by CAR |
| 271 | LVVVGACGV | KRAS peptide can be |
| | | targeted by CAR |
| 272 | LVVVGADGV | KRAS peptide can be |
| | | targeted by CAR |
| 273 | GGTFSSYA | Anti-KRAS_HCDR1 |
| 274 | IIPIFGKG | Anti-KRAS_HCDR2 |
| 275 | ARHIPTFSFDY | Anti-KRAS_HCDR3 |
| 276 | QVQLVQSGAEVKKPGSSVKVSCKASGGTFSSYAISWVRQAPGQGLEWMGGIIPIFGK | Anti-KRAS_V_{H} |
| | GNYPQKFQGRVTITADESTGTAYMELSSLRSEDTAVYYCARHIPTFSFDYWGQGTLV | |
| | TVSS | |
| 277 | SSNIGAGYD | Anti-KRAS_LCDR1 |
| 278 | GNS | Anti-KRAS_LCDR2 |
| 279 | QSYDSSLSGYV | Anti-KRAS_LCDR3 |
| 280 | QSVLTQPPSVSGAPGQRVTISCTGSSSNIGAGYDVHWYQQLPGTAPKLLIYGNSNRPS | Anti-KRAS_V_{L} |
| | GVPDRFSGSKSGTSASLAITGLQAEDEADYYCQSYDSSLSGYVFGTGTKVTVLG | |
| 281 | QSVLTQPPSVSGAPGQRVTISCTGSSSNIGAGYDVHWYQQLPGTAPKLLIYGNSNRPS | Anti-KRAS_scFv |
| | GVPDRFSGSKSGTSASLAITGLQAEDEADYYCQSYDSSLSGYVFGTGTKVTVLGSRGG | |
| | GGSGGGGSGGGGSLEMAQVQLVQSGAEVKKPGSSVKVSCKASGGTFSSYAISWVRQ | |
| | APGQGLEWMGGIIPIFGKGNYPQKFQGRVTITADESTGTAYMELSSLRSEDTAVYYCA | |
| | RHIPTFSFDYWGQGTLVTVSS | |
| 282 | GGTFSSYT | Anti-KRAS_HCDR1 |
| 283 | FIPISGTV | Anti-KRAS_HCDR2 |
| 284 | ARPLDWTEDI | Anti-KRAS_HCDR3 |
| 285 | QVQLVQSGAEVKKPGSSVKVSCKASGGTFSSYTINWVRQAPGQGLEWMGGFIPISGT | Anti-KRAS_V_{H} |
| | VNYAQKFQGRVTITADESTSTAYMELSSLRSEDTAVYYCARPLDWTEDIWGQGTLVT | |
| | VSS | |
| 286 | SSNIGAGYD | Anti-KRAS_LCDR1 |
| 287 | GNS | Anti-KRAS_LCDR2 |
| 288 | QSYDSSLSGSV | Anti-KRAS_LCDR3 |
| 289 | QSVLTQPPSVSGAPGQRVTISCTGSSSNIGAGYDVHWYQQLPGTAPKLLIYGNSNRPS | Anti-KRAS_V_{L} |
| | GVPDRFSGSKSGTSASLAITGLQAEDEADYYCQSYDSSLSGSVFGTGTKVTVLG | |
| 290 | QSVLTQPPSVSGAPGQRVTISCTGSSSNIGAGYDVHWYQQLPGTAPKLLIYGNSNRPS | Anti-KRAS_scFv |
| | GVPDRFSGSKSGTSASLAITGLQAEDEADYYCQSYDSSLSGSVFGTGTKVTVLGSRGG | |
| | GGSGGGGSGGGGSLEMAVQLVQSGAEVKKPGSSVKVSCKASGGTFSSYTINWVRQA | |
| | PGQGLEWMGGFIPISGTVNYAQKFQGRVTITADESTSTAYMELSSLRSEDTAVYYCAR | |
| | PLDWTEDIWGQGTLVTVSS | |
| 291 | GYTFTAYY | Anti-KRAS_HCDR1 |
| 292 | MNTNNGAT | Anti-KRAS_HCDR2 |
| 293 | ARGDISQDFADV | Anti-KRASHCDR3 |
| 294 | EVQLVQSGAEVKKPGASVKVSCKASGYTFTAYYLHWLRQAPGQGLEWMGWMNTN | Anti-KRAS_V_{H} |
| | NGATRYAQKFQDRVTMTRDTSINTAYMEMSGLSSDDTAMYYCARGDISQDFADVW | |
| | GQGTLVTVSS | |
| 295 | SGSIASNY | Anti-KRAS_LCDR1 |
| 296 | EDN | Anti-KRAS_LCDR2 |
| 297 | QSYDDINHWV | Anti-KRAS_LCDR3 |
| 298 | NFMLTQPHSVSESPGKTVTISCTGSSGSIASNYVQWYQQRPGSAPTILIYEDNKRPSGV | Anti-KRAS_V_{L} |
| | PDRFSGSIDSSSNSASLTISGLKTGDEADYYCQSYDDINHWVFGGGTKLTVLG | |
| 299 | NFMLTQPHSVSESPGKTVTISCTGSSGSIASNYVQWYQQRPGSAPTILIYEDNKRPSGV | Anti-KRAS_scFv |
| | PDRFSGSIDSSSNSASLTISGLKTGDEADYYCQSYDDINHWVFGGGTKLTVLGSRGGG | |
| | GSGGGGSGGGGSLEMAEVQLVQSGAEVKKPGASVKVSCKASGYTFTAYYLHWLRQ | |
| | APGQGLEWMGWMNTNNGATRYAQKFQDRVTMTRDTSINTAYMEMSGLSSDDTAM | |
| | YYCARGDISQDFADVWGQGTLVTVSS | |
| 300 | GYTFTAYY | Anti-KRAS_HCDR1 |
| 301 | MNTNNGAT | Anti-KRAS_HCDR2 |
| 302 | ARGDISQDFADV | Anti-KRAS_HCDR3 |
| 303 | EVQLVQSGAEVKKPGASVKVSCKASGYTFTAYYLHWLRQAPGQGLEWMGWMNTN | Anti-KRAS_V_{H} |
| | NGATRYAQKFQDRVTMTRDTSINTAYMEMSGLSSDDTAMYYCARGDISQDFADVW | |
| | GQGTLVTVSS | |
| 304 | SGSIASNY | Anti-KRAS_LCDR1 |
| 305 | EDN | Anti-KRAS_LCDR2 |
| 306 | QSYDDINHWV | Anti-KRAS_LCDR3 |
| 307 | NFMLTQPHSVSESPGKTVTISCTGSSGSIASNYVQWYQQRPGSAPTILIYEDNKRPSGV | Anti-KRAS_V_{L} |
| | PDRFSGSIDSSSNSASLTISGLKTGDEADYYCQSYDDINHWVFGGGTKLTVLG | |
| 308 | NFMLTQPHSVSESPGKTVTISCTGSSGSIASNYVQWYQQRPGSAPTILIYEDNKRPSGV | Anti-KRAS_scFv |
| | PDRFSGSIDSSSNSASLTISGLKTGDEADYYCQSYDDINHWVFGGGTKLTVLGSRGGG | |
| | GSGGGGSGGGGSLEMAEVQLVQSGAEVKKPGASVKVSCKASGYTFTAYYLHWLRQ | |
| | APGQGLEWMGWMNTNNGATRYAQKFQDRVTMTRDTSINTAYMEMSGLSSDDTAM | |
| | YYCARGDISQDFADVWGQGTLVTVSS | |
| 309 | GGSFSGYY | Anti-KRAS_HCDR1 |
| 310 | VNHSGNT | Anti-KRAS_HCDR2 |
| 311 | ARYFPPMIDV | Anti-KRAS_HCDR3 |
| 312 | QVQLQQWGAGLLKPSETLSLTCAVYGGSFSGYYWSWIRQSPGKGLEWIGEVNHSGN | Anti-KRAS_V_{H} |
| | TNYNPSLKSRVTISLDTSKNQFSLKLNSVTAADTAVYYCARYFPPMIDVWGQGTLVT | |
| | VSS | |
| 313 | SSNIENNY | Anti-KRAS_LCDR1 |
| 314 | DNN | Anti-KRAS_LCDR2 |
| 315 | GTWDSSLSAYV | Anti-KRAS_LCDR3 |
| 316 | GQSVVTQPPSVSAAPGQKVTISCSGSSSNIENNYVSWYQQLPGTAPKLLIYDNNKRPS | Anti-KRAS_V_{L} |
| | GIPDRFSGSKSGTSATLGITGLQTGDEADYYCGTWDSSLSAYVFGTGTKVTVLG | |
| 317 | QSVVTQPPSVSAAPGQKVTISCSGSSSNIENNYVSWYQQLPGTAPKLLIYDNNKRPSGI | Anti-KRAS_scFv |
| | PDRFSGSKSGTSATLGITGLQTGDEADYYCGTWDSSLSAYVFGTGTKVTVLGSRGGG | |
| | GSGGGGSGGGGSLEMAQVQLQQWGAGLLKPSETLSLTCAVYGGSFSGYYWSWIRQS | |
| | PGKGLEWIGEVNHSGNTNYNPSLKSRVTISLDTSKNQFSLKLNSVTAADTAVYYCAR | |
| | YFPPMIDVWGQGTLVTVSS | |
| 318 | GGSISSSSYY | Anti-KRAS_HCDR1 |
| 319 | INHSGST | Anti-KRAS_HCDR2 |
| 320 | ARYSHHVDSGGYDV | Anti-KRAS_HCDR3 |
| 321 | QLQLQESGPGLVKPSETLSLSCTVSGGSISSSSYYWGWIRQPPGKGLEWIGEINHSGST | Anti-KRAS_V_{H} |
| | NYNPSLKSRVTISVDTSKNQFSLKLSSVTAADTAVYYCARYSHHVDSGGYDVWGQG | |
| | TLVTVSS | |
| 322 | SSNIGNNY | Anti-KRAS_LCDR1 |
| 323 | DNN | Anti-KRAS_LCDR2 |
| 324 | GTWDSSLSAVV | Anti-KRAS_LCDR3 |
| 325 | QSVVTQPPSVSAAPGQKVTISCSGSSSNIGNNYVSWYQQLPRTAPRLLIYDNNKRPSGI | Anti-KRAS_V_{L} |
| | PDRFSASKSGTSATLGITGLQTGDEADYYCGTWDSSLSAVVFGGGTKLTVLG | |
| 326 | QSVVTQPPSVSAAPGQKVTISCSGSSSNIGNNYVSWYQQLPRTAPRLLIYDNNKRPSGI | Anti-KRAS_scFv |
| | PDRFSASKSGTSATLGITGLQTGDEADYYCGTWDSSLSAVVFGGGTKLTVLGSRGGG | |
| | GSGGGGSGGGGSLEMAQLQLQESGPGLVKPSETLSLSCTVSGGSISSSSYYWGWIRQP | |
| | PGKGLEWIGEINHSGSTNYNPSLKSRVTISVDTSKNQFSLKLSSVTAADTAVYYCARY | |
| | SHHVDSGGYDVWGQGTLVTVSS | |
| 327 | GGTFSSYG | Anti-KRAS_HCDR1 |
| 328 | IIPIFGTP | Anti-KRAS_HCDR2 |
| 329 | ARSYYGYFDG | Anti-KRAS_HCDR3 |
| 330 | EVQLVESGAEVKEPGSSVKVSCKASGGTFSSYGISWIRQAPGQGLEWMGEIIPIFGTPN | Anti-KRAS_V_{H} |
| | YAQKFQGRVTITADESTSTAYVELSSLRSDDTAVYYCARSYYGYFDGWGQGTLVTVS | |
| | S | |
| 331 | QDISNY | Anti-KRAS_LCDR1 |
| 332 | DAS | Anti-KRAS_LCDR2 |
| 333 | QQYKSYPLT | Anti-KRAS_LCDR3 |
| 334 | DIQMTQSPSSLSASVGDRVTITCQASQDISNYLNWYQQKPGKAPKLLIYDASNLETGV | Anti-KRAS_V_{L} |
| | PSRFSGSGSGTDFTFTISSLQPDDFATYYCQQYKSYPLTFGGGTKVEIKR | |
| 335 | DIQMTQSPSSLSASVGDRVTITCQASQDISNYLNWYQQKPGKAPKLLIYDASNLETGV | Anti-KRAS_scFv |
| | PSRFSGSGSGTDFTFTISSLQPDDFATYYCQQYKSYPLTFGGGTKVEIKRSRGGGGSGG | |
| | GGSGGGGSLEMAEVQLVESGAEVKEPGSSVKVSCKASGGTFSSYGISWIRQAPGQGL | |
| | EWMGEIIPIFGTPNYAQKFQGRVTITADESTSTAYVELSSLRSDDTAVYYCARSYYGY | |
| | FDGWGQGTLVTVSS | |
| 336 | GYTFTSYY | Anti-KRAS_HCDR1 |
| 337 | INPSGGST | Anti-KRAS_HCDR2 |
| 338 | ARSMYQYFLDS | Anti-KRAS_HCDR3 |
| 339 | EVQLVESGAEVKKPGASVKISCKASGYTFTSYYMHWVRQAPGQGLEWMGIINPSGGS | Anti-KRAS_V_{H} |
| | TSYAQKFQGRVTMTRDTSTSTVYMELSSLRSEDTAVYYCARSMYQYFLDSWGQGTL | |
| | VTVSS | |
| 340 | SSNIGAGYD | Anti-KRAS_LCDR1 |
| 341 | GNI | Anti-KRAS_LCDR2 |
| 342 | QSYDSNLSG | Anti-KRAS_LCDR3 |
| 343 | QSVVTQPPSVSGAPGQRVTISCTGSSSNIGAGYDVHWYQQLPGTAPKLLIYGNINRPSG | Anti-KRAS_V_{L} |
| | VPDRFSGSKSGTSASLAITGLQAEDEADYYCQSYDSNLSGYVFATGTKVTVLG | |
| 344 | QSVVTQPPSVSGAPGQRVTISCTGSSSNIGAGYDVHWYQQLPGTAPKLLIYGNINRPSG | Anti-KRAS_scFv |
| | VPDRFSGSKSGTSASLAITGLQAEDEADYYCQSYDSNLSGYVFATGTKVTVLGSRGG | |
| | GGSGGGGSGGGGSLEMAEVQLVESGAEVKKPGASVKISCKASGYTFTSYYMHWVRQ | |
| | APGQGLEWMGIINPSGGSTSYAQKFQGRVTMTRDTSTSTVYMELSSLRSEDTAVYYC | |
| | ARSMYQYFLDSWGQGTLVTVSS | |
| 345 | LTDAVKVMDL | PSA peptide can be |
| | | targeted by CAR |
| 346 | KLQCVDLHV | PSA peptide can be |
| | | targeted by CAR |
| 347 | VISNDVCAQV | PSA peptide can be |
| | | targeted by CAR |
| 348 | FLTPKKLQCV | PSA peptide can be |
| | | targeted by CAR |
| 349 | ALQCVDLHV | PSA peptide can be |
| | | targeted by CAR |
| 350 | KLACVDLHV | PSA peptide can be |
| | | targeted by CAR |
| 351 | KLQAVDLHV | PSA peptide can be |
| | | targeted by CAR |
| 352 | KLQCADLHV | PSA peptide can be |
| | | targeted by CAR |
| 353 | KLQCVALHV | PSA peptide can be |
| | | targeted by CAR |
| 354 | KLQCVDAHV | PSA peptide can be |
| | | targeted by CAR |
| 355 | KLQCVDLAV | PSA peptide can be |
| | | targeted by CAR |
| 356 | GGTFSSYA | Anti-PSA_HCDR1 |
| 357 | GFTFSSYA | Anti-PSA_HCDR1 |
| 358 | GYNFLNYG | Anti-PSA_HCDR1 |
| 359 | GYTFTGYY | Anti-PSA_HCDR1 |
| 360 | GGSFSDYY | Anti-PSA_HCDR1 |
| 361 | GYTFTSYG | Anti-PSA_HCDR1 |
| 362 | GGTFSSYA | Anti-PSA_HCDR1 |
| 363 | GYTFTSYG | Anti-PSA_HCDR1 |
| 364 | GYTFTSYY | Anti-PSA_HCDR1 |
| 365 | GYTFTGYF | Anti-PSA_HCDR1 |
| 366 | GGTFSSYA | Anti-PSA_HCDR1 |
| 367 | GYSFTSYW | Anti-PSA_HCDR1 |
| 368 | GYSFTSYW | Anti-PSA_HCDR1 |
| 369 | GYSFTSYR | Anti-PSA_HCDR1 |
| 370 | GYTFTNYG | Anti-PSA_HCDR1 |
| 371 | IIPIPGIT | Anti-PSA_HCDR2 |
| 372 | ISGSGGST | Anti-PSA_HCDR2 |
| 373 | ISTYTGNT | Anti-PSA_HCDR2 |
| 374 | FDPEDGET | Anti-PSA_HCDR2 |
| 375 | INHSGGT | Anti-PSA_HCDR2 |
| 376 | ISAYNGNT | Anti-PSA_HCDR2 |
| 377 | INPNSGGT | Anti-PSA_HCDR2 |
| 378 | ISAYNGNT | Anti-PSA_HCDR2 |
| 379 | FDPEDGET | Anti-PSA_HCDR2 |
| 380 | FDPEDGET | Anti-PSA_HCDR2 |
| 381 | IIPILGIA | Anti-PSA_HCDR2 |
| 382 | IYPGDSDT | Anti-PSA_HCDR2 |
| 383 | IYPGDSDT | Anti-PSA_HCDR2 |
| 384 | IDPSDSYT | Anti-PSA_HCDR2 |
| 385 | ARSYKWGSSLVDA | Anti-PSA_HCDR3 |
| 386 | ARNYYSQYWMMDL | Anti-PSA_HCDR3 |
| 387 | ARSSEYYTWDH | Anti-PSA_HCDR3 |
| 388 | ARYGFDY | Anti-PSA_HCDR3 |
| 389 | ARYNEYGSGYDK | Anti-PSA_HCDR3 |
| 390 | ARSSQYYVWDS | Anti-PSA_HCDR3 |
| 391 | ARWSYYYFQQFWSLDG | Anti-PSA_HCDR3 |
| 392 | ARTNYNKYDI | Anti-PSA_HCDR3 |
| 393 | ARYSYDY | Anti-PSA_HCDR3 |
| 394 | ARYSYDL | Anti-PSA_HCDR3 |
| 395 | ARVSQPVYGSSTYDI | Anti-PSA_HCDR3 |
| 396 | ARLVVPDAFDI | Anti-PSA_HCDR3 |
| 397 | ARWGSRGFLDAFDI | Anti-PSA_HCDR3 |
| 398 | ARWGLSWDGWGVTDY | Anti-PSA_HCDR3 |
| 399 | ARYNYDT | Anti-PSA_HCDR3 |
| 400 | ARSFGAGYDS | Anti-PSA_HCDR3 |
| 401 | ARYPWDH | Anti-PSA_HCDR3 |
| 402 | ARSSYYGYLSDG | Anti-PSA_HCDR3 |
| 403 | EVQLVQSGAEVKRPGSSVKVSCKASGGTFSSYAINWVRRAPGQGLEWMGKIIPIPGIT | Anti-PSA_V_{H} |
| | NYAQKFQDRVTFTADTSTNIAYMELSSLRSEDTAMYYCARSYKWGSSLVDAWGQGT | |
| | LVTVSS | |
| 404 | EVQLVESGGGLVQPGGSLRLSCAASGFTFSSYAMSWVRQAPGKGLEWVSAISGSGGS | Anti-PSA_V_{H} |
| | TYYADSVKGRFTISRDNSKNTLYLQMNSLRAEDTAVYYCARNYYSQYWMMDLWGQ | |
| | GTLVTVSS | |
| 405 | QVQLVQSGAEVKKPGDSVKVSCKPSGYNFLNYGINWVRQAPGQGLEWMGWISTYT | Anti-PSA_V_{H} |
| | GNTNYAQKLQGRVTFTTDTSTSTAYMEMRSLRSDDTAVYYCARSSEYYTWDHWGQ | |
| | GTLVTVSS | |
| 406 | EVQLVQSGAEVKKPGASVKVSCKASGYTFTGYYIHWVRQAPGQGLEWMGGFDPED | Anti-PSA_V_{H} |
| | GETIYAQKFQDRVTMTADTSTDTAYMELSSLRSEDTAVYYCARYGFDYWGQGTLVT | |
| | VSS | |
| 407 | QVQLQQWGAGLLKPSETLSLTCAVKGGSFSDYYWSWIRQPPGKGLEWIGEINHSGGT | Anti-PSA_V_{H} |
| | NYNPSLKSRVTISVDTSKNQFSLKLLSVTAADTAVYYCARYNEYGSGYDKWGQGTL | |
| | VTVSS | |
| 408 | QVQLVQSGAEVKKPGASVKVSCKASGYTFTSYGISWVRQAPGQGLEWMGWISAYN | Anti-PSA_V_{H} |
| | GNTNYAQKLQGRVTMTTDTSTSTAYMELRSLRSDDTAVYYCARSSQYYVWDSWGQ | |
| | GTLVTVSS | |
| 409 | QVQLVQSGAEVKKPGSSVKVSCKASGGTFSSYAISWVRQAPGQGLEWMGWINPNSG | Anti-PSA_V_{H} |
| | GTNYAQKFQGRVTMTRDTSISTAYMELSRLRSDDTAVYYCARWSYYYFQQFWSLDG | |
| | WGQGTLVTVSS | |
| 410 | QMQLVQSGSEVKKPGASVKVSCKASGYTFTSYGISWVRQAPGQGLEWMGWISAYN | Anti-PSA_V_{H} |
| | GNTDYVQKLQGRVTMTTDTSTNTAYMELGSLGSDDTAVYYCARTNYNKYDIWGQG | |
| | TLVTVSS | |
| 411 | QVQLVQSGAEVKKPGASVKVSCKASGYTFTSYYMHWVRQAPGQGLEWMGGFDPED | Anti-PSA_V_{H} |
| | GETIYAQKFQGRVTMTEDTSTDTAYMGLSSLRSEDTAVYYCARYSYDYWGQGTLVT | |
| | VSS | |
| 412 | EVQLVQSGAEVKKPGASVKVSCKASGYTFTGYFMHWVRQAPGQGLEWMGGFDPED | Anti-PSA_V_{H} |
| | GETIYAQKFQGRVTMTEDTSTDTAYMELSSLRSEDTAVYYCARYSYDLWGQGTLVT | |
| | VSS | |
| 413 | QVQLVQSGAEVKKPGSSVKVSCKASGGTFSSYAISWVRQAPGQGLEWMGRIIPILGIA | Anti-PSA_V_{H} |
| | NYAQKFQGRVTITADKSTSTAYMELSSLRSEDTAVYYCARVSQPVYGSSTYDIWGQG | |
| | TLVTVSS | |
| 414 | QMQLVQSGAEVKKPGESLKISCKGSGYSFTSYWIGWVRQMPGKGLEWMGIIYPGDS | Anti-PSA_V_{H} |
| | DTRYSPSFQGQVTISADKSISTAYLQWSSLKASDTAMYYCARLVVPDAFDIWGQGTM | |
| | VTVSS | |
| 415 | QVQLVQSGAEVKKPGESLKISCKGSGYSFTSYWIGWVRQMPGKGLEWMGIIYPGDSD | Anti-PSA_V_{H} |
| | TRYSPSFQGQVTISADKSISTAYLQWSSLKASDTAMYYCARWGSRGFLDAFDIWGQG | |
| | TMVTVSS | |
| 416 | EVQLVQSGAEVKKPGESLKISCKGSGYSFTSYRIGWVRQMPGKGLEWMGIIYPGDSD | Anti-PSA_V_{H} |
| | TRYSPSFQGQVTISADKSISTAYLQWSSLKASDTAMYYCARWGLSWDGWGVTDYW | |
| | GQGTLVTVSS | |
| 417 | QVQLVQSGAEVKKPGASVKVSCKASGYTFTSYYMHWVRQAPGKGLEWMGGFDPED | Anti-PSA_V_{H} |
| | GETIYAQKFQGRVTMTEDTSTDTAYMELSSLRSEDTAVYYCARYNYDTWGQGTLVT | |
| | VSS | |
| 418 | EVQLVQSGAEVKKPGESLKISCKGSGYSFTSYWIGWVRQMPGKGLEWMGRIDPSDSY | Anti-PSA_V_{H} |
| | TNYSPSFQGHVTISADKSISTAYLQWSSLKASDTAMYYCARSFGAGYDSWGQGTLVT | |
| | VSS | |
| 419 | QVQLVQSGAEVKKPGASVKVSCKASGYTFTSYYMHWVRQAPGKGLEWMGGFDPED | Anti-PSA_V_{H} |
| | GETIYAQKFQGRVTMTEDTSTDTAYMELSSLRSEDTAVYYCARYPWDHWGQGTLVT | |
| | VSS | |
| 420 | QVQLVQSGAEVKKPGASVKVSCKTSGYTFTNYGISWVRQAPGQGLEWMGWISAYN | Anti-PSA_V_{H} |
| | GNTNYAQNLQGRVTMTTDTSTSTAYMELRSLRSDDTAVYYCARSSYYGYLSDGWG | |
| | QGTLVTVSS | |
| 421 | NSNIGSNT | Anti-PSA_LCDR1 |
| 422 | SSNFGAGYD | Anti-PSA_LCDR1 |
| 423 | SSNIGAGYD | Anti-PSA_LCDR1 |
| 424 | TGAVTSGYY | Anti-PSA_LCDR1 |
| 425 | SYNIGNNY | Anti-PSA_LCDR1 |
| 426 | SSNFGAGYD | Anti-PSA_LCDR1 |
| 427 | SSNIGSNT | Anti-PSA_LCDR1 |
| 428 | SSNIGAGYD | Anti-PSA_LCDR1 |
| 429 | TGAVTSGYY | Anti-PSA_LCDR1 |
| 430 | TGAVTSGYY | Anti-PSA_LCDR1 |
| 431 | SSNLGSNS | Anti-PSA_LCDR1 |
| 432 | SSNIGNNY | Anti-PSA_LCDR1 |
| 433 | SSNIGNNY | Anti-PSA_LCDR1 |
| 434 | QSISSY | Anti-PSA_LCDR1 |
| 435 | TGTVTSTYY | Anti-PSA_LCDR1 |
| 436 | SSDVGGYNY | Anti-PSA_LCDR1 |
| 437 | SSNIGTNY | Anti-PSA_LCDR1 |
| 438 | SNN | Anti-PSA_LCDR2 |
| 439 | GDT | Anti-PSA_LCDR2 |
| 440 | GNS | Anti-PSA_LCDR2 |
| 441 | TTG | Anti-PSA_LCDR2 |
| 442 | DNN | Anti-PSA_LCDR2 |
| 443 | GNS | Anti-PSA_LCDR2 |
| 444 | STS | Anti-PSA_LCDR2 |
| 445 | DNH | Anti-PSA_LCDR2 |
| 446 | DNY | Anti-PSA_LCDR2 |
| 447 | DND | Anti-PSA_LCDR2 |
| 448 | AAS | Anti-PSA_LCDR2 |
| 449 | DVS | Anti-PSA_LCDR2 |
| 450 | STN | Anti-PSA_LCDR2 |
| 451 | ATWDDSLNGPV | Anti-PSA_LCDR3 |
| 452 | QSYDTSLSGSV | Anti-PSA_LCDR3 |
| 453 | QSYDSSLSGWV | Anti-PSA_LCDR3 |
| 454 | LLYSGGVWV | Anti-PSA_LCDR3 |
| 455 | GTWESSLSAYV | Anti-PSA_LCDR3 |
| 456 | QSYDSSLSGWV | Anti-PSA_LCDR3 |
| 457 | AAWDDSLNGRWV | Anti-PSA_LCDR3 |
| 458 | QSYDSSLSEV | Anti-PSA_LCDR3 |
| 459 | LLYYGGAYV | Anti-PSA_LCDR3 |
| 460 | LLYYGGAQWV | Anti-PSA_LCDR3 |
| 461 | AAWDDSLNSVV | Anti-PSA_LCDR3 |
| 462 | GTWDSSLSAGV | Anti-PSA_LCDR3 |
| 463 | GTWDSSLSSGV | Anti-PSA_LCDR3 |
| 464 | QQSYSTPFT | Anti-PSA_LCDR3 |
| 465 | LVFYGGVWV | Anti-PSA_LCDR3 |
| 466 | SSYTSSSRYV | Anti-PSA_LCDR3 |
| 467 | LLYYGGQGV | Anti-PSA_LCDR3 |
| 468 | AAWDDSLSGLYV | Anti-PSA_LCDR3 |
| 469 | QSVLTQPPSASGTPGQRVTLSCSGSNSNIGSNTVNWYQQLPGTNPKLLIYSNNQRPSG | Anti-PSA_V_{L} |
| | VPDRFSGSKSGTSASLAISGLQSEDEADYYCATWDDSLNGPVFGGGTKLTVLG | |
| 470 | QSVLTQPPSVSGAPGQRVTISCTGSSSNFGAGYDVHWYQQLPGAAPKLLIYGDTNRPS | Anti-PSA_V_{L} |
| | GVPDRFSGSKSGTSASLAITGLQAEDEADYYCQSYDTSLSGSVFGGGTKLTVLG | |
| 471 | QSVLTQPPSVSGAPGQRVTISCTGSSSNIGAGYDVHWYQQLPGTAPKLLIYGNSNRPS | Anti-PSA_V_{L} |
| | GVPDRFSGSKSGTSASLAITGLQAEDEADYYCQSYDSSLSGWVFGGGTKLTVLG | |
| 472 | QAVVTQEPSLTVSPGGTVTLTCASSTGAVTSGYYPNWFQLKPGQAPRALIYTTGKKH | Anti-PSA_V_{L} |
| | SWAPARFSGSLLGGKAALTLSGVQPEDEAEYYCLLYSGGVWVFGGGTKLTVLG | |
| 473 | QSVLTQPPSVSAAPGQKVTISCSGSSYNIGNNYVSWYQQLPGTAPKLLIYDNNKRPSGI | Anti-PSA_V_{L} |
| | PDRFSGSKSGTSATLGITGLQTGDEAEYYCGTWESSLSAYVFGTGTKVTVLG | |
| 474 | QSVVTQPPSVSGAPGQRVTISCTGSSSNFGAGYDVHWYQQLPGTAPKLLIYGNSNRPS | Anti-PSA_V_{L} |
| | GVPDRFSGSKSGTSASLAITGLQAEDEADYYCQSYDSSLSGWVFGGGTKLTVLG | |
| 475 | QAVLTQPPSASGTPGQRVTISCSGSSSNIGSNTVNWYQQLPGTAPKLLIYSNNQRPSGV | Anti-PSA_V_{L} |
| | PDRFSGSKSGTSASLAISGLQSEDEADYYCAAWDDSLNGRWVFGGGTKLTVLG | |
| 476 | QSVLTQPPSVSGAPGQRVTISCTGSSSNIGAGYDVHWYQQLPGTAPKLLIYGNSNRPS | Anti-PSA_V_{L} |
| | GVPDRFSGSKSGTSASLAITGLQAEDEADYYCQSYDSSLSEVFGTGTKVTVLG | |
| 477 | QAVVTQEPSLTVSPGGTVTLTCASSTGAVTSGYYPNWFQQKPGQAPRALIYSTSNKHS | Anti-PSA_V_{L} |
| | WTPARFSGSLLGGKAALTLSGVQPEDEAEYYCLLYYGGAYVFGTGTKVTVLG | |
| 478 | QTVVTQEPSLTVSPGGTVTLTCASSTGAVTSGYYPNWFQQKPGQAPRALIYSTSNKHS | Anti-PSA_V_{L} |
| | WTPARFSGSLLGGKAALTLSGVQPEDEAEYYCLLYYGGAQWVFGGGTKLTVLG | |
| 479 | QAVLTQPPSASGTPGQRVTISCSGRSSNLGSNSVNWYQQVPGTAPKLLIFDNHQRPSG | Anti-PSA_V_{L} |
| | VPDRFSGSKSGTSASLAISGLRSEDETDYYCAAWDDSLNSVVFGGGTKLTVLG | |
| 480 | QSVVTQPPSVSAAPGQKVTISCSGSSSNIGNNYVSWYQQLPGTAPKLLIYDNYKRPSGI | Anti-PSA_V_{L} |
| | PDRFSGSKSGTSATLGITGLQTGDEADYYCGTWDSSLSAGVFGGGTKLTVLG | |
| 481 | QSVVTQPPSVSAAPGQRVTISCSGSSSNIGNNYVSWYQQLPGAAPRLLIYDNDKRPSGI | Anti-PSA_V_{L} |
| | PDRFSGSKSGTSATLGITGLQTGDEADYYCGTWDSSLSSGVFGGGTKLTVLG | |
| 482 | DIQLTQSPSSLSASVGDRVTITCRASQSISSYLNWYQQKPGKAPKLLIYAASSLQSGVPS | Anti-PSA_V_{L} |
| | RFSGSGSGTDFTLTISSLQPEDFATYYCQQSYSTPFTFGPGTKVDIKR | |
| 483 | QAVVTQEPSLTVSPGGTVTLTCASSTGTVTSTYYPNWFQQKPGQAPRALIYSTSNRHS | Anti-PSA_V_{L} |
| | WTPARFSGSLLGGKAALTVSGVQPDDEAEYYCLVFYGGVWVFGGGTKLTVLG | |
| 484 | QSALTQPASVSGSPGQSITISCTGTSSDVGGYNYVSWYQQHPGKAPKLMIYDVSKRPS | Anti-PSA_V_{L} |
| | GVSNRFSGSKSGNTASLTISGLQAEDEADYYCSSYTSSSRYVFGTGTKVTVLG | |
| 485 | QAVVTQEPSLTVSPGGTVTLTCASSTGAVTSGYYPNWFQQKPGQAPRPLIYSTNNKHS | Anti-PSA_V_{L} |
| | WTPARFSGSLLGGKAALTLSGVQPEDEAEYYCLLYYGGQGVFGGGTKLTVLG | |
| 486 | LPVLTQPPSASGTPGQRVTISCSGSSSNIGTNYVYWYQQLPGTAPKLLIYSNNQRPSGV | Anti-PSA_V_{L} |
| | PDRFSGSKSGTSASLAISGLRSEDEADYYCAAWDDSLSGLYVFGTGTKVTVLG | |
| 487 | QSVLTQPPSVSGAPGQRVTISCTGSSSNIGAGYDVHWYQQLPGTAPKLLIYGNSNRPS | PSMA-A scFv |
| | GVPDRFSGSKSGTSASLAITGLQAEDEADYYCQSYDSSLSGYVFGTGTKVTVLGSRGG | |
| | GGSGGGGSGGGGSLEMAEVQLVQSGAEVKKPGESLKISCKGSGYSFTSYWIGWVRQ | |
| | MPGKGLEWMGIIYPGDSDTRYSPSFQGQVTISADKSISTAYLQWSSLKASDTAMYYC | |
| | ARSMGSSLYASSDVWGQGTLVTVSS | |
| 488 | QAVLTQPPSASGTPGQRVTISCSGSSSNIGSNTVNWYQQLPGTAPKLLMYSNNQRPSG | PSMA-B scFv |
| | VPDRFSGSKSGTSASLAISGLQSEDEADYYCAAWDDSLNGYVFGTGTKVTVLGSRGG | |
| | GGSGGGGSGGGGSLEMAEVQLVQSGAEMKKPGESLKISCKGSGYNFASYWVGWVR | |
| | QMPGKGLEWMGTIYPDDSDTRYGPAFQGQVTISADKSISTAYLQWSSLKASDTAMY | |
| | YCARDSYYGIDVWGQGTLVTVSS | |
| 489 | KNDAPVVQEPRRLSFRSTIYGSR | ROR1 peptide can be |
| | | targeted by CAR |
| 490 | AANCIRIGIPMADPI | ROR1 peptide can be |
| | | targeted by CAR |
| 491 | SSTGVLFVKFGPPPTASPG | ROR1 peptide can be |
| | | targeted by CAR |
| 492 | SNPMILMRLKLPNCE | ROR1 peptide can be |
| | | targeted by CAR |
| 493 | GGSLSSHGVS | Anti-ROR1_HCDR1 |
| 494 | RIIPMFGVTDYAQKFQD | Anti-ROR1_HCDR2 |
| 495 | ESRGATFEY | Anti-ROR1_HCDR3 |
| 496 | QVQLVQSGTEVKKPGSSVKVSCQASGGSLSSHGVSWLRQAPGQGLEWVGRIIPMFGV | Anti-ROR1_V_{H} |
| | TDYAQKFQDRVTITADKSTSTVYMELISLGSDDTAVYFCARESRGATFEYWGQGTLV | |
| | TVSS | |
| 497 | RASQSVSSSYLA | Anti-ROR1_LCDR1 |
| 498 | GASSRAT | Anti-ROR1_LCDR2 |
| 499 | QQYGSS | Anti-ROR1_LCDR3 |
| 500 | EIVLTQSPGTLSLSPGERATLSCRASQSVSSSYLAWYQQKPGQAPRLLIYGASSRATGIP | Anti-ROR1_V_{L} |
| | DRFSGSGSGTDFTLTISRLEPEDFAVYYCQQYGSSFGPGTKVDIKR | |
| 501 | GGSISSSSYYWG | Anti-ROR1_HCDR1 |
| 502 | SIYYSGSTYYNPSLKS | Anti-ROR1_HCDR2 |
| 503 | HDGTDAFDI | Anti-ROR1_HCDR3 |
| 504 | QLQLQESGPGLVKPSETLSLTCTVSGGSISSSSYYWGWIRQPPGKGLEWIGSIYYSGST | Anti-ROR1_V_{H} |
| | YYNPSLKSRVTISVDTSKNQFSLKLGSVTAADTAVYYCARHDGTDAFDIWGQGTTVT | |
| | VSS | |
| 505 | TGTSSDFGDYDYVS | Anti-ROR1_LCDR1 |
| 506 | DVSDRPS | Anti-ROR1_LCDR2 |
| 507 | SSLTTSSTLV | Anti-ROR1_LCDR3 |
| 508 | QSVLTQPASVSGSPGQSITISCTGTSSDFGDYDYVSWYQQHPGKAPKLMIYDVSDRPS | Anti-ROR1_V_{L} |
| | GVSNRFSGSKSGNTASLTISGLQAEDEADYFCSSLTTSSTLVFGGGTKLTVLG | |
| 509 | QLSLLMWIT | NY-ESO-1 peptide can |
| | | be targeted by CAR |
| 510 | SLLMWITQC | NY-ESO-1 peptide can |
| | | be targeted by CAR |
| 511 | SLLMWITQCFL | NY-ESO-1 peptide can |
| | | be targeted by CAR |
| 512 | SLLMWITQV | NY-ESO-1 peptide can |
| | | be targeted by CAR |
| 513 | ALLMWITQC | NY-ESO-1 peptide can |
| | | be targeted by CAR |
| 514 | SALMWITQC | NY-ESO-1 peptide can |
| | | be targeted by CAR |
| 515 | SLAMWITQC | NY-ESO-1 peptide can |
| | | be targeted by CAR |
| 516 | SLLAWITQC | NY-ESO-1 peptide can |
| | | be targeted by CAR |
| 517 | SLLMAITQC | NY-ESO-1 peptide can |
| | | be targeted by CAR |
| 518 | SLLMWATQC | NY-ESO-1 peptide can |
| | | be targeted by CAR |
| 519 | SLLMWIAQC | NY-ESO-1 peptide can |
| | | be targeted by CAR |
| 520 | GDTFSSYS | Anti-NY-ESO- |
| | | 1_HCDR1 |
| 521 | FIPNLNKG | Anti-NY-ESO- |
| | | 1_HCDR2 |
| 522 | ARDWSYSIDY | Anti-NY-ESO- |
| | | 1_HCDR3 |
| 523 | QVQLVQSGAEVKKPGSSVKVSCKASGDTFSSYSISWVRQAPGQGLEWMGRIIPILGIA | Anti-NY-ESO-1_V_{H} |
| | NYAQKYQGRVTLSADKSTSTSYMELNSLRSEDTAVYYCARDWSYSIDYWGQGTLVT | |
| | VSS | |
| 524 | SSNIGNNY | Anti-NY-ESO- |
| | | 1_LCDR1 |
| 525 | DNN | Anti-NY-ESO- |
| | | 1_LCDR2 |
| 526 | GTWDSSLSAWV | Anti-NY-ESO- |
| | | 1_LCDR3 |
| 527 | QSVVTQPPSVSAAPGQKVTISCSGSSSNIGNNYVSWYQQLPGTAPKLLIYDNNKRPSGI | Anti-NY-ESO-1_V_{L} |
| | PDRFSGSKSGTSATLGITGLQTGDEADYYCGTWDSSLSAWVFGGGTKLTVLG | |
| 528 | GYTFTSYG | Anti-NY-ESO- |
| | | 1_HCDR1 |
| 529 | ISAYNGNT | Anti-NY-ESO- |
| | | 1_HCDR2 |
| 530 | ARYSGYYAGDS | Anti-NY-ESO- |
| | | 1_HCDR3 |
| 531 | QVQLVQSGAEVKKPGASVKVSCKASGYTFTSYGISWVRQAPGQGLEWMGWISAYN | Anti-NY-ESO-1_V_{H} |
| | GNTNYAQKLQGRVTMTTDTSTSTAYMELRSLRSDDTAVYYCARYSGYYAGDSWGQ | |
| | GTLVTVSS | |
| 532 | SSNIGAGYD | Anti-NY-ESO- |
| | | 1_LCDR1 |
| 533 | GDT | Anti-NY-ESO- |
| | | 1_LCDR2 |
| 534 | QSYDSNLYTYV | Anti-NY-ESO- |
| | | 1_LCDR3 |
| 535 | QSVLTQPPSVSGAPGQRVTISCTGSSSNIGAGYDVHWYQQLPGTAPKLLIYGDTNRPS | Anti-NY-ESO-1_V_{L} |
| | GVPDRISGSKSGTSASLAITGLQAEDEADYYCQSYDSNLYTYVFGTGTKVTVLG | |
| 536 | GYTFTSYG | Anti-NY-ESO- |
| | | 1_HCDR1 |
| 537 | FIPNLNKG | Anti-NY-ESO- |
| | | 1_HCDR2 |
| 538 | ARGDYGSDQ | Anti-NY-ESO- |
| | | 1_HCDR3 |
| 539 | EVQLVESGAEVKRPGASVKVSCKASGYTFTSYGISWVRQAPGQGLEWMGRFIPNLNK | Anti-NY-ESO-1_V_{H} |
| | GNSAHKFEGRVSFTADKFTNTAYMELRGLKSDDTAVYYCARGDYGSDQWGQGTLV | |
| | TVSS | |
| 540 | SSNIGAGY | Anti-NY-ESO- |
| | | 1_LCDR1 |
| 541 | GNS | Anti-NY-ESO- |
| | | 1_LCDR2 |
| 542 | QSYDSSLSGSWV | Anti-NY-ESO- |
| | | 1_LCDR3 |
| 543 | QSVLTQPPSVSGAPGQRVTISCTGSSSNIGAGYDVHWYQQLPGTAPKLLIYGNSNRPS | Anti-NY-ESO-1_V_{L} |
| | GVPDRFSGSKSGTSASLAITGLQAEDEADYYCQSYDSSLSGSWVFGGGTKLTVLG | |
| 544 | GGTFSSYA | Anti-NY-ESO- |
| | | 1_HCDR1 |
| 545 | IIPIFGTA | Anti-NY-ESO- |
| | | 1_HCDR2 |
| 546 | ARYDSYVYDE | Anti-NY-ESO- |
| | | 1_HCDR3 |
| 547 | EVQLVQSGAEVKKPGSSVKVSCKASGGTFSSYAISWVRQAPGQGLEWMGGIIPIFGTA | Anti-NY-ESO-1_V_{H} |
| | NYAQKFQGRVTITADESTSTAYMELSSLRSEDTAVYYCARYDSYVYDEWGQGTLVT | |
| | VSS | |
| 548 | GSNIGAGYD | Anti-NY-ESO- |
| | | 1_LCDR1 |
| 549 | GNS | Anti-NY-ESO- |
| | | 1_LCDR2 |
| 550 | QSYDSSLSGWGI | Anti-NY-ESO- |
| | | 1_LCDR3 |
| 551 | QSVVTQPPSLSGAPGQRVTISCNGSGSNIGAGYDVHWYQQLPGTAPKLLIYGNSNRPS | Anti-NY-ESO-1 V_{L} |
| | GVPDRFSGSKSGTSASLAITGLQAEDEADYYCQSYDSSLSGWGIFGGGTKLTVLG | |
| 552 | GYTFTKYG | Anti-NY-ESO- |
| | | 1_HCDR1 |
| 553 | ISADSGKT | Anti-NY-ESO- |
| | | 1_HCDR2 |
| 554 | ARDDDS | Anti-NY-ESO- |
| | | 1_HCDR3 |
| 555 | QVQLVQSGPEVKKPGASMKVSCKASGYTFTKYGISWVRQAPGQGLEWMGWISADS | Anti-NY-ESO-1_V_{H} |
| | GKTSYAQNLQGRVSLTIDTSTATAYMELRSLRSDDTAVYYCARDDDSWGQGTLVTV | |
| | SS | |
| 556 | SSNIGNNY | Anti-NY-ESO- |
| | | 1_LCDR1 |
| 557 | DNN | Anti-NY-ESO- |
| | | 1_LCDR2 |
| 558 | GTWDSSLSAEV | Anti-NY-ESO- |
| | | 1_LCDR3 |
| 559 | QSVLTQPPSVSAAPGQKVTISCSGSSSNIGNNYVSWYQQLPGTAPKLLIYDNNKRPSGI | Anti-NY-ESO-1_V_{L} |
| | PDRFSGSKSGTSATLGITGLQTGDEADYYCGTWDSSLSAEVFGTGTKVTVLG | |
| 560 | GYTLTDLP | Anti-NY-ESO- |
| | | 1_HCDR1 |
| 561 | FDPEDGEI | Anti-NY-ESO- |
| | | 1_HCDR2 |
| 562 | ARYVPYVSYSDS | Anti-NY-ESO- |
| | | 1_HCDR3 |
| 563 | EVQLVQSGAEVKKPGASVKVSCKVSGYTLTDLPMHWVRQAPGKGLEWMGGFDPED | Anti-NY-ESO-1_V_{H} |
| | GEIIYAQKFQGRVTMTEDTFTDTAYVELSSLRSEDTAVYYCARYVPYVSYSDSWGQG | |
| | TLVTVSS | |
| 564 | QSLLHSNGYNY | Anti-NY-ESO- |
| | | 1_LCDR1 |
| 565 | LGS | Anti-NY-ESO- |
| | | 1_LCDR2 |
| 566 | MQALQTPYT | Anti-NY-ESO- |
| | | 1_LCDR3 |
| 567 | DVVMTQSPLSLPVTPGEPASISCRSSQSLLHSNGYNYLDWYLQKPGQSPQLLIYLGSN | Anti-NY-ESO-1_V_{L} |
| | RASGVPDRFSGSGSGTDFTLKISRVEAEDVGVYYCMQALQTPYTFGQGTKLEIKR | |
| 568 | GGTFSSYA | Anti-NY-ESO- |
| | | 1_HCDR1 |
| 569 | IIPIFGTA | Anti-NY-ESO- |
| | | 1_HCDR2 |
| 570 | ARSYWSWTPYDP | Anti-NY-ESO- |
| | | 1_HCDR3 |
| 571 | EVQLVQSGAEVKKPGASVKVSCKASGGTFSSYAISWVRQAPGQGLEWMGGIIPIFGT | Anti-NY-ESO-1_V_{H} |
| | ANYAQKFQGRVTITADKSTSTAYMELSSLRSEDTAVYYCARSYWSWTPYDPWGQGT | |
| | LVTVSS | |
| 572 | NIGSKS | Anti-NY-ESO- |
| | | 1_LCDR1 |
| 573 | YDS | Anti-NY-ESO- |
| | | 1_LCDR2 |
| 574 | QVWDSSSDHYV | Anti-NY-ESO- |
| | | 1_LCDR3 |
| 575 | LPVLTQPPSVSVAPGKTARITCGGNNIGSKSVHWYQQKPGQAPVLVIYYDSDRPSGIPE | Anti-NY-ESO-1_V_{L} |
| | RFSGSNSGNTATLTISRVEAGDEADYYCQVWDSSSDHYVFGTGTKVTVLG | |
| 576 | VLDGLDVLL | PRAME peptide can be |
| | | targeted by CAR |
| 577 | SLYSFPEPEA | PRAME peptide can be |
| | | targeted by CAR |
| 578 | ALYVDSLFFL | PRAME peptide can be |
| | | targeted by CAR |
| 579 | SLLQHLIGL | PRAME peptide can be |
| | | targeted by CAR |
| 580 | NLTHVLYPV | PRAME peptide can be |
| | | targeted by CAR |
| 581 | GGTFSSYA | Anti-PRAME_HCDR1 |
| 582 | IIPILGIA | Anti-PRAME_HCDR2 |
| 583 | ARHYGQWWDY | Anti-PRAMEHCDR3 |
| 584 | QVQLVQSGAEVRKPGASVKVSCKASGGTFSSYAISWVRQAPGQGLEWMGRIIPILGIA | Anti-PRAME_V_{H} |
| | NYAQKFQGRVTITADKSTSTAYMELSSLRSEDTAVYYCARHYGQWWDYWGQGTLV | |
| | TVSS | |
| 585 | SSNIGSNT | Anti-PRAME_LCDR1 |
| 586 | SNN | Anti-PRAME_LCDR2 |
| 587 | AAWDDSLNGSYV | Anti-PRAME_LCDR3 |
| 588 | QAVLTQPPSASGTPGQRVTISCSGSSSNIGSNTVNWYQQLPGTAPKLLIYSNNQRPSGV | Anti-PRAME_V_{L} |
| | PDRFSGSKSGTSASLAISGLQSEDEADYYCAAWDDSLNGSYVFGTGTKVTVLG | |
| 589 | GGTFSSHP | Anti-PRAME_HCDR1 |
| 590 | IIPMLDIP | Anti-PRAME_HCDR2 |
| 591 | ARGLYYYDY | Anti-PRAME_HCDR3 |
| 592 | QVQLVQSGAEVKKPGSSVKVSCKASGGTFSSHPISWVRQAPGQGLEWMGRIIPMLDIP | Anti-PRAME_V_{H} |
| | NNAQKFQGRVTITADKSTDTAYLELSSLTSEDTAVYYCARGLYYYDYWGQGTLVTV | |
| | SS | |
| 593 | TSNIGAGFD | Anti-PRAME_LCDR1 |
| 594 | GNT | Anti-PRAME_LCDR2 |
| 595 | QSYDRSLSTIL | Anti-PRAME_LCDR3 |
| 596 | QSVVTQPPAVSGALGQRVTISCTGTTSNIGAGFDVHWYQQRPGAAPKLLISGNTHRPS | Anti-PRAME_V_{L} |
| | GVPDRISGSKSGTLASLAITGLQAEDEADYYCQSYDRSLSTILFGGGTKLTVLG | |
| 597 | GGTFSSYA | Anti-PRAME_HCDR1 |
| 598 | IIPIFGIA | Anti-PRAME_HCDR2 |
| 599 | ARSMWYMDS | Anti-PRAME_HCDR3 |
| 600 | EVQLVESGAEVKKPGSSVKVSCKASGGTFSSYAISWVRQAPGQGLEWMGRIIPIFGIA | Anti-PRAME_V_{H} |
| | NYAQKFQGRVTITADKSTSTAYMELSSLRSEDTAVYYCARSMWYMDSWGQGTLVT | |
| | VSS | |
| 601 | SSNIGAGFD | Anti-PRAME_LCDR1 |
| 602 | GNS | Anti-PRAME_LCDR2 |
| 603 | QSYDSSLSGYV | Anti-PRAME_LCDR3 |
| 604 | QSVLTQPPSVSGAPGQRVTISCTGSSSNIGAGFDVHWYQQLPGTAPKLLIFGNSNRPSG | Anti-PRAME_V_{L} |
| | VPDRFSGSKSGTSASLAITGLQAEDEADYYCQSYDSSLSGYVFGSGTKVTVLG | |
| 605 | GYTFSSYG | Anti-PRAME_HCDR1 |
| 606 | ISPYNGNT | Anti-PRAME_HCDR2 |
| 607 | ARYSGYYYVDY | Anti-PRAME_HCDR3 |
| 608 | QVQLVQSGAEVKKPGASVKVSCKASGYTFSSYGISWVRQAPGQGLEWMGWISPYNG | Anti-PRAME_V_{H} |
| | NTNYAQNLQGRVTMTTDTSTTTAYMELRSLTSDDTAVYYCARYSGYYYVDYWGQG | |
| | TLVTVSS | |
| 609 | QSISSY | Anti-PRAME_LCDR1 |
| 610 | AAS | Anti-PRAME_LCDR2 |
| 611 | QQSYSTPRT | Anti-PRAME_LCDR3 |
| 612 | DIQLTQSPSSLSASVGDRVTITCRASQSISSYLNWYQQKPGKAPKLLIYAASSLQSGVPS | Anti-PRAME_V_{L} |
| | RFSGSGSGTDFTLTISSLQPEDFATYYCQQSYSTPRTFGQGTKVEIKR | |
| 613 | GGTFSSYA | Anti-PRAME_HCDR1 |
| 614 | IIPILGIA | Anti-PRAME_HCDR2 |
| 615 | ARQGYVWSEMDF | Anti-PRAME_HCDR3 |
| 616 | EVQLVESGAEVKKPGSSVKVSCKASGGTFSSYAISWVRQAPGQGLEWMGRIIPILGIA | Anti-PRAME_V_{H} |
| | NYAQKFQGRVTITADKSTSTAYMELSSLRSEDTAVYYCARQGYVWSEMDFWGQGTL | |
| | VTVSS | |
| 617 | NIGSKS | Anti-PRAME_LCDR1 |
| 618 | YDS | Anti-PRAME_LCDR2 |
| 619 | QVWDSITDHYV | Anti-PRAME_LCDR3 |
| 620 | KLLPVLTIPPSVSVAPGKTARITCGGNNIGSKSVHWYQQKPGQAPVLVIYYDSDRPSG | Anti-PRAME_V_{L} |
| | IPERFSGSNSGNTATLTISRVEAGDEADYYCQVWDSITDHYVFGTGTKVTVLG | |
| 621 | GYTFTSYY | Anti-PRAME_HCDR1 |
| 622 | INPSGGST | Anti-PRAME_HCDR2 |
| 623 | AAGSYYSLDI | Anti-PRAME_HCDR3 |
| 624 | EVQLVESGAEVKKPGASVKVSCKASGYTFTSYYMHWVRQAPGQGLEWMGIINPSGG | Anti-PRAME_V_{H} |
| | STSYAQKFQGRVTMTRDTSTSTVYMELSSLRSEDTAVYYCAAGSYYSLDIWGQGTM | |
| | VTVSS | |
| 625 | SGSIASNF | Anti-PRAME_LCDR1 |
| 626 | DDN | Anti-PRAME_LCDR2 |
| 627 | QSYDGSNVI | Anti-PRAME_LCDR3 |
| 628 | KLLPVLTQPHSVSESPGKTVTISCTGSSGSIASNFVQWYQQRPGSAPTTVIYDDNQRPS | Anti-PRAME_V_{L} |
| | GVPDRFSASIDRSSNSASLTISGLKTDDEADYYCQSYDGSNVIFGGGTKLTVLG | |
| 629 | GYTFSSYY | Anti-PRAME_HCDR1 |
| 630 | INPTSGST | Anti-PRAME_HCDR2 |
| 631 | ARSGGGYGDS | Anti-PRAME_HCDR3 |
| 632 | EVQLVQSGAEVEKPGASVKVSCKASGYTFSSYYMDWVRQAPGQGLEWMGRINPTSG | Anti-PRAME_V_{H} |
| | STTYAQKFQGRVTMTRDTSTFTVYMDLSSLRSEDTAVYYCARSGGGYGDSWGQGTL | |
| | VTVSS | |
| 633 | NFGSQS | Anti-PRAME_LCDR1 |
| 634 | YDQ | Anti-PRAME_LCDR2 |
| 635 | QVWDTYTDHVV | Anti-PRAME_LCDR3 |
| 636 | QPVLTQPPSVSVAPGETASVSCGGNNFGSQSVHWYQQKSGQAPLLVIYYDQDRPSEIP | Anti-PRAME_V_{L} |
| | ARFSGSKSGNTATLTISRVEAGDEADYYCQVWDTYTDHVVFGGGTKLTVLG | |
| 637 | RMFPNAPYL | WT1 peptide can be |
| | | targeted by CAR |
| 638 | GGTFSSYAIS | Anti-WT1_HCDR1 |
| 639 | GIIPIFGTANYAQKFQG | Anti-WT1_HCDR2 |
| 640 | RIPPYYGMDV | Anti-WT1_HCDR3 |
| 641 | QVQLVQSGAEVKKPGSSVKVSCKASGGTFSSYAISWVRQAPGQGLEWMGGIIPIFGTA | Anti-WT1_V_{H} |
| | NYAQKFQGRVTITADESTSTAYMELSSLRSEDTAVYYCARRIPPYYGMDVWGQGTTV | |
| | TVSS | |
| 642 | SGSSSNIGSNYVY | Anti-WT1_LCDR1 |
| 643 | RSNQRPS | Anti-WT1_LCDR2 |
| 644 | AAWDDSLNGVV | Anti-WT1_LCDR3 |
| 645 | QTVVTQPPSASGTPGQRVTISCSGSSSNIGSNYVYWYQQLPGTAPKLLIYRSNQRPSGV | Anti-WT1_V_{L} |
| | PDRFSGSKSGTSASLAISGPRSVDEADYYCAAWDDSLNGVVFGGGTKLTVLG | |
| 646 | QTVVTQPPSASGTPGQRVTISCSGSSSNIGSNYVYWYQQLPGTAPKLLIYRSNQRPSGV | Anti-WT1_scFv |
| | PDRFSGSKSGTSASLAISGPRSVDEADYYCAAWDDSLNGVVFGGGTKLTVLGSRGGG | |
| | GSGGGGSGGGSLEMAQVQLVQSGAEVKKPGSSVKVSCKASGGTFSSYAISWVRQAP | |
| | GQGLEWMGGIIPIFGTANYAQKFQGRVTITADESTSTAYMELSSLRSEDTAVYYCARR | |
| | IPPYYGMDVWGQGTTVTVSS | |
| 647 | GDSVSSNSAAWN | Anti-WT1_HCDR1 |
| 648 | RTYYGSKWYNDYAVSVKS | Anti-WT1_HCDR2 |
| 649 | GRLGDAFDI | Anti-WT1_HCDR3 |
| 650 | QVQLQQSGPGLVKPSQTLSLTCAISGDSVSSNSAAWNWIRQSPSRGLEWLGRTYYGS | Anti-WT1_V_{H} |
| | KWYNDYAVSVKSRITINPDTSKNQFSLQLNSVTPEDTAVYYCARGRLGDAFDIWGQG | |
| | TMVTVSS | |
| 651 | RASQSISSYLN | Anti-WT1_LCDR1 |
| 652 | AASSLQS | Anti-WT1_LCDR2 |
| 653 | QQSYSTPLT | Anti-WT1_LCDR3 |
| 654 | DIQMTQSPSSLSASVGDRVTITCRASQSISSYLNWYQQKPGKAPKLLIYAASSLQSGVP | Anti-WT1_V_{L} |
| | SRFSGSGSGTDFTLTISSLQPEDFATYYCQQSYSTPLTFGGGTKVDIKR | |
| 655 | DIQMTQSPSSLSASVGDRVTITCRASQSISSYLNWYQQKPGKAPKLLIYAASSLQSGVP | Anti-WT1_scFv |
| | SRFSGSGSGTDFTLTISSLQPEDFATYYCQQSYSTPLTFGGGTKVDIKRSRGGGGSGGG | |
| | GSGGGGSLEMAQVQLQQSGPGLVKPSQTLSLTCAISGDSVSSNSAAWNWIRQSPSRG | |
| | LEWLGRTYYGSKWYNDYAVSVKSRITINPDTSKNQFSLQLNSVTPEDTAVYYCARGR | |
| | LGDAFDIWGQGTMVTVSS | |
| 656 | GYSFTNFWIS | Anti-WT1_HCDR1 |
| 657 | RVDPGYSYSTYSPSFQG | Anti-WT1_HCDR2 |
| 658 | VQYSGYYDWFDP | Anti-WT1_HCDR3 |
| 659 | QMQLVQSGAEVKEPGESLRISCKGSGYSFTNFWISWVRQMPGKGLEWMGRVDPGYS | Anti-WT1_V_{H} |
| | YSTYSPSFQGHVTISADKSTSTAYLQWNSLKASDTAMYYCARVQYSGYYDWFDPWG | |
| | QGTLVTVSS | |
| 660 | SGSSSNIGSNTVN | Anti-WT1_LCDR1 |
| 661 | SNNQRPS | Anti-WT1_LCDR2 |
| 662 | AAWDDSLNGWV | Anti-WT1_LCDR3 |
| 663 | QAVVTQPPSASGTPGQRVTISCSGSSSNIGSNTVNWYQQVPGTAPKLLIYSNNQRPSG | Anti-WT1_V_{L} |
| | VPDRFSGSKSGTSASLAISGLQSEDEADYYCAAWDDSLNGWVFGGGTKLTVLG | |
| 664 | QAVVTQPPSASGTPGQRVTISCSGSSSNIGSNTVNWYQQVPGTAPKLLIYSNNQRPSG | Anti-WT1_scFv |
| | VPDRFSGSKSGTSASLAISGLQSEDEADYYCAAWDDSLNGWVFGGGTKLTVLGSRGG | |
| | GGSGGGGSGGGGSLEMAQMQLVQSGAEVKEPGESLRISCKGSGYSFTNFWISWVRQ | |
| | MPGKGLEWMGRVDPGYSYSTYSPSFQGHVTISADKSTSTAYLQWNSLKASDTAMYY | |
| | CARVQYSGYYDWFDPWGQGTLVTVSS | |
| 665 | GYNFSNKWIG | Anti-WT1_HCDR1 |
| 666 | IIYPGYSDITYSPSFQG | Anti-WT1_HCDR2 |
| 667 | HTALAGFDY | Anti-WT1_HCDR3 |
| 668 | QVQLVQSGAEVKKPGESLKISCKGSGYNFSNKWIGWVRQLPGRGLEWIAIIYPGYSDI | Anti-WT1_V_{H} |
| | TYSPSFQGRVTISADTSINTAYLHWHSLKASDTAMYYCVRHTALAGFDYWGLGTLVT | |
| | VSS | |
| 669 | RASQNINKWLA | Anti-WT1_LCDR1 |
| 670 | KASSLES | Anti-WT1_LCDR2 |
| 671 | QQYNSYAT | Anti-WT1_LCDR3 |
| 672 | DIQMTQSPSTLSASVGDRVTITCRASQNINKWLAWYQQRPGKAPQLLIYKASSLESGV | Anti-WT1_V_{L} |
| | PSRFSGSGSGTEYTLTISSLQPDDFATYYCQQYNSYATFGQGTKVEIKR | |
| 673 | DIQMTQSPSTLSASVGDRVTITCRASQNINKWLAWYQQRPGKAPQLLIYKASSLESGV | Anti-WT1_scFv |
| | PSRFSGSGSGTEYTLTISSLQPDDFATYYCQQYNSYATFGQGTKVEIKRSRGGGGSGG | |
| | GGSGGGGSLEMAQVQLVQSGAEVKKPGESLKISCKGSGYNFSNKWIGWVRQLPGRG | |
| | LEWIAIIYPGYSDITYSPSFQGRVTISADTSINTAYLHWHSLKASDTAMYYCVRHTALA | |
| | GFDYWGLGTLVTVSS | |
| 674 | GFTFDDYGMS | Anti-WT1_HCDR1 |
| 675 | GINWNGGSTGYADSVRG | Anti-WT1_HCDR2 |
| 676 | ERGYGYHDPHDY | Anti-WT1_HCDR3 |
| 677 | EVQLVQSGGGVVRPGGSLRLSCAASGFTFDDYGMSWVRQAPGKGLEWVSGINWNG | Anti-WT1_V_{H} |
| | GSTGYADSVRGRFTISRDNAKNSLYLQMNSLRAEDTALYYCARERGYGYHDPHDYW | |
| | GQGTLVTVSS | |
| 678 | GRNNIGSKSVH | Anti-WT1_LCDR1 |
| 679 | DDSDRPS | Anti-WT1_LCDR2 |
| 680 | QVWDSSSDHVV | Anti-WT1_LCDR3 |
| 681 | QSVVTQPPSVSVAPGKTARITCGRNNIGSKSVHWYQQKPGQAPVLVVYDDSDRPSGIP | Anti-WT1_V_{L} |
| | ERFSGSNSGNTATLTISRVEAGDEADYYCQVWDSSSDHVVFGGGTKLTVLG | |
| 682 | QSVVTQPPSVSVAPGKTARITCGRNNIGSKSVHWYQQKPGQAPVLVVYDDSDRPSGIP | Anti-WT1_scFv |
| | ERFSGSNSGNTATLTISRVEAGDEADYYCQVWDSSSDHVVFGGGTKLTVLGSRGGGG | |
| | SGGGGSGGSLEMAEVQLVQSGGGVVRPGGSLRLSCAASGFTFDDYGMSWVRQAPGK | |
| | GLEWVSGINWNGGSTGYADSVRGRFTISRDNAKNSLYLQMNSLRAEDTALYYCARE | |
| | RGYGYHDPHDYWGQGTLVTVSS | |
| 683 | GFSVSGTYMG | Anti-WT1_HCDR1 |
| 684 | LLYSGGGTYHPASLQG | Anti-WT1_HCDR2 |
| 685 | GGAGGGHFDS | Anti-WT1_HCDR3 |
| 686 | EVQLVETGGGLLQPGGSLRLSCAASGFSVSGTYMGWVRQAPGKGLEWVALLYSGGG | Anti-WT1_V_{H} |
| | TYHPASLQGRFIVSRDSSKNMVYLQMNSLKAEDTAVYYCAKGGAGGGHFDSWGQG | |
| | TLVTVSS | |
| 687 | TGSSSNIGAGYDVH | Anti-WT1_LCDR1 |
| 688 | GNSNRPS | Anti-WT1_LCDR2 |
| 689 | AAWDDSLNGYV | Anti-WT1_LCDR3 |
| 690 | QSVLTQPPSVSGAPGQRVTISCTGSSSNIGAGYDVHWYQQLPGTAPKLLIYGNSNRPS | Anti-WT1_V_{L} |
| | GVPDRFSGSKSGTSASLAISGLQSEDEADYYCAAWDDSLNGYVFGTGTKLTVLG | |
| 691 | QSVLTQPPSVSGAPGQRVTISCTGSSSNIGAGYDVHWYQQLPGTAPKLLIYGNSNRPS | Anti-WT1_scFv |
| | GVPDRFSGSKSGTSASLAISGLQSEDEADYYCAAWDDSLNGYVFGTGTKLTVLGSRG | |
| | GGGSGGGGSGGGGSLEMAEVQLVETGGGLLQPGGSLRLSCAASGFSVSGTYMGWVR | |
| | QAPGKGLEWVALLYSGGGTYHPASLQGRFIVSRDSSKNMVYLQMNSLKAEDTAVYY | |
| | CAKGGAGGGHFDSWGQGTLVTVSS | |
| 692 | RKSAPSTGGV | Histone H3.3 peptide |
| | | can be targeted by |
| | | CAR |
| 693 | RKSAPATGGV | Histone H3.3 peptide |
| | | can be targeted by |
| | | CAR |
| 694 | RMSAPSTGGV | Histone H3.3 peptide |
| | | can be targeted by |
| | | CAR |
| 695 | RMSAPATGGV | Histone H3.3 peptide |
| | | can be targeted by |
| | | CAR |
| 696 | AMSAPSTGGV | Histone H3.3 peptide |
| | | can be targeted by |
| | | CAR |
| 697 | RASAPSTGGV | Histone H3.3 peptide |
| | | can be targeted by |
| | | CAR |
| 698 | RMAAPSTGGV | Histone H3.3 peptide |
| | | can be targeted by |
| | | CAR |
| 699 | RMSAASTGGV | Histone H3.3 peptide |
| | | can be targeted by |
| | | CAR |
| 700 | RMSAPSAGGV | Histone H3.3 peptide |
| | | can be targeted by |
| | | CAR |
| 701 | RMSAPSTAGV | Histone H3.3 peptide |
| | | can be targeted by |
| | | CAR |
| 702 | RMSAPSTGAV | Histone H3.3 peptide |
| | | can be targeted by |
| | | CAR |
| 703 | RMSAPSTGGA | Histone H3.3 peptide |
| | | can be targeted by |
| | | CAR |
| 704 | AMSAPATGGV | Histone H3.3 peptide |
| | | can be targeted by |
| | | CAR |
| 705 | RASAPATGGV | Histone H3.3 peptide |
| | | can be targeted by |
| | | CAR |
| 706 | RMAAPATGGV | Histone H3.3 peptide |
| | | can be targeted by |
| | | CAR |
| 707 | RMSAAATGGV | Histone H3.3 peptide |
| | | can be targeted by |
| | | CAR |
| 708 | RMSAPAAGGV | Histone H3.3 peptide |
| | | can be targeted by |
| | | CAR |
| 709 | RMSAPATAGV | Histone H3.3 peptide |
| | | can be targeted by |
| | | CAR |
| 710 | RMSAPATGAV | Histone H3.3 peptide |
| | | can be targeted by |
| | | CAR |
| 711 | RMSAPATGGA | Histone H3.3 peptide |
| | | can be targeted by |
| | | CAR |
| 712 | GYSFTSYW | Anti-Histone |
| | | H3.3_HCDR1 |
| 713 | IYPGDSDT | Anti-Histone |
| | | H3.3_HCDR2 |
| 714 | ARGYDG | Anti-Histone |
| | | H3.3_HCDR3 |
| 715 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDGWGQGTLVTVSS | |
| 716 | QSLVYSNGNTY | Anti-Histone |
| | | H3.3_LCDR1 |
| 717 | EVS | Anti-Histone |
| | | H3.3_LCDR2 |
| 718 | MQGTHWPPT | Anti-Histone |
| | | H3.3_LCDR3 |
| 719 | DVVMTQSPLSLPVTLGQPASISCRSSQSLVYSNGNTYLNWFHQRPGQSPRRLIYEVSN | Anti-Histone H3.3_V_{L} |
| | RDSGVPDRFSGSGSGTDFTLKISRVEAEDVGVYYCMQGTHWPPTFGGGTKLEIKR | |
| 720 | GYSFTSYW | Anti-Histone |
| | | H3.3_HCDR1 |
| 721 | IYPGDSDT | Anti-Histone |
| | | H3.3_HCDR2 |
| 722 | ARGFDN | Anti-Histone |
| | | H3.3_HCDR3 |
| 723 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGFDNWGQGTLVTVSS | |
| 724 | QSLVYSNGNTY | Anti-Histone |
| | | H3.3_LCDR1 |
| 725 | KVS | Anti-Histone |
| | | H3.3_LCDR2 |
| 726 | MQGTYWPYT | Anti-Histone |
| | | H3.3_LCDR3 |
| 727 | EIVLTQSPLSLPVTLGQPASISCRSSQSLVYSNGNTYLSWFHQRPGQSPRRLIYKVSKRD | Anti-Histone H3.3_V_{L} |
| | SGVPDRFSGSGSGTDFTLKISRVEAEDVGVFYCMQGTYWPYTFGQGTKLEIKR | |
| 728 | GYSFTSYW | Anti-Histone |
| | | H3.3_HCDR1 |
| 729 | IYPGDSDT | Anti-Histone |
| | | H3.3_HCDR2 |
| 730 | ARGYDV | Anti-Histone |
| | | H3.3_HCDR3 |
| 731 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDVWGQGTLVTVSS | |
| 732 | QSLIYSNGNTY | Anti-Histone |
| | | H3.3_LCDR1 |
| 733 | KVS | Anti-Histone |
| | | H3.3_LCDR2 |
| 734 | MQGTHWPPT | Anti-Histone |
| | | H3.3_LCDR3 |
| 735 | DVVMTQSPLSLPVTLGQPASISCRSSQSLIYSNGNTYLTWFHQRPGQPPRRLIHKVSNR | Anti-Histone H3.3_V_{L} |
| | DSGVPDRFSGSGSGSDFTLKISRVEAEDVGIYYCMQGTHWPPTFGGGTKLEIKR | |
| 736 | GYSFTSYW | Anti-Histone |
| | | H3.3_HCDR1 |
| 737 | IYPGDSDT | Anti-Histone |
| | | H3.3_HCDR2 |
| 738 | ARGYDS | Anti-Histone |
| | | H3.3_HCDR3 |
| 739 | EVQLVQSGAEVKKPGESLKISCKGSGYSFTSYWIGWVRQMPGKGLEWMGIIYPGDSD | Anti-Histone H3.3_V_{H} |
| | TRYSPSFQGQVTISADKSISTAYLQWSSLKASDTAMYYCARGYDSWGQGTLVTVSS | |
| 740 | QSLIYSNGNTY | Anti-Histone |
| | | H3.3_LCDR1 |
| 741 | KVS | Anti-Histone |
| | | H3.3_LCDR2 |
| 742 | MQGTHWPPT | Anti-Histone |
| | | H3.3_LCDR3 |
| 743 | DVVMTQSPLSLPVTLGQPASISCRSSQSLIYSNGNTYLTWFHQRPGQPPRRLIHKVSNR | Anti-Histone H3.3_V_{L} |
| | DSGVPDRFSGSGSGSDFTLKISRVEAEDVGIYYCMQGTHWPPTFGGGTKLEIKR | |
| 744 | GLTFDRYA | Anti-Histone |
| | | H3.3_HCDR1 |
| 745 | ITGDGYYT | Anti-Histone |
| | | H3.3_HCDR2 |
| 746 | ARLSGIGRSSYDG | Anti-Histone |
| | | H3.3_HCDR3 |
| 747 | EVQLVESGGGLVQPGGSLRLSCAASGLTFDRYAMSWVRQAAGKGLERFSAITGDGY | Anti-Histone H3.3_V_{H} |
| | YTYYADSVKGRFTISRDNSKNTLYLQMNSLGAEDTAVYYCARLSGIGRSSYDGWGQ | |
| | GTLVTVSS | |
| 748 | SGINVGTYR | Anti-Histone |
| | | H3.3_LCDR1 |
| 749 | YKSDSDK | Anti-Histone |
| | | H3.3_LCDR2 |
| 750 | MIWHSSA | Anti-Histone |
| | | H3.3_LCDR3 |
| 751 | QAVLTQPSSLSASPGASASLTCTLRSGINVGTYRIYWYQQKPGSPPQYLLRYKSDSDK | Anti-Histone H3.3_V_{L} |
| | QQGSGVPSRFSGSKDASANAGILLISGLQSEDEADYYCMIWHSSAWVFGGGTKLTVL | |
| | G | |
| 752 | GYTFTSYT | Anti-Histone |
| | | H3.3_HCDR1 |
| 753 | ISPYNGNT | Anti-Histone |
| | | H3.3_HCDR2 |
| 754 | ARSWEHGFPYDE | Anti-Histone |
| | | H3.3_HCDR3 |
| 755 | QVQLVESGAEVKKPGASVKVSCKASGYTFTSYTITWVRQAPGQGLEWMGWISPYNG | Anti-Histone H3.3_V_{H} |
| | NTNYAQNLQGRVTMTTDTSTTTAYMELRSLTSDDTAVYYCARSWEHGFPYDEWGQ | |
| | GTLVTVSS | |
| 756 | SSNLGAGYD | Anti-Histone |
| | | H3.3_LCDR1 |
| 757 | ISPYNGNT | Anti-Histone |
| | | H3.3_LCDR2 |
| 758 | QSYDSSLSASV | Anti-Histone |
| | | H3.3_LCDR3 |
| 759 | QSVLTQPPSVSGAPGQRVTISCTGSSSNLGAGYDVHWYQQLPGTAPKVLVYFNNNRP | Anti-Histone H3.3_V_{L} |
| | SGVPDRFSGSKSGTSASLAITGLQAEDEADYYCQSYDSSLSASVFGTGTKVTVLG | |
| 760 | AGTFNRYS | Anti-Histone |
| | | H3.3_HCDR1 |
| 761 | IIPIIGVA | Anti-Histone |
| | | H3.3_HCDR2 |
| 762 | ARQEYSYAMDY | Anti-Histone |
| | | H3.3_HCDR3 |
| 763 | EVQLVQSGAEVRKPGSSVKVSCKASAGTFNRYSLSWVRQAPGQGLEWVGRIIPIIGVA | Anti-Histone H3.3_V_{H} |
| | DYAQKFQGRVTITADKSATTAYMELHSLRSEDTAVYYCARQEYSYAMDYWGQGTL | |
| | VTVSS | |
| 764 | SSNIGAGYD | Anti-Histone |
| | | H3.3_LCDR1 |
| 765 | GNN | Anti-Histone |
| | | H3.3_LCDR2 |
| 766 | QSYDTSLTPV | Anti-Histone |
| | | H3.3_LCDR3 |
| 767 | QSVLTQPPSVSGAPGQRVTIFCTGSSSNIGAGYDVHWYQQLPGTAPKLLIYGNNRPS | Anti-Histone H3.3_V_{L} |
| | GVPDRFSGSKSGTSASLAITGLQAEDEADYYCQSYDTSLTPVFGGGTKLTVLG | |
| 768 | GGTFSSYA | Anti-Histone |
| | | H3.3_HCDR1 |
| 769 | IIPIFGTA | Anti-Histone |
| | | H3.3_HCDR2 |
| 770 | ARSYWTFEYSEDS | Anti-Histone |
| | | H3.3_HCDR3 |
| 771 | QVQLVQSGAEVKKPGSSVKVSCKASGGTFSSYAISWVRQAPGQGLEWMGGIIPIFGTA | Anti-Histone H3.3_V_{H} |
| | NYAQKFQGRVTITADESTSTAYMELSSLRSEDTAVYYCARSYWTFEYSEDSWGQGTL | |
| | VTVSS | |
| 772 | SLNLGAGYD | Anti-Histone |
| | | H3.3_LCDR1 |
| 773 | ANT | Anti-Histone |
| | | H3.3_LCDR2 |
| 774 | QSYDNSLSGYV | Anti-Histone |
| | | H3.3_LCDR3 |
| 775 | QSVLTQPPSVSGAPGQRVTISCTGSSLNLGAGYDVHWYQQFPGTAPKLLIFANTNRPS | Anti-Histone H3.3_V_{L} |
| | GVPDRFSASKSGTSASLAITGLQAEDEADYFCQSYDNSLSGYVFGTGTKVTVLG | |
| 776 | GYTFTSYG | Anti-Histone |
| | | H3.3_HCDR1 |
| 777 | ISAYNGNT | Anti-Histone |
| | | H3.3_HCDR2 |
| 778 | ARYYESGYPFDW | Anti-Histone |
| | | H3.3_HCDR3 |
| 779 | EVQLVQSGAEVKKPGASVKVSCKASGYTFTSYGISWVRQAPGQGLEWMGWISAYNG | Anti-Histone H3.3_V_{H} |
| | NTNYAQKLQGRVTMTTDTSTSTAYMELRSLRSDDTAVYYCARYYESGYPFDWWGQ | |
| | GTLVTVSS | |
| 780 | TFNIGSNT | Anti-Histone |
| | | H3.3_LCDR1 |
| 781 | SNN | Anti-Histone |
| | | H3.3_LCDR2 |
| 782 | AAWDDSLSGHVV | Anti-Histone |
| | | H3.3_LCDR3 |
| 783 | SYVLTQPPSASGTPGQRVTISCSGSTFNIGSNTVNWYQQLPATAPKLLIYSNNQRPSGV | Anti-Histone H3.3_V_{L} |
| | PDRFSGSKSGTSASLAISGLQSEDAAAYYCAAWDDSLSGHVVFGGGTKLTVLG | |
| 784 | GYTFTGYY | Anti-Histone |
| | | H3.3_HCDR1 |
| 785 | FDPEDGET | Anti-Histone |
| | | H3.3_HCDR2 |
| 786 | ARSSWWSPVTYYDI | Anti-Histone |
| | | H3.3_HCDR3 |
| 787 | EVQLVQSGAEVKKPGASVKVSCKASGYTFTGYYMHWVRQAPGKGLEWMGGFDPED | Anti-Histone H3.3_V_{H} |
| | GETIYAQKFQGRVTMTEDTSTDTAYMELSSLRSEDTAVYYCARSSWWSPVTYYDIW | |
| | GQGTLVTVSS | |
| 788 | SLRSYY | Anti-Histone |
| | | H3.3_LCDR1 |
| 789 | AKS | Anti-Histone |
| | | H3.3_LCDR2 |
| 790 | NSRDSSGNR | Anti-Histone |
| | | H3.3_LCDR3 |
| 791 | SSELTQDPAVSVALGQTVRITCQGDSLRSYYANWYQQKPGQAPVLVIYAKSNRPSGIA | Anti-Histone H3.3_V_{L} |
| | DRFSGSSSGNTASLTITGAQAEDEADYYCNSRDSSGNRWVFGGGTKVTVLG | |
| 792 | GYTVTSYG | Anti-Histone |
| | | H3.3_HCDR1 |
| 793 | ISAYNGDT | Anti-Histone |
| | | H3.3_HCDR2 |
| 794 | ARSSLPFGVVPNAFDI | Anti-Histone |
| | | H3.3_HCDR3 |
| 795 | QMQLVQSGAEVKKPGASMKVSCKASGYTVTSYGLSWVRQAPGQGLEWVGWISAYN | Anti-Histone H3.3_V_{H} |
| | GDTIYAQKLQGRVTMTTDTSTSTAYMELRSLRSDDTAMYYCARSSLPFGVVPNAFDI | |
| | WGQGTMVTVSS | |
| 796 | SSNIGSNT | Anti-Histone |
| | | H3.3_LCDR1 |
| 797 | SNN | Anti-Histone |
| | | H3.3_LCDR2 |
| 798 | AAWDDSLNGYV | Anti-Histone |
| | | H3.3_LCDR3 |
| 799 | SYVLTQPPSASGTPGQRVTISCSGSSSNIGSNTVNWYQQLPGTAPKLLIYSNNQRPSGV | Anti-Histone H3.3_V_{L} |
| | PDRFSGSKSGTSASLAISGLQSEDEADYYCAAWDDSLNGYVFGTGTKVTVLG | |
| 800 | GYTFTDYY | Anti-Histone |
| | | H3.3_HCDR1 |
| 801 | INPHSGGT | Anti-Histone |
| | | H3.3_HCDR2 |
| 802 | AREDYSGSGSSDA | Anti-Histone |
| | | H3.3_HCDR3 |
| 803 | QMQLVQSGAEVKTTGASVRVSCKASGYTFTDYYLHWVRQAPGQGLEWMGWINPHS | Anti-Histone H3.3_V_{H} |
| | GGTNYAQKFQGRVTMTRDTSISTAYMELSSLRSDDTAVYYCAREDYSGSGSSDAWG | |
| | QGTLVTVSS | |
| 804 | NIGSKS | Anti-Histone |
| | | H3.3_LCDR1 |
| 805 | YDN | Anti-Histone |
| | | H3.3_LCDR2 |
| 806 | QVWNSSSDHYV | Anti-Histone |
| | | H3.3_LCDR3 |
| 807 | SYELTQPPSVSVAPGKTARITCGGNNIGSKSVNWYQQKPGQAPVLVIYYDNDRPSGIP | Anti-Histone H3.3_V_{L} |
| | ERFSGSNSGNTATLTISRVEAGDEADYYCQVWNSSSDHYVFGTGTKVTVLG | |
| 808 | NNNYYWT | Anti-MSLN_HCDR1 |
| 809 | YIYYSGSTFYNPSLKS | Anti-MSLN_HCDR2 |
| 810 | EDTMTGLDV | Anti-MSLN_HCDR3 |
| 811 | QVQLQESGPGLVKPSQTLSLTCTVSGGSINNNNYYWTWIRQHPGKGLEWIGYIYYSGS | Anti-MSLN_V_{H} |
| | TFYNPSLKSRVTISVDTSKTQFSLKLSSVTAADTAVYYCAREDTMTGLDVWGQGTTV | |
| | TVSS | |
| 812 | RASQSINNYLN | Anti-MSLN_LCDR1 |
| 813 | AASSLQS | Anti-MSLN_LCDR2 |
| 814 | QQTYSNPT | Anti-MSLN_LCDR3 |
| 815 | DIQMTQSPSSLSASVGDRVTITCRASQSINNYLNWYQQKPGKAPTLLIYAASSLQSGVP | Anti-MSLN_V_{L} |
| | SRFSGSRSGTDFTLTISSLQPEDFAAYFCQQTYSNPTFGQGTKVEVK | |
| 816 | KTITATGVLFVRLGP | ROR2 epitope |
| 817 | GYRFSKYW | Anti-ROR2_HCDR1 |
| 818 | IYPGDSDT | Anti-ROR2_HCDR2 |
| 819 | ARSFSSFIYDY | Anti-ROR2_HCDR3 |
| 820 | EVQLVQSGAEVKKPGESLKISCQGSGYRFSKYWIGWVRQMPGKGLEWMGIIYPGDSD | Anti-ROR2_V_{H} |
| | TRYSPSFQGQVTISADKSISTAYLQWSSLKASDTAMYYCARSFSSFIYDYWGQGTLVT | |
| | VSS | |
| 821 | GYSFSNYW | Anti-ROR2_HCDR1 |
| 822 | IYPDDSDT | Anti-ROR2_HCDR2 |
| 823 | VRPRGAFDI | Anti-ROR2_HCDR3 |
| 824 | QVQLVESGAEVKKPGESLKISCKASGYSFSNYWIGWVRQMPGKGLEWMGIIYPDDSD | Anti-ROR2_V_{H} |
| | TRYSPSFQGQVTISADKSISTAYLQWYSLKVADTAKYYCVRPRGAFDIWGQGTTVTV | |
| | SS | |
| 825 | GGSISSGGYY | Anti-ROR2_HCDR1 |
| 826 | IYYSGST | Anti-ROR2_HCDR2 |
| 827 | ARGGLYWTYSQDV | Anti-ROR2_HCDR3 |
| 828 | EVQLVESGPGLVKPSQTLSLTCTVSGGSISSGGYYWSWIRQHPGKGLEWIGYIYYSGS | Anti-ROR2_V_{H} |
| | TYYNPSLKSRVTISVDTSKNQFSLKLSSVTAADTAMYYCARGGLYWTYSQDVWGQG | |
| | TLVTVSS | |
| 829 | GFSLSTSGMS | Anti-ROR2_HCDR1 |
| 830 | EDWDDDK | Anti-ROR2_HCDR2 |
| 831 | ARGFYLAYGSYDS | Anti-ROR2_HCDR3 |
| 832 | QITLKESGPELVKPTQTLTLTCTFSGFSLSTSGMSVSWIRQPPGKALEWLARIDWDDD | Anti-ROR2_V_{H} |
| | KYYSTSLKTRLTISKDTSKNQVVLTMTNTDPVDTATYYCARGFYLAYGSYDSWGQG | |
| | TLVTVSS | |
| 833 | GYTFTNYY | Anti-ROR2_HCDR1 |
| 834 | INPTSGRT | Anti-ROR2_HCDR2 |
| 835 | ARSGYYWGVNGDQ | Anti-ROR2 HCDR3 |
| 836 | EVQLVQSGAEVKKPGASVKVSCKASGYTFTNYYIHWVRQAPGQGLEWMGIINPTSG | Anti-ROR2_V_{H} |
| | RTRYAQRFQGRVTMTRDTSTNTVYMDLSSLRSEDTAMYYCARSGYYWGVNGDQW | |
| | GQGTLVTVSS | |
| 837 | QSVSSN | Anti-ROR2_LCDR1 |
| 838 | GAS | Anti-ROR2_LCDR2 |
| 839 | QQYGRSPLT | Anti-ROR2_LCDR3 |
| 840 | ETTLTQSPGTLSVSPGERATLSCRASQSVSSNLAWYQQKRGQAPRLLIYGASTRATGIP | Anti-ROR2_V_{L} |
| | VRFSGSGSGTEFTLTISRLEPEDFAVYYCQQYGRSPLTFGGGTKVDIKR | |
| 841 | SSNIGAGHA | Anti-ROR2_LCDR1 |
| 842 | DNA | Anti-ROR2_LCDR2 |
| 843 | GTWDDSPSAYV | Anti-ROR2_LCDR3 |
| 844 | QSVLTQPPSVSGAPGQRVTISCTGSSSNIGAGHAVHWYQQLPGTAPKLLIYDNANRPS | Anti-ROR2_V_{L} |
| | GVPDRFSGSQSGTSASLAITGLQTGDEADYYCGTWDDSPSAYVFGTGTKVTVLG | |
| 845 | SSNIGSDY | Anti-ROR2_LCDR1 |
| 846 | RND | Anti-ROR2_LCDR2 |
| 847 | VAWDDSLSGYV | Anti-ROR2_LCDR3 |
| 848 | QPVLTQPPSASGTPGQRVTISCSGSSSNIGSDYVSWYQQLPGTAPKLLIYRNDQRPSGV | Anti-ROR2_V_{L} |
| | PDRFSGSKSGTSASLAISGLRSEDEADYYCVAWDDSLSGYVFGSGTKVTVLG | |
| 849 | SGDVGGYNY | Anti-ROR2_LCDR1 |
| 850 | DVN | Anti-ROR2_LCDR2 |
| 851 | SSYTSTSTV | Anti-ROR2_LCDR3 |
| 852 | QSALTQPASVSGSPGQSITISCTGTSGDVGGYNYVSWYQHHPGKAPKLIIYDVNKRPS | Anti-ROR2_V_{L} |
| | GFSDRFSGSKSGNTASLTISGLQAEDEADYYCSSYTSTSTVFGGGTKLTVLG | |
| 853 | NIGSKN | Anti-ROR2_LCDR1 |
| 854 | RDS | Anti-ROR2_LCDR2 |
| 855 | QVWDSSIVV | Anti-ROR2_LCDR3 |
| 856 | SYELTQPLSVSVALGQTARITCGGNNIGSKNVHWYQQKPGQAPVLVIYRDSNRPSGIP | Anti-ROR2_V_{L} |
| | ERFSGSNSGNTATLTISRAQAGDEADYYCQVWDSSIVVFGGGTKLTVLG | |
| 857 | GFTFSSYA | Anti-HER2_HCDR1 |
| 858 | ISGSGYST | Anti-HER2_HCDR2 |
| 859 | AKGFQYGSGSYYTHFDY | Anti-HER2_HCDR3 |
| 860 | EVQLLESGGGLVQPGGSLRLSCAASGFTFSSYAMTWVRQAPGKGLEWVSAISGSGYS | Anti-HER2_V_{H} |
| | TYYADSEKGRFTISRDNSKNTLYLQMNSLRAEDTAVYYCAKGFQYGSGSYYTHFDY | |
| | WGQGTLVTVSS | |
| 861 | QGISSW | Anti-HER2_LCDR1 |
| 862 | AAS | Anti-HER2_LCDR2 |
| 863 | QQYNSYPYT | Anti-HER2_LCDR3 |
| 864 | DIQMTQSPSSLSASVGDRVTITCRASQGISSWLAWYQQKPEKAPKSLIYAASSLQSGVP | Anti-HER2_V_{L} |
| | SRFSGSGSGTDFTLTISSLQPEDFATYYCQQYNSYPYTFGQGTKLEIK | |
| 865 | MKYLLPTAAAGLLLLAAQPAMAQVQLVQSGGGLVQPGRSLRLSCAASGFTFDDYAM | Anti-HER3_scFv |
| | HWVRQAPGKGLEWVSGISWNSGSIGYADSVKGRFTISRDNAKNSLYLQMNSLRPEDT | |
| | AVYYCARDLGAKQWLEGFDYWGQGTLVTVSSGGGGSGGGGSGGGGSNFMLTQDPA | |
| | VSVALGQTVRITCQGDSLRSYYASWYQQKPGQAPVLVIYGKNNRPSGIPDRFSGSTSG | |
| | NSASLTITGAQAEDEADYYCNSRDSSGNHWVFGGGTKVTVLGAAAEQKLISEEDLNG | |
| | AAHHHHHH | |
| 866 | GYAFTNYW | Anti-EpCAM_HCDR1 |
| 867 | IFPGSGNI | Anti-EpCAM_HCDR2 |
| 868 | ARLRNWDEPMDY | Anti-EpCAM_HCDR3 |
| 869 | EVQLLEQSGAELVRPGTSVKISCKASGYAFTNYWLGWVKQRPGHGLEWIGDIFPGSG | Anti-EPCAM_V_{H} |
| | NIHYNEKFKGKATLTADKSSSTAYMQLSSLTFEDSAVYFCARLRNWDEPMDYWGQG | |
| | TTVTVSS | |
| 870 | QSLLNSGNQKNY | Anti-EpCAM_LCDR1 |
| 871 | WAS | Anti-EpCAM_LCDR2 |
| 872 | QNDYSYPLT | Anti-EpCAM_LCDR3 |
| 873 | ELVMTQSPSSLTVTAGEKVTMSCKSSQSLLNSGNQKNYLTWYQQKPGQPPKLLIYWAA | Anti-EPCAM_V_{L} |
| | STRESGVPDRFTGSGSGTDFTLTISSVQAEDLAVYYCQNDYSYPLTFGAGTKLEIK | |
| 874 | GYIFTSYD | Anti-MUC1_HCDR1 |
| 875 | IFPGEGST | Anti-MUC1_HCDR2 |
| 876 | ARGDYYRRYFDL | Anti-MUC1_HCDR3 |
| 877 | QVKLQQSGTEVVKPGASVKLSCKASGYIFTSYDIDWVRQTPEQGLEWIGWIFPGEGST | Anti-MUC1_V_{H} |
| | EYNEKFKGRATLSVDKSSSTAYMELTRLTSEDSAVYFCARGDYYRRYFDLWGQGTT | |
| | VTVSS | |
| 878 | ASSSIRY | Anti-MUC1_LCDR1 |
| 879 | DTS | Anti-MUC1_LCDR2 |
| 880 | QEWSGYPYT | Anti-MUC1_LCDR3 |
| 881 | DIELTQSPALMSASPGERVTMTCSASSSIRYIYWYQQKPGSSPRLLIYDTSNVAPGVPFR | Anti-MUC1_V_{L} |
| | FSGSGSGTSYSLTINRMEAEDAATYYCQEWSGYPYTFGGGTKLELKRAAA | |
| 882 | GGSFSGYY | Anti-MUC16_HCDR1 |
| 883 | INHSGST | Anti-MUC16_HCDR2 |
| 884 | ARQSYITDS | Anti-MUC16_HCDR3 |
| 885 | QVQLQQWGAGLLKPSETLSLTCAVYGGSFSGYYWSWIRQPPGKGLEWIGEINHSGST | Anti-MUC16_V_{H} |
| | NYNPSLKSRVTISVDTSKNQFSLKLSSVTAADTAVYYCARQSYITDSWGQGTLVTVSS | |
| 886 | GGSFSGYY | Anti-MUC16_HCDR1 |
| 887 | INHSGST | Anti-MUC16_HCDR2 |
| 888 | RGSIASAY | Anti-MUC16_HCDR3 |
| 889 | QVQLQQWGAGLLKPSETLSLTCAVYGGSFSGYYWSWIRQPPGKGLEWIGEINHSGST | Anti-MUC16_V_{H} |
| | NYNPSLKSRIIMSVDTSKRQFSLKLRSATAADTAVYYCARWSPFSYKQMYDYWGQG | |
| | TLVTVSS | |
| 890 | QDVSKW | Anti-MUC16_LCDR1 |
| 891 | AAS | Anti-MUC16_LCDR2 |
| 892 | QQANSFPWT | Anti-MUC16_LCDR3 |
| 893 | DIQLTQSPSAVSASVGDRVTITCRASQDVSKWLAWYQQKPGKAPRLLISAASGLQSW | Anti-MUC16_V_{L} |
| | VPSRFSGSGSGTEFTLSISSLQPEDFATYYCQQANSFPWTFGQGTKVEIKR | |
| 894 | RGSIASAY | Anti-MUC16_LCDR1 |
| 895 | EDY | Anti-MUC16_LCDR2 |
| 896 | QSYDDNDHVI | Anti-MUC16_LCDR3 |
| 897 | NFMLTQPHSVSESPGKTVTISCTRSRGSIASAYVQWYQQRPGSAPITVIYEDYERPSEIP | Anti-MUC16_V_{L} |
| | DRFSGSIDSSSNSASLTISGLKTEDEADYYCQSYDDNDHVIFGGGTKVTVLG | |
| 898 | GFTFSSYA | Anti-MUC16_HCDR1 |
| 899 | ISSAGGYI | Anti-MUC16_HCDR2 |
| 900 | ARQGFGNYGDYYAMDY | Anti-MUC16_HCDR3 |
| 901 | EVKLQESGGGFVKPGGSLRVSCAASGFTFSSYAMSWVRLAPEMRLEWVATISSAGGY | Anti-MUC16_V_{H} |
| | IFYSDSVQGRFTISRDNAKNSLHLQMGSLRSGDTAMYYCARQGFGNYGDYYAMDY | |
| | WGQGTTVTVSS | |
| 902 | EVQLVESGGGLVKPGGSLRVSCAASGFTFSSYAMSWVRLAPGKGLEWVATISSAGGY | Anti-MUC16_V_{H} |
| | IFYSDSVQGRFTISRDNAKNSLYLQMNSLRAEDTAMYYCARQGFGNYGDYYAMDY | |
| | WGQGTLVTVSS | |
| 903 | GFSLSTVGMG | Anti-MUC16_HCDR1 |
| 904 | IWWDDEDK | Anti-MUC16_HCDR2 |
| 905 | TRIGTAQATDALDY | Anti-MUC16_HCDR3 |
| 906 | QVTLKESGPGILQPTQTLTLTCTFSGFSLSTVGMGVGWSRQPSGKGLEWLAHIWWDD | Anti-MUC16_V_{H} |
| | EDKYYNPALKSRLTITKDTSKNQVFLKITNVDTADTATYYCTRIGTAQATDALDYWG | |
| | QGTLVTVSS | |
| 907 | QVTLKESGPTLVKPTQTLTLTCTFSGFSLSTVGMGVGWSRQPSGKGLEWLAHIWWDD | Anti-MUC16_V_{H} |
| | EDKYYNPALKSRLTITKDTSKNQVVLTITNVDPVDTATYYCTRIGTAQATDALDYWG | |
| | QGTLVTVSS | |
| 908 | QSLLNSRTRKNQ | Anti-MUC16_LCDR1 |
| 909 | WAS | Anti-MUC16_LCDR2 |
| 910 | QQSYNLLT | Anti-MUC16_LCDR3 |
| 911 | DIELTQSPSSLAVSAGERVTMNCKSSQSLLNSRTRKNQLAWYQQKPGQSPELLIYWAS | Anti-MUC16_VL |
| | TRQSGVPDRFSGSGSGTDFTLTISSVQAEDVAVYYCQQSYNLLTFGPGTKLEIKR | |
| 912 | DIVLTQSPDSLAVSLGERVTMNCKSSQSLLNSRTRKNQLAWYQQKPGQSPELLIYWA | Anti-MUC16VL |
| | STRQSGVPDRFSGSGSGTDFTLTISSVQAEDVAVYYCQQSYNLLTFGQGTKLEIKR | |
| 913 | KSLLHSNGNTY | Anti-MUC16_LCDR1 |
| 914 | YMS | Anti-MUC16_LCDR2 |
| 915 | MQSLEYPLT | Anti-MUC16_LCDR3 |
| 916 | DIVMTQSAPSVPVTPGESVSISCRSSKSLLHSNGNTYLYWFLQKPGQSPQRLIYYMSNL | Anti-MUC16_VL |
| | ASGVPDRFSGRGSGTDFTLKISRVEAEDVGVYYCMQSLEYPLTFGGGTKLEIKR | |
| 917 | DIVMTQSALSLPVTPGEPVSISCRSSKSLLHSNGNTYLYWFLQKPGQSPQRLIYYMSNL | Anti-MUC16_VL |
| | ASGVPDRFSGSGSGTDFTLKISRVEAEDVGVYYCMQSLEYPLTFGGGTKLEIKR | |
| 918 | GYGLS | Anti-FRα_HCDR1 |
| 919 | MISSGGSYTYYAD | Anti-FRα_HCDR2 |
| 920 | HGDDPAWFAY | Anti-FRα_HCDR3 |
| 921 | EVQLVESGGGVVQPGRSLRLSCSASGFTFSGYGLSWVRQAPGKGLEWVAMISSGGSY | Anti-FRα_V_{H} |
| | TYYADSVKGRFAISRDNAKNTLFLQMDSLRPEDTGVYFCARHGDDPAWFAYWGQGT | |
| | PVTVSS | |
| 922 | EVQLVESGGGVVQPGRSLRLSCSASGFTFSGYGLSWVRQAPGKGLEWVAMISSGGSY | Anti-FRα_heavy chain |
| | TYYADSVKGRFAISRDNAKNTLFLQMDSLRPEDTGVYFCARHGDDPAWFAYWGQGT | |
| | PVTVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHTCPP | |
| | CPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQ | |
| | PREPQVYTLPPSRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| 923 | SVSSSISSNNLH | Anti-FRα_LCDR1 |
| 924 | GTSNLAS | Anti-FRα_LCDR2 |
| 925 | QQWSSYPYMYT | Anti-FRα_LCDR3 |
| 926 | DIQLTQSPSSLSASVGDRVTITCSVSSSISSNNLHWYQQKPGKAPKPWIYGTSNLASGV | Anti-FRα_VL |
| | PSRFSGSGSGTDYTFTISSLQPEDIATYYCQQWSSYPYMYTFGQGTKVEIK | |
| 927 | DIQLTQSPSSLSASVGDRVTITCSVSSSISSNNLHWYQQKPGKAPKPWIYGTSNLASGV | Anti-FRα_light chain |
| | PSRFSGSGSGTDYTFTISSLQPEDIATYYCQQWSSYPYMYTFGQGTKVEIKRTVAAPSV | |
| | FIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSENRGEC | |
| 928 | NYGVH | Anti-EGFR_HCDR1 |
| 929 | GVIWSGGNTDYNTPFT | Anti-EGFR_HCDR2 |
| 930 | RALTYYDYEFAYW | Anti-EGFR_HCDR3 |
| 931 | EVQLVESGGGLVQPGGSLRLSCAVSGFSLTNYGVHWVRQATGKGLEWLGVIWSGGN | Anti-EGFR_V_{H} |
| | TDYNTPFTSRLTISKENAKNSVYLQMNSLRAGDTAVYYCARALTYYDYEFAYWGQG | |
| | TMVTVSS | |
| 932 | RASQSIGTNIH | Anti-EGFR_LCDR1 |
| 933 | YASESIS | Anti-EGFR_LCDR2 |
| 934 | QQNNNWP | Anti-EGFR_LCDR3 |
| 935 | EIVLTQSPATLSLSPGERATLSCRASQSIGTNIHWYQQRPGQAPRLLIYYASESISGIPAR | Anti-EGFR_VL |
| | FSGSGSGTDFTLTISSLEPEDFAVYYCQQNNNWPTTFGGGTKVEIK | |
| 936 | DILLTQSPVILSVSPGERVSFSCRASQSIGTNIHWYQQRTNGSPRLLIKYASESISGIPSRF | Anti-EGFR scFv |
| | SGSGSGTDFTLSINSVESEDIADYYCQQNNNWPTTFGAGTKLELKRSRGGGGSGGGGS | |
| | GGGGSLEMAQVQLKQSGPGLVQPSQSLSITCTVSGFSLTNYGVHWVRQSPGKGLEWL | |
| | GVIWSGGNTDYNTPFTSRLSINKDNSKSQVFFKMNSLQSNDTAIYYCARALTYYDYEF | |
| | AYWGQGTLVTVSS | |
| 937 | GFTFRKFG | Anti- |
| | | EGFRVIII_HCDR1 |
| 938 | ISTGGYNT | Anti- |
| | | EGFRVIII_HCDR2 |
| 939 | TRGYSSTSYAMDY | Anti- |
| | | EGFRVIII_HCDR3 |
| 940 | QVKLQQSGGGLVKPGASLKLSCVTSGFTFRKFGMSWVRQTSDKRLEWVASISTGGY | Anti-EGFRVIII_V_{H} |
| | NTYYSDNVKGRFTISRENAKNTLYLQMSSLKSEDTALYYCTRGYSSTSYAMDYWGQ | |
| | GTTVTV | |
| 941 | TDIDDD | Anti- |
| | | EGFRVIII_LCDR1 |
| 942 | EGN | Anti- |
| | | EGFRVIII_LCDR2 |
| 943 | LQSFNVPLT | Anti- |
| | | EGFRVIII_LCDR3 |
| 944 | DIELTQSPASLSVATGEKVTIRCMTSTDIDDDMNWYQQKPGEPPKFLISEGNTLRPGVP | Anti-EGFRVIII_V_{L} |
| | SRFSSSGTGTDFVFTIENTLSEDVGDYYCLQSFNVPLTFGDGTKLEIK | |
| 945 | GFTFDDYA | Ant-HER3_HCDR1 |
| 946 | ISWNSGSI | Ant-HER3_HCDR2 |
| 947 | ARDLGAKQWLEGFDY | Ant-HER3_HCDR3 |
| 948 | MKYLLPTAAAGLLLLAAQPAMAQVQLVQSGGGLVQPGRSLRLSCAASGFTFDDYAM | Ant-HER3_V_{H} |
| | HWVRQAPGKGLEWVSGISWNSGSIGYADSVKGRFTISRDNAKNSLYLQMNSLRPEDT | |
| | AVYYCARDLGAKQWLEGFDYWGQGTLVTV | |
| 949 | SLRSYY | Ant-HER3_LCDR1 |
| 950 | GKN | Ant-HER3_LCDR2 |
| 951 | NSRDSSGNHWV | Ant-HER3_LCDR3 |
| 952 | NFMLTQDPAVSVALGQTVRITCQGDSLRSYYASWYQQKPGQAPVLVIYGKNNRPSGI | Ant-HER3_VL |
| | PDRFSGSTSGNSASLTITGAQAEDEADYYCNSRDSSGNHWVFGGGTKVTVLGAAAEQ | |
| | KLISEEDLNGAA | |
| 953 | MKYLLPTAAAGLLLLAAQPAMAQVQLVQSGGGLVQPGRSLRLSCAASGFTFDDYAM | Anti-HER3 scFv |
| | HWVRQAPGKGLEWVSGISWNSGSIGYADSVKGRFTISRDNAKNSLYLQMNSLRPEDT | |
| | AVYYCARDLGAKQWLEGFDYWGQGTLVTVSSGGGGSGGGGSGGGGSNFMLTQDPA | |
| | VSVALGQTVRITCQGDSLRSYYASWYQQKPGQAPVLVIYGKNNRPSGIPDRFSGSTSG | |
| | NSASLTITGAQAEDEADYYCNSRDSSGNHWVFGGGTKVTVLGAAAEQKLISEEDLNG | |
| | AAHHHHHH | |
| 954 | DYYMN | Anti-DLL3_HCDR1 |
| 955 | LIRNKANGYTTEYNASVKG | Anti-DLL3_HCDR2 |
| 956 | DSDGYYEYYFDY | Anti-DLL3_HCDR3 |
| 957 | MKLWLNWIFLVTLLNGIQCEVKLVESGGGLVQPGGSLSLSCAASGFTFTDYYMNWV | Anti-DLL3_Heavy |
| | RQPPGKALEWLALIRNKANGYTTEYNASVKGRFTISRDNSQNILYLQMNALRAEDSA | Chain |
| | TYYCARDSDGYYEYYFDYWGQGTTLTVSSASTKGPSVFPLAPSSKSTSGGTAALGCL | |
| | VKDYFPEPVTVSWNSGALTSGVHTFPAVLQSSGLYSLSSVVTVPSSSLGTQTYICNVN | |
| | HKPSNTKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSCSVMHEALH | |
| | NHYTQKSLSLSPGK | |
| 958 | RASQEISDYLS | Anti-DLL3_LCDR1 |
| 959 | AASTLDS | Anti-DLL3_LCDR2 |
| 960 | LQYASYPYT | Anti-DLL3_LCDR3 |
| 961 | MDMRVPAHVFGFLLLWFPGTRCDIQMTQSPSSLSASLGERVSLTCRASQEISDYLSWL | Anti-DLL3_Light |
| | QQKPDGTIKRLIFAASTLDSGVPKRFSGSRSGSDFSLSISSLESEDFADYYCLQYASYPY | Chain |
| | TFGSGTKLEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPVTKSFNRG | |
| | EC | |
| 962 | SGGYYWS | Anti-C-MET_HCDR1 |
| 963 | YIYYSGSTYYNPSLKS | Anti-C-MET_HCDR2 |
| 964 | LGPLGYCSSTSCPVTGEYYYYGMDV | Anti-C-MET_HCDR3 |
| 965 | MKHLWFFLLLVAAPRWVLSQVQLQESGPGLVKPSQTLSLTCTVSGGSISSGGYYWSW | Anti-C-MET_Heavy |
| | IRQHPGKGLEWIGYIYYSGSTYYNPSLKSRVTISVDTSKNQFSLKLSSVTAADTAVYYC | Chain |
| | ARDGPLGYCSSTSCPVTGEYYYYGMDVWGQGTTVTVSSASTKGPSVFPLAPCSRSTS | |
| | ESTAALGCLVKDYFPEPVTVSWNSGALTSGVHTFPAVLQSSGLYSLSSVVTVPSSNFG | |
| | TQTYTCNVDHKPSNTKVDKTVERKCCVECPPCPAPPVAGPSVFLFPPKPKDTLMISRT | |
| | PEVTCVVVDVSHEDPEVQFNWYVDGVEVHNAKTKPREEQFNSTFRVVSVLTVVHQD | |
| | WLNGKEYKCKVSNKGLPAPIEKTISKTKGQPREPQVYTLPPSREEMTKNQVSLTCLVK | |
| | GFYPSDIAVEWESNGQPENNYKTTPPMLDSDGSFFLYSKLTVDKSRWQQGNVFSCSV | |
| | MHEALHNHYTQKSLSLSPGK | |
| 966 | RASQSVSNNYLA | Anti-C-MET_LCDR1 |
| 967 | GASSRAT | Anti-C-MET_LCDR2 |
| 968 | QQYDISPMYS | Anti-C-MET_LCDR3 |
| 969 | METPAQLLFLLLLWLPDTTGEIVLTQSPGTLSLSPGERATLSCRASQSVSNNYLAWYQ | Anti-C-MET_Light |
| | QKPGQAPRLLIFGASSRATGIPDRFSGSGSGTDFTLTISRLEPEDFAVYYCQQYDISPMY | Chain |
| | SFGQGTKLEMKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPVTKSFNR | |
| | GEC | |
| 970 | GYTFTNYGMN | Anti-CD70_HCDR1 |
| 971 | WINTYTGEPTYADAFK | Anti-CD70_HCDR2 |
| 972 | DYGDYGMDY | Anti-CD70_HCDR3 |
| 973 | MAWVWTLLFLMAAAQSAGAQIQLVQSGPEVKKPGETVKISCKASGYTFTNYGMNW | Anti-CD70_V_{H} |
| | VKQAPGKGLKWMGWINTYTGEPTYADAFKGRFAFSLETSASTAYLQINNLKNEDTA | |
| | TYFCARDYGDYGMDYWGQGTSVTVSS | |
| 974 | RASKSVSTSGYSFMH | Anti-CD70_LCDR1 |
| 975 | LASNLES | Anti-CD70_LCDR2 |
| 976 | QHSREVPWT | Anti-CD70_LCDR3 |
| 977 | METDTLLLWVLLLWVPGSTGDIVLTQSPASLAVSLGQRATISCRASKSVSTSGYSFMH | Anti-CD70_V_{L} |
| | WYQQKPGQPPKLLIYLASNLESGVPARFSGSGSGTDFTLNIHPVEEEDAATYYCQHSR | |
| | EVPWTFGGGTKLEIKR | |

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. An immune cell comprising:
(a) a chimeric antigen receptor (CAR) comprising:
(i) an extracellular target-binding domain comprising an antibody moiety (a CAR antibody moiety);
(ii) a transmembrane domain (a CAR transmembrane domain); and
(iii) a primary signaling domain, and
(b) a chimeric stimulating receptor (CSR) comprising:
(i) a ligand-binding module that is capable of binding or interacting with a target ligand;
(ii) a transmembrane domain (a CSR transmembrane domain); and
(iii) a CD30 costimulatory domain,
wherein the CSR lacks a functional primary signaling domain.

2. The immune cell of claim 1, wherein the CD30 costimulatory domain comprises a sequence that can bind to an intracellular TRAF signaling protein, optionally wherein the sequence that can bind to an intracellular TRAF signaling protein corresponds to residues 561-573 or 578-586 of a full-length CD30 having the sequence of SEQ ID NO:65.

3. The immune cell of claim 1 or 2, wherein the CD30 costimulatory domain comprises a sequence that is at least 80%, 85%, 90%, 95%, or 100% identical to residues 561-573 or 578-586 of SEQ ID NO:65; or wherein the CD30 costimulatory domain comprises a sequence that is at least 70%, 75%, 80%, 85%, 90%, 95%, or 100% identical to the sequence of SEQ ID NO:75.

4. The immune cell of any one of claims 1 to 3, wherein the CSR comprises more than one CD30 costimulatory domain.

5. The immune cell of any one of claims 1 to 4, wherein the CSR further comprises at least one costimulatory domain which comprises the intracellular sequence of a costimulatory molecule that is different from CD30, optionally wherein the costimulatory molecule that is different from CD30 is selected from the group consisting of CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

6. The immune cell of any one of claims 1 to 5, wherein the CAR further comprises a costimulatory domain (a CAR costimulatory domain), optionally wherein the CAR costimulatory domain is derived from the intracellular domain of a costimulatory receptor, and further optionally wherein the costimulatory receptor is selected from the group consisting of CD30, CD27, CD28, 4-1BB (CD137), OX40, CD40, PD-1, ICOS, lymphocyte function-associated antigen-1 (LFA-1), CD2, CD7, LIGHT, NKG2C, B7-H3, and a ligand that specifically binds with CD83.

7. The immune cell of any one of claims 1 to 6, wherein (a) the ligand-binding module of the CSR is derived from the extracellular domain of a receptor; or (b) the ligand-binding module of the CSR comprises an antibody moiety (a CSR antibody moiety), optionally wherein the CSR antibody moiety is a single chain antibody fragment.

8. The immune cell of any one of claims 1 to 7, wherein the CAR antibody moiety is a single chain antibody fragment; and/or wherein the CAR antibody moiety and/or the CSR antibody moiety is a single chain Fv (scFv), a single chain Fab, a single chain Fab′, a single domain antibody fragment, a single domain multispecific antibody, an intrabody, a nanobody, or a single chain immunokine.

9. The immune cell of any one of claims 1 to 8, wherein the CAR antibody moiety and/or the CSR antibody moiety specifically binds to a disease-related antigen, optionally wherein the disease-related antigen is a cancer-related antigen or a virus-related antigen.

10. The immune cell of any one of claims 1 to 9, wherein the CAR antibody moiety and/or the CSR antibody moiety specifically binds to a cell surface antigen, optionally wherein the cell surface antigen is selected from the group consisting of protein, carbohydrate, and lipid; and/or optionally wherein the cell surface antigen is CD19, CD20, CD22, CD47, CD158e, GPC3, ROR1, ROR2, BCMA, GPRC5D, FcRL5, MUC16, MCT4, PSMA, or a variant or mutant thereof.

11. The immune cell of any one of claims 1 to 10, (a) wherein the CAR antibody moiety and the CSR antibody moiety specifically bind to the same antigen; or (b) wherein the CAR antibody moiety and/or the CSR antibody moiety specifically binds to a MHC-restricted antigen, optionally wherein the MHC-restricted antigen is a complex comprising a peptide and an MHC protein, and wherein the peptide is derived from a protein selected from the group consisting of WT-1, AFP, GPC3, HPV16-E7, NY-ESO-1, PRAME, EBV-LMP2A, HIV-1, KRAS, FoxP3, Histone H3.3, PSA, ROR1, and a variant or mutant thereof.

12. The immune cell of any one of claims 1 to 11, wherein:
(a) the CAR antibody moiety binds to CD19, and wherein the ligand-binding module of the CSR binds to CD19; or
(b) the CAR antibody moiety binds to CD22, and wherein the ligand-binding module of the CSR binds to CD22; or
(c) the CAR antibody moiety binds to CD20, and wherein the ligand-binding module of the CSR binds to CD20; or
(d) the CAR antibody moiety binds to CD19, and wherein the ligand-binding module of the CSR binds to CD22; or
(e) the CAR antibody moiety binds to CD19, and wherein the ligand-binding module of the CSR binds to CD20; or
(f) the CAR antibody moiety binds to CD22, and wherein the ligand-binding module of the CSR binds to CD20;
(g) the CAR antibody moiety binds to CD22, and wherein the ligand-binding module of the CSR binds to CD19; or
(h) the CAR antibody moiety binds to CD20, and wherein the ligand-binding module of the CSR binds to CD19; or
(i) the CAR antibody moiety binds to CD20, and wherein the ligand-binding module of the CSR binds to CD22; or
(j) the CAR antibody moiety and/or the ligand-binding module of the CSR binds to both CD19 and CD22; or
(k) the CAR antibody moiety and/or the ligand-binding module of the CSR binds to both CD19 and CD20; or
(l) the CAR antibody moiety and/or the ligand-binding module of the CSR binds to both CD20 and CD22; or
(m) the CAR antibody moiety and/or the ligand-binding module of the CSR binds to CD19, CD20, and CD22.

13. The immune cell of any one of claims 1 to 12, wherein:
(a) the CAR antibody moiety specifically binds to a complex comprising an alpha-fetoprotein (AFP) peptide and an MHC class I protein; or
(b) the CAR antibody moiety specifically binds to glypican 3 (GPC3); or
(c) the ligand-binding module of the CSR specifically binds to GPC3; or
(d) the CAR antibody moiety binds to a complex comprising an AFP peptide and an MHC class I protein, and wherein the ligand-binding module of the CSR binds to GPC3; or
(e) both the CAR antibody moiety and the ligand-binding module of the CSR bind to GPC3; or
(f) the CAR antibody moiety and the ligand-binding module of the CSR specifically bind to different epitopes on GPC3; or
(g) the CAR antibody moiety specifically binds to a complex comprising a KRAS peptide and an MHC class I protein; or
(h) the CAR antibody moiety specifically binds to a complex comprising a PSA peptide and an MHC class I protein; or
(i) the CAR antibody moiety specifically binds to a complex comprising a PSMA peptide and an MHC class I protein; or
(j) the CAR antibody moiety and/or the ligand-binding module of the CSR bind to ROR1; or
(k) the CAR antibody moiety specifically binds to a complex comprising a NY-ESO-1 peptide and an MHC class I protein; or
(l) the CAR antibody moiety specifically binds to a complex comprising a PRAME peptide and an MHC class I protein; or
(m) the CAR antibody moiety specifically binds to a complex comprising a WT1 peptide and an MHC class I protein; or
(n) the CAR antibody moiety specifically binds to a complex comprising a histone H3.3 peptide and an MHC class I protein; or
(o) the ligand-binding module of the CSR binds to an MSLN peptide; or
(p) the ligand-binding module of the CSR binds to a ROR2 peptide; or
(q) the ligand-binding module of the CSR binds to a HER2 peptide; or
(r) the ligand-binding module of the CSR binds to an EpCAM peptide; or
(s) the ligand-binding module of the CSR binds to a MUC1 peptide; or
(t) the ligand-binding module of the CSR binds to a MUC16 peptide; or
(u) the ligand-binding module of the CSR binds to an FRα peptide; or
(v) the ligand-binding module of the CSR binds to an EGFRVIII peptide; or
(w) the ligand-binding module of the CSR binds to an HER3 peptide; or
(x) the ligand-binding module of the CSR binds to a DLL3 peptide; or
(y) the ligand-binding module of the CSR binds to a c-Met peptide; or
(z) the ligand-binding module of the CSR binds to a CD70 peptide.

14. The immune cell of any one of claims 1 to 13, wherein:
(a) the CAR transmembrane domain is the transmembrane domain of CD30; or
(b) the CAR transmembrane domain is the transmembrane domain of CD8; and/or
(c) the CSR transmembrane domain is derived from the transmembrane domain of a TCR co-receptor or a T cell co-stimulatory molecule, optionally wherein the TCR co-receptor or T cell co-stimulatory molecule is selected from the group consisting of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, and CD154; or
(d) the CAR transmembrane domain and/or the CSR transmembrane domain is the transmembrane domain of CD8, 4-1BB, CD27, CD28, CD30, OX40, CD3ε, CD3ζ, CD45, CD4, CD5, CD9, CD16, CD22, CD33, CD37, CD64, CD80, CD86, CD134, CD137, or CD154; and/or
(e) the CAR transmembrane domain and/or the CSR transmembrane domain comprises an amino acid sequence selected from the group consisting of SEQ ID NOS:66-71.

15. The immune cell of any one of claims 1 to 14, wherein:
(a) the primary signaling domain comprises a sequence derived from the intracellular signaling sequence of a molecule selected from the group consisting of CD3ζ, TCRζ, FcRγ, FcRβ, CD3γ, CD3δ, CD3ε, CD5, CD22, CD79a, CD79b, and CD66d; and/or
(b) the primary signaling domain comprises a sequence that is at least 80%, 85%, 90%, 95%, or 100% identical to the sequence of SEQ ID NO:77.

16. The immune cell of any one of claims 1 to 15, further comprises a peptide linker between the extracellular target-binding domain and the transmembrane domain of the CAR; and/or further comprises a peptide linker between the transmembrane domain and the costimulatory domain of the CAR; and/or further comprises a peptide linker between the costimulatory domain and the primary signaling domain of the CAR; and/or further comprises a peptide linker between the ligand-binding module and the transmembrane domain of the CSR; and/or further comprises a peptide linker between the transmembrane domain and the CD30 costimulatory domain of the CSR.

17. The immune cell of any one of claims 1 to 16, wherein the expression of the CSR is inducible, optionally wherein the expression of the CSR is inducible upon activation of the immune cell.

18. The immune cell of any one of claims 1 to 17, wherein the immune cell is selected from the group consisting of a cytotoxic T cell, a helper T cell, a natural killer T cell, and a suppressor T cell.

19. One or more nucleic acids encoding the CAR and CSR comprised by the immune cell of any one of claims 1 to 18, wherein the CAR and CSR each consist of one or more polypeptide chains encoded by the one or more nucleic acids.

20. One or more vectors comprising the one or more nucleic acids of claim 19.

21. A pharmaceutical composition comprising: (a) the immune cell of any one of claims 1 to 18, the nucleic acid(s) of claim 19, or the vector(s) of claim 20, and (b) a pharmaceutically acceptable carrier or diluent.

22. A method of killing target cells, comprising:
contacting one or more target cells with the immune cell of any one of claims 1 to 18 under conditions and for a time sufficient so that the immune cells mediate killing of the target cells,
wherein the target cells express an antigen specific to the immune cell, and
wherein the immune cell expresses a low cell exhaustion level upon contacting the target cells,
optionally wherein the immune cell is a T cell; and/or
optionally wherein the target cells are cancer cells.

23. The method of claim 22, wherein:
(a) the immune cell expresses a low cell exhaustion level of an exhaustion marker selected from the group consisting of PD-1, TIM-3, TIGIT, and LAG-3; and/or
(b) the immune cell expresses a lower level of PD-1, TIM-3, TIGIT, or LAG-3 than corresponding immune cell expressing a CSR comprising a CD28 costimulatory domain; and/or
(c) the immune cell expresses a lower level of PD-1, TIM-3, TIGIT, or LAG-3 than corresponding immune cell expressing a CSR comprising a 4-1BB costimulatory domain.

24. The method of claim 22 or 23, wherein:
(a) the cancer cells are from a cancer selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer; and/or
(b) the cancer cells are hematological cancer cells; or
(c) the cancer cells are solid tumor cells.

25. The method of any one of claims 22 to 24, wherein the target cells are virus-infected cells, optionally wherein the virus-infected cells are from a viral infection caused by a virus selected from the group consisting of Cytomegalovirus (CMV), Epstein-Barr Virus (EBV), Hepatitis B Virus (HBV), Kaposi's Sarcoma associated herpesvirus (KSHV), Human papillomavirus (HPV), Molluscum contagiosum virus (MCV), Human T cell leukemia virus 1 (HTLV-1), HIV (Human immunodeficiency virus), and Hepatitis C Virus (HCV).

26. A method of treating a disease, the method comprising a step of administering to a subject the immune cell of any one of claims 1 to 18, the nucleic acid(s) of claim 19, or the vector(s) of claim 20, or the pharmaceutical composition of claim 21 to the subject.

27. The method of claim 26, wherein the disease is a viral infection or cancer, optionally wherein the cancer is a hematological cancer or a solid tumor cancer; and/or optionally wherein the cancer is selected from the group consisting of adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, ovarian cancer, prostate cancer, sarcoma, stomach cancer, uterine cancer, and thyroid cancer.

28. The method of claim 26 or 27, wherein:
(a) the subject has a higher density of the immune cell of any one of claims 1 to 18 in the solid tumor cancer than in the rest of the subject's body; and/or
(b) the subject has a higher density of the immune cell of any one of claims 1 to 18 in the peripheral blood of the subject as compared to treating the same type of disease with same number of immune cells expressing the same CAR and a corresponding CSR comprising a CD28 or 4-1BB costimulatory domain.

29. A method for preventing and/or reversing T cell exhaustion in a subject, comprising administering to the subject the nucleic acid(s) of claim 19, or the vector(s) of claim 20, or the pharmaceutical composition of claim 21 comprising the nucleic acid(s) or the vector(s) to the subject, optionally wherein the method decreases the expression of an exhaustion marker in a T cell, further optionally the exhaustion marker is selected from the group consisting of PD-1, TIM-3, TIGIT, and LAG-3.

30. A method of treating a solid tumor cancer in a subject with increased tumor infiltration as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell of any one of claims 1 to 18.

31. A method of treating a solid tumor cancer in a subject with increased tumor regression as compared to treating the same type of solid tumor cancer with immune cells expressing a CAR and a CSR comprising a CD28 or 4-1BB costimulatory domain, wherein the method comprises administering to the subject corresponding immune cells expressing the same CAR and a corresponding CSR comprising a CD30 costimulatory domain, and wherein the corresponding immune cells comprise the immune cell of any one of claims 1 to 18.

32. A method for generating central memory T cells in a subject, comprising administering to the subject the nucleic acid(s) of claim 19, or the vector(s) of claim 20, or the pharmaceutical composition of claim 21 comprising the nucleic acid(s) or the vector(s) to the subject, optionally wherein the method increases the number of central memory T cells and/or the percentage of central memory T cells among all T cells in the subject.

33. A method for generating central memory T cells in vitro comprising: contacting one or more target cells with the immune cell of any one of claims 1 to 18 under conditions and for a time sufficient so that the immune cell develops into central memory T cells, wherein the target cells express an antigen specific to the immune cell.

34. The method of claim 33, wherein:
(a) the method increases the number of central memory T cells and/or the percentage of central memory T cells among all T cells descended from the immune cell; and/or
(b) the method generates higher number of central memory T cells and/or higher percentage of central memory T cells than corresponding immune cell expressing a CSR comprising a CD28 costimulatory domain, optionally wherein the method generates at least 10%, 20%, 30%, 40%, 50%, 60%, 70%, 80%, 90%, 100%, 200%, 300%, 400%, or 500% higher number of central memory T cells and/or percentage of central memory T cells than corresponding immune cell expressing a CSR comprising a CD28 costimulatory domain.

35. The method of any one of claim 33 or 34, wherein the central memory T cells express high levels of CCR7 and low levels of CD45RA; and/or wherein the central memory T cells are CD8⁺ T cells.
